SEQUENCE LISTING (57 sequences)

SEQ ID NO: 1 - DNA, 21 bp, Artificial Sequence
  Synthetic siRNA sequence targeting the gene, CENPA
caccgttccaaaggcctgaaa

SEQ ID NO: 2 - DNA, 21 bp, Artificial Sequence
  Synthetic siRNA sequence targeting the gene, CENPA
cagagccatgactagatccaa

SEQ ID NO: 3 - DNA, 2554 bp, Homo sapiens
acaaggcagcctcgctcgagcgcaggccaatcggctttctagctagagggtttaactcctatttaaaaagaagaacctttgaattctaacggctgagctcttggaagacttgggtccttgggtcgcaggtgggagccgacgggtgggtagaccgtgggggatatctcagtggcggacgaggacggcggggacaaggggcggctggtcggagtggcggagcgtcaagtcccctgtcggttcctccgtccctgagtgtccttggcgctgccttgtgcccgcccagcgcctttgcatccgctcctgggcaccgaggcgccctgtaggatactgcttgttacttattacagctagagggtctcactccattgcccaggccagagtgcggggatatttgataagaaacttcagtgaaggccgggcgcggtggctcatgcccgtaatcccagcattttcggaggccgaggctggagtgcaatggtgtgatctcagctcactgcaacctctgcttcctgggtttaagtgattctcctgcctcagcctcccgagtagctgggattacaggcatcatggaccgatctaaagaaaactgcatttcaggacctgttaaggctacagctccagttggaggtccaaaacgtgttctcgtgactcagcaatttccttgtcagaatccattacctgtaaatagtggccaggctcagcgggtcttgtgtccttcaaattcttcccagcgcattcctttgcaagcacaaaagcttgtctccagtcacaagccggttcagaatcagaagcagaagcaattgcaggcaaccagtgtacctcatcctgtctccaggccactgaataacacccaaaagagcaagcagcccctgccatcggcacctgaaaataatcctgaggaggaactggcatcaaaacagaaaaatgaagaatcaaaaaagaggcagtgggctttggaagactttgaaattggtcgccctctgggtaaaggaaagtttggtaatgtttatttggcaagagaaaagcaaagcaagtttattctggctcttaaagtgttatttaaagctcagctggagaaagccggagtggagcatcagctcagaagagaagtagaaatacagtcccaccttcggcatcctaatattcttagactgtatggttatttccatgatgctaccagagtctacctaattctggaatatgcaccacttggaacagtttatagagaacttcagaaactttcaaagtttgatgagcagagaactgctacttatataacagaattggcaaatgccctgtcttactgtcattcgaagagagttattcatagagacattaagccagagaacttacttcttggatcagctggagagcttaaaattgcagattttgggtggtcagtacatgctccatcttccaggaggaccactctctgtggcaccctggactacctgccccctgaaatgattgaaggtcggatgcatgatgagaaggtggatctctggagccttggagttctttgctatgaatttttagttgggaagcctccttttgaggcaaacacataccaagagacctacaaaagaatatcacgggttgaattcacattccctgactttgtaacagagggagccagggacctcatttcaagactgttgaagcataatcccagccagaggccaatgctcagagaagtacttgaacacccctggatcacagcaaattcatcaaaaccatcaaattgccaaaacaaagaatcagctagcaaacagtcttaggaatcgtgcagggggagaaatccttgagccagggctgccatataacctgacaggaacatgctactgaagtttattttaccattgactgctgccctcaatctagaacgctacacaagaaatatttgttttactcagcaggtgtgccttaacctccctattcagaaagctccacatcaataaacatgacactctgaagtgaaagtagccacgagaattgtgctacttatactggttcataatctggaggcaaggttcgactgcagccgccccgtcagcctgtgctaggcatggtgtcttcacaggaggcaaatccagagcctggctgtggggaaagtgaccactctgccctgaccccgatcagttaaggagctgtgcaataaccttcctagtacctgagtgagtgtgtaacttattgggttggcgaagcctggtaaagctgttggaatgagtatgtgattctttttaagtatgaaaataaagatatatgtacagacttgtattttttctctggtggcattcctttaggaatgctgtgtgtctgtccggcaccccggtaggcctgattgggtttctagtcctccttaaccacttatctcccatatgagagtgtgaaaaataggaacacgtgctctacctccatttagggatttgcttgggatacagaagaggccatgtgtctcagagctgttaagggcttatttttttaaaacattggagtcatagcatgtgtgtaaactttaaatatgcaaataaataagtatctatgtctaaaaaaa

SEQ ID NO: 4 - DNA, 21 bp, Artificial Sequence
  Synthetic siRNA sequence targeting the gene, CENPE
caggttaatcctaccacacaa

SEQ ID NO: 5 - DNA, 8630 bp, Homo sapiens
taaatttaaaggcggggcggcctgtgagccctgaagtgccggccgcggagggtcctggccattttcctgggaccagttcagcctgataggatggcggaggaaggagccgtggccgtctgcgtgcgagtgcggccgctgaacagcagagaagaatcacttggagaaactgcccaagtttactggaaaactgacaataatgtcatttatcaagttgatggaagtaaatccttcaattttgatcgtgtctttcatggtaatgaaactaccaaaaatgtgtatgaagaaatagcagcaccaatcatcgattctgccatacaaggctacaatggtactatatttgcctatggacagactgcttcaggaaaaacatataccatgatgggttcagaagatcatttgggagttatacccagggcaattcatgacattttccaaaaaattaagaagtttcctgatagggaatttctcttacgtgtatcttacatggaaatatacaatgaaaccattacagatttactctgtggcactcaaaaaatgaaacctttaattattcgagaagatgtcaataggaatgtgtatgttgctgatctcacagaagaagttgtatatacatcagaaatggctttgaaatggattacaaagggagaaaagagcaggcattatggagaaacaaaaatgaatcaaagaagcagtcgttctcataccatctttaggatgattttggaaagcagagagaagggtgaaccttctaattgtgaaggatctgttaaggtatcccatttgaatttggttgatcttgcaggcagtgaaagagctgctcaaacaggcgctgcaggtgtgcggctcaaggaaggctgtaatataaatcgaagcttatttattttgggacaagtgatcaagaaacttagtgatggacaagttggtggtttcataaattatcgagatagcaagttaacacgaattctccagaattccttgggaggaaatgcaaagacacgtattatctgcacaattactccagtatcttttgatgaaacacttactgctctccagtttgccagtactgctaaatatatgaagaatactccttatgttaatgaggtatcaactgatgaagctctcctgaaaaggtatagaaaagaaataatggatcttaaaaaacaattagaggaggtttctttagagacgcgggctcaggcaatggaaaaagaccaattggcccaacttttggaagaaaaagatttgcttcagaaagtacagaatgagaaaattgaaaacttaacacggatgctggtgacctcttcttccctcacgttgcaacaggaattaaaggctaaaagaaaacgaagagttacttggtgccttggcaaaattaacaaaatgaagaactcaaactatgcagatcaatttaatataccaacaaatataacaacaaaaacacataagctttctataaatttattacgagaaattgatgaatctgtctgttcagagtctgatgttttcagtaacactcttgatacattaagtgagatagaatggaatccagcaacaaagctactaaatcaggagaatatagaaagtgagttgaactcacttcgtgctgactatgataatctggtattagactatgaacaactacgaacagaaaaagaagaaatggaattgaaattaaaagaaaagaatgatttggatgaatttgaggctctagaaagaaaaactaaaaaagatcaagagatgcaactaattcatgaaatttcgaacttaaagaatttagttaagcatgcagaagtatataatcaagatcttgagaatgaactcagttcaaaagtagagctgcttagagaaaaggaagaccagattaagaagctacaggaatacatagactctcaaaagctagaaaatataaaaatggacttgtcatactcattggaaagcattgaagacccaaaacaaatgaagcagactctgtttgatgctgaaactgtagcccttgatgccaagagagaatcagcctttcttagaagtgaaaatctggagctgaaggagaaaatgaaagaacttgcaactacatacaagcaaatggaaaatgatattcagttatatcaaagccagttggaggcaaaaaagaaaatgcaagttgatctggagaaagaattacaatctgcttttaatgagataacaaaactcacctcccttatagatggcaaagttccaaaagatttgctctgtaatttggaattggaaggaaagattactgatcttcagaaagaactaaataaagaagttgaagaaaatgaagctttgcgggaagaagtcattttgctttcagaattgaaatctttaccttctgaagtagaaaggctgaggaaagagatacaagacaaatctgaagagctccatataataacatcagaaaaagataaattgttttctgaagtagttcataaggagagtagagttcaaggtttacttgaagaaattgggaaaacaaaagatgacctagcaactacacagtcgaattataaaagcactgatcaagaattccaaaatttcaaaacccttcatatggactttgagcaaaagtataagatggtccttgaggagaatgagagaatgaatcaggaaatagttaatctctctaaagaagcccaaaaatttgattcgagtttgggtgctttgaagaccgagctttcttacaagacccaagaacttcaggagaaaacacgtgaggttcaagaaagactaaatgagatggaacagctgaaggaacaattagaaaatagagattctacgctgcaaactgtagaaagggagaaaacactgattactgagaaactgcagcaaactttagaagaagtaaaaactttaactcaagaaaaagatgatctaaaacaactccaagaaagcttgcaaattgagagggaccaactcaaaagtgatattcacgatactgttaacatgaatatagatactcaagaacaattacgaaatgctcttgagtctctgaaacaacatcaagaaacaattaatacactaaaatcgaaaatttctgaggaagtttccaggaatttgcatatggaggaaaatacaggagaaactaaagatgaatttcagcaaaagatggttggcatagataaaaaacaggatttggaagctaaaaatacccaaacactaactgcagatgttaaggataatgagataattgagcaacaaaggaagatattttctttaatacaggagaaaaatgaactccaacaaatgttagagagtgttatagcagaaaaggaacaattgaagactgacctaaaggaaaatattgaaatgaccattgaaaaccaggaagaattaagacttcttggggatgaacttaaaaagcaacaagagatagttgcacaagaaaagaaccatgccataaagaaagaaggagagctttctaggacctgtgacagactggcagaagttgaagaaaaactaaaggaaaagagccagcaactccaagaaaaacagcaacaacttcttaatgtacaagaagagatgagtgagatgcagaaaaagattaatgaaatagagaatttaaagaatgaattaaagaacaaagaattgacattggaacatatggaaacagagaggcttgagttggctcagaaacttaatgaaaattatgaggaagtgaaatctataaccaaagaaagaaaagttctaaaggaattacagaagtcatttgaaacagagagagaccaccttagaggatatataagagaaattgaagctacaggcctacaaaccaaagaagaactaaaaattgctcatattcacctaaaagaacaccaagaaactattgatgaactaagaagaagcgtatctgagaagacagctcaaataataaatactcaggacttagaaaaatcccataccaaattacaagaagagatcccagtgcttcatgaggaacaagagttactgcctaatgtgaaagaagtcagtgagactcaggaaacaatgaatgaactggagttattaacagaacagtccacaaccaaggactcaacaacactggcaagaatagaaatggaaaggctcaggttgaatgaaaaatttcaagaaagtcaggaagagataaaatctctaaccaaggaaagagacaaccttaaaacgataaaagaagcccttgaagttaaacatgaccagctgaaagaacatattagagaaactttggctaaaatccaggagtctcaaagcaaacaagaacagtccttaaatatgaaagaaaaagacaatgaaactaccaaaatcgtgagtgagatggagcaattcaaacccaaagattcagcactactaaggatagaaatagaaatgctcggattgtccaaaagacttcaagaaagtcatgatgaaatgaaatctgtagctaaggagaaagatgacctacagaggctgcaagaagttcttcaatctgaaagtgaccagctcaaagaaaacataaaagaaattgtagctaaacacctggaaactgaagaggaacttaaagttgctcattgttgcctgaaagaacaagaggaaactattaatgagttaagagtgaatctttcagagaaggaaactgaaatatcaaccattcaaaagcagttagaagcaatcaatgataaattacagaacaagatccaagagatttatgagaaagaggaacaatttaatataaaacaaattagtgaggttcaggaaaaagtgaatgaactgaaacaattcaaggagcatcgcaaagccaaggattcagcactacaaagtatagaaagtaagatgctcgagttgaccaacagacttcaagaaagtcaagaagaaatacaaattatgattaaggaaaaagaggaaatgaaaagagtacaggaggcccttcagatagagagagaccaactgaaagaaaacactaaagaaattgtagctaaaatgaaagaatctcaagaaaaagaatatcagtttcttaagatgacagctgtcaatgagactcaggagaaaatgtgtgaaatagaacacttgaaggagcaatttgagacccagaagttaaacctggaaaacatagaaacggagaatataaggttgactcagatactacatgaaaaccttgaagaaatgagatctgtaacaaaagaaagagatgaccttaggagtgtggaggagactctcaaagtagagagagaccagctcaaggaaaaccttagagaaactataactagagacctagaaaaacaagaggagctaaaaattgttcacatgcatctgaaggagcaccaagaaactattgataaactaagagggattgtttcagagaaaacaaatgaaatatcaaatatgcaaaaggacttagaacactcaaatgatgccttaaaagcacaggatctgaaaatacaagaggaactaagaattgctcacatgcatctgaaagagcagcaggaaactattgacaaactcagaggaattgtttctgagaagacagataaactatcaaatatgcaaaaagatttagaaaattcaaatgctaaattacaagaaaagattcaagaacttaaggcaaatgaacatcaacttattacgttaaaaaaagatgtcaatgagacacagaaaaaagtgtctgaaatggagcaactaaagaaacaaataaaagaccaaagcttaactctgagtaaattagaaatagagaatttaaatttggctcagaaacttcatgaaaaccttgaagaaatgaaatctgtaatgaaagaaagagataatctaagaagagtagaggagacactcaaactggagagagaccaactcaaggaaagcctgcaagaaaccaaagctagagatctggaaatacaacaggaactaaaaactgctcgtatgctatcaaaagaacacaaagaaactgttgataaacttagagaaaaaatttcagaaaagacaattcaaatttcagacattcaaaaggatttagataaatcaaaagatgaattacagaaaaagatccaagaacttcagaaaaaagaacttcaactgcttagagtgaaagaagatgtcaatatgagtcataaaaaaattaatgaaatggaacagttgaagaagcaatttgaggcccaaaacttatctatgcaaagtgtgagaatggataacttccagttgactaagaaacttcatgaaagccttgaagaaataagaattgtagctaaagaaagagatgagctaaggaggataaaagaatctctcaaaatggaaagggaccaattcatagcaaccttaagggaaatgatagctagagaccgacagaaccaccaagtaaaacctgaaaaaaggttactaagtgatggacaacagcaccttacggaaagcctgagagaaaagtgctctagaataaaagagcttttgaagagatactcagagatggatgatcattatgagtgcttgaatagattgtctcttgacttggagaaggaaattgaattccaaaaagagctttcaatgagagttaaagcaaacctctcacttccctatttacaaaccaaacacattgaaaaactttttactgcaaaccagagatgctccatggaattccacagaatcatgaagaaactgaagtatgtgttaagctatgttacaaaaataaaagaagaacaacatgaatccatcaataaatttgaaatggattttattgatgaagtggaaaagcaaaaggaattgctaattaaaatacagcaccttcaacaagattgtgatgtaccatccagagaattaagggatctcaaattgaaccagaatatggatctacatattgaggaaattctcaaagatttctcagaaagtgagttccctagcataaagactgaatttcaacaagtactaagtaataggaaagaaatgacacagtttttggaagagtggttaaatactcgttttgatatagaaaagcttaaaaatggcatccagaaagaaaatgataggatttgtcaagtgaataacttctttaataacagaataattgccataatgaatgaatcaacagagtttgaggaaagaagtgctaccatatccaaagagtgggaacaggacctgaaatcactgaaagagaaaaatgaaaaactatttaaaaactaccaaacattgaagacttccttggcatctggtgcccaggttaatcctaccacacaagacaataagaatcctcatgttacatcaagagctacacagttaaccacagagaaaattcgagagctggaaaattcactgcatgaagctaaagaaagtgctatgcataaggaaagcaagattataaagatgcagaaagaacttgaggtgactaatgacataatagcaaaacttcaagccaaagttcatgaatcaaataaatgccttgaaaaaacaaaagagacaattcaagtacttcaggacaaagttgctttaggagctaagccatataaagaagaaattgaagatctcaaaatgaagcttgtgaaaatagacctagagaaaatgaaaaatgccaaagaatttgaaaaggaaatcagtgctacaaaagccactgtagaatatcaaaaggaagttataaggctattgagagaaaatctcagaagaagtcaacaggcccaagatacctcagtgatatcagaacatactgatcctcagccttcaaataaacccttaacttgtggaggtggcagcggcattgtacaaaacacaaaagctcttattttgaaaagtgaacatataaggctagaaaaagaaatttctaagttaaagcagcaaaatgaacagctaataaaacaaaagaatgaattgttaagcaataatcagcatctttccaatgaggtcaaaacttggaaggaaagaacccttaaaagagaggctcacaaacaagtaacttgtgagaattctccaaagtctcctaaagtgactggaacagcttctaaaaagaaacaaattacaccctctcaatgcaaggaacggaatttacaagatcctgtgccaaaggaatcaccaaaatcttgtttttttgatagccgatcaaagtctttaccatcacctcatccagttcgctattttgataactcaagtttaggcctttgtccagaggtgcaaaatgcaggagcagagagtgtggattctcagccaggtccttggcacgcctcctcaggcaaggatgtgcctgagtgcaaaactcagtagactcctctttgtcacttctctggagatccagcattccttatttggaaatgactttgtttatgtgtctatccctggtaatgatgttgtagtgcagcttaatttcaattcagtctttactttgccactagagttgaaagataagggaacaggaaatgaatgcattgtggtaatttagaatggtgatagcaataccttcttcttgcatatggtaatacttttaaaagttgaattgttttatttatttgtatattttgtaaagaataaagttattgaaagaaatgtaaagttatctacatgacttagcatattccaaagcataatacatacattaatataaaacatcattttattaacaaaattgtaaatgtttttaataccttacacattcaataaatgtttagtagttctgaatcaccaaaaaaaaaaaaaaaaaa

SEQ ID NO: 6 - DNA, 21 bp, Artificial Sequence
  Synthetic siRNA sequence targeting the gene, CENPN
atcagtgatgctgccctgtta

SEQ ID NO: 7 - DNA, 2265 bp, Homo sapiens
gagcgacgcccacggcctgtctcggccaccagcgtgttccagcgagcgcccagccacctcgctcgcagcctccccagcgcagcagcccggctgtgggcctgcggcagccgggtcttcctggtccccacctcctggggccgacgggcggcaggaaggggctcggcgggacgcgccatcagggacctgaggaggaacaacggaacgcgttcggaacggcctggactcccgagactcacccgactcgtggccacaccgggagaactgaagcggcagtagccggcggagacgcccgacccgaaggccggctgctagggagcagacagctgaaccgcttgccagacgccgaaacccagtgacgccctccaccgctccaccgtgctcccggctcctcgcccccgccgcccgcgggccccaaggcgcatgcgccgcctgtcctggaggggcccatttccgtccgtcgtggggggaggcacagtgagtccactggggcacggcagcgtctaagccacaagccgagcacataagccaggtcctaacggagcctatgtgtaagtccactactggtgcaaggttgcacacttctaagaagagcggcgtggggggctcggcgaccttcgcttcagtcgctcccccgtgcagtcccctgtgcccaagacacagcctgatgcttgtgctccggtgggcggagcttggaggcggcgggaactgcaattggtggctttgaaggcgcggcgagcgggaacagctcttgaggagtgagactgcaggagatgtgggccgtgccaaagagatggatgagactgttgctgagttcatcaagaggaccatcttgaaaatccccatgaatgaactgacaacaatcctgaaggcctgggattttttgtctgaaaatcaactgcagactgtaaatttccgacagagaaaggaatctgtagttcagcacttgatccatctgtgtgaggaaaagcgtgcaagtatcagtgatgctgccctgttagacatcatttatatgcaatttcatcagcaccagaaagtttgggatgtttttcagatgagtaaaggaccaggtgaagatgttgacctttttgatatgaaacaatttaaaaattcgttcaagaaaattcttcagagagcattaaaaaatgtgacagtcagcttcagagaaactgaggagaatgcagtctggattcgaattgcctggggaacacagtacacaaagccaaaccagtacaaacctacctacgtggtgtactactcccagactccgtacgccttcacgtcctcctccatgctgaggcgcaatacaccgcttctgggtcaggcgctgacaattgctagcaaacaccatcagattgtgaaaatggacctgagaagtcggtatctggactctcttaaggctattgtttttaaacagtataatcagacctttgaaactcacaactctacgacacctctacaggaaagaagccttggactagatataaatatggattcaaggatcattcatgaaaacatagtagaaaaagagagagtccaacgaataactcaagaaacatttggagattatcctcaaccacaactagaatttgcacaatataagcttgaaacgaaattcaaaagtggtttaaatgggagcatcttggctgagagggaagaacccctccgatgcctaataaagttctctagcccacatcttctggaagcattgaaatccttagcaccagcggcacttgtttgcaggatccaaaagctgctctgctattctggaagccacagtcaagggacccaggacccgagcagctggcagaaggacttgtaccttctgtttgtcccattgtatccaagatgttaaaatcatgatgtcacaacatttagacatttaaatggttaatcaaatgtatgtctctcctgatatacagctaactgctatagactaatgcaggcctccttgttaaaatggaccctttgttactcaaatttgaccaatcttggaagcttggatgtgcactgacttgacaaccactttttgtgggacataacactctagacacaggtgcttctagctctaaggggaacagataactaatctgtggccaaccaaccaatgattaatcagctatgctgcctcggatcttgatcaaaaaaggaaaatgtgaaaagtgatacacaaaatggcatcactttggttcagagctctaaaatggagttgggaagccattctaagaaggactgcctgcacaatctgcaacttgcaaaacacaaaaa

SEQ ID NO: 8 - DNA, 21 bp, Artificial Sequence
  Synthetic siRNA sequence targeting the gene, DDX39
ccaggtgataatcttcgtcaa

SEQ ID NO: 9 - DNA, 21 bp, Artificial Sequence
  Synthetic siRNA sequence targeting the gene, DDX39
caggaccggtttgaagttaat

SEQ ID NO: 10 - DNA, 1534 bp, Homo sapiens
ggaagcgcagcaactcgtgtctgagcgcccggcggaaaaccgaagttggaagtgtctcttagcagcgcgcggagaagaacggggagccagcatcatggcagaacaggatgtggaaaacgatcttttggattacgatgaagaggaagagccccaggctcctcaagagagcacaccagctccccctaagaaagacatcaagggatcctacgtttccatccacagctctggcttccgggactttctgctgaagccggagctcctgcgggccatcgtggactgtggctttgagcatccttctgaggtccagcatgagtgcattccccaggccatcctgggcatggacgtcctgtgccaggccaagtccgggatgggcaagacagcggtcttcgtgctggccaccctacagcagattgagcctgtcaacggacaggtgacggtcctggtcatgtgccacacgagggagctggccttccagatcagcaaggaatatgagcgcttttccaagtacatgcccagcgtcaaggtgtctgtgttcttcggtggtctctccatcaagaaggatgaagaagtgttgaagaagaactgtccccatgtcgtggtggggaccccgggccgcatcctggcgctcgtgcggaataggagcttcagcctaaagaatgtgaagcactttgtgctggacgagtgtgacaagatgctggagcagctggacatgcggcgggatgtgcaggagatcttccgcctgacaccacacgagaagcagtgcatgatgttcagcgccaccctgagcaaggacatccggcctgtgtgcaggaagttcatgcaggatcccatggaggtgtttgtggacgacgagaccaagctcacgctgcacggcctgcagcagtactacgtcaaactcaaagacagtgagaagaaccgcaagctctttgatctcttggatgtgctggagtttaaccaggtgataatcttcgtcaagtcagtgcagcgctgcatggccctggcccagctcctcgtggagcagaacttcccggccatcgccatccaccggggcatggcccaggaggagcgcctgtcacgctatcagcagttcaaggatttccagcggcggatcctggtggccaccaatctgtttggccgggggatggacatcgagcgagtcaacatcgtctttaactacgacatgcctgaggactcggacacctacctgcaccgggtggcccgggcgggtcgctttggcaccaaaggcctagccatcacttttgtgtctgacgagaatgatgccaaaatcctcaatgacgtccaggaccggtttgaagttaatgtggcagaacttccagaggaaatcgacatctccacatacatcgagcagagccggtaaccaccacgtgccagagccgcccacccggagccgcccgcatgcagcttcacctcccctttccaggcgccactgttgagaagctagagattgtatgagaataaacttgttattatggaaaaaaaaaaaaaaaaaaaaaaaaaaaaaaaaaaaaaaaaa

SEQ ID NO: 11 - DNA, 21 bp, Artificial Sequence
  Synthetic siRNA sequence targeting the gene, DEPDC1
ttccgtagtctaagataacta

SEQ ID NO: 12 - DNA, 5356 bp, Homo sapiens
tatgctattcaaatcggcggcggggccaacggttgtgccgagactcgccactgccgcggccgctgggcctgagtgtcgccttcgccgccatggacgccaccgggcgctgacagacctatggagagtcagggtgtgcctcccgggccttatcgggccaccaagctgtggaatgaagttaccacatcttttcgagcaggaatgcctctaagaaaacacagacaacactttaaaaaatatggcaattgtttcacagcaggagaagcagtggattggctttatgacctattaagaaataatagcaattttggtcctgaagttacaaggcaacagactatccaactgttgaggaaatttcttaagaatcatgtaattgaagatatcaaagggaggtggggatcagaaaatgttgatgataacaaccagctcttcagatttcctgcaacttcgccacttaaaactctaccacgaaggtatccagaattgagaaaaaacaacatagagaacttttccaaagataaagatagcatttttaaattacgaaacttatctcgtagaactcctaaaaggcatggattacatttatctcaggaaaatggcgagaaaataaagcatgaaataatcaatgaagatcaagaaaatgcaattgataatagagaactaagccaggaagatgttgaagaagtttggagatatgttattctgatctacctgcaaaccattttaggtgtgccatccctagaagaagtcataaatccaaaacaagtaattccccaatatataatgtacaacatggccaatacaagtaaacgtggagtagttatactacaaaacaaatcagatgacctccctcactgggtattatctgccatgaagtgcctagcaaattggccaagaagcaatgatatgaataatccaacttatgttggatttgaacgagatgtattcagaacaatcgcagattattttctagatctccctgaacctctacttacttttgaatattacgaattatttgtaaacattttggttgtttgtggctacatcacagtttcagatagatccagtgggatacataaaattcaagatgatccacagtcttcaaaattccttcacttaaacaatttgaattccttcaaatcaactgagtgccttcttctcagtctgcttcatagagaaaaaaacaaagaagaatcagattctactgagagactacagataagcaatccaggatttcaagaaagatgtgctaagaaaatgcagctagttaatttaagaaacagaagagtgagtgctaatgacataatgggaggaagttgtcataatttaatagggttaagtaatatgcatgatctatcctctaacagcaaaccaaggtgctgttctttggaaggaattgtagatgtgccagggaattcaagtaaagaggcatccagtgtctttcatcaatcttttccgaacatagaaggacaaaataataaactgtttttagagtctaagcccaaacaggaattcctgttgaatcttcattcagaggaaaatattcaaaagccattcagtgctggttttaagagaacctctactttgactgttcaagaccaagaggagttgtgtaatgggaaatgcaagtcaaaacagctttgtaggtctcagagtttgcttttaagaagtagtacaagaaggaatagttatatcaatacaccagtggctgaaattatcatgaaaccaaatgttggacaaggcagcacaagtgtgcaaacagctatggaaagtgaactcggagagtctagtgccacaatcaataaaagactctgcaaaagtacaatagaactttcagaaaattctttacttccagcttcttctatgttgactggcacacaaagcttgctgcaacctcatttagagagggttgccatcgatgctctacagttatgttgtttgttacttcccccaccaaatcgtagaaagcttcaacttttaatgcgtatgatttcccgaatgagtcaaaatgttgatatgcccaaacttcatgatgcaatgggtacgaggtcactgatgatacataccttttctcgatgtgtgttatgctgtgctgaagaagtggatcttgatgagcttcttgctggaagattagtttctttcttaatggatcatcatcaggaaattcttcaagtaccctcttacttacagactgcagtggaaaaacatcttgactacttaaaaaagggacatattgaaaatcctggagatggactatttgctcctttgccaacttactcatactgtaagcagattagtgctcaggagtttgatgagcaaaaagtttctacctctcaagctgcaattgcagaacttttagaaaatattattaaaaacaggagtttacctctaaaggagaaaagaaaaaaactaaaacagtttcagaaggaatatcctttgatatatcagaaaagatttccaaccacggagagtgaagcagcactttttggtgacaaacctacaatcaagcaaccaatgctgattttaagaaaaccaaagttccgtagtctaagataactaactgaattaaaaattatgtaatacttgtggaactttgataaatgaagccatatctgagaatgtagctactcaaaaggaagtctgtcattaataaggtatttctaaataaacacattatgtaaggaagtgccaaaatagttatcaatgtgagactcttaggaaactaactagatctcaattgagagcacataacaatagatgataccaaatactttttgtttttaacacagctatccagtaaggctatcatgatgtgtgctaaaattttatttacttgaattttgaaaactgagctgtgttagggattaaactataattctgttcttaaaagaaaatttatctgcaaatgtgcaagttctgagatattagctaatgaattagttgtttggggttacttctttgtttctaagtataagaatgtgaagaatatttgaaaactcaatgaaataattctcagctgccaaatgttgcactcttttatatattctttttccacttttgatctatttatatatatgtatgtgtttttaaaatatgtgtatattttatcagatttggttttgccttaaatattatccccaattgcttcagtcattcatttgttcagtatatatattttgaattctagttttcataatctattagaagatggggatataaaagaagtataaggcaatcatatattcattcaaaagatatttatttagcaactgctatgtgcctttcgttgttccagatatgcagagacaatgataaataaaacatataatctcttccataaggtatttattttttaatcaagggagatacacctatcagatgtttaaaataacaacactacccactgaaatcagggcatatagaatcattcagctaaagagtgacttctatgatgatggaacaggtctctaagctagtggttttcaaactggtacacattagactcacccgaggaattttaaaacagcctatatgcccagggcctaacttacactaattaaatctgaattttggggatgttgtatagggattagtattttttttaatctaggtgattccaatattcagccaactgtgagaatcaatggcctaaatgctttttataaacatttttataagtgtcaagataatggcacattgactttattttttcattggaagaaaatgcctgccaagtataaatgactctcatcttaaaacaaggttcttcaggtttctgcttgattgacttggtacaaacttgaagcaagttgccttctaatttttactccaagattgtttcatatctattccttaagtgtaaagaaatatataatgcatggtttgtaataaaatcttaatgtttaatgactgttctcatttctcaatgtaatttcatactgtttctctataaaatgatagtattccatttaacattactgatttttattaaaaacctggacagaaaattataaattataaatatgactttatcctggctataaaattattgaaccaaaatgaattctttctaaggcatttgaatactaaaacgtttattgtttatagatatgtaaaatgtggattatgttgcaaattgagattaaaattatttggggttttgtaacaatataattttgcttttgtattatagacaaatatataaataataaaggcaggcaactttcatttgcactaatgtacatgcaattgagattacaaaatacatggtacaatgctttaataacaaactctgccagtcaggtttgaatcctactgtgctattaactagctagtaaactcagacaagttacttaacttctctaagccccagttttgttatctataaaatgaatattataatagtacctctttttaggattgcgaggattaagcaggataatgcatgtaaagtgttagcacagtgtctcacatagaataagcactctataaatattttactagaatcacctaggattatagcactagaagagatcttagcaaaaatgtggtcctttctgttgctttggacagacatgaaccaaaacaaaattacggacaattgatgagccttattaactatcttttcattatgagacaaaggttctgattatgcctactggttgaaattttttaatctagtcaagaaggaaaatttgatgaggaaggaaggaatggatatcttcagaagggcttcgcctaagctggaacatggatagattccattctaacataaagatctttaagttcaaatatagatgagttgactggtagatttggtggtagttgctttctcgggatataagaagcaaaatcaactgctacaagtaaagaggggatggggaaggtgttgcacatttaaagagagaaagtgtgaaaaagcctaattgtgggaatgcacaggtttcaccagatcagatgatgtctggttattctgtaaattatagttcttatcccagaaattactgcctccaccatccctaatatcttctaattggtatcatataatgacccactcttcttatgttatccaaacagttatgtggcatttagtaatggaatgtacatggaatttcccactgacttacctttctgtccttgggaagcttaaactctgaatcttctcatctgtaaaatgtgaattaaagtatctacctaactgagttgtgattgtagtgaaagaaaggcaatatatttaaatcttgaatttagcaagcccacgcttgatttttatgtcctttcctcttgccttgtattgagtttaagatctctactgattaaaactcttttgctatcaaaaaaaaaaaaaaaaaaaaaaaaa

SEQ ID NO: 13 - DNA, 21 bp, Artificial Sequence
  Synthetic siRNA sequence targeting the gene, EXO1
atggatgtactttaccttcta

SEQ ID NO: 14 - DNA, 21 bp, Artificial Sequence
  Synthetic siRNA sequence targeting the gene, EXO1
cagatgtagcacgtaattcaa

SEQ ID NO: 15 - DNA, 3140 bp, Homo sapiens
cccgtgttctgcgttgccggccgtgggtgctctggccacagtgagttaggggcgtcggagcgggtttctccaaccgcaatcggctccgctcaaggggaggaggagagtcccttctcggaaggcctaaggaaacgtgtcgtctggaatgggcttgggggccacgcctgcacatctccgcgagacagagggataaagtgaagatggtgctgttattgttacctcgagtgccacatgcgacctctgagatatgtacacagtcattcttactatcgcactcagccattcttactacgctaaagaagaaataattattcgaggatatttgcctggcccagaagaaacttatgtaaatttcatgaactattatatccgttttcctcggagtgagagaaaactctttttagatatcatctgagaggtagttaatttggcaccatggggatacagggattgctacaatttatcaaagaagcttcagaacccatccatgtgaggaagtataaagggcaggtagtagctgtggatacatattgctggcttcacaaaggagctattgcttgtgctgaaaaactagccaaaggtgaacctactgataggtatgtaggattttgtatgaaatttgtaaatatgttactatctcatgggatcaagcctattctcgtatttgatggatgtactttaccttctaaaaaggaagtagagagatctagaagagaaagacgacaagccaatcttcttaagggaaagcaacttcttcgtgaggggaaagtctcggaagctcgagagtgtttcacccggtctatcaatatcacacatgccatggcccacaaagtaattaaagctgcccggtctcagggggtagattgcctcgtggctccctatgaagctgatgcgcagttggcctatcttaacaaagcgggaattgtgcaagccataattacagaggactcggatctcctagcttttggctgtaaaaaggtaattttaaagatggaccagtttggaaatggacttgaaattgatcaagctcggctaggaatgtgcagacagcttggggatgtattcacggaagagaagtttcgttacatgtgtattctttcaggttgtgactacctgtcatcactgcgtgggattggattagcaaaggcatgcaaagtcctaagactagccaataatccagatatagtaaaggttatcaagaaaattggacattatctcaagatgaatatcacggtaccagaggattacatcaacgggtttattcgggccaacaataccttcctctatcagctagtttttgatcccatcaaaaggaaacttattcctctgaacgcctatgaagatgatgttgatcctgaaacactaagctacgctgggcaatatgttgatgattccatagctcttcaaatagcacttggaaataaagatataaatacttttgaacagatcgatgactacaatccagacactgctatgcctgcccattcaagaagtcatagttgggatgacaaaacatgtcaaaagtcagctaatgttagcagcatttggcataggaattactctcccagaccagagtcgggtactgtttcagatgccccacaattgaaggaaaatccaagtactgtgggagtggaacgagtgattagtactaaagggttaaatctcccaaggaaatcatccattgtgaaaagaccaagaagtgcagagctgtcagaagatgacctgttgagtcagtattctctttcatttacgaagaagaccaagaaaaatagctctgaaggcaataaatcattgagcttttctgaagtgtttgtgcctgacctggtaaatggacctactaacaaaaagagtgtaagcactccacctaggacgagaaataaatttgcaacatttttacaaaggaaaaatgaagaaagtggtgcagttgtggttccagggaccagaagcaggtttttttgcagttcagattctactgactgtgtatcaaacaaagtgagcatccagcctctggatgaaactgctgtcacagataaagagaacaatctgcatgaatcagagtatggagaccaagaaggcaagagactggttgacacagatgtagcacgtaattcaagtgatgacattccgaataatcatattccaggtgatcatattccagacaaggcaacagtgtttacagatgaagagtcctactcttttgagagcagcaaatttacaaggaccatttcaccacccactttgggaacactaagaagttgttttagttggtctggaggtcttggagatttttcaagaacgccgagcccctctccaagcacagcattgcagcagttccgaagaaagagcgattcccccacctctttgcctgagaataatatgtctgatgtgtcgcagttaaagagcgaggagtccagtgacgatgagtctcatcccttacgagaaggggcatgttcttcacagtcccaggaaagtggagaattctcactgcagagttcaaatgcatcaaagctttctcagtgctctagtaaggactctgattcagaggaatctgattgcaatattaagttacttgacagtcaaagtgaccagacctccaagctatgtttatctcatttctcaaaaaaagacacacctctaaggaacaaggttcctgggctatataagtccagttctgcagactctctttctacaaccaagatcaaacctctaggacctgccagagccagtgggctgagcaagaagccggcaagcatccagaagagaaagcatcataatgccgagaacaagccggggttacagatcaaactcaatgagctctggaaaaactttggatttaaaaaagattctgaaaagcttcctccttgtaagaaacccctgtccccagtcagagataacatccaactaactccagaagcggaagaggatatatttaacaaacctgaatgtggccgtgttcaaagagcaatattccagtaaatgcagactgctgcaaagcttttgcctgcaagagaatctgatcaatttgaagtccctgtttgggaatgaggcacttatcagcatgaagaattttttctcattctgtgccattttaaaaatagaatacattttgtatattaactttaaaaaaaaaaaaaaaaaaa

SEQ ID NO: 16 - DNA, 21 bp, Artificial Sequence
  Synthetic siRNA sequence targeting the gene, EXOSC9
tggcaaatacgtgtagaccta

SEQ ID NO: 17 - DNA, 1644 bp, Homo sapiens
gatgacgtaattttcctgcgcctcggggcgagcagcggcgcgcaaggaaagatcgggttccgtttttcccgcggattctggtgcctgtggggccggtgacccaacaccatgaaggaaacgccactctcaaactgcgaacgccgcttcctactccgtgccatcgaagagaagaagcggctggatggcagacaaacctatgattataggaacatcaggatctcatttggaacagattacggatgctgcattgtggaacttggaaaaacaagagttcttggacaggtttcctgtgaacttgtgtctccaaaactcaatcgggcaacagaaggtattcttttttttaaccttgaactctctcagatggccgctccagctttcgaacctggcaggcagtcagatctcttggtgaagttgaatcgactcatggaaagatgtctaagaaattcgaagtgtatagacactgagtctctctgtgttgttgctggtgaaaaggtttggcaaatacgtgtagacctacatttattaaatcatgatggaaatattattgatgctgccagcattgctgcaatcgtggccttatgtcatttccgaagacctgatgtctctgtccaaggagatgaagtaacactgtatacacctgaagagcgtgatcctgtaccattaagtatccaccacatgcccatttgtgtcagttttgcctttttccagcaaggaacatatttattggtggatcccaatgaacgagaagaacgtgtgatggatggcttgctggtgattgccatgaacaaacatcgagagatttgtactatccagtccagtggtgggataatgctactaaaagatcaagttctgagatgcagtaaaatcgctggtgtgaaagtagcagaaattacagagctaatattgaaagctttggagaatgaccaaaaagtaaggaaagaaggtggaaagtttggttttgcagagtctatagcaaatcaaaggatcacagcatttaaaatggaaaaggcccctattgatacctcggatgtagaagaaaaagcagaagaaatcattgctgaagcagaacctccttcagaagttgtttctacacctgtgctatggactcctggaactgcccaaattggagagggagtagaaaactcctggggtgatcttgaagactctgagaaggaagatgatgaaggcggtggtgatcaagctatcattcttgatggtataaaaatggacactggagtagaagtctctgatattggaagccaagagctggggtttcaccatgttggccagactggactcgagttcctgacctcagatgctcccataatactctcagatagtgaagaagaagaaatgatcattttggaaccagacaagaatccaaagaaaataagaacacagaccaccagtgcaaaacaagaaaaagcaccaagtaaaaagccagtgaaaagaagaaaaaagaagagagctgccaattaaagctaacagttgtatatctgtatatataactattaaaagggatatttattccattctgagaaccctgggtattttttattcacaaatccattataaaatctagcaggattttaaaaatagttttttgtttttaatgtgctttaaaataataaaccttctggagca

SEQ ID NO: 18 - DNA, 21 bp, Artificial Sequence
  Synthetic siRNA sequence targeting the gene, KIF14
atggttaatcgtgctccagaa

SEQ ID NO: 19 - DNA, 21 bp, Artificial Sequence
  Synthetic siRNA sequence targeting the gene, KIF14
tagggtcttagtaacattctt

SEQ ID NO: 20 - DNA, 7293 bp, Homo sapiens
ctggggagccggcgctggaggtggtgagtggcgtggggactgtgtcgagggggtccccaaggtgccggaccctgcggaggggcgaagtttcggcactggggagggcgtgcggacgctttccctacaggcgaccactgctctgcgggcgggtggtcttagctccagtcccccattcagttcctcagcattccaggtcggcggcgaaggggtccccgaacgaagggcgcaaggcagcgtctctgctgggaccgggaagccggacttcagggcctctcggcccgtgggcttctccccgagtctccccgagtcggttggcattaagagtttagcagatactttcagaaatggatacataagaaatggctggaaatcaaatgaatgtccaaagaagagcttagggtcttagtaacattcttttttaaaataactgtctgccaaaatgtcattacacagtactcataatagaaataacagcggtgatattcttgatattccttcttcccaaaatagttcatcactgaatgccctcacccacagtagccgacttaagctgcatttgaagtcggatatgtcagaatgtgaaaatgatgatccattattgagatctgcaggtaaagtcagagacataaatagaacttatgttatttctgccagtagaaaaacagcagacatgccccttacccctaatcctgtaggtagattggcacttcagaggagaactacaaggaacaaagaatcatctttgcttgttagtgagttggaagacacaactgaaaaaacagcagaaacacgtcttacattacaacgtcgtgctaaaacagattctgcagaaaagtggaaaacagctgaaatagattctgtcaaaatgacactgaatgtgggaggtgaaacagaaaataatggtgtttctaaggaaagtagaacaaatgtaaggattgtaaataatgctaaaaactcttttgttgcctcttctgtacctttagatgaagatccacaggtcattgaaatgatggctgataagaaatacaaagaaacattttctgcccccagtagagcaaatgaaaatgttgcacttaagtactcaagtaatagaccacccattgcttccctgagtcagactgaagttgttagatcaggacacttgacaacgaaacctactcagagcaagttggatatcaaagtgttgggaacaggaaacttgtatcatagaagtattgggaaggaaattgcaaaaacttcaaataaatttgggagcttagaaaaaagaacacctacaaaatgtacaacagaacacaaactgacaacaaagtgcagcctgcctcagcttaagagcccagctccatcaatactgaagaatagaatgtctaaccttcaagttaaacaaagaccaaaaagttcctttcttgcaaataaacaggaaagatccgcagaaaatacaattcttcccgaagaagaaactgtagttcagaacacctctgcaggaaaagaccccttaaaagtagagaatagtcaagtgacagtggcagtacgcgtaagacctttcaccaagagagagaagattgaaaaagcatcccaggtagtcttcatgagtgggaaagaaataactgtggaacaccctgacacgaaacaagtttataattttatttatgatgtttcattctggtcttttgatgaatgtcatcctcactacgctagccagacaactgtctatgagaagctagcagcaccactcctagaaagagccttcgaaggcttcaatacctgtctttttgcttatggtcagactggctctggaaaatcatatacgatgatgggatttagtgaagaaccaggaataattccaagattttgtgaagatcttttttctcaagtagccagaaaacaaacccaagaggtcagctatcacattgaaatgagcttctttgaagtatataatgaaaaaattcacgaccttctggtttgtaaagatgaaaatgggcagagaaagcaaccactgagagtgagggaacatcctgtttatggaccatatgttgaagcactgtcaatgaacattgtcagttcttacgctgatatccagagttggctagaattgggaaataaacaaagagctactgctgctactggtatgaatgataaaagttcccgatctcattcagttttcaccctggtgatgacccagaccaagacagaatttgtggaaggggaagaacacgatcacagaataacaagtcgaattaacctaatagatctggcaggcagtgagcgctgctctacggctcacactaatggagatcgactaaaggaaggtgtgagtattaataagtccttgctaactttgggaaaagttatatctgcactttcggaacaagcaaaccaaaggagtgtttttattccttatcgtgaatctgttcttacatggctgttaaaagaaagtctgggtggaaattcaaaaactgcaatgattgctacgattagtcccgctgccagcaacatagaagaaacattaagcacacttagatatgctaaccaagcccgtttaatagtcaacattgctaaagtaaatgaagatatgaacgctaagttaattagagaattgaaggcagaaattgcaaagctaaaagctgctcagagaaacagtcggaatattgaccctgaacgatacaggctctgtcggcaagaaataacatccttaagaatgaaactgcatcaacaggagagagacatggcagaaatgcaaagagtgtggaaagaaaagtttgaacaagctgaaaaaagaaaacttcaagaaacaaaagagttacagaaagcaggaattatgtttcaaatggacaatcatttaccaaaccttgttaatctgaatgaagatccacaactatctgagatgctgctatatatgataaaagaaggaacaactacagttggaaagtataaaccaaactcaagccatgatattcagttatctggggtgctgattgctgatgatcattgtactatcaaaaattttggtgggacagtgagtattatcccagttggggaagcaaagacatatgtaaatggaaaacatattttggaaatcacagtattacgtcatggtgatcgagtgattcttggtggagatcattattttagatttaatcatccagtagaagtccagaaaggaaaaaggccatctggaagagatactcctataagtgagggtccaaaagactttgaatttgcaaaaaatgagttgctcatggcacagagatcacaacttgaagcagaaataaaagaggctcagttgaaggcaaaggaagaaatgatgcaaggaatccagattgcaaaagaaatggctcagcaagagctttcttctcaaaaagctgcatatgaaagcaaaataaaagcactggaagcagaactgagagaagagtctcaaaggaaaaaaatgcaggaaataaataaccagaaggctaatcacaaaattgaggaattagaaaaggcaaagcagcatcttgaacaggaaatatatgtcaacaaaaagcgattagaaatggaaacattggctacaaaacaggctttagaagaccatagcatccgccatgcaagaattctggaagctttagaaactgaaaagcaaaaaattgctaaagaagtacaaattctacagcagaatcggaataatagggataaaacttttacagtgcagacaacttggagctctatgaaactctcaatgatgattcaggaagccaatgctatcagcagcaaattgaaaacatactatgtttttggcagacatgatatatcagataaaagtagttctgacacttctattcgggttcgtaacctgaaactaggaatctcaacattctggagtctggaaaagtttgaatctaaacttgcagcaatgaaagaactttatgagagtaatggtagtaacaggggtgaagatgccttttgtgatcctgaagatgaatgggaacccgacattacagatgcaccagtttcttcactttctagaaggaggagtaggagtttgatgaagaacagaagaatttctggttgtttacatgacatacaagtccatccaattaagaatttgcattcttcacattcatcaggtttaatggacaaatcaagcactatttactcaaattcagcagagtcctttcttcctggaatttgcaaagaattgattggttcttcgttagatttttttggacagagttatgatgaagaaagaactatagcagacagcctaattaatagttttcttaaaatttataatgggctatttgccatttccaaggctcatgaagaacaagatgaagaaagtcaagataacttgttttcttctgatcgagcaatccagtcacttactattcagactgcatgtgcttttgagcagctagtagtgctaatgaaacactggctgagtgatttactgccttgtaccaacatagcaagacttgaggatgagttgagacaagaagttaaaaaactgggaggctacttacagttatttttgcagggatgctgtttggatatttcatcaatgataaaagaggctcaaaagaatgcaatccaaattgtacaacaagctgtaaagtatgtggggcagttagcagttctgaaagggagcaagctacattttctagaaaacggtaacaataaagctgccagtgtccaggaggaattcatggatgctgtttgtgatggtgtaggcttaggaatgaagattttattagattctggactggaaaaagcaaaagaacttcagcatgaactctttaggcagtgtacaaaaaatgaggttaccaaagaaatgaaaactaatgccatgggattgattagatctcttgaaaacatctttgctgaatcgaaaattaaaagtttcagaaggcaagtacaagaagaaaactttgaataccaagatttcaagaggatggttaatcgtgctccagaattcttaaagttaaaacattgcttagagaaagctattgaaattattatttctgcactgaaaggatgccatagtgatataaatcttctccagacttgtgttgaaagtattcgcaacttggccagtgatttttacagtgacttcagtgtgccttctacttctgttggcagctatgagagtagagtaactcacattgtccaccaggaactagaatctctagctaagtctctcctcttttgttttgaatctgaagaaagccctgatttgttgaaaccctgggaaacttataatcaaaataccaaagaagaacaccaacaatctaaatcaagcgggattgacggcagtaagaataaaggtgtaccaaagcgtgtctatgagctccatggctcatccccagcagtgagctcagaggaatgcacacccagtaggattcagtgggtgtgaatactgatgtgtaggcacttttatgaccacccatgaaagaaaaagaacacttgctcggtaattttctttatgcaggagagtttaagagaaatcagcacagatatttcaaaaaagtccatgtctttttatctttaaaatatctatttatcaaaggccagacacagtggctcacgcctgtaatcccagcactttgggaggcgggcagatcacaaggtcaggagtttgagaccggcctggccaacatggcgaaaccccgtctctactaaaaatacaaaaatttgctgggcatggtggcgcgtgcctgtaatcccagctactaggggggctgaggcaggaggatcgcttgaacctgagaggcagaggttgcagtgagccaagatcatgccactttactccagtctgagcaacagaacgagacttagtcaaaataaataaataaataaataaataaataaataaataaataaataaaatatatttttatctttaaagtgtttaacattggtatactgtctgtagttggttcattagtcgtttataaagggttattttctcatgagtggaaacctgaacaatcagttacctttgtgcctatgccttctctctcctcagacagctgggatgtttatggtgaaatggcctgtacaagtttaactaagacaacttaacttgcattgttaatcaaaaattcttttctcaaagggttaactggttgccattttgaatagtatgttcaagggtgtagcttcctgtttctttccaaattataagtagctacctaaatatagtataattatatattaataatatggcttgctggcacagtagtttaccctgttatctgtgtttcataatgggggctgtatgaatattatttaaaactaataaaatgttgccagaattatactaaactgttggatgagattaggagatcagaggctggaccttctcttgataatgcttgttttgttaaaggtataatgaaataatttgtatatgatttgatgaagattaaagacccttattttccacagctttaaaaaaaaacctttatttatgatcaagtaataaagataatattctacttgtgggatcttacattacggaaatagtttgacgtttttgacctcaagagtatgtataatttgaagagatactttgtaactatgcttgggtgatattgagcagttcctaaagaataattcatttaaaaaaaaagaagaaaaaaaaagaagaattcatttaaataacctgatcctttcatttgcccttttcgaatttacagatactacttgtacatttggcataactagttgaaattggccattcgtaccatgaataaatctgatagtttccttgttaggaagagattgtaagtaaatacagtcattgcagtcagaacagtattagtgaaccttgtgtggtgttttcaagctctttaaaatggtacaatgtagcacatttgctttcatttctttttttatttttggcatttgaccttgtattctttctgaagctctatatgtgtttttattagtcaataatctggcaagtagcactttgcctgtgcagtttgctggagtgtagatgtacatatgaggatttcccgggaggtgcacttctttgaagaacttcctaaagtacctgtatagtagttttcatcttaatattcagtatttaatcttcagtttgtgctttgtaaactcatgacttaattggtcagaaactttttagtgtctttataaaattttgtatacatatttatactaaacacattgtgatactgtatttgaatgaatggtgaaaaaatatttgctattggaattatgtgcactgacaagaaatgttataaagagaatgcctttaataaatcttttcagcattagaattgaaaaaaaaaaaaaaaa

SEQ ID NO: 21 - DNA, 21 bp, Artificial Sequence
  Synthetic siRNA sequence targeting the gene, KIF23
aaggctgaagattatgaagaa

SEQ ID NO: 22 - DNA, 21 bp, Artificial Sequence
  Synthetic siRNA sequence targeting the gene, KIF23
cagaagttgaagtgaaatcta

SEQ ID NO: 23 - DNA, 3636 bp, Homo sapiens
gcagagcaccgcgccttagccgcgaagttctagttcttgctgccggtcctaacgtcccgcagtcttcgccagccagccgtcccgcatgcgcgtttgggcggcgtggagcctgctgccatgaagtcagcgagagctaagacaccccggaaacctaccgtgaaaaaagggtcccaaacgaaccttaaagacccagttggggtatactgtagggtgcgcccactgggctttcctgatcaagagtgttgcatagaagtgatcaataatacaactgttcagcttcatactcctgagggctacagactcaaccgaaatggagactataaggagactcagtattcatttaaacaagtatttggcactcacaccacccagaaggaactctttgatgttgtggctaatcccttggtcaatgacctcattcatggcaaaaatggtcttctttttacatatggtgtgacgggaagtggaaaaactcacacaatgactggttctccaggggaaggagggctgcttcctcgttgtttggacatgatctttaacagtatagggtcatttcaagctaaacgatatgttttcaaatctaatgataggaatagtatggatatacagtgtgaggttgatgccttattagaacgtcagaaaagagaagctatgcccaatccaaagacttcttctagcaaacgacaagtagatccagagtttgcagatatgataactgtacaagaattctgcaaagcagaagaggttgatgaagatagtgtctatggtgtatttgtctcttatattgaaatatataataattacatatatgatctattggaagaggtgccgtttgatcccataaaacccaaacctccacaatctaaattgcttcgtgaagataagaaccataacatgtatgttgcaggatgtacagaagttgaagtgaaatctactgaggaggcttttgaagttttctggagaggccagaaaaagagacgtattgctaatacccatttgaatcgtgagtccagccgttcccatagcgtgttcaacattaaattagttcaggctcccttggatgcagatggagacaatgtcttacaggaaaaagaacaaatcactataagtcagttgtccttggtagatcttgctggaagtgaaagaactaaccggaccagagcagaagggaacagattacgtgaagctggtaatattaatcagtcactaatgacgctaagaacatgtatggatgtcctaagagagaaccaaatgtatggaactaacaagatggttccatatcgagattcaaagttaacccatctgttcaagaactactttgatggggaaggaaaagtgcggatgatcgtgtgtgtgaaccccaaggctgaagattatgaagaaaacttgcaagtcatgagatttgcggaagtgactcaagaagttgaagtagcaagacctgtagacaaggcaatatgtggtttaacgcctgggaggagatacagaaaccagcctcgaggtccagttggaaatgaaccattggttactgacgtggttttgcagagttttccacctttgccgtcatgcgaaattttggatatcaacgatgagcagacacttccaaggctgattgaagccttagagaaacgacataacttacgacaaatgatgattgatgagtttaacaaacaatctaatgcttttaaagctttgttacaagaatttgacaatgctgttttaagtaaagaaaaccacatgcaagggaaactaaatgaaaaggagaagatgatctcaggacagaaattggaaatagaacgactggaaaagaaaaacaaaactttagaatataagattgagattttagagaaaacaactactatctatgaggaagataaacgcaatttgcaacaggaacttgaaactcagaaccagaaacttcagcgacagttttctgacaaacgcagattagaagccaggttgcaaggcatggtgacagaaacgacaatgaagtgggagaaagaatgtgagcgtagagtggcagccaaacagctggagatgcagaataaactctgggttaaagatgaaaagctgaaacaactgaaggctattgttactgaacctaaaactgagaagccagagagaccctctcgggagcgagatcgagaaaaagttactcaaagatctgtttctccatcacctgtgcctctttctagtaactatattgctcagatttccaacggccagcaactcatgagccagccacagctacataggcgctctaactcttgcagcagcatttctgtagcttcctgtatttcggaatgggagcagaaaattcctacgtacaacacacctctcaaagtcacatctattgcaaggcgtaggcagcaggagccaggacaaagcaaaacttgtatcgtgtcagacagaaggcgagggatgtactggactgaaggcagggaggtggttcctacattcagaaatgagatagaaatagaagaggatcattgcggcaggttactctttcaacctgatcagaacgcaccaccaattcgtctccgacacagacgatcacgctctgcaggagacagatgggtagatcataagcccgcctctaacatgcaaactgaaacagtcatgcagccacatgtccctcatgccatcacagtatctgttgcaaatgaaaaggcactagctaagtgtgagaagtacatgctgacccaccaggaactagcctccgatggggagattgaaactaaactaattaagggtgatatttataaaacaaggggtggtggacaatctgttcagtttactgatattgagactttaaagcaagaatcaccaaatggtagtcgaaaacgaagatcttccacagtagcacctgcccaaccagatggtgcagagtctgaatggaccgatgtagaaacaaggtgttctgtggctgtggagatgagagcaggatcccagctgggacctggatatcagcatcacgcacaacccaagcgcaaaaagccatgaactgacagtcccagtactgaaagaacattttcatttgtgtggatgatttctcgaaagccatgccagaagcagtcttccaggtcatcttgtagaactccagctttgttgaaaatcacggacctcagctacatcatacactgacccagagcaaagctttccctatggttccaaagacaactagtattcaacaaaccttgtatagtatatgttttgccatatttaatattaatagcagaggaagactccttttttcatcactgtatgaattttttataatgtttttttaaaatatatttcatgtatacttataaactaattcacacaagtgtttgtcttagatgattaaggaagactatatctagatcatgtctgattttttattgtgacttctccagccctggtctgaatttcttaaggttttataaacaaatgctgctatttattagctgcaagaatgcactttagaactatttgacaattcagactttcaaaataaagatgtaaatgactggccaataataaccattttaggaaggtgttttgaattctgtatgtatatattcactttctgacatttagatatgccaaaagaattaaaatcaaaagcactaagaaataaaaaaaaaaaaaaaaaaa

SEQ ID NO: 24 - DNA, 21 bp, Artificial Sequence
  Synthetic siRNA sequence targeting the gene, LMNB2
cgcctacaagttcacgcccaa

SEQ ID NO: 25 - DNA, 4653 bp, Homo sapiens
catcttgcagccggcggcgcggattgaatgagcccgccgagcccgggccgccgtcgggagcagcgcaggccgcgagccgccgccaccatggccacgccgctgcccggccgcgcgggcgggcccgccacgccgctgtcgcccacgcgcctgtcgcggctgcaggagaaggaggagctgcgcgagctcaacgaccgcctggcgcactacatcgaccgcgtccgcgcgctggagctggagaacgaccggctcctgctcaagatctcagagaaggaggaggtgaccacgcgcgaggtgagtggcatcaaggcgctgtacgagtcggagctggccgatgcccggagagtcctggatgagacggctcgagagcgtgcccggctgcagatagagattgggaagctgagggcagagttggacgaggtcaacaagagcgccaagaagagggagggcgagcttacggtggcccagggccgtgtgaaggacctggagtccctgttccaccggagcgaggtggagctggcagctgccctcagcgacaagcgcggcctggagagtgacgtggctgagctgcgggcccagctggccaaggccgaggacggtcatgcagtggccaaaaagcagctggagaaggagacgctgatgcgtgtggacctggagaaccgctgccagagcctgcaggaggagctggacttccggaagagtgtgttcgaggaggaggtgcgggagacgcggcggcggcacgagcggcgcctggtggaggtggacagcagccggcagcaggagtacgacttcaagatggcacaggcgctggaggagctgcggagccagcacgacgagcaagtgcggctctacaagctggagctggagcagacctaccaggccaagctggacagcgccaagctgagctctgaccagaacgacaaggcggccagtgcggctcgcgaggagctgaaggaggcccgcatgcgcctggagtccctcagctaccagctctccggcctccagaagcaggccagtgccgctgaagatcgcattcgggagctggaggaggccatggccggggagcgggacaagttccggaagatgctggacgccaaggagcaggagatgacggagatgcgggacgtgatgcagcagcagctggccgagtaccaggagctgctggacgtgaagctggccctggacatggagatcaacgcctaccggaagctcctggagggcgaggaggagaggctgaagctgtcccccagcccatcctcgcgcgtcaccgtctcacgagccacctcgagcagcagcggcagcttgtccgccaccgggcgcctgggccgcagtaagcggaagcggctggaggtggaggagcccttgggcagcggcccaagcgtcctgggcacgggcacgggtggcagcggtggcttccacctggcccagcaggcctcggcctcgggtagcgtcagcatcgaggagatcgacctggagggcaagtttgtgcagctcaagaacaactcggacaaggatcagtctctggggaactggagaatcaagaggcaggtcttggagggggaggagatcgcctacaagttcacgcccaagtacatcctgcgcgccggccagatggtcacggtgtgggcagctggtgcgggggtggcccacagccccccctcgacgctggtgtggaagggccagagcagctggggcacgggcgagagcttccgcaccgtcctggttaacgcggatggcgaggaagtggccatgaggactgtgaagaagtcctcggtgatgcgtgagaatgagaatggggaggaagaggaggaggaagccgagtttggcgaggaggatcttttccaccaacagggggacccgaggaccacctcaagaggctgctacgtgatgtgaacccacactcctcatccacacacctttctttacccagagccactgaaaactatttttatatcattggctttctttagttcttgatacatttctagagaatttctaagcgaactgccagaacgtgtgggtgggtctcccccagccctccctcctggcgggtctcctccagcctcacttcgctgccacttcgccgctgccccggagacttttcaatcccaccccactcctcatctcaccatttggtcaaattggaagcccagggccaggacccggaggtttagaagatgcttgggcttggagggaggagggccggcgaggctagcgaggggacaggagacggccctgctgcggacggagcgcggaaactgcgtaggaattcagtggtggtgggtttttttaaggctttctacaaaaccaaattcagaatccaggcgtcgacctggtggggcccggggccaagcctgcattctggctgcccagcttcggacagcgggaactcctcaggcagccacgcagcgggtgtgggccagcatggggatggcgtggcccccagggcgggttttcactccgctgcctgggcttccagattcccgttctggcagcgcaccggccgggtttctcggaccgttgactttatttgggggagttttcccgcagttcagttcctgactgtgcaaggccaacagggcaggggaggggaagacctggggaaggaagaatgaggacagtcccgtcgtaagacctgtcacaacaataagcagggaggggagatgtggaggggacacatctggttgccttggaggcagaagctgtgagtttcagaacagctgtctgcagggaacgccaccatgttgaccctctggaggagagcgctgtggagcccctcccgtgttccagctccgtctgccctgtgcctatatatacacatgcgtctatccatactgtgcttttatctgtgattttctcgctgaaaccatgtttctcagacaggccaaggccacctgactcctatcacgacgcacccaagcccctcagtccagcttcccaatgcctggcaccccccttcggcaatagctcaccgtttacaccctccctcatagatacacagaagttatttttttaatggatatttatttttttacattggtcagtacacaggtcaggagctcacgccagggccttgaggacaggctgaccctcctccccggggtggcgtggggctggggcaccctccgacggcagagcctccttcagaaagtgcagctcaagtcttaaagacaccaaaactgagccatgggcacgcgccgtctccgggccatggcgttcactgcagggcgggggcggcaccgctcccctgtgactgcatcccgcctccctggggacctgcctgtggcaggaaggaatggggggccccagccccaggccgggaaggagccagcggccgacaaagcagaaacacccgctgctccacgtagcccctgctcgctgtccttgctctcagaagtcccggtcccatgtagatagaggggggcgcatcttaccaaagcatttcctcctggaggctacgccgctgtgctcccagtcaggcggctggtagggagctttgcctgccccggggataccctctgccagccgctggaagtgggaatgctggcgacagactgtgtcctctttcccaccttcatagcaggaatcacccggacccgactggctgggcttcgtgctagcgagggttctgggggtgggtcttggtgatcttgtcctatggggagtctgcagtggtctcagccacatcctatgtattttggctctggaggagcaaagctgtatcctggagttggtctgtgatttgccgacagccttgcaggctgggctcagggacaaagtcccccccaaaacccgcaggtcctcatgtccagacgctgcccagtcctgtcctgaaaacagcacgccccaggcccacagaaccccccaccctacatttgccttgggtggagctgggggtggtcctaggactgcgggtgcccttagctgaagggggtggggagaagcgtggactgggcagcctgtgggtaattggaggttcattgagaattgagtctttggaaacactaagaaaatcaaatttttaaaagttatttatggcctgggaaacaatttgcatttgtccccaaatacgcttagctgtgtgccgcttagaacgatgcgaaaccatccctctgtgtaagcccgtgccgtgtgactcgaagcctagcgccctccctgcgaagcatcagacgccacccagccctggggggaggcccacgcctgctggaccaacgcgggttctggggtgcacagcgccaggttaacgctgaagcctgccccgctgagcccaggagccgggaggcctgcgggctgacccagaatccgatcatgcacctgtcctcatgccagcggctttggctggggttggtctgaagcctgcacgcggcagttctttgttaaagatctgagggactcctcagtcctggggcgtcgccgcctgcagcctcttccaagccctgcgtccagcgagcgtcacagcacaacctgcaaaaacggagctgggctgcagctggggctggcatggactttcatttcagagattcggtttttaagaagatgcatgcctaatgtgttcttttttttttccaatgatttgtaatatacattttatgactggaaacttttttgtacaacactccaataaacattttgattttaaaaaaaaaaaaaaaaaaa

SEQ ID NO: 26 - DNA, 21 bp, Artificial Sequence
  Synthetic siRNA sequence targeting the gene, MAD2L1
atggatatttgtactgtttaa

SEQ ID NO: 27 - DNA, 1453 bp, Homo sapiens
cgcaaaggacctgacgacgtgctgcgtcgttacttttgaaacgcttggcggggaagtgctgttggagccgctgtggttgctgtccgcggagtggaagcgcgtgcttttgtttgtgtccctggccatggcgctgcagctctcccgggagcagggaatcaccctgcgcgggagcgccgaaatcgtggccgagttcttctcattcggcatcaacagcattttatatcagcgtggcatatatccatctgaaacctttactcgagtgcagaaatacggactcaccttgcttgtaactactgatcttgagctcataaaatacctaaataatgtggtggaacaactgaaagattggttatacaagtgttcagttcagaaactggttgtagttatctcaaatattgaaagtggtgaggtcctggaaagatggcagtttgatattgagtgtgacaagactgcaaaagatgacagtgcacccagagaaaagtctcagaaagctatccaggatgaaatccgttcagtgatcagacagatcacagctacggtgacatttctgccactgttggaagtttcttgttcatttgatctgctgatttatacagacaaagatttggttgtacctgaaaaatgggaagagtcgggaccacagtttattaccaattctgaggaagtccgccttcgttcatttactactacaatccacaaagtaaatagcatggtggcctacaaaattcctgtcaatgactgaggatgacatgaggaaaataatgtaattgtaattttgaaatgtggttttcctgaaatcaagtcatctatagttgatatgttttatttcattggttaatttttacatggagaaaaccaaaatgatacttactgaactgtgtgtaattgttccttttatttttttggtacctatttgacttaccatggagttaacatcatgaatttattgcacattgttcaaaaggaaccaggaggtttttttgtcaacattgtgatgtatattcctttgaagatagtaactgtagatggaaaaacttgtgctataaagctagatgctttcctaaatcagatgttttggtcaagtagtttgactcagtataggtagggagatatttaagtataaaatacaacaaaggaagtctaaatattcagaatctttgttaaggtcctgaaagtaactcataatctataaacaatgaaatattgctgtatagctccttttgaccttcatttcatgtatagttttccctattgaatcagtttccaattatttgactttaatttatgtaacttgaacctatgaagcaatggatatttgtactgtttaatgttctgtgatacagaactcttaaaaatgttttttcatgtgttttataaaatcaagttttaagtgaaagtgaggaaataaagttaagtttgttttaaatttgtcttaaaaaaaaaaaaaaaaaaa

SEQ ID NO: 28 - DNA, 21 bp, Artificial Sequence
  Synthetic siRNA sequence targeting the gene, MAD2L1BP
gaggagatgctgaagaagaaa

SEQ ID NO: 29 - DNA, 21 bp, Artificial Sequence
  Synthetic siRNA sequence targeting the gene, MAD2L1BP
ctcccagatagaactacttga

SEQ ID NO: 30 - DNA, 1550 bp, Homo sapiens
gggaatttagagcctcgaggcctggggtggggacgcgaggacaccagcgtagaagagcttacatcagaatcgagctttgtgggcgctccgggatttggccctttagcgcggatcctagacaacaggttttggacctcgagagctgcagaactgaggctactggtgccgccagcctgctggctccgcctctgcctcagtttcttcccctatggcccgcgtgccgctggggcggagtctcactctgtcacccaggctggagcacaatggcatgacctcagctcaccacaacttccgcctcccaggttcaagggattctcctgcctcagcctcccaagtagctgagattatagatttggagtggtatgagaagtccgaagaaactcacgcctcccagatagaactacttgagacaagctctacgcaggaacctctcaacgcttcggaggccttttgcccaagagactgcatggtaccagtggtgtttcctgggcctgtgagccaggaaggctgctgtcagtttacttgtgaacttctaaagcatatcatgtatcaacgccagcagctccctctgccctatgaacagcttaagcacttttaccgaaaaccttctccccaggcagaggagatgctgaagaagaaacctcgggccaccactgaggtgagcagcaggaaatgccaacaagccctggcagaactggagagtgtcctcagccacctggaggacttctttgcacggacactagtaccgcgagtgctgattctccttgggggcaatgccctaagccccaaggagttctatgaactcgacttgtctctgctggccccctacagcgtggaccagagcctgagcacagcagcttgtttgcgccgtctcttccgagccatattcatggctgatgcctttagcgagcttcaggctcctccactcatgggcaccgtcgtcatggcacagggacaccgcaactgtggagaagattggtttcgacccaagctcaactatcgagtgcccagccggggccataaactgactgtgaccctgtcatgtggcagaccttccatccgaaccacggcttgggaagactacatttggttccaggcaccagtgacatttaaaggcttccgcgagtgaatgagtgcttcttaatcctaaaaacacaatggctgaattatctttctccatgtggcgctgaatcacccatctggtttggagctagagttgcttcctggtgagagaggaagcaactctccttctggttgtctgcctcccctcagatttcctgataggctgatggcatgtggctgtgactgtgactgtaatcattgctgaacaacatctctttgaatcaaaggttgattttcccagagggtgctgggtcaggcatttctattaggagttggaaagcaaaaatgggtccatagacactctatggaggtgtccctttctgctctttgctgtgtcctttcagaatttttaccaggaacataatgtggatgtgacttatgaacttaaatataaaataaatagattcttattatattttcctgaaaaa

SEQ ID NO: 31 - DNA, 21 bp, Artificial Sequence
  Synthetic siRNA sequence targeting the gene, NCAPD2
cacccgaattgtccagcagaa

SEQ ID NO: 32 - DNA, 4806 bp, Homo sapiens
agagccgcgggtgagatccccagccctgtgagcctgtaggagtagaatggctccccaaatgtatgagttccatctgccattatccccagaggagttgttgaaaagtggaggggtgaatcagtatgttgtgcaagaggtactgtccatcaaacatcttccaccacagcttagagcttttcaggctgcctttcgagctcaggggcccctggctatgctgcagcactttgatactatctacagcattttgcatcactttcgaagtatagatcctggcctcaaagaagatactctgcaattcctgataaaagtggtatcccgccactcccaggagcttccagctatcctggatgatacaactttgagtggatcagatagaaacgcccatctaaatgccctcaaaatgaactgttatgctctgatacgtctcctggaatcctttgagaccatggccagccagacaaaccttgtggacctggaccttggtgggaagggtaagaaagctcggaccaaggcagcccatggctttgactgggaagaagagaggcaaccaattcttcagcttttaacacagctacttcagttggacatccgtcacctgtggaaccactcaataattgaagaagaatttgtcagtttggttactggctgttgctaccgccttctggagaatcccaccattaatcaccagaagaaccgccccactcgggaagccataacacacctgcttggtgtagccttgacccgttataaccatatgctcagtgctacagtgaagatcatccagatgctgcagcactttgaacacctggcacctgtactggttgcagccgtgagtctatgggcaactgactatggaatgaagagcatagtgggagagattgtaagagagattggacaaaagtgtccccaagagctgagtcgagacccttcagggacaaagggctttgcagcattcctgacagaactagcagaacgtgtcccagctatcctgatgtccagcatgtgcattttgctagatcacctggatggagaaaattacatgatgcgtaatgctgtgctggcagccatggcggagatggtgctgcaggttctcagtggcgatcaactggaagcagcagcccgagacaccagagaccagttcttggatactttacaagcccatggccatgatgtcaactcctttgtgcggagccgtgttttgcagctcttcacccgaattgtccagcagaaggctctccccctgacacgtttccaggcagtggtggctttagctgtgggacgtctggcagacaagtcagtgctagtatgtaaaaatgccatccagctgctggccagttttctagccaataatcctttctcctgcaagcttagtgatgctgaccttgccggaccactgcagaaggagacccagaaattacaagagatgagggcccagaggcgaactgcagcagcttctgcagtgctggacccagaggaggagtgggaagccatgctgccagagttgaagtctaccctgcagcagcttctacagcttccccagggagaggaggagattcctgagcaaattgccaatacagagacaactgaagatgtgaaaggacgcatctatcaactgcttgccaaagctagttacaaaaaggccatcattctcactcgagaagccacaggccacttccaggagtccgaacccttcagtcatatagacccagaggagtcagaggagaccaggctcttgaatatcttaggacttatcttcaaaggcccagcagcttccacacaagaaaagaatccccgggagtctacaggaaacatggtcacaggacagactgtctgtaaaaataaacccaatatgtcggatcctgaggaatccaggggaaatgatgaactagtgaagcaggagatgctggtacagtatctgcaggatgcctacagcttctcccggaagattacagaggccattggcatcatcagcaagatgatgtatgaaaacacaactacagtggtgcaggaggtgattgaattctttgtgatggtcttccaatttggggtaccccaggccctgtttggggtgcgccgtatgctgcctctcatctggtctaaggagcctggtgtccgggaagccgtgcttaatgcctaccgccaactctacctcaaccccaaaggggactctgccagagccaaggcccaggctttgattcagaatctctctctgctgctagtggatgcctcggttgggaccattcagtgtcttgaggaaattctctgtgagtttgtgcagaaggatgagttgaaaccagcagtgacccagctgctgtgggagcgggccaccgagaaggtcgcctgctgtcctctggagcgctgttcctctgtcatgcttcttggcatgatggcacgaggaaagccagaaattgtgggaagcaatttagacacactggtgagcatagggctggatgagaagtttccacaggactacaggctggcccagcaggtgtgccatgccattgccaacatctcggacaggagaaagccttctctgggcaaacgtcacccccccttccggctgcctcaggaacacaggttgtttgagcgactgcgggagacagtcacaaaaggctttgtccacccagacccactctggatcccattcaaagaggtggcagtgaccctcatttaccaactggcagagggccccgaagtgatctgtgcccagatattgcagggctgtgcaaaacaggccctggagaagctagaagagaagagaaccagtcaggaggacccgaaggagtcccccgcaatgctccccactttcctgttgatgaacctgctgtccctggctggggatgtggctctgcagcagctggtccacttggagcaggcagtgagtggagagctctgccggcgccgagttctccgggaagaacaggagcacaagaccaaagatcccaaggagaagaatacgagctctgagaccaccatggaggaggagctggggctggttggggcaacagcagatgacacagaggcagaactaatccgtggcatctgcgagatggaactgttggatggcaaacagacactggctgcctttgttccactcttgcttaaagtctgtaacaacccaggcctctatagcaacccagacctctctgcagctgcttcacttgcccttggcaagttctgcatgatcagtgccactttctgcgactcccagcttcgtcttctgttcaccatgctggaaaagtctccacttcccattgtccggtctaacctcatggttgccactggggatctggccatccgctttcccaatctggtggacccctggactcctcatctgtatgctcgtctccgggaccctgctcagcaagtgcggaaaacagcggggctggtgatgacccacctgatcctcaaggacatggtgaaggtgaaggggcaggtcagcgagatggcggtgctgctcatcgaccccgagcctcagattgctgccctggccaagaacttcttcaatgagctctcccacaagggcaacgcaatctataatctccttccagatatcatcagccgcctgtcagaccccgagctgggggtggaggaagagcctttccacaccatcatgaaacagctcctctcctacatcaccaaggacaagcagacagagagcctggtggaaaagctgtgtcagcggttccgcacatcccgaactgagcggcagcagcgagacctggcctactgtgtgtcacagctgcccctcacagagcgaggcctccgtaagatgcttgacaattttgactgttttggagacaaactgtcagatgagtccatcttcagtgcttttttgtcagttgtaggcaagctgcgacgtggggccaagcctgagggcaaggctataatagatgaatttgagcagaagcttcgggcctgtcataccagaggtttggatggaatcaaggagcttgagattggccaagcaggtagccagagagcgccatcagccaagaaaccatccactggttctaggtaccagcctctggcttctacagcctcagacaatgactttgtcacaccagagccccgccgtactacccgtcggcatccaaacacccagcagcgagcttccaaaaagaaacccaaagttgtcttctcaagtgatgagtccagtgaggaagatctttcagcagagatgacagaagacgagacacccaagaaaacaactcccattctcagagcatcggctcgcaggcacagatcctaggaagtctgttcctgtcctccctgtgcagggtatcctgtagggtgacctggaattcgaattctgtttcccttgtaaaatatttgtctgtctcttttttttaaaaaaaaaaaaggccgggcactgtggctcacgcctgtaatcccagcactttgcgataccaaggcgggtggataacctgaggtagggagttcgagaccagcctgaccaacatggagaaaccccatctctactaaaaataaaaaattagccgggcgtattggcgtgcgcctgtaatcccagctactcaagaggctgaggcaggagaatcgcctgaacccagaggcggaggttgtagtgagccgaaatcacaccattgcactccagcttgggcaacaatagcgaacctccatctcaaattaaaaaaaaaatgcctacacgctctttaaaatgcaaggctttctcttaaattagcctaactgaactgcgttgagctgcttcaactttggaatatatgtttgccaatctccttgttttctaatgaataaatgtttttatatacttttagacattttttc

SEQ ID NO: 33 - DNA, 21 bp, Artificial Sequence
  Synthetic siRNA sequence targeting the gene, KNTC2
ccgagaccacttaatgacaaa

SEQ ID NO: 34 - DNA, 21 bp, Artificial Sequence
  Synthetic siRNA sequence targeting the gene, KNTC2
tccctgggtcgtgtcaggaaa

SEQ ID NO: 35 - DNA, 2209 bp, Homo sapiens
actgcgcgcgtcgtgcgtaatgacgtcagcgccggcggagaatttcaaattcgaacggctttggcgggccgaggaaggacctggtgttttgatgaccgctgtcctgtctagcagatacttgcacggtttacagaaattcggtccctgggtcgtgtcaggaaactggaaaaaaggtcataagcatgaagcgcagttcagtttccagcggtggtgctggccgcctctccatgcaggagttaagatcccaggatgtaaataaacaaggcctctatacccctcaaaccaaagagaaaccaacctttggaaagttgagtataaacaaaccgacatctgaaagaaaagtctcgctatttggcaaaagaactagtggacatggatcccggaatagtcaacttggtatattttccagttctgagaaaatcaaggacccgagaccacttaatgacaaagcattcattcagcagtgtattcgacaactctgtgagtttcttacagaaaatggttatgcacataatgtgtccatgaaatctctacaagctccctctgttaaagacttcctgaagatcttcacatttctttatggcttcctgtgcccctcatacgaacttcctgacacaaagtttgaagaagaggttccaagaatctttaaagaccttgggtatccttttgcactatccaaaagctccatgtacacagtgggggctcctcatacatggcctcacattgtggcagccttagtttggctaatagactgcatcaagatacatactgccatgaaagaaagctcacctttatttgatgatgggcagccttggggagaagaaactgaagatggaattatgcataataagttgtttttggactacaccataaaatgctatgagagttttatgagtggtgccgacagctttgatgagatgaatgcagagctgcagtcaaaactgaaggatttatttaatgtggatgcttttaagctggaatcattagaagcaaaaaacagagcattgaatgaacagattgcaagattggaacaagaaagagaaaaagaaccgaatcgtctagagtcgttgagaaaactgaaggcttccttacaaggagatgttcaaaagtatcaggcatacatgagcaatttggagtctcattcagccattcttgaccagaaattaaatggtctcaatgaggaaattgctagagtagaactagaatgtgaaacaataaaacaggagaacactcgactacagaatatcattgacaaccagaagtactcagttgcagacattgagcgaataaatcatgaaagaaatgaattgcagcagactattaataaattaaccaaggacctggaagctgaacaacagaagttgtggaatgaggagttaaaatatgccagaggcaaagaagcgattgaaacacaattagcagagtatcacaaattggctagaaaattaaaacttattcctaaaggtgctgagaattccaaaggttatgactttgaaattaagtttaatcccgaggctggtgccaactgccttgtcaaatacagggctcaagtttatgtacctcttaaggaactcctgaatgaaactgaagaagaaattaataaagccctaaataaaaaaatgggtttggaggatactttagaacaattgaatgcaatgataacagaaagcaagagaagtgtgagaactctgaaagaagaagttcaaaagctggatgatctttaccaacaaaaaattaaggaagcagaggaagaggatgaaaaatgtgccagtgagcttgagtccttggagaaacacaagcacctgctagaaagtactgttaaccaggggctcagtgaagctatgaatgaattagatgctgttcagcgggaataccaactagttgtgcaaaccacgactgaagaaagacgaaaagtgggaaataacttgcaacgtctgttagagatggttgctacacatgttgggtctgtagagaaacatcttgaggagcagattgctaaagttgatagagaatatgaagaatgcatgtcagaagatctctcggaaaatattaaagagattagagataagtatgagaagaaagctactctaattaagtcttctgaagaatgaagataaaatgttgatcatgtatatatatccatagtgaataaaattgtctcagtaaagtgtaaaaaaaaaaaaaaaaaaaaaaaaaaaaaaaaaaaaaa

SEQ ID NO: 36 - DNA, 21 bp, Artificial Sequence
  Synthetic siRNA sequence targeting the gene, PBK
aagtgtggcttgcgtaaataa

SEQ ID NO: 37 - DNA, 21 bp, Artificial Sequence
  Synthetic siRNA sequence targeting the gene, PBK
tcagtagttattagactctaa

SEQ ID NO: 38 - DNA, 1899 bp, Homo sapiens
agcgcgcgactttttgaaagccaggagggttcgaattgcaacggcagctgccgggcgtatgtgttggtgctagaggcagctgcagggtctcgctgggggccgctcgggaccaattttgaagaggtacttggccacgacttattttcacctccgacctttccttccaggcggtgagactctggactgagagtggctttcacaatggaagggatcagtaatttcaagacaccaagcaaattatcagaaaaaaagaaatctgtattatgttcaactccaactataaatatcccggcctctccgtttatgcagaagcttggctttggtactggggtaaatgtgtacctaatgaaaagatctccaagaggtttgtctcattctccttgggctgtaaaaaagattaatcctatatgtaatgatcattatcgaagtgtgtatcaaaagagactaatggatgaagctaagattttgaaaagccttcatcatccaaacattgttggttatcgtgcttttactgaagccaatgatggcagtctgtgtcttgctatggaatatggaggtgaaaagtctctaaatgacttaatagaagaacgatataaagccagccaagatccttttccagcagccataattttaaaagttgctttgaatatggcaagagggttaaagtatctgcaccaagaaaagaaactgcttcatggagacataaagtcttcaaatgttgtaattaaaggcgattttgaaacaattaaaatctgtgatgtaggagtctctctaccactggatgaaaatatgactgtgactgaccctgaggcttgttacattggcacagagccatggaaacccaaagaagctgtggaggagaatggtgttattactgacaaggcagacatatttgcctttggccttactttgtgggaaatgatgactttatcgattccacacattaatctttcaaatgatgatgatgatgaagataaaacttttgatgaaagtgattttgatgatgaagcatactatgcagcgttgggaactaggccacctattaatatggaagaactggatgaatcataccagaaagtaattgaactcttctctgtatgcactaatgaagaccctaaagatcgtccttctgctgcacacattgttgaagctctggaaacagatgtctagtgatcatctcagctgaagtgtggcttgcgtaaataactgtttattccaaaatatttacatagttactatcagtagttattagactctaaaattggcatatttgaggaccatagtttcttgttaacatatggataactatttctaatatgaaatatgcttatattggctataagcacttggaattgtactgggttttctgtaaagttttagaaactagctacataagtactttgatactgctcatgctgacttaaaacactagcagtaaaacgctgtaaactgtaacattaaattgaatgaccattacttttattaatgatctttcttaaatattctatattttaatggatctactgacattagcactttgtacagtacaaaataaagtctacatttgtttaaaacactgaaccttttgctgatgtgtttatcaaatgataactggaagctgaggagaatatgcctcaaaaagagtagctccttggatacttcagactctggttacagattgtcttgatctcttggatctcctcagatctttggtttttgctttaatttattaaatgtattttccatactgagtttaaaatttattaatttgtaccttaagcatttcccagctgtgtaaaaacaataaaactcaaataggatgataaagaataaaggacactttgggtaccagaaaaaaaaaaaaaaaaaaaaaaaaaaaaaaaaaaaaaaaaaaaaaaa

SEQ ID NO: 39 - DNA, 21 bp, Artificial Sequence
  Synthetic siRNA sequence targeting the gene, PLK1
ccggatcaagaagaatgaata

SEQ ID NO: 40 - DNA, 21 bp, Artificial Sequence
  Synthetic siRNA sequence targeting the gene, PLK1
cgcgggcaagattgtgcctaa

SEQ ID NO: 41 - DNA, 2204 bp, Homo sapiens
gagcggtgcggaggctctgctcggatcgaggtctgcagcgcagcttcgggagcatgagtgctgcagtgactgcagggaagctggcacgggcaccggccgaccctgggaaagccggggtccccggagttgcagctcccggagctccggcggcggctccaccggcgaaagagatcccggaggtcctagtggacccacgcagccggcggcgctatgtgcggggccgctttttgggcaagggcggctttgccaagtgcttcgagatctcggacgcggacaccaaggaggtgttcgcgggcaagattgtgcctaagtctctgctgctcaagccgcaccagagggagaagatgtccatggaaatatccattcaccgcagcctcgcccaccagcacgtcgtaggattccacggctttttcgaggacaacgacttcgtgttcgtggtgttggagctctgccgccggaggtctctcctggagctgcacaagaggaggaaagccctgactgagcctgaggcccgatactacctacggcaaattgtgcttggctgccagtacctgcaccgaaaccgagttattcatcgagacctcaagctgggcaaccttttcctgaatgaagatctggaggtgaaaataggggattttggactggcaaccaaagtcgaatatgacggggagaggaagaagaccctgtgtgggactcctaattacatagctcccgaggtgctgagcaagaaagggcacagtttcgaggtggatgtgtggtccattgggtgtatcatgtataccttgttagtgggcaaaccaccttttgagacttcttgcctaaaagagacctacctccggatcaagaagaatgaatacagtattcccaagcacatcaaccccgtggccgcctccctcatccagaagatgcttcagacagatcccactgcccgcccaaccattaacgagctgcttaatgacgagttctttacttctggctatatccctgcccgtctccccatcacctgcctgaccattccaccaaggttttcgattgctcccagcagcctggaccccagcaaccggaagcccctcacagtcctcaataaaggcttggagaaccccctgcctgagcgtccccgggaaaaagaagaaccagtggttcgagagacaggtgaggtggtcgactgccacctcagtgacatgctgcagcagctgcacagtgtcaatgcctccaagccctcggagcgtgggctggtcaggcaagaggaggctgaggatcctgcctgcatccccatcttctgggtcagcaagtgggtggactattcggacaagtacggccttgggtatcagctctgtgataacagcgtgggggtgctcttcaatgactcaacacgcctcatcctctacaatgatggtgacagcctgcagtacatagagcgtgacggcactgagtcctacctcaccgtgagttcccatcccaactccttgatgaagaagatcaccctccttaaatatttccgcaattacatgagcgagcacttgctgaaggcaggtgccaacatcacgccgcgcgaaggtgatgagctcgcccggctgccctacctacggacctggttccgcacccgcagcgccatcatcctgcacctcagcaacggcagcgtgcagatcaacttcttccaggatcacaccaagctcatcttgtgcccactgatggcagccgtgacctacatcgacgagaagcgggacttccgcacataccgcctgagtctcctggaggagtacggctgctgcaaggagctggccagccggctccgctacgcccgcactatggtggacaagctgctgagctcacgctcggccagcaaccgtctcaaggcctcctaatagctgccctcccctccggactggtgccctcctcactcccacctgcatctggggcccatactggttggctcccgcggtgccatgtctgcagtgtgccccccagccccggtggctgggcagagctgcatcatccttgcaggtgggggttgctgtgtaagttatttttgtacatgttcgggtgtgggttctacagccttgtccccctccccctcaaccccaccatatgaattgtacagaatatttctattgaattcggaactgtcctttccttggctttatgcacattaaacagatgtgaatattcaaaaaaaaaaaaaaaaaaaaaaaaaaaaaaaaaaa

SEQ ID NO: 42 - DNA, 21 bp, Artificial Sequence
  Synthetic siRNA sequence targeting the gene, PRC1
aagcttcagatccaaatcgat

SEQ ID NO: 43 - DNA, 3128 bp, Homo sapiens
gcttcgccccgtggcgcggtttgaaattttgcggggctcaacggctcgcggagcggctacgcggagtgacatcgccggtgtttgcgggtggttgttgctctcggggccgtgtggagtaggtctggacctggactcacggctgcttggagcgtccgccatgaggagaagtgaggtgctggcggaggagtccatagtatgtctgcagaaagccctaaatcaccttcgggaaatatgggagctaattgggattccagaggaccagcggttacaaagaactgaggtggtaaagaagcatatcaaggaactcctggatatgatgattgctgaagaggaaagcctgaaggaaagactcatcaaaagcatatccgtctgtcagaaagagctgaacactctgtgcagcgagttacatgttgagccatttcaggaagaaggagagacgaccatcttgcaactagaaaaagatttgcgcacccaagtggaattgatgcgaaaacagaaaaaggagagaaaacaggaactgaagctacttcaagagcaagatcaagaactgtgcgaaattctttgtatgccccactatgatattgacagtgcctcagtgcccagcttagaagagctgaaccagttcaggcaacatgtgacaactttgagggaaacaaaggcttctaggcgtgaggagtttgtcagtataaagagacagatcatactgtgtatggaagaattagaccacaccccagacacaagctttgaaagagatgtggtgtgtgaagacgaagatgccttttgtttgtctttggagaatattgcaacactacaaaagttgctacggcagctggaaatgcagaaatcacaaaatgaagcagtgtgtgaggggctgcgtactcaaatccgagagctctgggacaggttgcaaatacctgaagaagaaagagaagctgtggccaccattatgtctgggtcaaaggccaaggtccggaaagcgctgcaattagaagtggatcggttggaagaactgaaaatgcaaaacatgaagaaagtgattgaggcaattcgagtggagctggttcagtactgggaccagtgcttttatagccaggagcagagacaagcttttgcccctttctgtgctgaggactacacagaaagtctgctccagctccacgatgctgagattgtgcggttaaaaaactactatgaagttcacaaggaactctttgaaggtgtccagaagtgggaagaaacctggaggcttttcttagagtttgagagaaaagcttcagatccaaatcgatttacaaaccgaggaggaaatcttctaaaagaagaaaaacaacgagccaagctccagaaaatgctgcccaagctggaagaagagttgaaggcacgaattgaattgtgggaacaggaacattcaaaggcatttatggtgaatgggcagaaattcatggagtatgtggcagaacaatgggagatgcatcgattggagaaagagagagccaagcaggaaagacaactgaagaacaaaaaacagacagagacagagatgctgtatggcagcgctcctcgaacacctagcaagcggcgaggactggctcccaatacaccgggcaaagcacgtaagctgaacactaccaccatgtccaatgctacggccaatagtagcattcggcctatctttggagggacagtctaccactcccccgtgtctcgacttcctccttctggcagcaagccagtcgctgcttccacctgttcagggaagaaaacaccccgtactggcaggcatggagccaacaaggagaacctggagctcaacggcagcatcctgagtggtgggtaccctggctcggcccccctccagcgcaacttcagcattaattctgttgccagcacctattctgagtttgcgaaggatccgtccctctctgacagttccactgttgggcttcagcgagaactttcaaaggcttccaaatctgatgctacttctggaatcctcaattcaaccaacatccagtcctgagaagccctgatcagtcaaccagctgtggcttcctgtgcctagactggacctaattatatgggggtgactttagtttttcttcagcttaggcgtgcttgaaaccttggccaggttccatgaccatgggcctaacttaaagatgtgaatgagtgttacagttgaaagcccatcataggtttagtggtcctaggagacttggttttgacttatatacatgaaaagtttatggcaagaagtgcaaattttagcatatggggcctgacttctctaccacataattctacttgctgaagcatgatcaaagcttgttttatttcaccactgtaggaaaatgattgactatgcccatccctgggggtaattttggcatgtatacctgtaactagtaattaacatcttttttgtttaggcatgttcaattaatgctgtagctatcatagctttgctcttacctgaagccttgtccccaccacacaggacagccttcctcctgaagagaatgtctttgtgtgtccgaagttgagatggcctgccctactgccaaagaggtgacaggaaggctgggagcagctttgttaaattgtgttcagttctgttacacagtgcattgccctttgttgggggtatgcatgtatgaacacacatgcttgtcggaacgctttctcggcgtttgtcccttggctctcatctcccccattcctgtgcctactttgcctgagttcttctacccccgcagttgccagccacattgggagtctgtttgttccaatgggttgagctgtctttgtcgtggagatctggaactttgcacatgtcactactggggaggtgttcctgctctagcttccacgatgaggcgccctctttacctatcctctcaatcactactcttcttgaagcactattatttattcttccgctgtctgcctgcagcagtactactgtcaacatagtgtaaatggttctcaaaagcttaccagtgtggacttggtgttagccacgctgtttactcatacagtacgtgtcctgtttttaaaatatacaattattcttaaaaataaattaaaatctgtatacttacatttcaaaaagaaaaaaaaaaaaaaaa

SEQ ID NO: 44 - DNA, 21 bp, Artificial Sequence
  Synthetic siRNA sequence targeting the gene, RFC3
tagcaccattgcaagtaacta

SEQ ID NO: 45 - DNA, 1463 bp, Homo sapiens
agtgacgtcacgagatttggagctcgcgggaaaacttgtctctgcgttgtggggaggacgcgcgctcgcgcgggattttcaagcgtaggcccccgggaactcgagctgccatgagcctctgggtggacaagtatcggccctgctccttgggacggctggactatcacaaggagcaggcggcccagctgcggaacctggtgcagtgtggtgactttcctcatctgttagtgtacggaccatcaggtgctggaaaaaagacaagaattatgtgtattctacgtgaactttatggtgttggagtggaaaaattgagaattgaacatcagaccatcacaactccatctaaaaaaaaaattgaaattagcaccattgcaagtaactaccaccttgaagttaatcctagtgatgctggaaatagtgaccgagtagtcattcaggagatgttgaaaacagtggcacaatcacaacaacttgaaacaaactctcaaagggattttaaagtggtattattgacagaagttgacaaactcaccaaagatgctcagcatgccttgcgaagaaccatggaaaaatatatgtctacctgcagattgatcttgtgctgcaattctacatctaaagtgatcccacctattcgtagtaggtgcttggcggttcgtgtgcctgctcccagcattgaagatatttgccacgtgttatctactgtgtgtaagaaggaaggtctgaatcttccttcacaactggctcatagacttgcagagaagtcttgtagaaatctcagaaaagccctgcttatgtgtgaagcctgcagagtgcaacaatatccttttactgcagatcaagaaatccctgagacagattgggaggtgtatctgagggagactgcaaatgctattgtcagtcagcaaactccacaaaggctccttgaagttcgtggaaggctgtatgagcttctaactcattgtattcctcctgagataataatgaaggcatgtaaggaggaatcaagaagctgtgacatattctgaggacctccaaagaacaacttgctactgactggacttctctcatcttttagcatgtgaagttttgcaaatataatccactttgctctgaatgtcctgacacctgcttcagagctcaagattcagactccagcctgaatgttatttcctgagaaaatccctccctgacccactgtgctggttaggtccacctgctaatcacatccacaaaaccctctgcttctccttcaaaacactcatctcctctgtaatgatcagatgaactcatgtctttctcagaggacaataaatttgatgaatggggaccatggaagtttatttccatctgtatccctacatctaactcatctgctggtacctatttgtcatgtatttaataagtacaactacctatgccaccctgcaataaaatgatattgttactttctcttctttaaa

SEQ ID NO: 46 - DNA, 21 bp, Artificial Sequence
  Synthetic siRNA sequence targeting the gene, RRM2
cacaccatgaattgtccgtaa

SEQ ID NO: 47 - DNA, 21 bp, Artificial Sequence
  Synthetic siRNA sequence targeting the gene, RRM2
gcgggattaaacagtccttta

SEQ ID NO: 48 - DNA, 3412 bp, Homo sapiens
aggcgcagccaatgggaagggtcggaggcatggcacagccaatgggaagggccggggcaccaaagccaatgggaagggccgggagcgcgcggcgcgggagatttaaaggctgctggagtgaggggtcgcccgtgcaccctgtcccagccgtcctgtcctggctgctcgctctgcttcgctgcgcctccactatgctctccctccgtgtcccgctcgcgcccatcacggacccgcagcagctgcagctctcgccgctgaaggggctcagcttggtcgacaaggagaacacgccgccggccctgagcgggacccgcgtcctggccagcaagaccgcgaggaggatcttccaggagcccacggagccgaaaactaaagcagctgcccccggcgtggaggatgagccgctgctgagagaaaacccccgccgctttgtcatcttccccatcgagtaccatgatatctggcagatgtataagaaggcagaggcttccttttggaccgccgaggaggtggacctctccaaggacattcagcactgggaatccctgaaacccgaggagagatattttatatcccatgttctggctttctttgcagcaagcgatggcatagtaaatgaaaacttggtggagcgatttagccaagaagttcagattacagaagcccgctgtttctatggcttccaaattgccatggaaaacatacattctgaaatgtatagtcttcttattgacacttacataaaagatcccaaagaaagggaatttctcttcaatgccattgaaacgatgccttgtgtcaagaagaaggcagactgggccttgcgctggattggggacaaagaggctacctatggtgaacgtgttgtagcctttgctgcagtggaaggcattttcttttccggttcttttgcgtcgatattctggctcaagaaacgaggactgatgcctggcctcacattttctaatgaacttattagcagagatgagggtttacactgtgattttgcttgcctgatgttcaaacacctggtacacaaaccatcggaggagagagtaagagaaataattatcaatgctgttcggatagaacaggagttcctcactgaggccttgcctgtgaagctcattgggatgaattgcactctaatgaagcaatacattgagtttgtggcagacagacttatgctggaactgggttttagcaaggttttcagagtagagaacccatttgactttatggagaatatttcactggaaggaaagactaacttctttgagaagagagtaggcgagtatcagaggatgggagtgatgtcaagtccaacagagaattcttttaccttggatgctgacttctaaatgaactgaagatgtgcccttacttggctgattttttttttccatctcataagaaaaatcagctgaagtgttaccaactagccacaccatgaattgtccgtaatgttcattaacagcatctttaaaactgtgtagctacctcacaaccagtcctgtctgtttatagtgctggtagtatcaccttttgccagaaggcctggctggctgtgacttaccatagcagtgacaatggcagtcttggctttaaagtgaggggtgaccctttagtgagcttagcacagcgggattaaacagtcctttaaccagcacagccagttaaaagatgcagcctcactgcttcaacgcagattttaatgtttacttaaatataaacctggcactttacaaacaaataaacattgtttgtactcacaaggcgataatagcttgatttatttggtttctacaccaaatacattctcctgaccactaatgggagccaattcacaattcactaagtgactaaagtaagttaaacttgtgtagactaagcatgtaatttttaagttttattttaatgaattaaaatatttgttaaccaactttaaagtcagtcctgtgtatacctagatattagtcagttggtgccagatagaagacaggttgtgtttttatcctgtggcttgtgtagtgtcctgggattctctgccccctctgagtagagtgttgtgggataaaggaatctctcagggcaaggagcttcttaagttaaatcactagaaatttaggggtgatctgggccttcatatgtgtgagaagccgtttcattttatttctcactgtattttcctcaacgtctggttgatgagaaaaaattcttgaagagttttcatatgtgggagctaaggtagtattgtaaaatttcaagtcatccttaaacaaaatgatccacctaagatcttgcccctgttaagtggtgaaatcaactagaggtggttcctacaagttgttcattctagttttgtttggtgtaagtaggttgtgtgagttaattcatttatatttactatgtctgttaaatcagaaattttttattatctatgttcttctagattttacctgtagttcatacttcagtcacccagtgtcttattctggcattgtctaaatctgagcattgtctagggggatcttaaactttagtaggaaaccatgagctgttaatacagtttccattcaaatattaatttcagaatgaaacataatttttttttttttttttgagatggagtctcgctctgttgcccaggctggagtgcagtggcgcgattttggctcactgtaacctccatctcctgggttcaagcaattctcctgtctcagcctccctagtagctgggactgcaggtatgtgctaccacacctggctaatttttgtatttttagtagagatggagtttcaccatattggtcaggctggtcttgaactcctgacctcaggtgatccacccacctcggcctcccaaagtgctgggattgcaggcgtgataaacaaatattcttaatagggctactttgaattaatctgcctttatgtttgggagaagaaagctgagacattgcatgaaagatgatgagagataaatgttgatcttttggccccatttgttaattgtattcagtatttgaacgtcgtcctgtttattgttagttttcttcatcatttattgtatagacaatttttaaatctctgtaatatgatacattttcctatcttttaagttattgttacctaaagttaatccagattatatggtccttatatgtgtacaacattaaaatgaaaggctttgtcttgcattgtgaggtacaggcggaagttggaatcaggttttaggattctgtctctcattagctgaataatgtgaggattaacttctgccagctcagaccatttcctaatcagttgaaagggaaacaagtatttcagtctcaaaattgaataatgcacaagtcttaagtgattaaaataaaactgttcttatgtcagtttcaaaaaaaaaaaaaaaaaa

SEQ ID NO: 49 - DNA, 21 bp, Artificial Sequence
  Synthetic siRNA sequence targeting the gene, SMC4
taccatcgtagaaatcaataa

SEQ ID NO: 50 - DNA, 21 bp, Artificial Sequence
  Synthetic siRNA sequence targeting the gene, SMC4
cagcgtttaatagagcaagaa

SEQ ID NO: 51 - DNA, 5142 bp, Homo sapiens
taggcgccattttcgagtgaaggacccggagccgaaacaccggtaggagcggggaggtgggtactacacaaccgtctccagccttggtctgagtggactgtcctgcagcgaccatgccccgtaaaggcacccagccctccactgcccggcgcagagaggaagggccgccgccgccgtcccctgacggcgccagcagcgacgcggagcctgagccgccgtccggccgcacggagagcccagccaccgccgcagagactgcaagtgaggaacttgataatagaagtttagaagagattttgaacagcattcctcctcccccgcctccagcaatgaccaatgaagctggagctcctcggcttatgataactcatattgtaaaccagaacttcaaatcctatgctggggagaaaattctgggacctttccataagcgcttttcctgtattatcgggccaaatggcagtggcaaatccaatgttattgattctatgctttttgtgtttggctatcgagcacaaaaaataagatctaaaaaactctcagtattaatacataattctgatgaacacaaggacattcagagttgtacagtagaagttcattttcaaaagataattgataaggaaggggatgattatgaagtcattcctaacagtaatttctatgtatccagaacggcctgcagagataatacttctgtctatcacataagtggaaagaaaaagacatttaaggatgttggaaatcttcttcgaagccatggaattgacttggaccataatagatttttaattttacagggtgaagttgaacaaattgctatgatgaaaccaaaaggccagactgaacacgatgagggtatgcttgaatatttagaagatataattggttgtggacggctaaatgaacctattaaagtcttgtgtcggagagttgaaatattaaatgaacacagaggagagaagttaaacagggtaaagatggtggaaaaggaaaaggatgccttagaaggagagaaaaacatagctatcgaatttcttaccttggaaaatgaaatatttagaaaaaagaatcatgtttgtcaatattatatttatgagttgcagaaacgaattgctgaaatggaaactcaaaaggaaaaaattcatgaagataccaaagaaattaatgagaagagcaatatactatcaaatgaaatgaaagctaagaataaagatgtaaaagatacagaaaagaaactgaataaaattacaaaatttattgaggagaataaagaaaaatttacacagctagatttggaagatgttcaagttagagaaaagttaaaacatgccacgagtaaagccaaaaaactggagaaacaacttcaaaaagataaagaaaaggttgaagaatttaaaagtatacctgccaagagtaacaatatcattaatgaaacaacaaccagaaacaatgccctcgagaaggaaaaagagaaagaagaaaaaaaattaaaggaagttatggatagccttaaacaggaaacacaagggcttcagaaagaaaaagaaagtcgagagaaagaacttatgggtttcagcaaatcggtaaatgaagcacgttcaaagatggatgtagcccagtcagaacttgatatctatctcagtcgtcataatactgcagtgtctcaattaactaaggctaaggaagctctaattgcagcttctgagactctcaaagaaaggaaagctgcaatcagagatatagaaggaaaactccctcaaactgaacaagaattaaaggagaaagaaaaagaacttcaaaaacttacacaagaagaaacaaactttaaaagtttggttcatgatctctttcaaaaagttgaagaagcaaagagctcattagcaatgaatcgaagtagggggaaagtccttgatgcaataattcaagaaaaaaaatctggcaggattccaggaatatatggaagattgggggacttaggagccattgatgaaaaatacgacgtggctatatcatcctgttgtcatgcactggactacattgttgttgattctattgatatagcccaagaatgtgtaaacttccttaaaagacaaaatattggagttgcaacctttataggtttagataagatggctgtatgggcgaaaaagatgaccgaaattcaaactcctgaaaatactcctcgtttatttgatttagtaaaagtaaaagatgagaaaattcgccaagctttttattttgctttacgagataccttagtagctgacaacttggatcaagccacaagagtagcatatcaaaaagatagaagatggagagtggtaactttacagggacaaatcatagaacagtcaggtacaatgactggtggtggaagcaaagtaatgaaaggaagaatgggttcctcacttgttattgaaatctctgaagaagaggtaaacaaaatggaatcacagttgcaaaacgactctaaaaaagcaatgcaaatccaagaacagaaagtacaacttgaagaaagagtagttaagttacggcatagtgaacgagaaatgaggaacacactagaaaaatttactgcaagcatccagcgtttaatagagcaagaagaatatttgaatgtccaagttaaggaacttgaagctaatgtacttgctacagcccctgacaaaaaaaagcagaaattgctagaagaaaacgttagtgctttcaaaacagaatatgatgctgtggctgagaaagctggtaaagtagaagctgaggttaaacgcttacacaataccatcgtagaaatcaataatcataaactcaaggcccaacaagacaaacttgataaaataaataagcaattagatgaatgtgcttctgctattactaaagcccaagtagcaatcaagactgctgacagaaaccttcaaaaggcacaagactctgtcttgcgtacagagaaagaaataaaagatactgagaaagaggtggatgacctaacagcagagctgaaaagtcttgaggacaaagcagcagaggtcgtaaagaatacaaatgctgcagaggaatccttaccagagatccagaaagaacatcgcaatctgcttcaagaattaaaagttattcaagaaaatgaacatgctcttcaaaaagatgcacttagtattaagttgaaacttgaacaaatagatggtcacattgctgaacataattctaaaataaaatattggcacaaagagatttcaaaaatatcactgcatcctatagaagataatcctattgaagagatttcggttctaagcccagaggatcttgaagcgatcaagaatccagattctataacaaatcaaattgcacttttggaagcccggtgtcatgaaatgaaaccaaacctcggtgccatcgcagagtataaaaagaaggaagaattgtatttgcaacgggtagcagaattggacaaaattacttatgaaagagacagttttagacaggcatatgaagatcttcggaaacaaaggcttaatgaatttatggcaggtttttatataataacaaataaattaaaggaaaattaccaaatgcttactttgggaggggacgccgaactcgagcttgtagacagcttggatcctttctctgaaggaatcatgttcagtgttcgaccacctaagaaaagttggaaaaagatcttcaacctttcgggaggagagaaaacacttagttcattggctttagtatttgctcttcaccactacaagcccactcccctttacttcatggatgagattgatgcagcccttgattttaaaaatgtgtccattgttgcattttatatatatgaacaaacaaaaaatgcacagttcataataatttctcttcgaaataatatgtttgagatttcggatagacttattggaatttacaagacatacaacataacaaaaagtgttgctgtaaatccaaaagaaattgcatctaagggactttgttgaactttatgctgaagattcttcaagttgattcagtgtattactgatttttttctatttgtaaaggattatgagttgtataaaatacatactccctaaactagatcatgaaactggtttctgttttatgcagttgtcatttgtaaagtctaataaaatattctctataattgcttctagattacaaaaatatgacaatcttgtaagtagcagactatggagaaaaatgagttacctggagggtcaggtaacttgccaaactaaaaagtatgttagttgaggcaaagtcctaagcaaggttgtgctatcaaggctcagcataccttcgtgggcctttgatttaccaacactggaaatgcctgccaactaatcttggatagattctttaaggcattccacttagcttgccagttgagacaatcaccacagttattacccaaatactatgaacatatttttgtaaaccagtcattctgaattatagtgatgagaatttaaatatatgcttttctagaatttgatgtttgaccatttatgacttaattaccagagagccagtaaattaggacagtgtttcaacaagcctaggctatctcgtaagttgaaaaatatcccactatagttgcttcatgagtatgaagtaagatggcctctgatttacactggttcaatttacaaattttcaactttatgataggtttatccgggtactaaatgcatttcaacttgatagtttcaacttatgataggtttaccaggatgtagtcccactgttgaggagcatctatttaggggttaattactttagtaataagtggaaagtaagataccttgagtaatgtttgcctataaaattgtcagcgtatttttacactattggctcaagaatgttataatgctaagggacataagttggcaaccacttggtttttggaaggactttcggtattgtattagaagtctgccctagctgttaaatttctgggtatttatcctaaggaattaattaaagagttaattgttcctttcttcagtgggccattgttttagatatttaaaaaatccaacagtttctatcataatgtaactgtaaaaatgtaaacacattattagcatggacttttaaataaagatttaaagaaagcaaaaaaaaaaaaaaaaaaaa

SEQ ID NO: 52 - DNA, 21 bp, Artificial Sequence
  Synthetic siRNA sequence targeting the gene, TEX10
ctccgaatttatgatccacaa

SEQ ID NO: 53 - DNA, 3042 bp, Homo sapiens
tttgaaaacacgctccgggagctagagcctgaggtcggcggcgcacgctgttgccccgtgggcttctgctccctcgcttgtcttctcgggcttctcgccccggccgcggccgggtcctcagtagtcgagaatgactaaaaaaagaaaacgccaacatgattttcaaaaagtgaaattgaaagttggtaaaaagaagcccaagttacaaaatgctactcctacaaactttaaaacaaagactatacatctgcctgagcaactcaaagaggatggaacacttccaacaaacaatagaaaacttaacataaaggatttgctgtcacagatgcatcactacaatgctggggttaaacaaagtgctcttcttggacttaaagaccttttgtctcaatacccatttataattgatgcacacctttcaaacatattaagtgaagtgactgctgtgtttacagataaagatgctaatgtacgattagcagcagttcaacttcttcaattcctggcccccaaaatacgagctgaacaaatttctccattttttcctttggtaagtgcccatctctctagtgccatgactcacattactgaaggaattcaggaggactctttaaaagttttggacattctgctggaacagtacccagctctaattactggccgtagcagcatattgcttaagaattttgtagaacttatttctcatcagcagctgtccaaaggactgataaatagagacagatcccagtcctggatactttctgtaaatcctaatcggagactcacttctcagcaatggaggctgaaagtcttagtgagactcagtaaattccttcaggccttggcagatggatccagtaggttgagagaaagtgaaggacttcaggaacagaaagaaaatccccatgccactagcaactccatttttatcaactggaaggaacatgccaacgaccagcaacacatccaggtttatgaaaatgggggttcacagccaaatgtcagttcacagttcaggctacggtatctggttggaggactgagtggtgtggatgaaggcctgtcatctactgaaaacctgaaaggatttattgagataataattccattgctaattgaatgctgggttgaagctgtacctccacaactagctactcctgttgggaatggtatagaacgagaacctctacaggttatgcagcaagttcttaatattatttcccttctgtggaaactctctaaacaacaggatgaaacccataaattggagtcatggcttcgaaagaactaccttattgattttaaacaccattttatgagtcgttttccatatgtcttaaaagaaataaccaagcacaaaaggaaagagccaaataaaagcatcaagcattgcacagttctctccaataacatagatcgtctcttactgaatttaacactgtctgatatcatggtctccctggcaaatgcgtcaaccttgcagaaggattgcagttggatagaaatgataaggaaatttgtaacagagacccttgaagatggctctaggctaaatagtaagcaactgaacagattgctgggagtatcctggaggttaatgcaaatacagccaaacagagaggacacagagactcttattaaggcagtttatacattatatcagcagaggggccttatccttccagttcggactttgttattgaagtttttcagtaaaatctatcagacagaagaactgagatcttgtagattcagatatcgtagtaaagtgttatcccgttggctggctggcttaccattgcaacttgctcatcttggctcccgaaatcctgagctctctacacagcttatcgatatcattcataccgctgcagcacgagcaaataaagaattactaaaaagtttacaagctactgccctccgaatttatgatccacaagaaggtgctgtggtggttctccctgcagactctcagcagcgtttggttcagcttgtatatttcctacccagtctgccggctgatttgctttctcggttaagtcgttgctgtattatgggaagactcagttcaagtttggctgccatgcttatcgggatactgcacatgagatcatcattttctgggtggaagtattcagctaaagactggttgatgagtgatgtagactatttcagcttcttattttccacacttacagggttttcgaaagaggagttgacttggcttcagagccttcgaggagttcctcatgtcatccagacacagctttcccctgtgcttctctaccttacagatttggatcaatttttacaccactgggatgtaacagaggcagtttttcacagtttattggttattcctgcccgaagtcagaactttgacatcttgcaaagtgccatcagtaagcatttggttgggttgactgtaattcctgacagcacggctggctgtgtttttggtgttatctgtaagctcctggatcatacttgtgtagttagtgagactctactgccatttctggcttcttgttgctacagtcttctttattttctgctcactatagagaaaggggaagcagaacatctaagaaagagggacaagctgtggggggtctgtgtctccatcctggctctcttgcctcgagtcctcaggttgatgctgcagagcctgcgggtgaacagagttgggcctgaggagctgcctgttgtgggccagctgcttcgactgctgcttcagcatgcacccctcaggactcatatgttgaccaatgcgatcttggtgcagcagatcatcaagaatatcacgacattgaagagtggaagtgttcaggaacagtggctcacagacttacattactgctttaacgtgtatatcactgggcatccccaagggcccagtgcactggctacagtgtattgaagaggccatagtacctcctgtttgaagttgtttattcacatctatcttatttgaagaaaaagactgatgtaatagatctttgtcattaaagctgaacttttaaaaaaaaaaaaaaaaaaaaa

SEQ ID NO: 54 - DNA, 21 bp, Artificial Sequence
  Synthetic siRNA sequence targeting the gene, TPX2
aaggctaataatgagatgtaa

SEQ ID NO: 55 - DNA, 3685 bp, Homo sapiens
agtggactcacgcaggcgcaggagactacacttcccaggaactccgggccgcgttgttcgctggtacctccttctgacttccggtattgctgcggtctgtagggccaatcgggagcctggaattgctttcccggcgctctgattggtgcattcgactaggctgcctgggttcaaaatttcaacgatactgaatgagtcccgcggcgggttggctcgcgcttcgttgtcagatctgaggcgaggctaggtgagccgtgggaagaaaagagggagcagctagggcgcgggtctccctcctcccggagtttggaacggctgaagttcaccttccagcccctagcgccgttcgcgccgctaggcctggcttctgaggcggttgcggtgctcggtcgccgcctaggcggggcagggtgcgagcaggggcttcgggccacgcttctcttggcgacaggattttgctgtgaagtccgtccgggaaacggaggaaaaaaagagttgcgggaggctgtcggctaataacggttcttgatacatatttgccagacttcaagatttcagaaaaggggtgaaagagaagattgcaactttgagtcagacctgtaggcctgatagactgattaaaccacagaaggtgacctgctgagaaaagtggtacaaatactgggaaaaacctgctcttctgcgttaagtgggagacaatgtcacaagttaaaagctcttattcctatgatgccccctcggatttcatcaatttttcatccttggatgatgaaggagatactcaaaacatagattcatggtttgaggagaaggccaatttggagaataagttactggggaagaatggaactggagggctttttcagggcaaaactcctttgagaaaggctaatcttcagcaagctattgtcacacctttgaaaccagttgacaacacttactacaaagaggcagaaaaagaaaatcttgtggaacaatccattccgtcaaatgcttgttcttccctggaagttgaggcagccatatcaagaaaaactccagcccagcctcagagaagatctcttaggctttctgctcagaaggatttggaacagaaagaaaagcatcatgtaaaaatgaaagccaagagatgtgccactcctgtaatcatcgatgaaattctaccctctaagaaaatgaaagtttctaacaacaaaaagaagccagaggaagaaggcagtgctcatcaagatactgctgaaaagaatgcatcttccccagagaaagccaagggtagacatactgtgccttgtatgccacctgcaaagcagaagtttctaaaaagtactgaggagcaagagctggagaagagtatgaaaatgcagcaagaggtggtggagatgcggaaaaagaatgaagaattcaagaaacttgctctggctggaatagggcaacctgtgaagaaatcagtgagccaggtcaccaaatcagttgacttccacttccgcacagatgagcgaatcaaacaacatcctaagaaccaggaggaatataaggaagtgaactttacatctgaactacgaaagcatccttcatctcctgcccgagtgactaagggatgtaccattgttaagcctttcaacctgtcccaaggaaagaaaagaacatttgatgaaacagtttctacatatgtgccccttgcacagcaagttgaagacttccataaacgaacccctaacagatatcatttgaggagcaagaaggatgatattaacctgttaccctccaaatcttctgtgaccaagatttgcagagacccacagactcctgtactgcaaaccaaacaccgtgcacgggctgtgacctgcaaaagtacagcagagctggaggctgaggagctcgagaaattgcaacaatacaaattcaaagcacgtgaacttgatcccagaatacttgaaggtgggcccatcttgcccaagaaaccacctgtgaaaccacccaccgagcctattggctttgatttggaaattgagaaaagaatccaggagcgagaatcaaagaagaaaacagaggatgaacactttgaatttcattccagaccttgccctactaagattttggaagatgttgtgggtgttcctgaaaagaaggtacttccaatcaccgtccccaagtcaccagcctttgcattgaagaacagaattcgaatgcccaccaaagaagatgaggaagaggacgaaccggtagtgataaaagctcaacctgtgccacattatggggtgccttttaagccccaaatcccagaggcaagaactgtggaaatatgccctttctcgtttgattctcgagacaaagaacgtcagttacagaaggagaagaaaataaaagaactgcagaaaggggaggtgcccaagttcaaggcacttcccttgcctcattttgacaccattaacctgccagagaagaaggtaaagaatgtgacccagattgaacctttctgcttggagactgacagaagaggtgctctgaaggcacagacttggaagcaccagctggaagaagaactgagacagcagaaagaagcagcttgtttcaaggctcgtccaaacaccgtcatctctcaggagccctttgttcccaagaaagagaagaaatcagttgctgagggcctttctggttctctagttcaggaaccttttcagctggctactgagaagagagccaaagagcggcaggagctggagaagagaatggctgaggtagaagcccagaaagcccagcagttggaggaggccagactacaggaggaagagcagaaaaaagaggagctggccaggctacggagagaactggtgcataaggcaaatccaatacgcaagtaccagggtctggagataaagtcaagtgaccagcctctgactgtgcctgtatctcccaaattctccactcgattccactgctaaactcagctgtgagctgcggataccgcccggcaatgggacctgctcttaacctcaaacctaggaccgtcttgctttgtcattgggcatggagagaacccatttctccagacttttacctacccgtgcctgagaaagcatacttgacaactgtggactccagttttgttgagaattgttttcttacattactaaggctaataatgagatgtaactcatgaatgtctcgattagactccatgtagttacttcctttaaaccatcagccggccttttatatgggtcttcactctgactagaatttagtctctgtgtcagcacagtgtaatctctattgctattgccccttacgactctcaccctctccccactttttttaaaaattttaaccagaaaataaagatagttaaatcctaagatagagattaagtcatggtttaaatgaggaacaatcagtaaatcagattctgtcctcttctctgcataccgtgaatttatagttaaggatccctttgctgtgagggtagaaaacctcaccaactgcaccagtgaggaagaagactgcgtggattcatggggagcctcacagcagccacgcagcaggctctgggtggggctgccgttaaggcacgttctttccttactggtgctgataacaacagggaaccgtgcagtgtgcattttaagacctggcctggaataaatacgttttgtctttccctcaaaaaaaaaaaaaaaaaaaaaaa

SEQ ID NO: 56 - DNA, 63 bp, Artificial sequence
  HJURP shRNA forward synthetic sequence
gatccccgagcgattcatcttcatcattcaagagatgatgaagatgaatcgctctttttggaa

SEQ ID NO: 57 - DNA, 64 bp, Artificial sequence
  HJURP shRNA reverse synthetic sequence
agcttttccaaaaagagcgattcatcttcatcatctcttgaatgatgaagatgaatcgctcggg

(19) United States
(12) Patent Application Publication
(10) Pub. No.: US 2015/0080410 A1
(43) Pub. Date: Mar. 19, 2015

(54) Cancer Specific Mitotic Network

(71) Applicant: The Regents of the University of California, Oakland, CA (US)

(72) Inventors: Zhi Hu, Lake Oswego, OR (US); Jian-hua Mao, Moraga, CA (US); Wen-Lin Kuo, Lin-Kou Town (TW); Ge Huang, Lake Oswego, OR (US); Joe W. Gray, Lake Oswego, OR (US)

(73) Assignee: THE REGENTS OF THE UNIVERSITY OF CALIFORNIA, Oakland, CA (US)

(21) Appl. No.: 14/281,888

(22) Filed: May 19, 2014

Related U.S. Application Data
(63) Continuation of application No. 13/291,992, filed on Nov. 8, 2011, now abandoned.
Application No. 13/291,992 is a continuation-in-part of application No. PCT/US2010/034274, filed on May 10, 2010.
(60) Provisional application No. 61/176,840, filed on May 8, 2009.
(60) Provisional application No. 61/220,555, filed on Jun. 25, 2009.
(60) Provisional application No. 61/285,159, filed on Dec. 9, 2009.

(51) Int. Cl. C12Q 1/68; A61K 31/437; A61K 31/496

(52) U.S. Cl. CPC C12Q 1/6886; A61K 31/496; A61K 31/437; C12Q 2600/118; C12Q 2600/158; C12Q 2600/16
USPC 514/254.06; 514/300

(57) ABSTRACT

Developed here is a mitotic network comprising a signature of up to 54 genes, and including also sub-sets of genes within the signature, which can identify members by requiring higher correlation values for a signature gene. The present mitotic network provides for methods for prognosis and diagnosis of various cancers. The mitotic network is conserved across cancers exhibiting aberrant mitotic activity and several genes in the network act as therapeutic targets. Development of other inhibitors of mitosis can apply expression values of the genes in the mitotic network from patient tissue to select patients during clinical validation of the new drugs.

### CROSS-REFERENCE TO RELATED APPLICATIONS

This application is a continuation-in-part of International Patent Application No. PCT/US10/34274 filed on May 10, 2010, which claims priority to U.S. Provisional Patent Application No. 61/176,840, filed on May 8, 2009, U.S. Provisional Patent Application No. 61/220,555, filed on Jun. 25, 2009, and U.S. Provisional Patent Application No. 61/285,159 filed on Dec. 9, 2009, all of which are hereby incorporated by reference in their entirety.

### STATEMENT OF GOVERNMENTAL SUPPORT

This work was supported under Contract No. DE-ACO2-05CH11231 awarded by the Department of Energy, and under Grant No. CA 126551 awarded by the National Institutes of Health/National Cancer Institute. The work is also supported in part under work for others Agreement (WFO) LB 06-002417 from Glaxo Smith Kline. The government has certain rights in the invention.

### REFERENCE TO SEQUENCE LISTING

The attached sequence listing in paper form is hereby incorporated by reference.

### FIELD OF THE INVENTION

The invention relates to gene profiling to identify and prognose disease conditions and to direct treatment.

### BACKGROUND OF THE INVENTION

Mitosis is the process in which a eukaryotic cell divides the chromosomes in its cell nucleus, into two identical sets in two daughter nuclei. It is generally followed immediately by cytokinesis, which divides the nuclei, cytoplasm, organelles and cell membrane into two daughter cells containing roughly equal shares of these cellular components. Mitosis and cytokinesis together define the mitotic (M) phase of the cell cycle—the division of the mother cell into two daughter cells, genetically identical to each other and to their parent cell. Mitosis occurs exclusively in eukaryotic cells, including mammalian cells.

The process of mitosis is complex and highly regulated. The sequence of events is divided into phases, corresponding to the completion of one set of activities and the start of the next. These stages are prophase, prometaphase, metaphase, anaphase and telophase. During the process of mitosis the pairs of chromosomes condense and attach to fibers that pull the sister chromatids to opposite sides of the cell. The cell then divides in cytokinesis, to produce two identical daughter cells. Because cytokinesis usually occurs in conjunction with mitosis, “mitosis” is often used interchangeably with “mitotic phase”.

Errors in mitosis can either kill a cell through apoptosis or cause mutations that may lead to cancer. Although errors in mitosis are rare, the process may go wrong, especially during early cellular divisions in the zygote. Mitotic errors can be especially dangerous to the organism because future offspring from this parent cell will carry the same disorder.

In non-disjunction, a chromosome may fail to separate during anaphase. One daughter cell will receive both sister chromosomes and the other will receive none. This results in the former cell having three chromosomes coding for the same thing (two sisters and a homologue), a condition known as trisomy, and the latter cell having only one chromosome (the homologous chromosome), a condition known as monosomy. These cells are considered aneuploidic cells and these abnormal cells can cause cancer.

Mitosis is a traumatic process. The cell goes through dramatic changes in ultrastructure, its organelles disintegrate and reform in a matter of hours, and chromosomes are jostled constantly by probing microtubules. Occasionally, chromosomes may become damaged. An arm of the chromosome may be broken and the fragment lost, causing deletion. The fragment may incorrectly reattach to another, non-homologous chromosome, causing translocation. It may reattach to the original chromosome, but in reverse orientation, causing inversion. Or, it may be treated erroneously as a separate chromosome, causing chromosomal duplication. The effects of these genetic abnormalities depend on the specific nature of the error. It may range from no noticeable effect, cancer induction, or organism death.

Functional studies of the mitotic apparatus reveal an intricate network of structural proteins, molecular motors, regulatory kinases and phosphatases that regulate entry into and progression through mitosis. It is now known that deregulation of aspects of this network leads to increased genome instability, carcinogenesis and tumor progression. This knowledge, plus appreciation of the fact that increased mitotic activity is a hallmark of aggressive cancers have stimulated development of small molecule inhibitors of several mitotic apparatus proteins as anticancer agents and some are now entering clinical trials. Toyoshima-Morimoto, F., Taniguchi, E., Shinya, N., Iwamatsu, A. & Nishida, E. Nature 410, 215-20 (2001), Barr, F. A., Sillje, H. H. & Nigg, E. A. Nat. Rev. Mol. Cell. Biol. 5, 429-440 (2004), McInnes, C. et al. Nat. Chem. Biol. 2, 608-617 (2006), Winkles, J. A. & Alberts, G. F. Oncogene 24, 260-266 (2005), Yamada, S. et al. Oncogene 23, 5901-5911(2004).

### SUMMARY OF THE INVENTION

The invention provides for a method comprising, identifying in a cell from tissue of a patient a gene signature comprising increased expression of genes in Table 4, Table 4 comprising genes of a mitotic network, and assigning a score to the signature. The method further comprising, calculating a score from expression data of genes in Table 4 based on a weighted average of mRNA expression of the genes in the signature. The calculating step further comprising, determining the weighted average for a gene in the signature from a regression coefficient for the gene, the regression coefficient established from an expression level of the gene in a cancer cell line and a sensitivity of the cancer cell line to an inhibitor of mitosis.

The invention further provides a method comprising, identifying in cell from tissue of a patient an expression level of a gene selected from MELK, SMC4, TEX10, AURKA, HJURP, BUB1, RFC3, and CCNB2, the genes comprising a signature, and forming a score for the gene signature specific to the patient, wherein the score is adapted to inform medical treatment, the treatment comprising administering an inhibitor of mitosis.

In one aspect, a gene signature of a cell from patient tissue including expression levels of genes from a mitotic network comprising the genes in Table 4, and a score for the signature derived by comparison to expression of the genes in a reference cell, wherein the score is adapted to inform a determination selected from diagnosis, prognosis, and treatment of the patient.

In one embodiment, a method comprising, blocking expression in a cancer cell of a gene in a cancer specific mitotic network using a test drug, and detecting cell death, wherein cell death indicates the test drug is a potential anti-mitotic anti-cancer therapeutic.

In another embodiment, a method comprising, over-expressing a gene in Table 4 in a germ cell of a mammal to form a transgenic mammal, observing the transgenic mammal for tumor production, and screening for an expression inhibitor of a gene in Table 4 by observing tumor reduction.

In another embodiment, a set of candidate siRNA therapeutic targets: PLK1, SMC4, PBK, KIF14, NCAPD2, RRM2, CENPA, KNTC2, KIF23, RFC3, EXO1, LMNB2, TEX10, DEPDC1, DDX39, MAD2L1, C10orf13, FAM64A, TPX2, AURKA, TTK.

### DESCRIPTION OF THE DRAWINGS

FIG. 1 shows that the inhibitors of PLK1, CENPE and AURKA selectively target the basal subtype of human breast cancer cells. The GI50 Values of GSK461364 (PLK1 inhibitor), GSK923295 (CENPE inhibitor) and GSK1070916 (AURKA inhibitor) in breast cancer cells and non-malignant mammary epithelial cells were ranked according to GI50 values. The calculation of GI50 is shown in methods.

FIG. 2 shows the mitotic gene transcript network is conserved in breast cancer cells. The subset of 272 Affymetrix gene probes that reached a statistical significant correlation with either PLK1, CENPE, or AURKB in 50-53 breast cancer cell lines were selected to construct mitotic gene network using ExpressionCorrelation software. The significant p-Value was calculated by 1000 permutations. Network figures were generated by using Cytoscape version 2.6.1 (<http://www.cytoscape.org>). The edges connect significantly correlated genes. The mitotic gene network confirmed in primary breast tumors as dataset1 (Quigley, D. A. et al. Nature (2009))and dataset2 (Strebhardt, K. & Ullrich, A. Nat. Rev. Cancer 6, 321-330 (2006)).

FIGS. 3A-D shows that the mitotic network activity (MNA) is higher in basal subtype of breast cancer and is associated with prognosis in breast cancer cell lines. Mitotic network activity is defined as the sum of transcriptional expression levels of 54 genes. FIG. 3A: The mitotic network activity is higher in IDC(invasive ductal carcinoma) than that of normal breast tissues, p<0.001 (GEO accession numbers, GSE GSE10780) FIG. 3B: The mitotic network activity is significant associated with mitotic counts which is a histopathological mitotic activity index to measure mitotic activity (GEO accession numbers, GSE19) FIG. 3C, D: Breast cancer cell lines clustered based on mitotic apparatus gene transcript network (FIG. 2) using unsupervised cluster analysis. All genes in the mitotic network expressed higher level in basal subtypes than in luminal subtypes of breast cancers, p<0.001. Each row in the heat map represents a gene, and each column represents a cell line or tumor sample. As shown in the color bar, gray indicates higher gene expression, and light gray indicates lower gene expression. Higher mitotic network activity is significantly associated with poorer prognosis in four cohorts of patients with breast cancer. In breast cancer cell lines(FIG. 3D), the mitotic network activity is significantly higher in basal subtype in comparison of luminal subtype.

FIG. 4 shows that the mitotic network activity is higher in basal subtype of breast cancer and is associated with prognosis in primary breast tumors clustered based on mitotic apparatus gene transcript network (see FIG. 3) using unsupervised cluster analysis. All genes in the mitotic network expressed higher level in basal subtypes than in luminal subtypes of breast cancers, p<0.001. Each row in the heat map represents a gene, and each column represents a cell line or tumor sample. As shown in the color bar, gray indicates higher gene expression, and lightgray indicates lower gene expression. Higher mitotic network activity is significantly associated with poorer prognosis in four cohorts of patients with breast cancer. In primary breast cancers (panels 4B and 4D), the mitotic network activity is significantly higher in basal subtype in comparison of other types (e.g., normal-like, luminal, erbb2 positive). FIGS. 4A and B shows the mitotic network activity for dataset1(p<0.001) and FIGS. 4C and 4D shows the mitotic network activity for Curtis dataset, p<0.0001 using ANOVA.

FIGS. 5A-D show Kaplan-Meier curves for tumors with the highest ⅓^{rd} of MNAI values and lowest ⅓^{rd} of MNAI values. Higher mitotic network activity is significantly associated with reduced survival duration in four independent breast cancer studies using log-rank tests. A, Dataset 1, p<0.05. B, Dataset 2 (GEO accession number, GSE1456), p<0.0005 C, Dataset 3 (GEO accession number, GSE1456), p<0.0001. D, Dataset 4 (GEO accession number, GSE4922), p<0.0001. Dataset 1: Chin, K. et al. Genomic and transcriptional aberrations linked to breast cancer pathophysiologies. Cancer Cell 10, 529-541 (2006), Dataset 2: GSE2034 (Wang, Y. et al. Gene-expression profiles to predict distant metastasis of lymph-node-negative primary breast cancer. Lancet 365, 671-679 (2005)), Dataset 3: GSE1456 (Pawitan, Y. et al. Gene expression profiling spares early breast cancer patients from adjuvant therapy: derived and validated in two population-based cohorts. Breast Cancer Res 7, R953-964 (2005)), and Dataset 4: GSE4922(Ivshina, A. V. et al. Genetic reclassification of histologic grade delineates new clinical subtypes of breast cancer. Cancer Res 66, 10292-10301 (2006)). Data were pre-processed as described in the original publication.

FIGS. 6A, 6B and 6C show correlations between GI₅₀ values and MNA values. Network activity can predict PLK1, CENPE, AURKB inhibitor sensitivity. Higher activity associated with higher sensitivity(left panel of FIGS. 6A, 6B and). The distribution of GI50 values of inhibitors of PLK1, CENPE, AURKB in basal and luminal type cells (right panel of FIG. 6A, 6B and 6c). Responses in MNAI high and MNAI low cell lines determined using two-tailed Manu-Whitney U test.

FIGS. 7A and 7B show that the mitotic gene transcript network is conserved in human malignant diseases. The mitotic gene transcript network is conserved in lung cancer (GEO accession number, GSE3141)′ ovarian cancer (GEO accession number GSE3149), Wilms tumor (GEO accession number GSE10320), prostate cancer (GEO accession number GSE8218) (Bild A H, Yao G, Chang J T, et al. Oncogenic pathway signatures in human cancers as a guide to targeted therapies. Nature 2006; 439:353-357), Glioblastomas (GEO accession number GSE13041; Lee Y, Scheck A C, Cloughesy T F, Lai A et al. Gene expression analysis of glioblastomas identifies the major molecular basis for the prognostic benefit of younger age. BMC Med Genomics 2008 Oct. 21; 1:52), Acute lymphoblastic leukemia (GEO accession number GSE12417; Metzeler K H, Hummel M, Bloomfield C D, Spiekermann K et al. An 86-probe-set gene-expression signature predicts survival in cytogenetically normal acute myeloid leukemia. Blood 2008 Nov. 15; 112(10):4193-201), Acute Myelogenous leukemia (GEO accession number, GSE12417), Lymphoblast Cell lines (GEO accession number, GSE11582; Choy E, Yelensky R, Bonakdar S, Plenge R M et al. Genetic analysis of human traits in vitro: drug response and gene expression in lymphoblastoid cell lines. PLoS Genet 2008 November; 4(11):e1000287). (see also references 1-8 below).

FIGS. 8A, 8B and 8C are graphs of dosage response and show no synergistic effect on therapy with combination of two inhibitors of mitosis. Dose response to GSK461364, GSK923295 and GSK1070916 was assayed individually or in combination in different breast cancer subtypes. FIG. 8A is a graph of response to single and combination doses of PLK1 and AURKB inhibitors; FIG. 8B is a graph of response to single and combination doses of PLK1 and CENPE inhibitors; and FIG. 8C is a graph of response to single and combination doses of CENPE and AURKB inhibitors.

FIGS. 9A, 9B and 9C are graphs showing mitotic network activity in 79 different types of tissues (GEO accession numbers, GSEGSE7307).

FIGS. 10A and 10B are bar charts showing knockdown of multiple mitotic genes can inhibit cell growth and Brdu incorporation indicating that these genes play important role in cell growth.

FIGS. 11A, 11B and 11C are bar charts showing knockdown of multiple mitotic genes can inhibit cell growth breast cancer cell lines MDAMB231, BT549 and HCC1569. Cells were transiently transfected 10 nM of siRNAs targeting mitotic genes. FIGS. 11A and 11B. Cell viability measured after 72 hours and normalized to non-specific siRNA which served as a negative control. siRNAs that induced a growth inhibition significantly lower than that achieved using a control siRNA (two sided t-test p<0,05) are indicated. FIG. 11C. Quantification of mRNA levels after siRNA knockdown. All the levels were normalized to mRNA levels after treatment with a control siRNA. These knockdown experiments show the following genes as candidate siRNA therapeutic targets: PLK1, SMC4, PBK, KIF14, NCAPD2, RRM2, CENPA, KNTC2, KIF23, RFC3, EXO1, LMNB2, TEX10, DEPDC1, DDX39, MAD2L1, C10orf13, FAM64A, TPX2, AURKA, TTK.

FIGS. 12 and 13 shows the genetic alteration (losses or gains) associated with expression of FOXM1. FIG. 12 summarizes genetic loci(losses or gains) significantly associated with the expression of mitotic network genes are illustrated in dataset of 824 breast cancer tumor samples. Each row represents a gene in mitotic network and each column represents a chromosomal locus defined SNP6 copy number data. P-values indicating the significant of the association were based an ANOVA test, where red denotes genetic alterations strongly associated with the expression of mitotic network genes (p<10⁻¹⁰), and blue indicates significant, but slightly weaker associations (10⁻¹⁰<p<10⁻⁷). The common loci that are significantly associated with the expression of mitotic network genes include: 5 (77-100 Mb), 8 (23-33 Mb), 8 (115-147 Mb), 10 (0-20 Mb), 12 (0-4 Mb), and 17 (77-89 Mb). FIG. 13 is a heatmap representing narrowed genetic alterations on chromosomes 8q(120-132 Mb), 10p(0-17.8 Mb) and 12p(0-4 Mb) and 17q(65.4-78.6 Mbp) where samples have been ordered by decreased MNAI. These regions encode the transcription factors, MYC, ZEB1, FOXM1, and SOX9 each of which has predicted binding sites in multiple genes comprising the 54 mitotic apparatus network.

FIGS. 14A, 14B, 14C, 14D, and 14E show HJURP is overexpressed in human breast cancer cell lines and primary breast tumors. (A) Protein levels of HJURP (Holliday junction recognition protein) in a large panel of human breast cancer cell lines and immortalized non-malignant mammary epithelial cells were assessed by Western blotting. Samples 30, 31 and 62 are immortalized non-malignant mammary epithelial cells 184A1N4, 184B5 and S1 respectively. (B) Normalized quantification of HJURP protein levels in the cell lines using Scion Image software are shown. The arrows indicate the immortalized non-malignant mammary epithelial cells 184A1N4, 184B5, and S1 respectively. The line shows M+1.95*SE where M is mean of 184A1N4, 184B5 and S1 protein levels and SE is standard error of 184A1N4, 184B5 and S1 protein levels. Protein level above this line was defined as overexpression. About 50% breast cancer cell lines have overexpression of HJURP. (C) FIG. 1c shows the correlation between mRNA and protein levels of HJURP in human breast cancer cell lines. HJURP expression is measured as log₂ (probe intensities) by Affymetrix microarray. The detail for protein quantification refers to Materials and Methods. R was Spearman's rho correlation coefficient. The two-tailed P-value was obtained from Spearman correlation test. (D) The HJURP protein level has a negative and significant correlation with the doubling times of cell lines. (E) HJURP mRNA expression level is significantly evaluated in invasive ductal carcinomas (IDC) in comparison to normal breast ducts. HJURP mRNA expression is assessed by Affymetrix microarray. HJURP expression is measured as log₂ (probe intensities). The microarray data were found in Gene Expression Omnibus (GEO) database GEO accession numbers [GEO:GSE10780] [16].

FIGS. 15A-I show association of HJURP mRNA levels with clinic and pathological factors in patients with breast cancer. There was no significant association between HJUPR mRNA levels and (A) ERBB2 (erythroblastic leukemia viral oncogene homolog 2) status, or (B) lymph node status, or (C) pathological stage or (D) tumor size. There were significant higher mRNA levels of HJURP in (E) estrogen receptor (ER) negative patients, (F) progesterone receptor (PR) negative patients; higher mRNA levels of HJURP were significantly associated with (G) high SBR grade, (H) younger age, and (I) Ki67 proliferation indices. HJURP expression is measured as log₂ (probe intensities) by Affymetrix microarray. The two-tailed P-values were obtained by Mann-Whitney U test for ERBB2, lymph node, ER and PR status, Kruskal-Wallis H test for pathological stage and SBR grade, and Spearman correlation for size, age, and Ki67 proliferation indices.

FIGS. 16A and 16B show the impact of HJURP expression and Ki67 proliferation indices on the disease-free and overall survival. FIG. 3 shows Kaplan-Meier survival curves for breast cancer patients according to tumor expression of HJURP. The patients from each cohort were divided into a group with high (top one-third), moderate (middle one-third) and low (bottom one-third) level of HJURP expression. HJURP expression is measured log₂ (probe intensities) as in the microarray. The same criteria were used for Ki67 proliferation indices. HJURP mRNA expression was a significant prognostic factor for disease-free and overall survival, whereas Ki67 proliferation indices were not significantly associated with prognosis. (A) Kaplan-Meier survival curves for disease-free and overall survival are presented, while (B) shows the Kaplan-Meier survival curves for disease-free and overall survival based on Ki67 proliferation indices. The P-values shown were obtained from a long-rank test.

FIGS. 17A, 17B, and 17C show validation of the association between HUJRP mRNA and prognosis in three independent cohorts. Kaplan-Meier survival curves for breast cancer patients according to tumor expression of HJURP are shown. The patients from each cohort were divided into a group with high (top one-third), moderate (middle one-third) and low (bottom one-third) level of HJURP expression. HJURP expression is measured log₂ (probe intensities) as in the microarray. The significant association between HJURP mRNA and disease-free and overall survival was validated in three independent cohorts of patients with breast cancer. FIGS. 17A, 17B, and 17C show the Kaplan-Meier survival curves for disease-free and overall survival in Dataset 1 (GSE1456), Dataset 2 (GSE7390) and Dataset 3 (NM) respectively. The P-values shown were obtained from a long-rank test.

FIGS. 18A and 18B show the validation of the association between HJURP mRNA and disease-free survival in another two independent cohorts. Kaplan-Meier survival curves for breast cancer patients according to tumor expression of HJURP are shown. The patients from each cohort were divided into a group with high (top one-third), moderate (middle one-third) and low (bottom one-third) level of HJURP expression. HJURP expression is measured log₂ (probe intensities) as in the microarray. The significant association between HJURP mRNA and disease-free survival was further validated in two independent cohorts of patients with breast cancer. FIGS. 18A and 18B show the Kaplan-Meier survival curves for disease-free survival in Dataset 4 (GSE2034) and Dataset 5 (GSE4922). The P-values shown were obtained from a long-rank test

FIGS. 19A and 19B shows the expression level of HJURP is a predictive factor for radiotherapy sensitivity. Kaplan-Meier survival curves for breast cancer patients according to radiotherapy treatment are presented. FIG. 19A shows the survival curves for disease-free survival, while FIG. 19B shows survival curves for overall survival. The P-values shown were obtained from a long-rank test.

FIGS. 20A-20F shows the HJURP mRNA level in breast cancer cell lines predicts the sensitivity to radiation treatment. FIG. 20A shows the percent of viable cells at 72 hours after different doses of radiation in breast cancer cell line MDAMB231 with a high level of HJURP and T47D with a low level of HJURP. MDAMD231 cells are more sensitive to radiation treatment than T47D cells. FIG. 20B shows the fold change of apoptosis in comparison to control (no radiation) at 72 hours after the different dose of radiation in breast cancer cell line MDAMB231 and T47D. There are more apoptosis in MDAMB231 cells than T47D cells. FIG. 20C shows the percent of viable cells at 72 hours after the different dose of radiation in breast cancer cell line BT20 with high level of HJURP and MCF10A with low level of HJURP. BT20 cells are more sensitive to radiation treatment than MCF10A cells. FIG. 20D shows the fold change of apoptosis in comparison to control (no radiation) at 72 hours after the different dose of radiation in breast cancer cell line BT20 and MCF10A. There are more apoptosis in BT20 cells than MCF10A cells. FIG. 20E shows HJURP protein levels are down-regulated by shRNA in MDAMB231 breast cancer cell lines. FIG. 20F shows that MDAMB231 breast cancer cells with shRNA against HJURP reduce the sensitivity to radiation.

FIGS. 21A-21F show the correlation between HJURP and CENPA in mRNA levels. There is a highly significant and positive correlation between HJURP and CENPA in mRNA levels within human breast cancer cell lines (FIG. 21A), Primary breast tumors (FIG. 21B), Dataset 1 (FIG. 21C), Dataset2 (FIG. 21D), Dataset4 (FIG. 21E), and Dataset5 (FIG. 21F). R shown is Spearman's rho correlation coefficient.

### DETAILED DESCRIPTION

The time and cost to conduct a clinical trial for a cancer drug can be reduced if the trial can select for likely responders to the drug. Any test to select likely responders needs to be quick, simple and provide a clear indicator of patient response potential. A specific biological test that can be conducted on patient biopsy tissue or using patient blood is ideal.

An efficient strategy for identifying biological markers of a condition, including cancer, is to identify a process or pathway that can be disrupted in the progression of the disease, and determine if there is a set or signature of genes that are characteristically expressed (generally over-expressed or under-expressed) in tissue of patients that have the condition.

In recognition of the heterogeneity of cancer, drug companies are increasingly targeting specific sub types of the disease in their efforts to make new drugs. Pathways known to be active during the condition are potential sources of protein targets for treatment. Aberrant expression of genes normally associated with mitosis have been associated with cancer. Accordingly, these genes are thought to be reasonable drug targets Inhibitors of PLK1, CENPE, and AURKB (all genes known to be active in mitosis) are in clinical trials for treating breast cancer. Others specifically targeting mitosis also exist. With regard to specificity even within the cancer tissue type, the agents GSK461364, GSK923295 and GSK1070916 which target PLK1, CENPE and AURKB, respectively, were found to be most effective against basal cell breast cancer subtypes in a panel of 50 breast cancer cell lines that included both basal and luminal cell lines.

The cancer specific mitotic network described herein preferentially weights genes that are over-expressed in breast cancer cell lines, where that cell line has shown preclinical sensitivity to an inhibitor of another mitotic target in the network. The assumption is that expression of a gene that correlates well to the expression of a target of an inhibitor that is effective in that cell (i.e., the cell is sensitive to the inhibitor) justifies a stronger weight to the gene in the network when establishing a score from the expression data in a particular patient.

A further correlation is also made in that the cell has a reduced survival rate because the mitotic inhibitors were most effective in basal cell derived cancer cell lines, and basal cell cancer patients have a lower survival rate. Increased expression of that gene in a patient sample will have a greater weight than a gene not so well correlated to expression of the target gene. An index can be developed from the expression data derived from a patient's tissue sample. A cancer specific mitotic apparatus network index (MANI) is defined that is associated with reduced survival and with responsiveness to an inhibitor such as, GSK461364, GSK923295 and GSK1070916. The three drugs tested were not synergistic in the breast cancer cell panel indicating that all are acting to inhibit aspects of the same biological process. A high score on the MANI directs use of mitotic inhibitors of the mitotic apparatus network. Several of the mitotic inhibitors used together (in sequence, serially, or rotation) may prevent the development of target specific resistance.

Detailed here is a discovery that identifies of a set of genes forming a cancer specific mitotic network. Over-expression of any one of these genes indicates that there is a disruption in the mitotic process of the cancer cell. While the mitotic process is generic to all cells, including normal non-cancerous cells, some of the genes participating in mitosis (i.e., genes in the “mitotic pathway” or genes that are co-regulated to be expressed or function in mitosis) manifest a collective over-expression. Over-expression of all of these 54 genes was observed in many cancers indicating that the cancer specific mitotic network identifies a common expression pattern in cancer. Moreover, the mitotic apparatus network was also found to be coordinately regulated in cancers of the breast, lung, ovary, prostate, brain, blood, and kidney (Wilms' tumor) as well as in immortalized lymphoblast cel lines from normal human populations and normal skin samples from corrses between M. spretus and Mus musculus mouse strains.

Subsets of genes in the mitotic network may also provide expression signatures that inform a cancer condition in a patient. The likely subsets of over-expressed genes that would associate to form a signature within the mitotic network are the 54 genes in Table 4 that are related by their function in the process of mitosis. Phases of mitosis such as prophase, prometaphase, metaphase, anaphase and telophase may define subsets of genes from the original 54 gene signature that themselves are signatures and are useful for diagnosis, prognosis, treatment and screening. For example, genes that act in prophase may form a signature for early mitotic activity within the mitotic network.

The 54 genes in the mitotic network are consistently over-expressed in many cancers, making this 54 gene signature and its subsets applicable to many cancers. Over-expression is determined relative to expression levels of the same genes in normal tissue. Over-expression of genes in the cancer specific mitotic network characterizes the cancer without purporting to identify the actual cause of the cancer. Knowledge of over-expression of the genes in the mitotic network can inform decisions and strategies for both the medical practitioner and the drug designer.

The mitotic network was determined using inhibitors of 3 members of the network. About 50 publically available cancer cell lines were treated with each of the 3 inhibitors (inhibitors of PLK1, CENPE, and AURKB), to determine a responsiveness of each cell to each inhibitor. The method of developing the mitotic network can be applied across all cancer conditions, so that other pathways may also be used to develop a drug to treat cancer by using an inhibitor of one gene active in the pathway to select for genes that correlate strongly with expression patterns of the target gene in cells that are found to be sensitive to the inhibitor treatment.

Human non-malignant and breast cancer cell lines, established from normal and human breast cancer samples, are publically available and were used in the development of the mitotic network. The source of these cells includes nearly 55 well-characterized breast cell lines with genomic information (i.e. gene copy numbers of various genes) and gene expression data such as gene signatures characteristic of each cell line. Information that directs optimal incubation conditions are detailed in The cell incubational condition of the cell lines was shown reference (Neve, R. M. et al. Cancer Cell 10, 515-527 (2006).

The inhibitor compounds used were small molecule inhibitors. Theses small-molecule inhibitors for PLK1 (GSK461364), CENPE(GSK923295) and AURKB (GSK1070916) were provided by GlaxoSmithKline Inc. Stock solutions were made at a concentration of 10 mM in DMSO and stored at −20° C. Compounds were diluted (1:5 serial dilution), ranging between 0.0768 nM to30 μM.

The cell viability and growth assay and dose response (GI50) experiments were conducted following dose-response curves that were determined according to the National Cancer Institute (NCI), National Institute of Health (NIH) guidelines. In brief, cell suspensions were plated into 96-well plates in 100 μl growth media. Inoculates were allowed a pre-incubation period of 24 hours at 37° C. for stabilization. Cells were treated with 9 doses in triplicates for 72 hours with GSK461364 or GSK1070916. Cell proliferation was measured with CellTiter-Glo® Luminescent Cell Viability Assay (Promega, Madison, Wis.). After subtraction of the baseline (the viability of the cells just before treatment, time 0), the absorbance was plotted. Total growth inhibition doses and 50% growth inhibition doses GI50 were calculated by GraphPad Prism4 software (GraphPad Software, Inc., La Jolla, Calif.).

Statistical analysis was conducted using the correlation of expression of genes in the cell lines before treatment with the inhibitors. The genes were identified using Affymetrix probes. Significant correlation means that in any given cell line, correlations were looked for that showed either an increase of expression parallel with the inhibitor used on the cell, or a decrease of expression parallel with the inhibitor target (i.e. PLK1, CENPE, or AURKB), or the opposite expression (increased when the inhibitor target expression is decreased or decreased when the inhibitor target is increased). The statistical variations of these 3 options were computer simulated out 1000 times (Pearson 1000 permutations test) to generate a true correlating gene, or to reject it. Genes expressed in a cell that was either resistant or responsive to treatment with the cellular GI50 values of GSK461364, GSK1070916 and GSK923925 were examined by Pearson correlation test. The correlations of expression patterns each of the three targets in all 50 cell lines were added together and the result was a network of 275 genes (272 correlated genes and 3 target genes with which the correlations were made), the expression patterns of which were specific to the inhibitor target expression of these three mitotic inhibitors. The list of genes was selected using the Pearson Correlation Coefficient with PLK1, CENPE, or AURKB in Affymetrix expression microarray data generated from a panel of 53 human breast cancer cell lines. The correlation cut-off was determined by 1000 permutations test.

Tumor profiles were clustered using the mitotic network genes. To evaluate differences in disease-free survival (DFS), Kaplan-Maier survival curves for the sets of patients were examined. All statistical analyses were performed using the Statistical Package for the Social Sciences version 11.5 (SPSS, Inc., Chicago, Ill.).

To construct the final cancer specific mitotic network, it was determined which of these mitotic network genes in the larger set of 275 were expressed in breast cancer tissue. The 275 genes were searched within two sets of expression data from breast tumor tissue, and the resulting set of genes was the 54 gene network shown in Table 4. All genes in the network were expressed above a certain basal level, by comparison to expression levels to a reference cell, e.g. expression of the same gene in a normal non-cancerous cell. Functional annotation of the mitotic network was gotten from Gene Ontology data base for the 54 genes (see Table 3).

A score for expression in a given patient sample can be accomplished in any number of ways, depending on the assumption made before designing the methodology. The expression values of 54 genes are measured in a patient sample. The mRNA expression for each gene receives a weight, and the expression values of all these weighted genes are added and divided by 54 (i.e. averaged). The weight assigned to each gene is determined based on a regression coefficient for the gene. The regression coefficient is established from the original expression level determinations in all the cancer cells lines across treatment with each of the three inhibitors of mitosis that were used for the development of the mitotic network. Thus, effectively, the more the gene correlated to expression levels of the target inhibitor in cells treated with the inhibitor of the target, and the more sensitive that cell was to the inhibitor, the higher the weight assigned to that gene in the mitotic network score calculation. Expression levels were simplified to the log₂ratio of absolute expression values.

With the particular scoring system devised in this invention, a score of between about 2 and 12 for a given patient sample would be expected in practice. A score of anything above about 4 is suspect of indicating cancerous tissue. Levels of expression tending to indicate cancer were in the range from about 5 to about 12. A score for a signature of gene expression will generally be premised on some of the same assumptions and conditions that developed identification of the signature, but does not always have to be. A score for the mitotic network could be calculated any number of ways while maintaining the principles on which development of the mitotic network is based. of determining over-expression, which is called the score.

The gene ontology statistics tool BiNGO (Maere, S., Heymans, K. & Kuiper, M. Bioinformatics 21, 3448-3449 (2005)) was used to test whether the gene transcripts were enriched for particular functional groups.

A relevance network was constructed using Expression Correlation found at the website <URL: http://baderlab.org/Software/ExpressionCorrelation>. Any correlation above or below given threshold values, was displayed as an ‘edge’ between two ‘nodes’ (the nodes are the two genes). Network figures were generated using Cytoscape version 2.6.1 (<URL:http://www.cytoscape.org>). The mitotic network of 54 genes was formed by selecting from the 272 genes correlated by expression with expression of the inhibitor targets in the breast cancer cell lines (for a total of 275 genes with expression data).

The following is a non-limiting list of uses that the expression data from tissue of a patient that shows increased levels (above levels seen in a reference cell) of expression of the 54 genes in the mitotic network (see Table 4 for the list of the 54 genes in the mitotic network) can provide including:
1. identify cancer in the patient: expression data from patient tissue indicating over-expression of the 54 genes (i.e. a high score) in the mitotic network means that the patient has cancer.
2. distinguish the patient's cancer from other types: overexpression of the 54 genes in the mitotic network indicates that the patient has a type of cancer that is affecting mitosis or caused by abnormal mitosis pathway activation.
3. identify how far along the cancer has progressed (because the overexpression of these genes increases as the disease worsens).
4. select a patient population for a clinical trial of a drug developed as an inhibitor of any of the 54 genes, but screening for patients over-expressing the genes in the mitotic network, assuming that this population will be responsive to an inhibitor of mitosis acting through inhibition of one or more of the genes in this network.
5. choose a target for drug development: although it has been known that inhibition of mitosis generally might be a good strategy for cancer drug discovery and development, it has not been known which targets of all the genes in the mitotic process would be best to target; the present invention identifies the subset of genes involved in mitosis which are consistently overexpressed in many cancers. The strength of this discovery is in the conservation observed by the inventors of this set of 54 genes across many tissues and cancer types.
6. screen the genes in a population of cancer patients that will respond to drugs. With clinical trials of new cancer drugs, it is important to understand the molecular features of tumors most likely to respond so that early clinical trials can be conducted in tumor subtypes most likely to benefit and so that molecular markers intended to predict response can be tested in these early clinical trials. It is also important to understand the extent to which inhibitors of diverse aspects of the mitotic apparatus are clinically equivalent since they attack the same overall biological process. Conducting efficient clinical trials relies on the ability to have sensitive monitors of whether a patient is responding to the drug being tested. In addition, it would be advantageous to be able to screen patients before the trial for whether they will be a likely responder to the drug. It also would be useful to be able to monitor early changes in the patient that correlate with levels of a disease state or effectiveness of a therapy, for example, earlier diagnosis and treatment, or shift in treatment upon changing prognosis that would signal a need to modify the regimen for maximum effectiveness.

Several generalities can be made for this invention. The cancer to which the mitotic network can be applied (i.e. for diagnosis, prognosis, and informing treatment decisions) can be any cancer shown to have active genes in the mitotic pathway. For example, such cancers as epithelial cancer, lymph cancer, sarcoma, carcinoma, and gliomas. Thus, each of the 54 genes in the mitotic network is a potential target for developing a therapeutic for cancer.

In one embodiment, the therapeutic can be a small molecule inhibitor, or an interfering RNA, such as a small inhibitory RNA or a short hairpin RNA. Interfering RNAs available from <http://www.genelink.com> and other commercial entities. Transgenic mammals can be developed using one of the transgenes of the genes listed in table 4. The transgenic mammal would be designed to over express a gene from table 4, and the developing transgenic mammal would be observed for tumor formation. If tumor formation occurs in the transgenic mammal, then a screening process for finding an inhibitor of the gene used to make the transgenic mammal would begin. Likely candidate inhibitors could be tested in the transgenic mammal for an ability to prevent tumor formation, or regress existing tumors.

In one embodiment, the methods used and described herein to develop the cancer specific mitotic network can be applied to developing any signature for any condition. Accordingly, the process is outlined as identifying a pathway active in a condition, contacting a cell from tissue (or a cell line) manifesting the condition with an inhibitor of a gene in the pathway. This gene (the target gene) is then used to find other genes in the pathway that correlate with its expression in cells that respond to its inhibition. Accordingly, the signature resulting from the pathway is a set of genes (i.e. one or more genes in addition to the initial gene for which an inhibitor was made) that correlate well to expression levels of the target gene in cells that respond to inhibition of the target gene. Developing a score for this signature is accomplished by using an average of mRNA expression levels of the genes in the signature across a plurality of cell lines (or a defined unit that can be tested for responsiveness to the inhibitor), and each mRNA value for each gene in the signature is weighted based on how closely the gene expression correlates to expression of the gene (target or reference gene) for which an inhibitor was made, the correlations only being used from expression data in cells that are responsive to the inhibitor. Such a signature can be developed using one target (and one inhibitor), but would become more robust with the addition of each additional target gene and inhibitor in the pathway. As with the mitotic network (the 54 gene signature as applied to any condition), and the cancer specific mitotic network (the 54 gene network as applied to a cancer condition), a signature developed by the method just described can inform diagnosis and prognosis of the condition, and can inform treatment decisions of the condition.

In another embodiment, the invention provides a set of molecular determinants of responses to small molecule inhibitors of aurora kinase B (AURKB) (GSK1070916), Polo-like kinase 1 (PLK1) (GSK461364) and the centrosome associated protein E (CENPE) (GSK923295). Although the three proteins targeted by these drugs contribute to mitotic function, their contributions differ substantially and some contribute in addition to processes other than mitosis.

PLK1 is a 68 kDa protein comprises a serine/threonine kinase domain and a polo-box domain that targets Plk1 within the mitotic apparatus. PLK1 participates in many aspects of mitosis including regulation of mitotic checkpoints, centrosome maturation, specification of the cleavage plane, spindle assembly, the removal of cohesins from chromosome arms and cytokinesis. Interestingly, PLK1 functions throughout the cell cycle acting as a target and regulator of DNA damage responses (Takaki, 2008). Deregulation of Plk1 has been observed in a wide range of human malignancies. PLK1 inhibitors have been observed to inhibit mitotic progression and to induce apoptosis. GSK461364 is a selective PLK1 inhibitor that is now being evaluated in Phase I clincial trials in patients with Non-Hodgkins lymphoma.

AURKB is a 39 kDa protein that is a component of the chromosome passenger complex that acts to regulate kinetochore-microtubule interactions. It is also required for spindle checkpoint function and is involved in regulating the cleavage of polar spindle microtubules and the onset of cytokinesis during mitosis Inhibitors of AURKB seem to override a mitotic checkpoint and drive cells with mitotic aberrations through the mitotic process presumably resulting in subsequent death as a result of failed cytokinesis (Keen N and Taylor S, 2009). GSK1070916 is a reversible and ATP-competitive inhibitor of AURKB (Anderson, Biochem J 2009).

CENPE is a 312 kDa protein comprising of a kinesin motor domain tethered to a globular COOH-terminal domain via an extended rod. CENPE contributes to coordination of the interaction of microtubules with an anaphase promoting complex. Loss of CENPE function results in arrest in prometaphase and apoptotic death (Wood K W, 2008). GSK923295 is an allosteric inhibitor of the kinesin motor domain of CENPE that is now undergoing clinical evaluation.

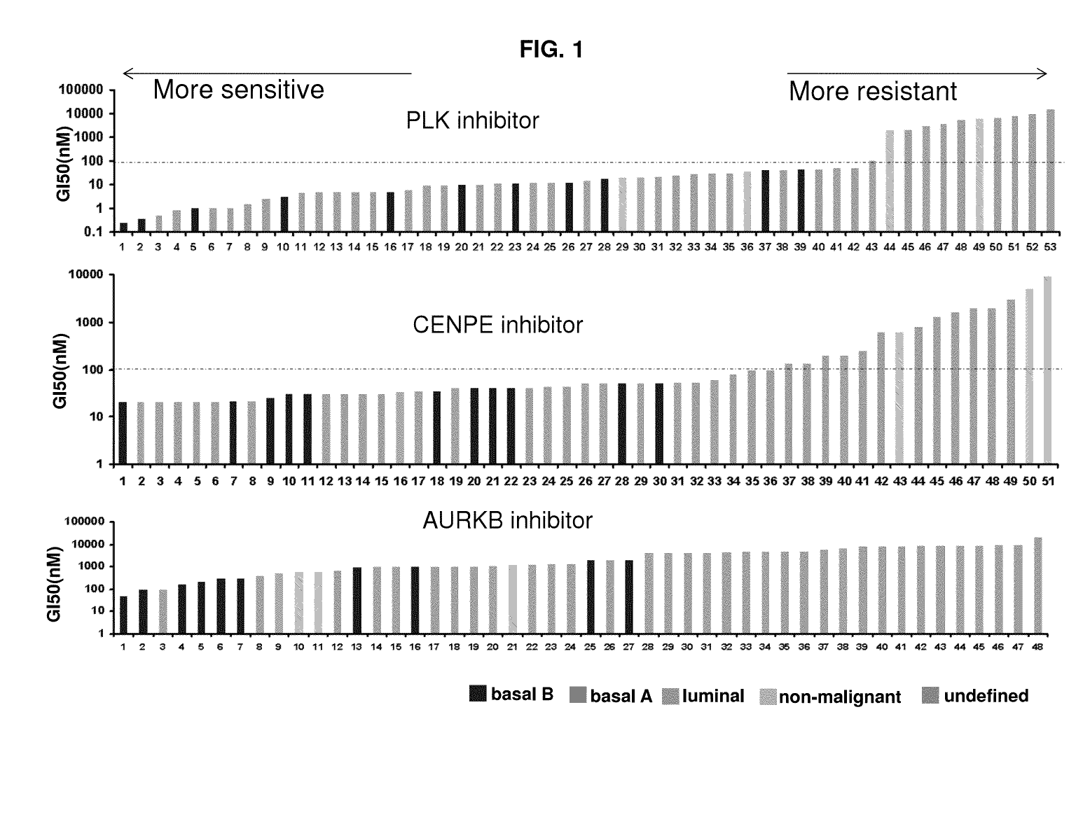

Quantitative measurements of the concentrations of GSK1070916, GS K461364 and GSK923295 needed to inhibit growth by 50% (GI₅₀) in a panel of 53 breast cancer cell lines showed the GI₅₀s varied widely between cell lines. On average, cell lines representing basal-like breast cancers were more responsive to all three mitotic apparatus drugs (see FIG. 1) than those representing luminal subtype breast cancers. However, the responses were still quite variable within the basal and luminal subtypes. Considering the molecular and biological diversity between the cell lines and the differences in proteins targeted by the three drugs, it is remarkable that the responses to the three drugs among the lines were significantly correlated (see Table 1).

TABLE 1
Pearson correlation coefficients (and significance)
between cell line responses across 53 breast cell
lines derived from tumor and normal tissues

| | GSK1070916 | GSK461364 | GSK923295 |
|---|---|---|---|
| | Targets AURKB | Targets PLK1 | Targets CENPE |
| GSK1070916 | 1.0 | 0.326* (0.024^{#}) | 0.323 (0.025) |
| (AURKB) | | | |
| GSK461364 | | 1.0 | 0.444 (0.01) |
| (PLK1) | | | |
| GSK923295 | | | 1.0 |
| (CENPE) | | | |

*Pearson correlation coefficient;
^{#}p-value.

These data suggest that the molecular processes responsible for the responses to these three drugs are similar in spite of the molecular differences between the three drug targets. This motivated an effort to identify a molecular signature associated with the common response that might be used to select patients that would be most likely to respond clinically.

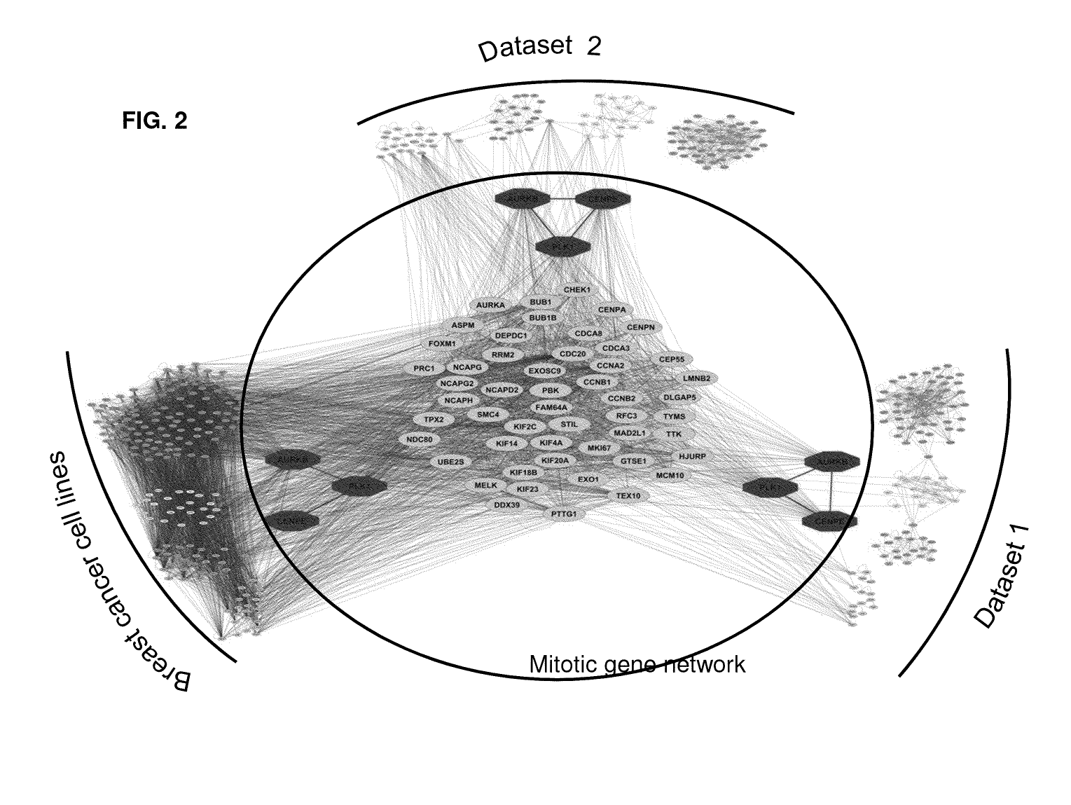

To accomplish this, we analyzed Affymetrix gene expression profiles for 53 human breast cancer cell lines, see Neve, R. M. et al. Cancer Cell 10, 515-527 (2006) for methodology and hereby incorporated by reference, to define a network of genes having transcript levels that were significantly correlated with PLK1, CENPE, and A URKB transcript levels and with each other (see FIG. 2) in the same cancer cells. This process resulted in a network of 272 genes as detected by 272 of the Affymetrix many gene probes (p-value=2.6×10⁻⁶ based on 1000 permutation tests) that were used to probe the cell lines. See Table 2.

TABLE 2
Mitotic Gene Network (272 genes)
272 genes significantly correlated with PLK1, CENPE or
AURKB expression in breast cancer cells = total 275 genes

| | | |
|---|---|---|
| 1 | 200035_at | DULLARD |
| 2 | 200054_at | ZNF259 |
| 3 | 200600_at | MSN |
| 4 | 200634_at | PFN1 |
| 5 | 200670_at | XBP1 |
| 6 | 200804_at | TMBIM6 |
| 7 | 200815_s_at | PAFAH1B1 |
| 8 | 201215_at | PLS3 |
| 9 | 201272_at | AKR1B1 |
| 10 | 201276_at | RAB5B |
| 11 | 201427_s_at | SEPP1 |
| 12 | 201528_at | RPA1 |
| 13 | 201529_s_at | RPA1 |
| 14 | 201530_x_at | EIF4A1 |
| 15 | 201564_s_at | FSCN1 |
| 16 | 201584_s_at | DDX39 |
| 17 | 201663_s_at | SMC4 |
| 18 | 201697_s_at | DNMT1 |
| 19 | 201727_s_at | ELAVL1 |
| 20 | 201767_s_at | ELAC2 |
| 21 | 201770_at | SNRPA |
| 22 | 201774_s_at | NCAPD2 |
| 23 | 201844_s_at | RYBP |
| 24 | 201846_s_at | RYBP |
| 25 | 201983_s_at | EGFR |
| 26 | 202078_at | COPS3 |
| 27 | 202106_at | GOLGA3 |
| 28 | 202154_x_at | TUBB4 |
| 29 | 202159_at | FARSA |
| 30 | 202240_at | PLK1 |
| 31 | 202440_s_at | ST5 |
| 32 | 202454_s_at | ERBB3 |
| 33 | 202580_x_at | FOXM1 |
| 34 | 202589_at | TYMS |
| 35 | 202636_at | RNF103 |
| 36 | 202690_s_at | SNRPD1 |
| 37 | 202705_at | CCNB2 |
| 38 | 202734_at | TRIP10 |
| 39 | 202779_s_at | UBE2S |
| 40 | 202870_s_at | CDC20 |
| 41 | 202900_s_at | NUP88 |
| 42 | 203009_at | BCAM |
| 43 | 203065_s_at | CAV1 |
| 44 | 203306_s_at | SLC35A1 |
| 45 | 203317_at | PSD4 |
| 46 | 203324_s_at | CAV2 |
| 47 | 203362_s_at | MAD2L1 |
| 48 | 203418_at | CCNA2 |
| 49 | 203554_x_at | PTTG1 |
| 50 | 203701_s_at | TRMT1 |
| 51 | 203755_at | BUB1B |
| 52 | 203764_at | DLGAP5 |
| 53 | 203787_at | SSBP2 |
| 54 | 203871_at | SENP3 |
| 55 | 203895_at | PLCB4 |
| 56 | 203896_s_at | PLCB4 |
| 57 | 203906_at | IQSEC1 |
| 58 | 203961_at | NEBL |
| 59 | 203962_s_at | NEBL |
| 60 | 204088_at | P2RX4 |
| 61 | 204127_at | RFC3 |
| 62 | 204133_at | RRP9 |
| 63 | 204162_at | NDC80 |
| 64 | 204240_s_at | SMC2 |
| 65 | 204290_s_at | ALDH6A1 |
| 66 | 204317_at | GTSE1 |
| 67 | 204318_s_at | GTSE1 |
| 68 | 204420_at | FOSL1 |
| 69 | 204492_at | ARHGAP11A |
| 70 | 204567_s_at | ABCG1 |
| 71 | 204603_at | EXO1 |
| 72 | 204623_at | TFF3 |
| 73 | 204667_at | FOXA1 |
| 74 | 204709_s_at | KIF23 |
| 75 | 204822_at | TTK |
| 76 | 204825_at | MELK |
| 77 | 204887_s_at | PLK4 |
| 78 | 204942_s_at | ALDH3B2 |
| 79 | 204951_at | RHOH |
| 80 | 204962_s_at | CENPA |
| 81 | 204977_at | DDX10 |
| 82 | 205019_s_at | VIPR1 |
| 83 | 205046_at | CENPE |
| 84 | 205061_s_at | EXOSC9 |
| 85 | 205085_at | ORC1L |
| 86 | 205135_s_at | NUFIP1 |
| 87 | 205150_s_at | KIAA0644 |
| 88 | 205151_s_at | KIAA0644 |
| 89 | 205217_at | TIMM8A |
| 90 | 205248_at | DOPEY2 |
| 91 | 205251_at | PER2 |
| 92 | 205339_at | STIL |
| 93 | 205349_at | GNA15 |
| 94 | 205393_s_at | CHEK1 |
| 95 | 205394_at | CHEK1 |
| 96 | 205527_s_at | GEMIN4 |
| 97 | 205594_at | ZNF652 |
| 98 | 205652_s_at | TTLL1 |
| 99 | 205891_at | ADORA2B |
| 100 | 206034_at | SERPINB8 |
| 101 | 206364_at | KIF14 |
| 102 | 206445_s_at | PRMT1 |
| 103 | 206546_at | SYCP2 |
| 104 | 206571_s_at | MAP4K4 |
| 105 | 207030_s_at | CSRP2 |
| 106 | 207038_at | SLC16A6 |
| 107 | 207127_s_at | HNRNPH3 |
| 108 | 207949_s_at | ICA1 |
| 109 | 208079_s_at | AURKA |
| 110 | 208405_s_at | CD164 |
| 111 | 208456_s_at | RRAS2 |
| 112 | 208636_at | ACTN1 |
| 113 | 208637_x_at | ACTN1 |
| 114 | 208782_at | FSTL1 |
| 115 | 208789_at | PTRF |
| 116 | 208790_s_at | PTRF |
| 117 | 208827_at | PSMB6 |
| 118 | 208910_s_at | C1QBP |
| 119 | 208977_x_at | TUBB2A |
| 120 | 209110_s_at | RGL2 |
| 121 | 209161_at | PRPF4 |
| 122 | 209191_at | TUBB6 |
| 123 | 209195_s_at | ADCY6 |
| 124 | 209343_at | EFHD1 |
| 125 | 209350_s_at | GPS2 |
| 126 | 209408_at | KIF2C |
| 127 | 209464_at | AURKB |
| 128 | 209494_s_at | PATZ1 |
| 129 | 209642_at | BUB1 |
| 130 | 209747_at | TGFB3 |
| 131 | 209773_s_at | RRM2 |
| 132 | 209832_s_at | CDT1 |
| 133 | 210008_s_at | MRPS12 |
| 134 | 210024_s_at | UBE2E3 |
| 135 | 210052_s_at | TPX2 |
| 136 | 210108_at | CACNA1D |
| 137 | 210178_x_at | FUSIP1 |
| 138 | 210457_x_at | HMGA1 |
| 139 | 210463_x_at | TRMT1 |
| 140 | 210547_x_at | ICA1 |
| 141 | 210652_s_at | TTC39A |
| 142 | 210829_s_at | SSBP2 |
| 143 | 210916_s_at | CD44 |
| 144 | 210933_s_at | FSCN1 |
| 145 | 211034_s_at | C12orf51 |
| 146 | 211084_x_at | PRKD3 |
| 147 | 211126_s_at | CSRP2 |
| 148 | 211160_x_at | ACTN1 |
| 149 | 211519_s_at | KIF2C |
| 150 | 211750_x_at | TUBA1A |
| 151 | 211787_s_at | EIF4A1 |
| 152 | 211954_s_at | IPO5 |
| 153 | 211964_at | COL4A2 |
| 154 | 211982_x_at | XPO6 |
| 155 | 212021_s_at | MKI67 |
| 156 | 212097_at | CAV1 |
| 157 | 212099_at | RHOB |
| 158 | 212148_at | PBX1 |
| 159 | 212151_at | PBX1 |
| 160 | 212181_s_at | NUDT4 |
| 161 | 212183_at | NUDT4 |
| 162 | 212190_at | SERPINE2 |
| 163 | 212378_at | GART |
| 164 | 212441_at | KIAA0232 |
| 165 | 212442_s_at | LASS6 |
| 166 | 212446_s_at | LASS6 |
| 167 | 212450_at | SECISBP2L |
| 168 | 212508_at | MOAP1 |
| 169 | 212590_at | RRAS2 |
| 170 | 212789_at | NCAPD3 |
| 171 | 212841_s_at | PPFIBP2 |
| 172 | 212856_at | DIP |
| 173 | 212949_at | NCAPH |
| 174 | 212956_at | TBC1D9 |
| 175 | 213172_at | TTC9 |
| 176 | 213198_at | ACVR1B |
| 177 | 213226_at | EXOSC9 |
| 178 | 213302_at | PFAS |
| 179 | 213308_at | SHANK2 |
| 180 | 213412_at | TJP3 |
| 181 | 213441_x_at | SPDEF |
| 182 | 213476_x_at | TUBB4 |
| 183 | 213651_at | INPP5J |
| 184 | 213784_at | RABL4 |
| 185 | 214214_s_at | C1QBP |
| 186 | 214266_s_at | PDLIM7 |
| 187 | 214404_x_at | SPDEF |
| 188 | 214433_s_at | SELENBP1 |
| 189 | 214700_x_at | RIF1 |
| 190 | 214710_s_at | CCNB1 |
| 191 | 214746_s_at | ZNF467 |
| 192 | 214784_x_at | XPO6 |
| 193 | 215113_s_at | SENP3 |
| 194 | 215942_s_at | GTSE1 |
| 195 | 216602_s_at | FARSA |
| 196 | 216952_s_at | LMNB2 |
| 197 | 217099_s_at | GEMIN4 |
| 198 | 217368_at | RP11-385M4.4 |
| 199 | 217640_x_at | C18orf24 |
| 200 | 217943_s_at | MAP7D1 |
| 201 | 217979_at | TSPAN13 |
| 202 | 217992_s_at | EFHD2 |
| 203 | 217996_at | PHLDA1 |
| 204 | 218009_s_at | PRC1 |
| 205 | 218035_s_at | RBM47 |
| 206 | 218104_at | TEX10 |
| 207 | 218156_s_at | TSR1 |
| 208 | 218204_s_at | FYCO1 |
| 209 | 218355_at | KIF4A |
| 210 | 218502_s_at | TRPS1 |
| 211 | 218512_at | WDR12 |
| 212 | 218542_at | CEP55 |
| 213 | 218574_s_at | LMCD1 |
| 214 | 218584_at | TCTN1 |
| 215 | 218662_s_at | NCAPG |
| 216 | 218663_at | NCAPG |
| 217 | 218710_at | TTC27 |
| 218 | 218726_at | HJURP |
| 219 | 218755_at | KIF20A |
| 220 | 218770_s_at | TMEM39B |
| 221 | 218828_at | PLSCR3 |
| 222 | 218854_at | SART2 |
| 223 | 218918_at | MAN1C1 |
| 224 | 219098_at | MYBBP1A |
| 225 | 219148_at | PBK |
| 226 | 219204_s_at | SRR |
| 227 | 219206_x_at | TMBIM4 |
| 228 | 219223_at | C9orf7 |
| 229 | 219555_s_at | CENPN |
| 230 | 219562_at | RAB26 |
| 231 | 219570_at | KIF16B |
| 232 | 219588_s_at | NCAPG2 |
| 233 | 219918_s_at | ASPM |
| 234 | 219956_at | GALNT6 |
| 235 | 220173_at | C14orf45 |
| 236 | 220192_x_at | SPDEF |
| 237 | 220258_s_at | WDR79 |
| 238 | 220295_x_at | DEPDC1 |
| 239 | 220306_at | FAM46C |
| 240 | 220651_s_at | MCM10 |
| 241 | 220658_s_at | ARNTL2 |
| 242 | 221024_s_at | SLC2A10 |
| 243 | 221260_s_at | CSRNP2 |
| 244 | 221436_s_at | CDCA3 |
| 245 | 221510_s_at | GLS |
| 246 | 221520_s_at | CDCA8 |
| 247 | 221561_at | SOAT1 |
| 248 | 221588_x_at | ALDH6A1 |
| 249 | 221589_s_at | ALDH6A1 |
| 250 | 221591_s_at | FAM64A |
| 251 | 221598_s_at | MED27 |
| 252 | 221655_x_at | EPS8L1 |
| 253 | 221676_s_at | CORO1C |
| 254 | 221832_s_at | LUZP1 |
| 255 | 221845_s_at | CLPB |
| 256 | 221849_s_at | LOC90379 |
| 257 | 221880_s_at | FAM174B |
| 258 | 221934_s_at | DALRD3 |
| 259 | 221987_s_at | TSR1 |
| 260 | 222039_at | KIF18B |
| 261 | 222125_s_at | P4HTM |
| 262 | 35148_at | TJP3 |
| 263 | 35666_at | SEMA3F |
| 264 | 40093_at | BCAM |
| 265 | 44563_at | WRAP53 |
| 266 | 50376_at | ZNF444 |
| 267 | 50965_at | RAB26 |
| 268 | 51158_at | FAM174B |
| 269 | 51176_at | MED27 |
| 270 | 56197_at | PLSCR3 |
| 271 | 61874_at | C9orf7 |
| 272 | 91826_at | EPS8R1 |

Ontology is the philosophical study of the nature of being, existence or reality in general, as well as of the basic categories of being and their relations. Gene Ontology, or GO, is a major bioinformatics initiative to unify the representation of gene and gene product attributes across all species. The Gene Ontology project provides an ontology of defined terms representing gene product properties. The ontology covers three domains; cellular component, the parts of a cell or its extracellular environment; molecular function, the elemental activities of a gene product at the molecular level, such as binding or catalysis; and biological process, operations or sets of molecular events with a defined beginning and end, pertinent to the functioning of integrated living units: cells, tissues, organs, and organisms. Each GO term within the ontology has a term name, which may be a word or string of words; a unique alphanumeric identifier; a definition with cited sources; and a namespace indicating the domain to which it belongs. The GO vocabulary is designed to be species-neutral, and includes terms applicable to prokaryotes and eukaryotes, single and multicellular organisms.

As expected Gene Ontology analysis showed many of the genes (e.g. AURKA, CDCA8, BUB1) were involved in mitotic processes (see Table 3).

The genes expressed (272) correlating to PLK1, CENPE, and AURKB expression (each of the three lead genes involved in a different aspect of mitosis) are classifiable by gene ontology classification methodology in largely mitosis and mitosis-related protein activities in the context of cellular activity.

TABLE 3
Gene Ontology (GO) of Expression Network of 272 Genes
Correlated with PLK1, CENPE or AURKB expression

| GO ID | Description | Gene |
|---|---|---|
| 7067 | mitosis | KIF23 TUBB2A AURKA CEP55 AURKB PTTG1 KIF2C |
| | | CDCA8 NCAPH C18ORF24 NCAPG2 NCAPG BUB1 |
| | | PAFAH1B1 FOSL1 CCNA2 ASPM CDCA3 TUBB4 DLGAP5 |
| | | TPX2 CENPE CDC20 NDC80 PLK PBK SMC2 NCAPD3 SMC4 |
| | | NCAPD2 CCNB1 MAD2L1 CCNB2 BUB1B |
| 279 | M phase | KIF23 TUBB2A CHEK1 AURKA CEP55 AURKB PTTG1 |
| | | SYCP2 RPA1 KIF2C CDCA8 NCAPH C18ORF24 NCAPG2 |
| | | NCAPG BUB1 PAFAH1B1 FOSL1 CCNA2 ASPM CDCA3 |
| | | TUBB4 EXO1 DLGAP5 TPX2 CENPE NDC80 CDC20 PLK |
| | | PBK SMC2 NCAPD3 SMC4 NCAPD2 CCNB1 MAD2L1 |
| | | CCNB2 BUB1B |
| 87 | M phase of mitotic | KIF23 TUBB2A AURKA CEP55 AURKB PTTG1 KIF2C |
| | cell cycle | CDCA8 NCAPH C18ORF24 NCAPG2 NCAPG BUB1 |
| | | PAFAH1B1 FOSL1 CCNA2 ASPM CDCA3 TUBB4 DLGAP5 |
| | | TPX2 CENPE CDC20 NDC80 PLK PBK SMC2 NCAPD3 SMC4 |
| | | NCAPD2 CCNB1 MAD2L1 CCNB2 BUB1B |
| 278 | mitotic cell cycle | KIF23 PRC1 TUBB2A TTK CHEK1 AURKA CEP55 AURKB |
| | | PTTG1 GTSE1 ACVR1B KIF2C CDCA8 NCAPH C18ORF24 |
| | | PSMB6 NCAPG2 NCAPG BUB1 PAFAH1B1 FOSL1 CCNA2 |
| | | ASPM CDCA3 TUBB4 DLGAP5 TPX2 CENPE NDC80 CDC20 |
| | | PLK PBK SMC2 NCAPD3 SMC4 NCAPD2 CCNB1 MAD2L1 |
| | | CCNB2 BUB1B |
| 22403 | cell cycle phase | KIF23 TUBB2A CHEK1 AURKA CEP55 AURKB PTTG1 |
| | | SYCP2 GTSE1 RPA1 ACVR1B KIF2C CDCA8 NCAPH |
| | | C18ORF24 NCAPG2 NCAPG BUB1 PAFAH1B1 FOSL1 |
| | | CCNA2 ASPM CDCA3 TUBB4 EXO1 DLGAP5 TPX2 CENPE |
| | | NDC80 CDC20 PLK PBK SMC2 NCAPD3 SMC4 NCAPD2 |
| | | CCNB1 MAD2L1 CCNB2 BUB1B |
| 22402 | cell cycle process | KIF23 PRC1 TUBB2A TTK CHEK1 AURKA CEP55 AURKB |
| | | PTTG1 SYCP2 GTSE1 RPA1 ACVR1B KIF2C CDCA8 NCAPH |
| | | C18ORF24 PSMB6 NCAPG2 NCAPG BUB1 PAFAH1B1 |
| | | FOSL1 CCNA2 ASPM CDCA3 TUBB4 EXO1 DLGAP5 TPX2 |
| | | CENPE NDC80 CDC20 PLK PBK SMC2 NCAPD3 SMC4 |
| | | NCAPD2 CCNB1 MAD2L1 CCNB2 BUB1B |
| 7049 | cell cycle | KIF23 PRC1 TUBB2A TTK CHEK1 AURKA CEP55 AURKB |
| | | PTTG1 SYCP2 GTSE1 CDT1 RPA1 ACVR1B KIF2C CDCA8 |
| | | NCAPH C18ORF24 PSMB6 NCAPG2 NCAPG BUB1 |
| | | PAFAH1B1 FOSL1 CCNA2 ASPM CDCA3 TUBB4 EXO1 |
| | | MKI67 DLGAP5 TPX2 CENPE NDC80 CDC20 PLK PBK |
| | | SMC2 NCAPD3 GPS2 SMC4 NCAPD2 CCNB1 CCNB2 |
| | | MAD2L1 RIF1 BUB1B |
| 51301 | cell division | KIF23 PRC1 AURKB PTTG1 CEP55 SYCP2 NCAPH CDCA8 |
| | | C18ORF24 NCAPG NCAPG2 BUB1 PAFAH1B1 CCNA2 |
| | | ASPM CDCA3 CENPE CDC20 NDC80 PLK SMC2 NCAPD3 |
| | | SMC4 NCAPD2 CCNB1 MAD2L1 CCNB2 BUB1B |
| 6996 | organelle | KIF23 CAV2 CAV1 KIF4A RAB5B PRC1 TUBB2A TTK |
| | organization and | AURKA PTTG1 SYCP2 GTSE1 KIF2C PFN1 MOAP1 NCAPH |
| | biogenesis | CENPA NCAPG2 NCAPG DULLARD TUBB6 PAFAH1B1 |
| | | DOPEY2 TUBA1A TRIP10 GEMIN4 PLS3 TUBB4 KIF14 |
| | | EXOSC9 TSR1 DLGAP5 FSCN1 KIF18B ACTN1 CENPE |
| | | NDC80 RRP9 C20ORF23 DIP SMC2 HMGA1 NCAPD3 SMC4 |
| | | TIMM8A NCAPD2 BUB1B KIF20A |
| 16359 | mitotic chromosome | NCAPH NCAPG NCAPG2 DLGAP5 CENPE NDC80 SMC2 |
| | segregation | NCAPD3 SMC4 NCAPD2 |
| 819 | sister chromatid | NCAPH NCAPG NCAPG2 DLGAP5 CENPE NDC80 SMC2 |
| | segregation | NCAPD3 SMC4 NCAPD2 |
| 7017 | microtubule-based | KIF14 KIF23 KIF4A PRC1 TUBB2A KIF18B TTK CENPE |
| | process | AURKA NDC80 C20ORF23 GTSE1 KIF2C BUB1B TUBB6 |
| | | PAFAH1B1 TUBA1A TUBB4 KIF20A |
| 16043 | cell organization and | KIF23 PDLIM7 RAB5B PRC1 TUBB2A SNRPD1 TTK AURKA |
| | biogenesis | PTTG1 GTSE1 EFHD1 KIF2C TUBB6 TUBA1A PLS3 TUBB4 |
| | | EGFR KIF14 EXOSC9 ACTN1 RRP9 C20ORF23 HMGA1 |
| | | NCAPD3 NCAPD2 TIMM8A BUB1B FUSIP1 CAV2 KIF4A |
| | | CAV1 SYCP2 PFN1 NCAPH MOAP1 NCAPG NCAPG2 |
| | | CENPA DULLARD PAFAH1B1 DOPEY2 TRIP10 GEMIN4 |
| | | COL4A2 TSR1 DLGAP5 FSCN1 KIF18B CENPE NDC80 DIP |
| | | SMC2 SMC4 CORO1C PLSCR3 KIF20A |
| 7059 | chromosome | NCAPH NCAPG NCAPG2 DLGAP5 CENPE NDC80 PTTG1 |
| | | SMC2 NCAPD3 SMC4 NCAPD2 |

It was then assessed the extent to which the list or network of genes were present in primary breast tumors by searching for PLK1, CENPE, and AURKB associated genes expressed in two independent Affymetrix transcriptional profile data sets measured for primary breast cancers, see Chin, K. et al. Cancer Cell 10, 529-541 (2006), and Wang, Y. et al. Lancet 365, 671-679 (2005).

Chin et al. explored the roles of genome copy number abnormalities (CNAs) in breast cancer pathophysiology by identifying associations between recurrent CNAs, gene expression, and clinical outcome in a set of aggressively treated early-stage breast tumors. It shows that the recurrent CNAs differ between tumor subtypes defined by expression pattern and that stratification of patients according to outcome can be improved by measuring both expression and copy number, especially high-level amplification. Sixty-six genes deregulated by the high-level amplifications were determined to be potential therapeutic targets.

Wang et al. explored genome-wide measures of gene expression to identify patterns of gene activity that sub-classify tumors and might provide a better means than is currently available for individual risk assessment in patients with lymph-node-negative breast cancer. Using Affymetrix Human U133a GeneChips, the expression of 22000 transcripts from total RNA of frozen tumour samples from 286 lymph-node-negative patients who had not received adjuvant systemic treatment. In a set of 115 tumours, a 76-gene signature was identified consisting of 60 genes for patients positive for oestrogen receptors (ER) and 16 genes for ER-negative patients.

In order to adapt the initially determined mitotic network of 275 genes (Table 2) to other types of cancer, to come up with a mitotic signature specific for that cancer, the 275 genes can be further reduced to a smaller list (as just demonstrated with breast cancer) using one or more data sets from other sources, such as gene expression signatures or lists from the other cancers, for example as for colon, melanoma, and lung cancer.

Also, because the mitosis-related breast cancer gene signature (54 genes) developed shows overlap in expression with expression profiles of other cancers, the 54 gene cancer specific mitotic network is expandable to use for other cancers. In one embodiment, the mitotic network gene expression profiles can be used to provide signatures for other epithelial cancers such as prostate, colon, ovarian, pancreatic, lung, skin (melanoma), esophageal cancers, gynecological cancers, hepatocellular carcinoma, renal cell carcinoma, and small cell lung carcinoma, etc. As such the mitotic profile may relate to these cancers for treatment directed to mitosis-mediated abnormal cell growth and activity.

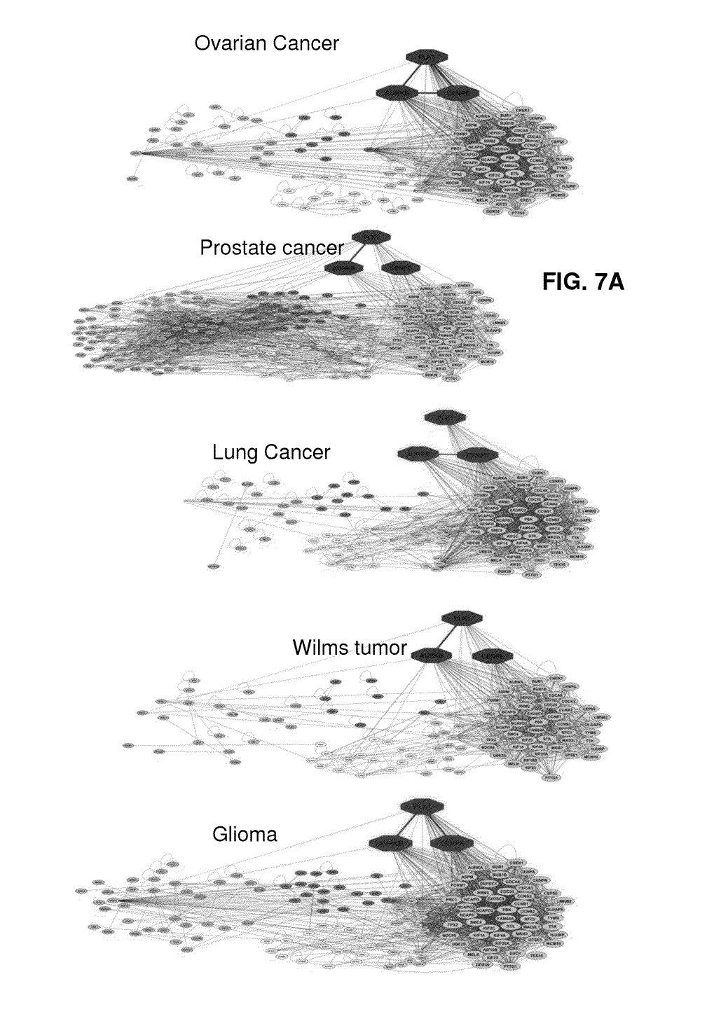

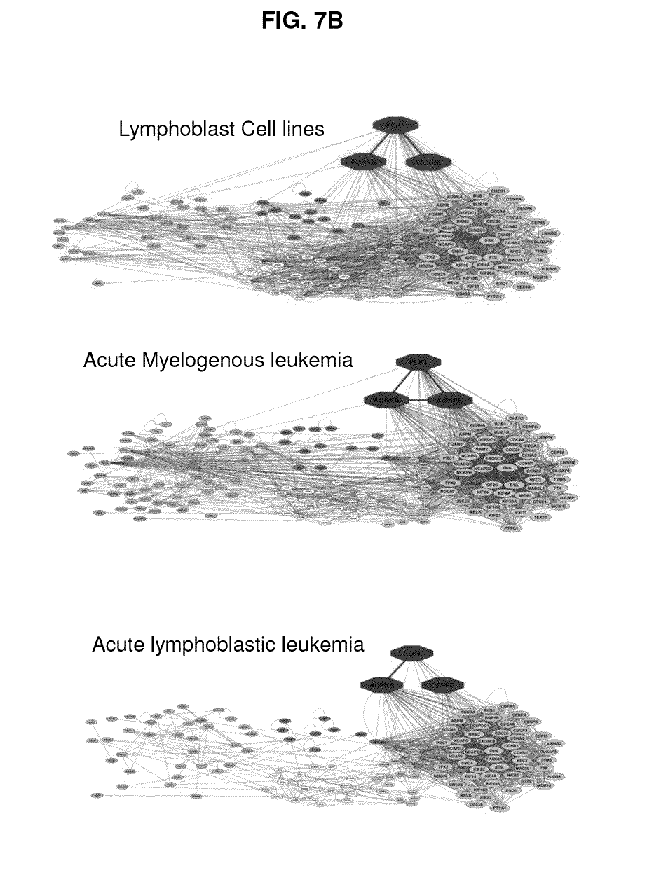

As shown in FIG. 2 this resulted in a mitotic activity network of 54 transcripts most of which are known to be associated with aspects of the mitotic process that was present in all three data sets. The resulting assimilation of these three data sets is the list of 54 genes, as shown in Table 4. The network of 54 genes was also apparent in transcript profiles measured for cancers of the lung, ovarian, prostate, brain, Wilms tumor; and human blood malignancies (e.g. lymphoma and leukemia) (see FIG. 7). It was even present in transcript profiles measured for normal skin samples from crosses between M. spretus and Mus musculus strains, see Quigley, D. A. et al. Nature (2009), which ties the 54 gene expression pattern to melanoma.

In Table 4 below, the mitotic gene network, a list of 54 genes (herein sometimes referred to as “the 54-gene set” or “the mitotic network genes”), formed from the convergence of several other larger lists including expression of genes that correlated with target protein expression of PLK1, AURKB, and CENPE, and two other independent expression networks from cancer tissue. The column under the capital letter D in Table 4 indicates whether the protein represents a druggable target (i.,e., is the target a likely candidate for a small molecule inhibitor, or peptidomimetic or the like based on structural analysis of a binding site or the structure and activity of the molecule that is known, or by siRNA studies conducted as in the Examples). Assessment of drugability is determined based on research by others indicating a binding site or the structure of the molecule that could be used to identify binders and blockers of the activity of the protein or by siRNA studies conducted as in the Examples. A “Y” in that column indicates that the protein (or its gene) is a likely candidate for a target in developing a therapeutic that would act on an element of the mitosis pathway, so defined herein.

In one embodiment, the candidate genes as therapeutic targets are also herein referred to as “the 18-gene set” and include, AURKA, AURKB, BUB1, CENPE, CHEK1, FOXM1, PBK, PLK1, MELK, TTK, TPX2, TYMS, KIF23, KIF20, KIF2C, EXOSC9, PTTG1 and PRC1. The GenBank Accession entries and gene sequences listed in Table 4 are hereby incorporated by reference in their entirety for all purposes.

In another embodiment, the candidate genes as therapeutic targets, also referred to herein as “the 22-gene set” and include: PLK1, SMC4, PBK, KIF14, NCAPD2, RRM2, CENPA, KNTC2, KIF23, RFC3, EXO1, LMNB2, TEX10, DEPDC1, DDX39, MAD2L1, C10orf13, FAM64A, TPX2, AURKA, TTK. The GenBank Accession entries and gene sequences listed in Table 7 are hereby incorporated by reference in their entirety for all purposes

TABLE 4
Mitotic Network (54 genes)

| Gene | | Accession | Version and Gem | | |
|---|---|---|---|---|---|
| Symbol | Gene full name | number | ID | Reference | D |
| AURKA | Homo sapiens aurora | NM_003600 | NM_003600.2 | [symbol] L 2008 | Y |
| | kinase A, transcript | | GI:38327561 | | |
| | variant 2 | | | | |
| AURKB | Homo sapiens aurora | NM_004217 | NM_004217.2 | Shannon K B 2002 | Y |
| | kinase B (AURKB) | | GI:83776599 | | |
| BUB1 | Homo sapiens BUB1 | NM_004336 | NM_004336.3 | Kang J 2008 | Y |
| | budding uninhibited by | | GI:211938448 | | |
| | benzimidazoles 1 homolog | | | | |
| | (yeast) | | | | |
| CENPE | Homo sapiens centromere | NM_001813 | NM_001813.2 | Yardimici H 2008 | Y |
| | protein E | | GI:71061467 | | |
| CHEK1 | Homo sapiens CHK1 | NM_001274 | NM_001274.4 | Rodriguez R 2005 | Y |
| | checkpoint homolog | | GI:166295191 | | |
| | (S. pombe) | | | | |
| FOXM1 | Homo sapiens forkhead | NM_202002 | NM_202002.1 | Wonsey D R 2005; | Y |
| | box M1 (FOXM1), | | GI:42544166 | Fu Z 2008 | |
| | transcript variant 1 | | | | |
| MELK | Homo sapiens maternal | NM_014791 | NM_014791.2 | Badouel C 2006 | Y |
| | embryonic leucine zipper | | GI:41281490 | | |
| | kinase | | | | |
| PBK | MAPKK-like protein | NM_018492 | NM_018492.2 | Gaudet S 2000; | Y |
| | kinase; PDZ-binding | | GI:18490990 | Simons-Evelyn M | |
| | kinase; T-LAK cell- | | | 2001 | |
| | originated protein kinase; | | | | |
| PLK1 | Homo sapiens polo-like | NM_005030 | NM_005030.3 | Archambault V | Y |
| | kinase 1 | | GI:34147632 | 2009; Kishi K | |
| | | | | 2009; Fu Z 2008 | |
| TTK | Homo sapiens TTK protein | NM_003318 | NM_003318.3 | Liu S T 2003 | Y |
| | kinase | | GI:34303964 | | |
| TYMS | Homo sapiens thymidylate | NM_001071 | NM_001071.2 | Kemming D 2006; | Y |
| | synthetase | | GI:186972144 | Le X 2004 | |
| ASPM | Homo sapiens asp | NM_018136 | NM_018136.4 | Bond J 2002 | |
| | (abnormal spindle) | | GI:194248058 | | |
| | homolog, microcephaly | | | | |
| | associated (Drosophila) | | | | |
| BUB1B | Homo sapiens BUB1 | NM_001211 | NM_001211.5 | Lampson M A | |
| | budding uninhibited by | | GI:168229167 | 2004 | |
| | benzimidazoles 1 homolog | | | | |
| | beta (yeast) | | | | |
| CCNA2 | Homo sapiens cyclin A2 | NM_001237 | NM_001237.3 | Wolthuis R 2008 | |
| | | | GI:166197663 | | |
| CCNB1 | Homo sapiens cyclin B1 | NM_031966 | NM_031966.2 | Allan L A 2007 | |
| | | | GI:34304372 | | |
| CCNB2 | Homo sapiens cyclin B2 | NM_004701 | NM_004701.2 | Bellanger S 2007 | |
| | | | GI:10938017 | | |
| CDC20 | Homo sapiens cell division | NM_001255 | NM_001255.2 | Liu H 2007 | |
| | cycle 20 homolog | | GI:118402581 | | |
| | (S. cerevisiae) | | | | |
| CDCA3 | cell division cycle | NM_031299 | NM_031299.4 | Ayad N G 2003 | |
| | associated 3(also named | | GI:188497626 | | |
| | TOME-1) | | | | |
| CDCA8 | Homo sapiens cell division | NM_018101 | NM_018101.2 | Slattery S D 2008 | |
| | cycle associated 8 | | GI:51593099 | | |
| CENPA | centromere protein A, | NM_001809 | NM_001809.3 | Black B E 2007, | |
| | isoform a | | GI:109637780 | McClelland S E | |
| | | | | 2007 | |
| CENPN | centromere protein N | NM_001100625 | NM_001100625.1 | Foltz D R 2006 | |
| | | | GI:154800484 | | |
| CEP55 | centrosomal protein 55 kDa | NM_018131 | NM_018131.4 | Zhao W M 2006; | |
| | | | GI:187608536 | Morita E 2007 | |
| DDX39 | DEAD (Asp-Glu-Ala-Asp) | NM_005804 | NM_005804.2 | | |
| | box polypeptide 39 | | GI:21040370 | | |
| DEPDC1 | DEP domain containing 1 | NM_017779 | NM_017779.4 | | |
| | | | GI:166295186 | | |
| DLGAP5 | Homo sapiens discs, large | NM_014750 | NM_014750.4 | | |
| | (Drosophila) homolog- | | GI:226371666 | | |
| | associated protein 5 | | | | |
| EXO1 | exonuclease 1 | NM_006027.3 | NM_006027.3 | Fiorentini P 1997 | |
| | | | GI:39995070 | | |
| EXOSC9 | Homo sapiens exosome | NM_001034194 | NM_001034194.1 | | Y |
| | component 9 (EXOSC9) | | GI:77812671 | | |
| FAM64A | family with sequence | NM_019013 | NM_019013.1 | | |
| | similarity 64, member A | | GI:9506604 | | |
| GTSE1 | Homo sapiens G-2 and S- | NM_016426 | NM_016426.5 | Monte M 2003 | |
| | phase expressed 1 | | GI:194294557 | | |
| HJURP | Homo sapiens Holliday | NM_018410 | NM_018410.3 | Kato T 2007 | |
| | junction recognition | | GI:83816963 | | |
| | protein | | | | |
| KIF14 | kinesin family member 14 | NM_014875 | NM_014875.2 | Carleton M 2006; | |
| | | | GI:208610006 | Corson T W 2007 | |
| KIF18B | Homo sapiens kinesin | NM_001080443 | NM_001080443.1 | Miki H 2005 | |
| | family member 18B | | GI:122937288 | | |
| KIF20A | Homo sapiens kinesin | NM_005733 | NM_005733.2 | Neef R 2003 | Y |
| | family member 20A | | GI:195539383 | | |
| KIF23 | Homo sapiens kinesin | NM_138555 | NM_138555.1 | Lee K S 1995 | Y |
| | family member 23 | | GI:20143966 | | |
| | (KIF23), transcript | | | | |
| | variant 1 | | | | |
| KIF2C | Homo sapiens kinesin | NM_006845 | NM_006845.3 | Manning A L 2007 | Y |
| | family member 2C | | GI:166795249 | | |
| KIF4A | Homo sapiens kinesin | NM_012310 | NM_012310.3 | Mazumdar M 2004 | |
| | family member 4A | | GI:116686121 | | |
| LMNB2 | lamin B2 | NM_032737 | NM_032737.2 | Tsai M Y, 2006 | |
| | | | GI:27436950 | | |
| MAD2L1 | MAD2 mitotic arrest | NM_002358 | NM_002358.3 | Tighe A, 2008; | |
| | deficient-like 1 | | GI:194688136 | Lee S H, 2008 | |
| MCM10 | minichromosome | NM_182751 | NM_182751.1 | Park J H, 2008 | |
| | maintenance complex | | GI:33383236 | | |
| | component 10 | | | | |
| MKI67 | antigen identified by | NM_002417 | NM_002417.4 | Schluter C 1993 | |
| | monoclonal antibody Ki- | | GI:225543213 | | |
| | 67(Ki-67) | | | | |
| NCAPD2 | non-SMC condensin I | NM_014865 | NM_014865.3 | Ball A R Jr, 2002 | |
| | complex, subunit D2 | | GI:178056551 | | |
| NCAPG | Homo sapiens non-SMC | NM_022346 | NM_022346.3 | Murphy L A 2008 | |
| | condensin I complex, | | GI:50658080 | | |
| | subunit G | | | | |
| NCAPG2 | Homo sapiens non-SMC | NM_017760 | NM_017760.5 | | |
| | condensin II complex, | | GI:116812585 | | |
| | subunit G2 | | | | |
| NCAPH | Homo sapiens non-SMC | NM_015341 | NM_015341.3 | | |
| | condensin I complex, | | GI:81295814 | | |
| | subunit H | | | | |
| NDC80 | NDC80 homolog, | NM_006101 | NM_006101.2 | McCleland M L | |
| | kinetochore complex | | GI:215820615 | 2003, Wei R R | |
| | component (S. cerevisiae) | | | 2007 | |
| PRC1 | Homo sapiens protein | NM_199414 | NM_199414.1 | Jiang W, 1998 | Y |
| | regulator of cytokinesis 1 | | GI:40807444 | | |
| | (PRC1) | | | | |
| PTTG1 | Homo sapiens pituitary | NM_004219 | NM_004219.2 | Zou 1999; Ying H | Y |
| | tumor-transforming 1 | | GI:11038651 | 2006 | |
| RFC3 | replication factor C | NM_181558 | NM_181558.2 | Shimada M 1999 | |
| | (activator 1) 3, 38 kDa | | GI:108773788 | | |
| RRM2 | ribonucleotide reductase | NM_001034.2 | NM_001034.2 | PMID: 12615712 | |
| | M2 polypeptide | | GI:215490080 | | |
| SMC4 | structural maintenance of | NM_001002800 | NM_001002800.1 | Hagstrom K A | |
| | chromosomes 4 | | GI:50658062 | 2002; Steffensen S | |
| | | | | 2001 | |
| STIL | Homo sapiens SCL/TAL1 | NM_001048166 | NM_001048166.1 | Erez A, 2004; | |
| | interrupting locus (STIL) | | GI:115298662 | Campaner S 2005 | |
| TEX10 | testis expressed 10 | NM_017746 | NM_017746.3 | | |
| | | | GI:239787837 | | |
| TPX2 | Homo sapiens TPX2, | NM_012112 | NM_012112.4 | Bayliss R 2003 | Y |
| | microtubule-associated, | | GI:40354199 | | |
| | homolog (Xenopus laevis) | | | | |
| UBE2S | ubiquitin-conjugating | NM_014501 | NM_014501.2 | Dephoure N 2008 | |
| | enzyme E2S | | GI:112382376 | | |

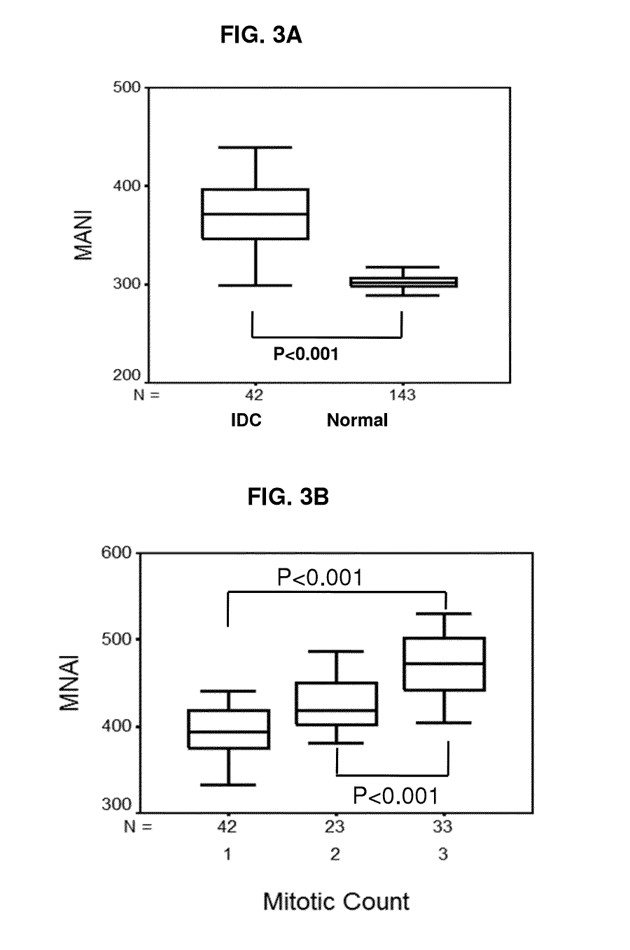

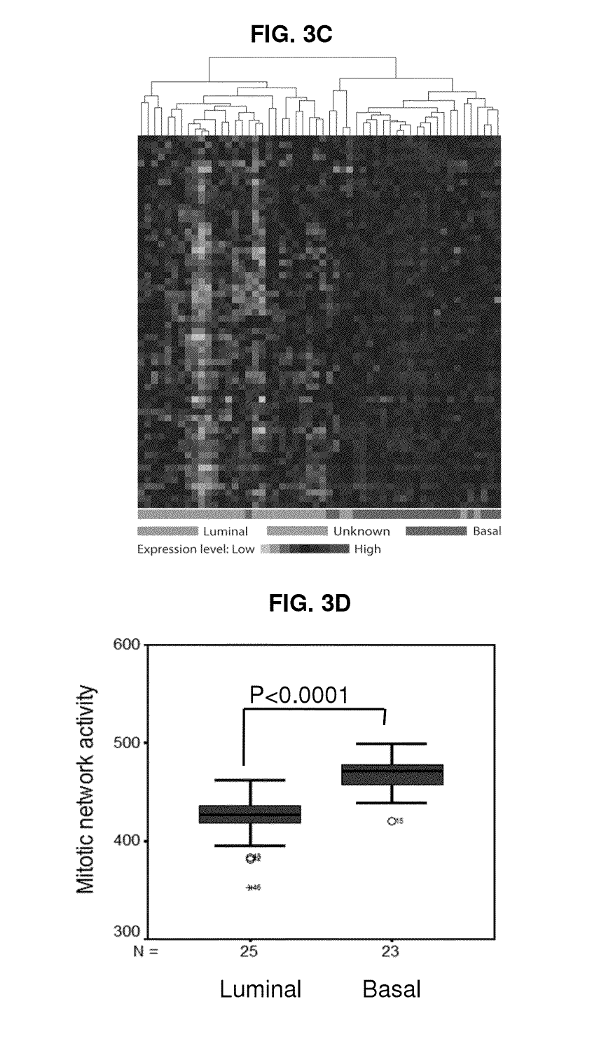

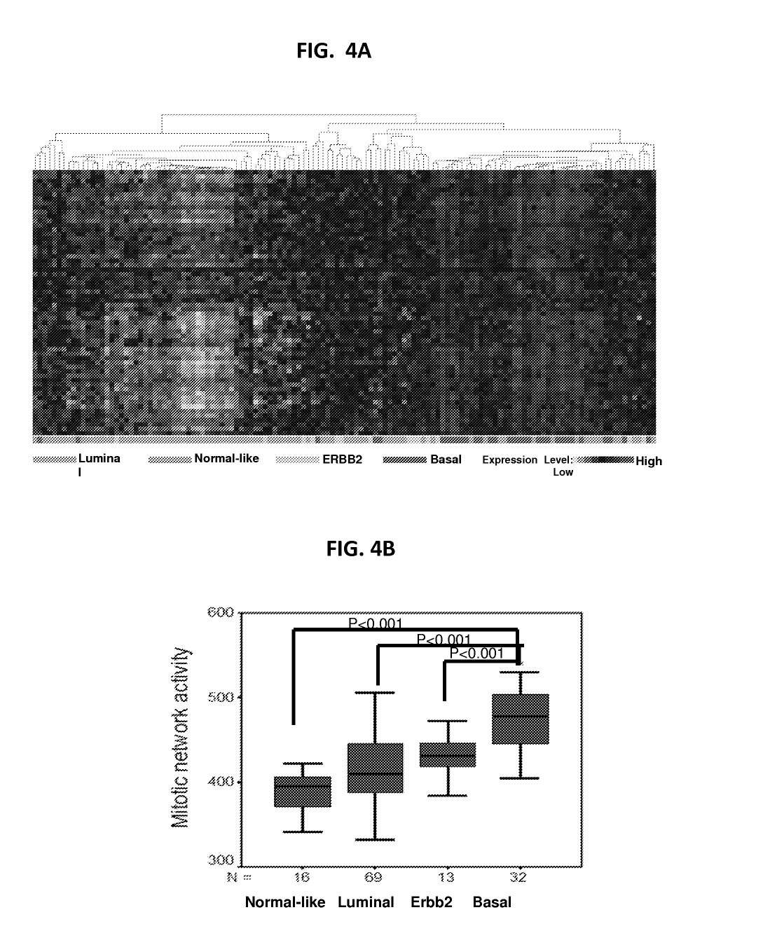

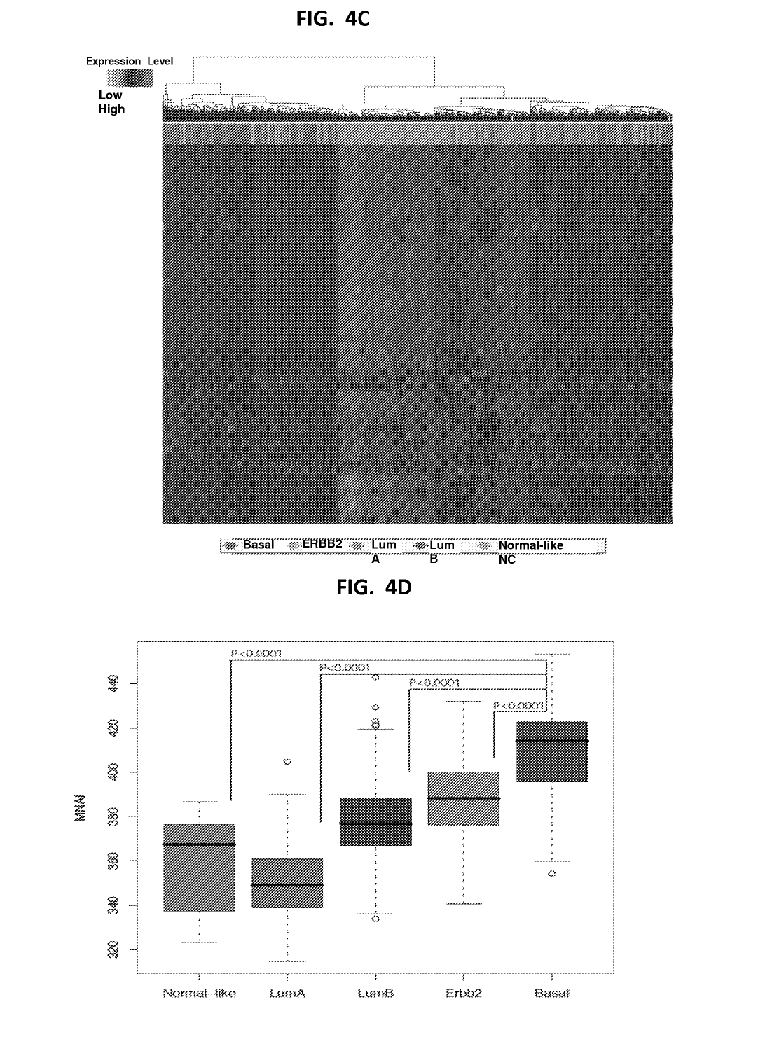

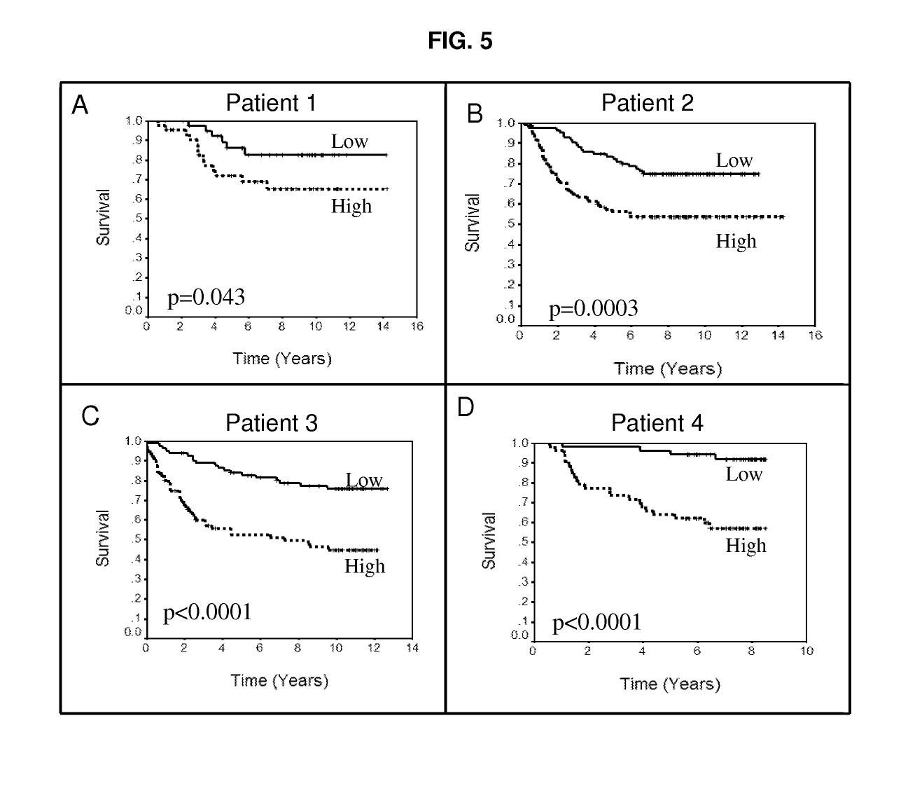

Having demonstrated the existence of a mitotic network that is active in a subset of breast cancer cell lines and tumors, the inventors stratified breast cancer cell lines and primary tumors using unsupervised hierarchical clustering of the 54 mitotic apparatus genes. FIGS. 3 and 4 show that the clusters obtained using the 54 mitotic apparatus network gene set are similar to those obtained using hierarchical clustering using previously published intrinsically variable genes, with basal line cell lines and tumors showing consistently higher expression of the mitotic apparatus genes than the luminal subtypes. This is consistent with histopathological measurements of mitotic activity by Manders et al (Breast Cancer Research and Treatment 77, 1573-7217, 2003) in which a mitotic activity index (MAI=the number of mitotic figures in 10 high power fields) was reported to be negatively correlated with ER and PgR status. Since that study also showed that a high MAI predicted a reduced relapse free survival and overall survival, the association of a high mitotic apparatus network activity with outcome was tested. For this, a mitotic network activity index (MNAI) was defined for prognosis (MNAIFP) as the sum of the transcriptional response of the 54 coordinately regulated mitotic apparatus genes. Generally, herein the mitotic network activity is defined as the sum of mRNAexpression levels of the 54 genes in Table 4. MNAI was significantly elevated in tumors relative to most normal breast tissues. However, the MNAI varied substantially among tumors and in normal tissues. The mRNA levels of mitotic apparatus genes and the MNAI were both significantly higher in basal cell lines (FIG. 3a) and tumors (FIG. 3b-c) as compared to luminal subtype cell lines and tumors. In rank order, the MNAI was lowest in luminal A tumors with pregressively increasing MNAI values for luminal B tumors, Erbb2 positive tumors and then basal-like breast tumors (FIG. 3c). FIG. 5 shows four different breast tumor cohorts that patients with tumors in the top third of MNAIFPs had significantly shorter disease free survival than did those with tumors in the lowest third of MNAIFPs (p=0.043, 0.0003, <0.0001 and <0.0001 for cohort 1 to 4 respectively). The MNAI was also significantly associated with the mitotic activity index (MAI) defined as the number of mitotic events in 10 high power fields (Manders, P., Bult, P., Sweep, C. G., Tjan-Heijnen, V. C. & Beex, L. V. The prognostic value of the mitotic activity index in patients with primary breast cancer who were not treated with adjuvant systemic therapy. Breast Cancer Res Treat 77, 77-84 (2003)) and overall growth rate in cell lines (FIG. 3A). The MAI was similarly found to be associated with reduced relapse free survival and overall survival.

In another embodiment, the following genes make up a subset of the mitotic network and act as a signature (herein referred to as “the 8-gene set”) to inform whether a patient will be responsive to one of the GSK PLK1, CENPE, and AURKB inhibitors. Elimination of data from any one of the genes can reduce the list (e.g. from 8 to 6 genes or less) where the treatment proposed will include only two of the inhibitors.

TABLE 5
Mitotic Network Genes which Predict Response to
GSK PLK1, CENPE, and AURKB inhibitors (8 genes)

| Gene | | |
|---|---|---|
| Symbol | Gene full name | Accession No. |
| MELK | Homo sapiens maternal embryonic | NM_014791 |
| | leucine zipper kinase | |
| SMC4 | structural maintenance of chromosomes 4 | NM_001002800 |
| TEX 10 | testis expressed 10 | NM_017746 |
| AURKA | Homo sapiens aurora kinase A, | NM_003600 |
| | transcript variant 2 | |
| HJURP | Homo sapiens Holliday junction | NM_018410 |
| | recognition protein | |
| BUB1 | Homo sapiens BUB1 | NM_004336 |
| RFC3 | replication factor C (activator 1) 3, | NM_181558 |
| | 38 kDa | |
| CCNB2 | Homo sapiens cyclin B2 | NM_004701 |

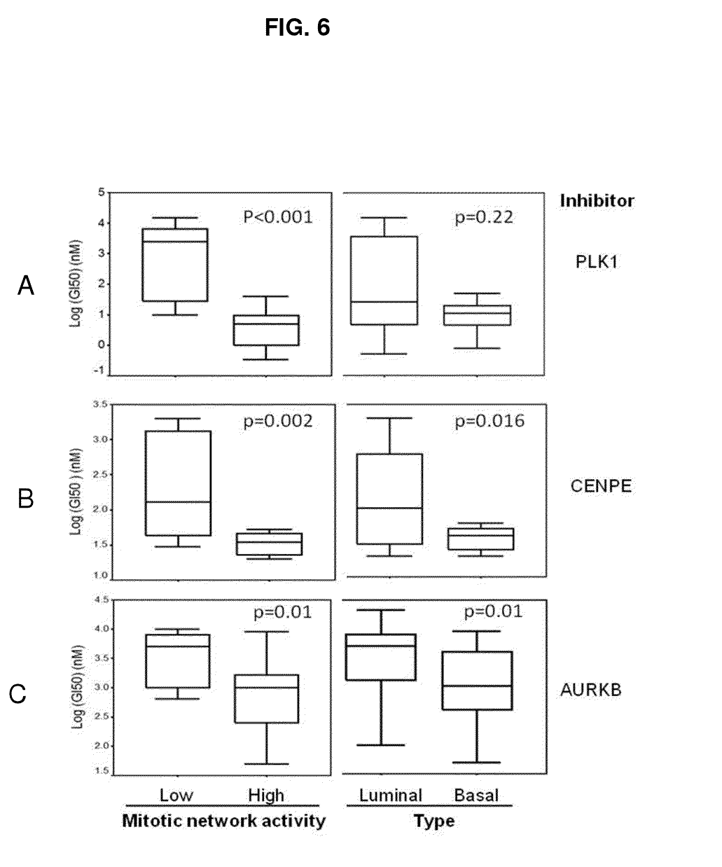

The existence of variable mitotic network activity signature in a wide range of cell lines and tumors suggests that those with the highest MNAIs will be most likely to respond to drugs that target within the mitotic network. We optimally minimized the number of genes for prediction of drug sensitivities based on regression analysis using −log(GI50) as dependent variable and 54 genes mRNA expression level as independent variable and identified a 8-gene mitotic network activity index for drug (MANIFD). FIGS. 6a-c support this idea, showing strong and statistically significant correlations between MNAIFDs and quantitative responses to GSK1070916, GSK461364 and GSK923295. This result suggests that tumors with high MNAIFDs will respond best to drugs that target elements of the network independent of target function and that early clinical trials of drugs targeted to genes comprising the 54 gene network might be best directed toward tumors with high MNAIs.

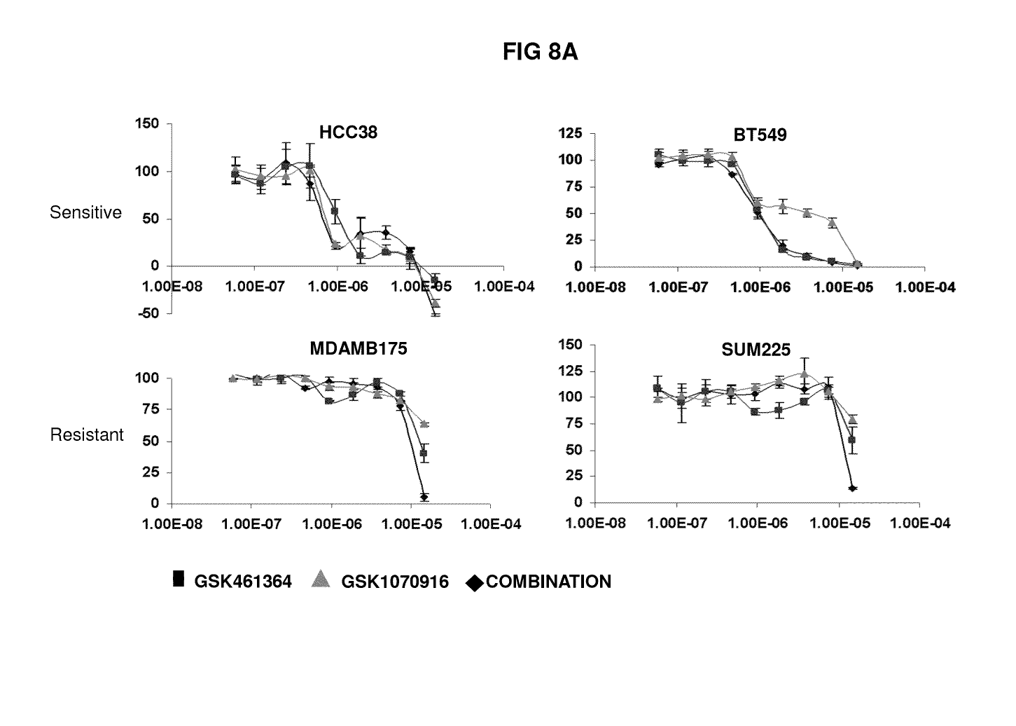

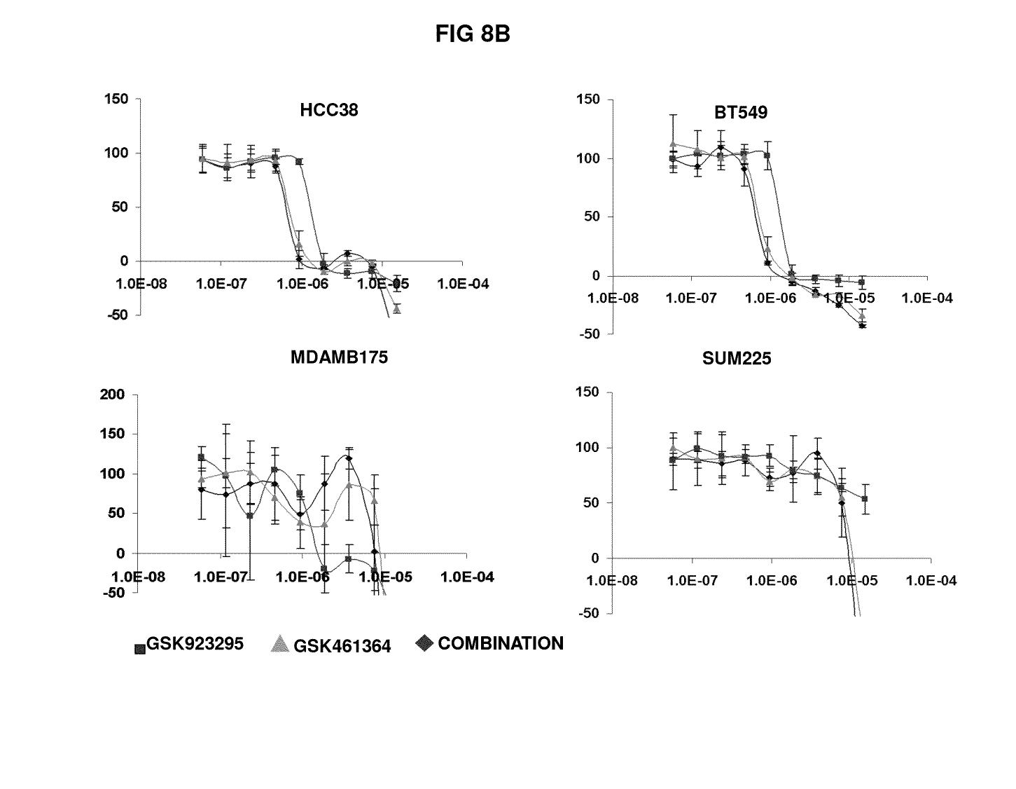

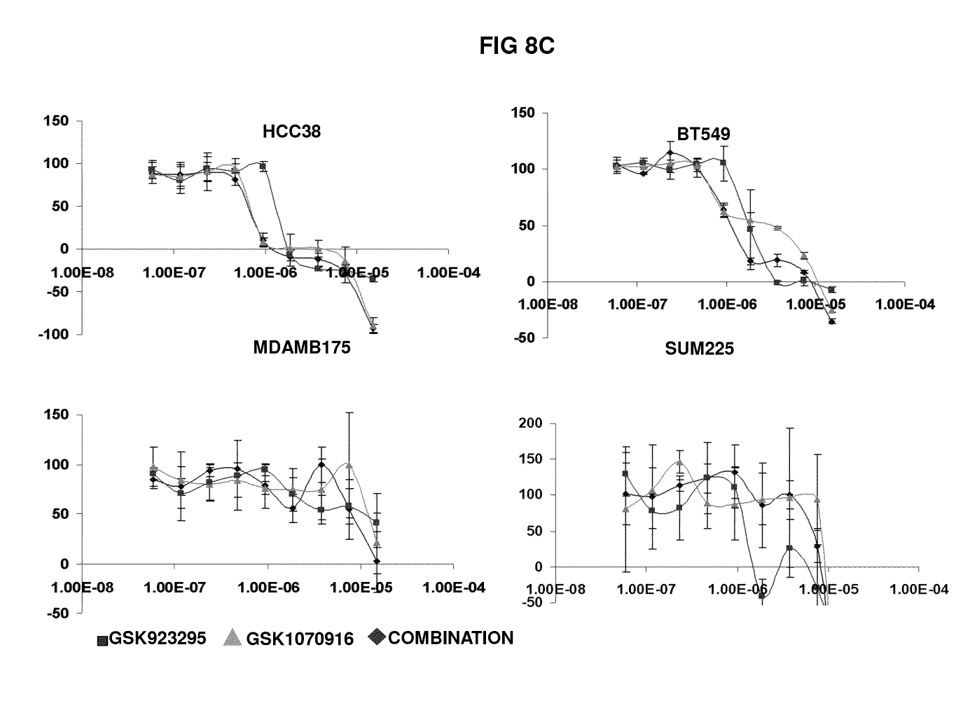

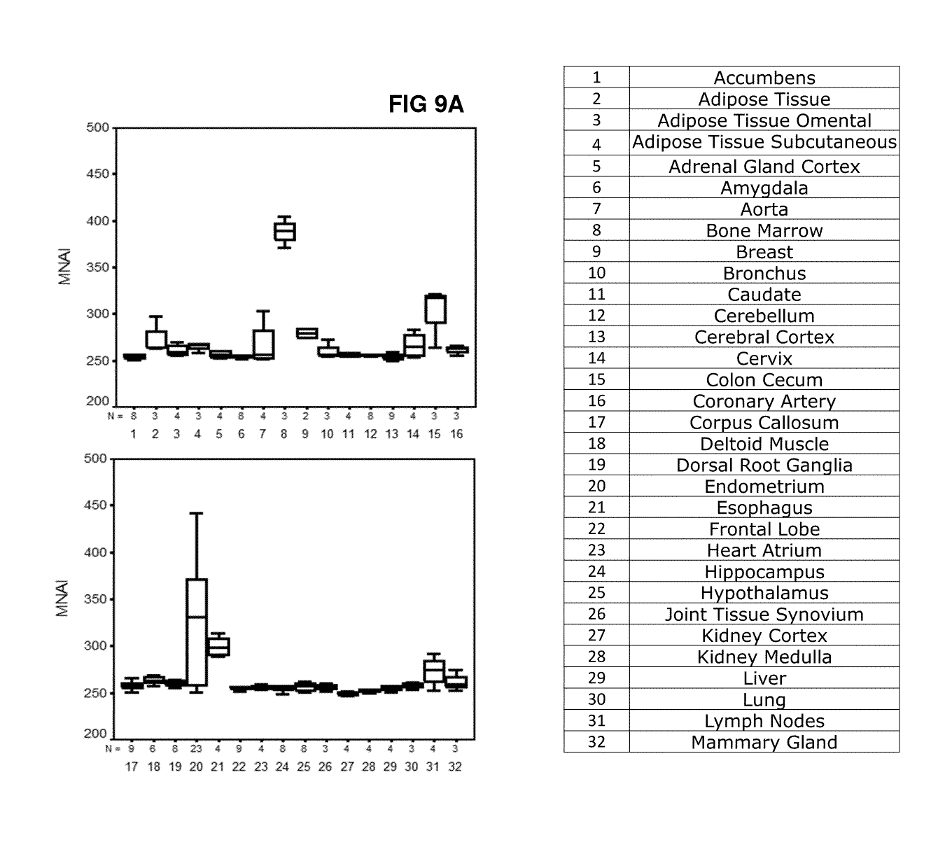

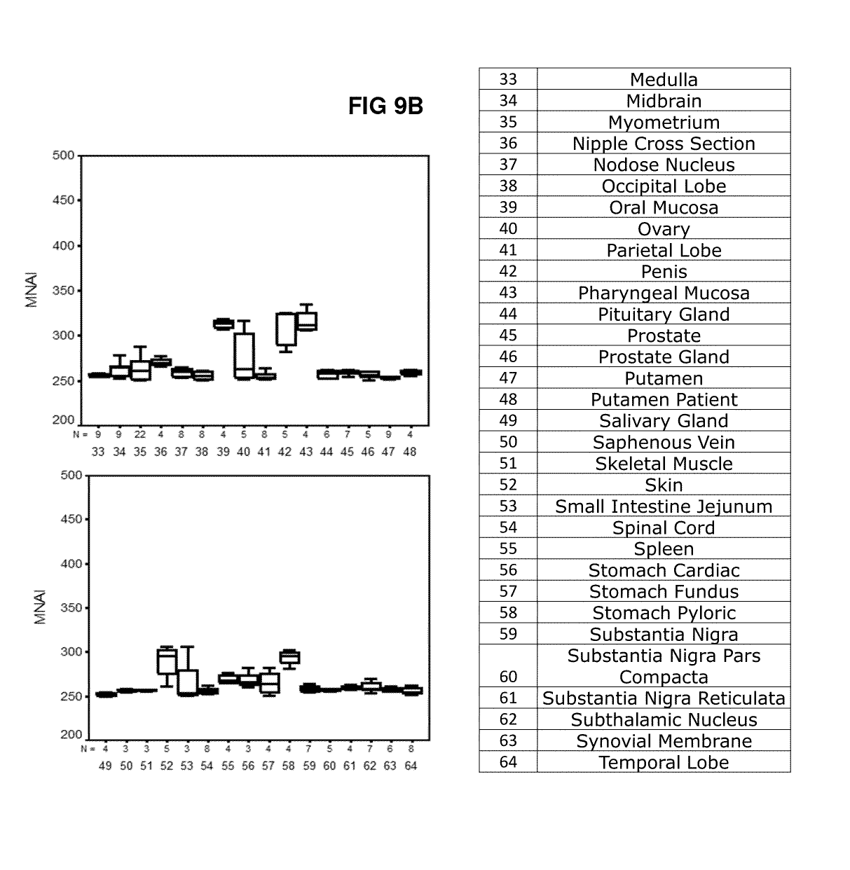

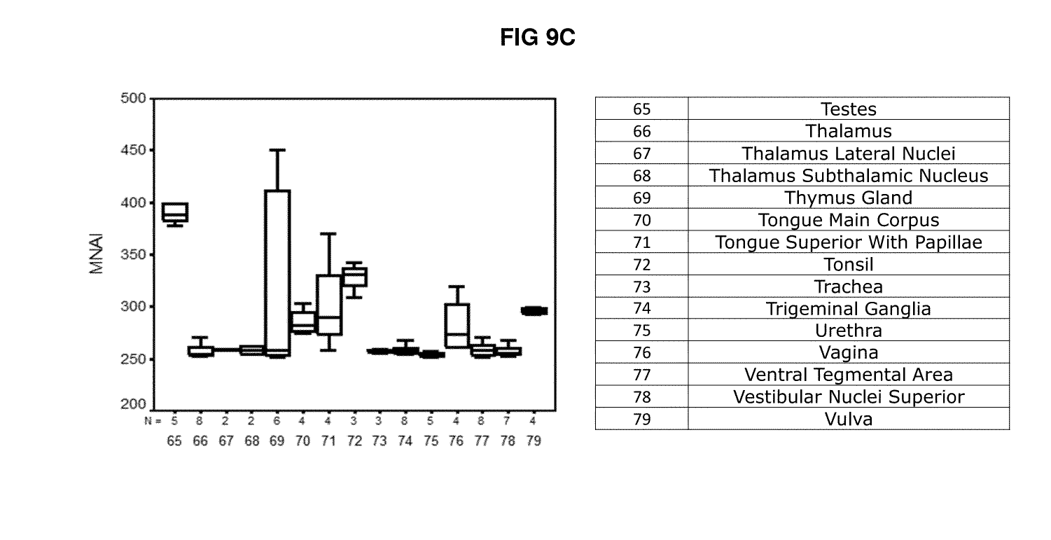

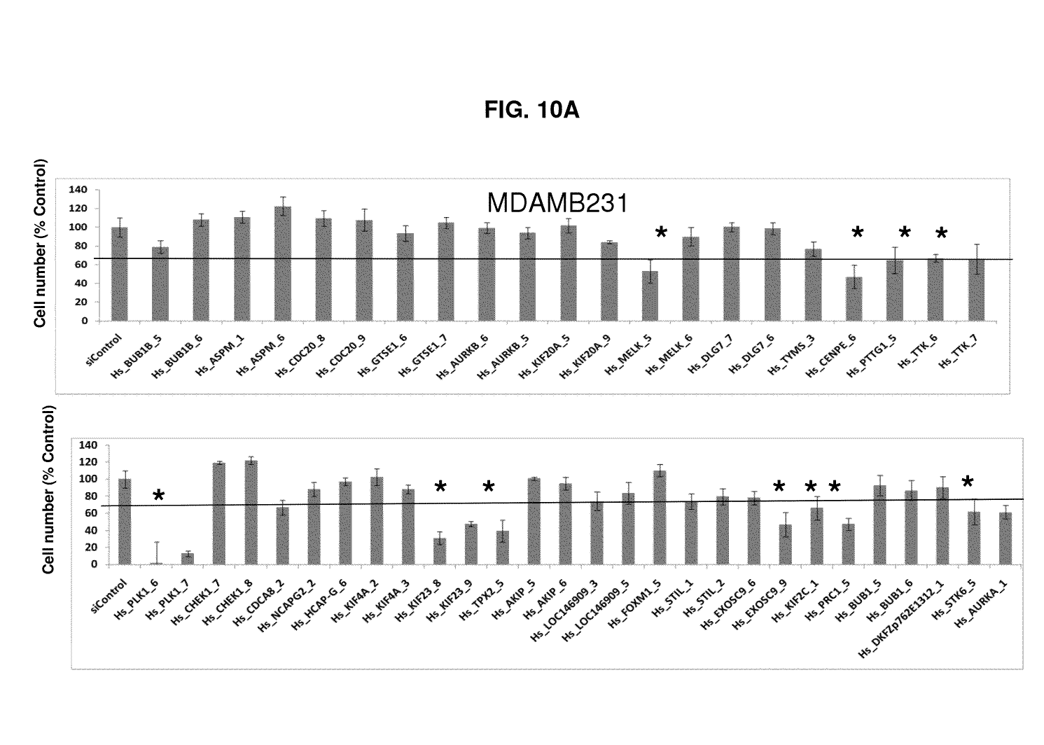

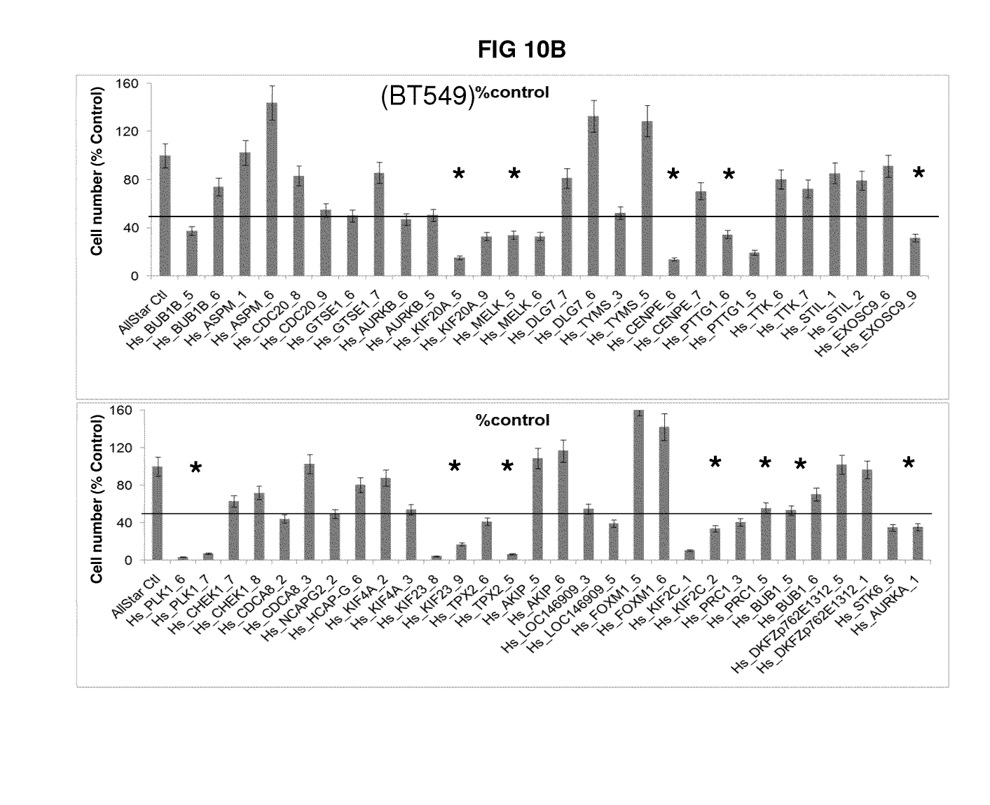

The data indicating the strong correlations between responses to the three drugs among the lines described in Table 1 suggests that drugs that attack within the mitotic apparatus network may be clinically and biologically equivalent. In that case, combinations of mitotic apparatus inhibitors would not be expected to show additive or synergistic effects. We tested this by treatment of sensitive and resistant breast cancer cell lines with GSK461364 or GSK1070916 or GSK923295 alone and in combination two of them at different doses. FIG. 8 details results from testing the combination of two compounds, indicating that two drugs together did not increase response in either sensitive or resistant cells. Since toxicity does not appear to be additive, combinations of drugs targeting the mitotic apparatus might be deployed either together or sequentially to counter development of drug resistance. That being the case, deployment of drugs targeting other genes in the mitotic apparatus network might contribute to development of a mitotic apparatus therapeutic armamentarium that could effectively counter development of drug resistance and provide durable treatments that will be effective against metastasis. Small molecular inhibitors have already been reported as under development for AURKA (MLN8054), CHEK2 (PD-321852). Protein motif assessment of the 54 genes in the mitotic network defined here suggests the mitotic checkpoint kinase, TTK; thymidylate synthase, TYMS; forkhead box M1, FOXM1; the serine/threonine kinase, MELK, the MAPKK-like protein kinase, PBK; and the protein kinease, BUB1 as druggable genes in the network. Work developing the mitotic network informs that inhibitors of these genes will be preferentially be effective in tumors with high MNAIs and are thus top candidates for drug development.

The 54 gene mitotic apparatus network is transcriptionally active in a subset of tumor and normal tissues of diverse types, and this increased activity correlates with reduced survival of patients having the condition that manifests this kind of expression activity. Mitotic network activity is defined as the sum of transcriptional expression levels of the 54-gene set. Small molecule inhibitors GSK461364, GS K923295 and GSK1070916 that target the network genes PLK1, CENPE and AURKB are preferentially effective in cell lines with high mitotic network activity. A sub-set of the mitotic network made up of about 6 to 8 genes can be used to identify patients most likely to respond to drugs that attack within the mitotic activity network. In breast cancer, basal subtype cancers tend to have high MNAIs.

Thus, the invention further provides for compositions and methods of detection for prognosis and diagnosis of disease and whether such patient will respond to specific therapies. In further embodiment, the invention also provides such methods for treating a patient.

In one embodiment, the invention provides for a method for identifying a cancer patient suitable for treatment with an inhibitor of mitotic activity, said method comprising the steps of: (a) measuring the expression level of at least one gene selected from the group consisting of the genes encoding MELK, SMC4, TEX10, AURKA, HJURP, BUB1, RFC3, and CCNB2 in a sample from the patient; and (b) comparing the expression level of said gene from the patient with the expression level of the gene in a normal tissue sample or a reference expression level (such as the average expression level of the gene in a cell line panel or a cancer cell or tumor panel, or the like), wherein an increase in the expression level or a decrease of expression of at least one gene selected from the group consisting of the genes encoding MELK, SMC4, TEX10, AURKA, HJURP, BUB1, RFC3, and CCNB2 indicates the patient is suitable for treatment with a mitotic network inhibitor Inhibitors that target mitotic activity include inhibitors against PLK1, CENPE and AURKB genes, such small molecule inhibitors GSK461364, GSK923295 and GSK1070916 that target the network genes PLK1, CENPE and AURKB compound.

In some embodiments, the methods further comprising measuring expression levels of at least 6, 7 or 8 of the genes in the group MELK, SMC4, TEX10, AURKA, HJURP, BUB1, RFC3, and CCNB2.

In other embodiments, the methods further comprising measuring expression levels of genes from the group, PLK1, SMC4, PBK, KIF14, NCAPD2, RRM2, CENPA, KNTC2, KIF23, RFC3, EXO1, LMNB2, TEX10, DEPDC1, DDX39, MAD2L1, C10orf13, FAM64A, TPX2, AURKA, and/or TTK.

In some embodiments of the invention, the method further comprises (c) measuring the expression level of a gene encoding PLK1, CENPE, or AURKB in a sample from the patient, and (d) comparing the expression level of the gene encoding PLK1, CENPE, or AURKB and the expression level of the gene encoding PLK1, CENPE, or AURKB in the normal tissue sample or a reference expression level (such as the average expression level of the gene in a cell line panel or a cancer cell or tumor panel, or the like), wherein an increase in the expression level of PLK1, CENPE, or AURKB indicates the patient is suitable for treatment with a PLK1, CENPE, or AURKB inhibitor, such as the GSK461364 compound.

In another embodiment, a method for identifying a cancer patient suitable for treatment with an inhibitor of mitotic activity, said method comprising the steps of: (a) measuring the expression level of at least one gene selected from the 18-gene set in a sample from the patient; and (b) comparing the expression level of said gene from the patient with the expression level of the gene in a normal tissue sample or a reference expression level (such as the average expression level of the gene in a cell line panel or a cancer cell or tumor panel, or the like), wherein an increase in the expression level or a decrease of expression of at least one gene selected from the 18-gene set indicates the patient is suitable for treatment with a mitotic network inhibitor.

In another embodiment, a method for identifying a cancer patient suitable for treatment with an inhibitor of mitotic activity, said method comprising the steps of: (a) measuring the expression level of at least one gene selected from the mitotic network 54-gene set in a sample from the patient; and (b) comparing the expression level of said gene from the patient with the expression level of the gene in a normal tissue sample or a reference expression level (such as the average expression level of the gene in a cell line panel or a cancer cell or tumor panel, or the like), wherein an increase in the expression level or a decrease of expression of at least one gene selected from the 54-gene set indicates the patient is suitable for treatment with a mitotic network inhibitor.

For these detection methods, if expression levels are increased in all or at least a sufficient number (e.g., greater than 30%, 40% or 50%, etc.) of the tested genes (i.e., cells having high mitotic network activity), then a determination can be made that the patient has a cancer that is likely of basal or basal-like subtype and a more aggressive treatment regimen may be adopted.

In another embodiment, a prognostic method for predicting the outcome of a patient by detection of high mitotic network activity in a patient tissue or biopsy. Thus, detection of increased expression of the mitotic network genes indicates the presence of aggressive cancers, i.e., the presence of cells in the tissue that will increase tumor progression and metastasize to other tissues. In some embodiments, the patient is lymph-node negative.

The expression level of a gene is measured by measuring the amount or number of molecules of mRNA or transcript in a cell. The measuring can comprise directly measuring the mRNA or transcript obtained from a cell, or measuring the cDNA obtained from an mRNA preparation thereof. Such methods of extracting the mRNA or transcript from a cell, or preparing the cDNA thereof are well known to those skilled in the art. In other embodiments, the expression level of a gene can be measured by measuring or detecting the amount of protein or polypeptide expressed, such as measuring the amount of antibody that specifically binds to the protein in a dot blot or Western blot. The proteins described in the present invention can be overexpressed and purified or isolated to homogeneity and antibodies raised that specifically bind to each protein. Such methods are well known to those skilled in the art.

The expression level of a gene is measured from a sample from the patient that comprises essentially a cancer cell or cancer tissue of a cancer tumor. Such methods for obtaining such samples are well known to those skilled in the art. When the cancer is breast cancer, the expression level of a gene is measured from a sample from the patient that comprises essentially a breast cancer cell or breast cancer tissue of a breast cancer tumor.

The cancer patient is either a patient who is known to be high MNAI-positive, that is, overexpresses a mitotic network protein(s), or is not known whether patient is high MNAI-positive or not. When the patient is not known whether to be high MNAI-positive or not, the status of the patient is to be determined.

Methods of assaying for protein overexpression include methods that utilize immunohistochemistry (IHC) and methods that utilize fluorescence in situ hybridization (FISH). A commercially available IHC test, for example, is PathVysion® (Vysis Inc., Downers Grove, Ill.). A commercially available FISH test is DAKO HercepTest® (DAKO Corp., Carpinteria, Calif.). The expression level of a gene encoding a mitotic network gene can be measured using an oligonucleotide derived from the nucleotide sequences of the GenBank Accession numbers indicated above in Table 4.

In some embodiments of the invention, the nucleotide sequence of a suitable fragment of the gene is used, or an oligonucleotide derived thereof. The length of the oligonucleotide of any suitable length. A suitable length can be at least 10 nucleotides, 20 nucleotides, 50 nucleotides, 100 nucleotides, 200 nucleotides, or 400 nucleotides, and up to 500 nucleotides or 700 nucleotides. A suitable nucleotide is one which binds specifically to a nucleic acid encoding the target gene and not to the nucleic acid encoding another gene.

In other embodiments, detection by increased expression is carried out by quantitative PCR, expression or transcription profiling, array comparative genomic hybridization (array CGH), or other techniques known and employed in the art. Methods for such detection are described in co-pending U.S Patent Application Publication Nos. 20050118634, 20060292591, and 20080312096, hereby incorporated by reference.

Methods of preparing probes are well known to those of skill in the art (see, e.g. Sambrook et al., Molecular Cloning: A Laboratory Manual (2nd ed.), Vols. 1-3, Cold Spring Harbor Laboratory, (1989) or Current Protocols in Molecular Biology, F. Ausubel et al., ed. Greene Publishing and Wiley-Interscience, New York (1987)), which are hereby incorporated by reference.

The probes are most easily prepared by combining and labeling one or more constructs. Prior to use, constructs are fragmented to provide smaller nucleic acid fragments that easily penetrate the cell and hybridize to the target nucleic acid. Fragmentation can be by any of a number of methods well known to hose of skill in the art. Preferred methods include treatment with a restriction enzyme to selectively cleave the molecules, or alternatively to briefly heat the nucleic acids in the presence of Mg²⁺. Probes are preferably fragmented to an average fragment length ranging from about 50 bp to about 2000 bp, more preferably from about 100 bp to about 1000 bp and most preferably from about 150 bp to about 500 bp.

Methods of labeling nucleic acids are well known to those of skill in the art. Preferred labels are those that are suitable for use in in situ hybridization. The nucleic acid probes may be detectably labeled prior to the hybridization reaction. Alternatively, a detectable label which binds to the hybridization product may be used. Such detectable labels include any material having a detectable physical or chemical property and have been well-developed in the field of immunoassays.

As used herein, a “label” is any composition detectable by spectroscopic, photochemical, biochemical, immunochemical, or chemical means. Useful labels in the present invention include radioactive labels(e.g., ³²P, ¹²⁵I, ¹⁴C, ³H, and ³⁵S), fluorescent dyes (e.g. fluorescein, rhodamine, Texas Red, etc.), electron-dense reagents (e.g. gold), enzymes (as commonly used in an ELISA), colorimetric labels (e.g. colloidal gold), magnetic labels (e.g. DYNABEADS™), and the like. Examples of labels which are not directly detected but are detected through the use of directly detectable label include biotin and dioxigenin as well as haptens and proteins for which labeled antisera or monoclonal antibodies are available.

The particular label used is not critical to the present invention, so long as it does not interfere with the in situ hybridization of the stain. However, stains directly labeled with fluorescent labels (e.g. fluorescein-12-dUTP, Texas Red-5-dUTP, etc.) are preferred for chromosome hybridization.

A direct labeled probe, as used herein, is a probe to which a detectable label is attached. Because the direct label is already attached to the probe, no subsequent steps are required to associate the probe with the detectable label. In contrast, an indirect labeled probe is one which bears a moiety to which a detectable label is subsequently bound, typically after the probe is hybridized with the target nucleic acid.

In addition the label must be detectable in as low copy number as possible thereby maximizing the sensitivity of the assay and yet be detectible above any background signal. Finally, a label must be chosen that provides a highly localized signal thereby providing a high degree of spatial resolution when physically mapping the stain against the chromosome. Particularly preferred fluorescent labels include fluorescein-12-dUTP and Texas Red-5-dUTP.

The labels may be coupled to the probes in a variety of means known to those of skill in the art. In a preferred embodiment the nucleic acid probes will be labeled using nick translation or random primer extension (Rigby, et al. J. Mol. Biol., 113: 237 (1977) or Sambrook, et al., Molecular Cloning—A Laboratory Manual, Cold Spring Harbor Laboratory, Cold Spring Harbor, N.Y. (1985)).

One of skill in the art will appreciate that the probes of this invention need not be absolutely specific for the targeted 1q21 region of the genome. Rather, the probes are intended to produce “staining contrast”. “Contrast” is quantified by the ratio of the probe intensity of the target region of the genome to that of the other portions of the genome. For example, a DNA library produced by cloning a particular chromosome (e.g. chromosome 7) can be used as a stain capable of staining the entire chromosome. The library contains both sequences found only on that chromosome, and sequences shared with other chromosomes. Roughly half the chromosomal DNA falls into each class. If hybridization of the whole library were capable of saturating all of the binding sites on the target chromosome, the target chromosome would be twice as bright (contrast ratio of 2) as the other chromosomes since it would contain signal from the both the specific and the shared sequences in the stain, whereas the other chromosomes would only be stained by the shared sequences. Thus, only a modest decrease in hybridization of the shared sequences in the stain would substantially enhance the contrast. Thus contaminating sequences which only hybridize to non-targeted sequences, for example, impurities in a library, can be tolerated in the stain to the extent that the sequences do not reduce the staining contrast below useful levels.

In some embodiments, amplification is detected through the hybridization of a probe of a mitotic network gene to a target nucleic acid (e.g. a chromosomal sample) in which it is desired to screen for the amplification. Suitable hybridization formats are well known to those of skill in the art and include, but are not limited to, variations of Southern Blots, in situ hybridization and quantitative amplification methods such as quantitative PCR (see, e.g. Sambrook, supra., Kallioniemi et al., Proc. Natl Acad Sci USA, 89: 5321-5325 (1992), and PCR Protocols, A Guide to Methods and Applications, Innis et al., Academic Press, Inc. N.Y., (1990)).

In situ Hybridization. In another embodiment, high mitotic network activity or amplification of a gene in the mitotic network is identified using in situ hybridization.

Generally, in situ hybridization comprises the following major steps: (1) fixation of tissue or biological structure to analyzed; (2) prehybridization treatment of the biological structure to increase accessibility of target DNA, and to reduce nonspecific binding; (3) hybridization of the mixture of nucleic acids to the nucleic acid in the biological structure or tissue; (4) posthybridization washes to remove nucleic acid fragments not bound in the hybridization and (5) detection of the hybridized nucleic acid fragments. The reagent used in each of these steps and their conditions for use vary depending on the particular application.

In some applications it is necessary to block the hybridization capacity of repetitive sequences. In this case, human genomic DNA is used as an agent to block such hybridization. The preferred size range is from about 200 bp to about 1000 bases, more preferably between about 400 to about 800 bp for double stranded, nick translated nucleic acids.

Hybridization protocols for the particular applications disclosed here are described in Pinkel et al. Proc. Natl. Acad. Sci. USA, 85: 9138-9142 (1988) and in EPO Pub. No. 430,402. Suitable hybridization protocols can also be found in Methods in Molecular Biology Vol. 33, In Situ Hybridization Protocols, K. H. A. Choo, ed., Humana Press, Totowa, N.J., (1994). In a particularly preferred embodiment, the hybridization protocol of Kallioniemi et al., ERBB2 amplification in breast cancer analyzed by fluorescence in situ hybridization. Proc Natl Acad Sci USA, 89: 5321-5325 (1992) is used.

Typically, it is desirable to use dual color FISH, in which two probes are utilized, each labeled by a different fluorescent dye. A test probe that hybridizes to the region of interest is labeled with one dye, and a control probe that hybridizes to a different region is labeled with a second dye. A nucleic acid that hybridizes to a stable portion of the chromosome of interest, such as the centromere region, is often most useful as the control probe. In this way, differences between efficiency of hybridization from sample to sample can be accounted for.

The FISH methods for detecting chromosomal abnormalities can be performed on nanogram quantities of the subject nucleic acids. Paraffin embedded tumor sections can be used, as can fresh or frozen material. Because FISH can be applied to the limited material, touch preparations prepared from uncultured primary tumors can also be used (see, e.g., Kallioniemi, A. et al., Cytogenet. Cell Genet. 60: 190-193 (1992)). For instance, small biopsy tissue samples from tumors can be used for touch preparations (see, e.g., Kallioniemi, A. et al., Cytogenet. Cell Genet. 60: 190-193 (1992)). Small numbers of cells obtained from aspiration biopsy or cells in bodily fluids (e.g., blood, urine, sputum and the like) can also be analyzed. For prenatal diagnosis, appropriate samples will include amniotic fluid and the like.

It is preferred that the assay is validated by application to a larger sample for validation in a retrospective analysis of paraffin embedded samples from a large sample of moderate and high risk breast cancers (˜60% five year survival), the majority treated with platinum based therapy and a large sample of high risk cancers treated with cisplatinum and taxane.

In another embodiment, the assay can be used to determine the efficacy of traditional, current and new treatment protocols.

In another embodiment, elevated gene expression is detected using quantitative PCR. Primers can be created using the sequences of genes identified Table 4, to detect sequence amplification by signal amplification in gel electrophoresis. As is known in the art, primers or oligonucleotides are generally 15-40 bp in length, and usually flank unique sequence that can be amplified by methods such as polymerase chain reaction (PCR) or reverse transcriptase PCR (RT-PCR, also known as real-time PCR). Methods for RT-PCR and its optimization are known in the art. An example is the PROMEGA PCR Protocols and Guides, found at URL:<http://www.promega.com/guides/per_guide/default.htm>, and hereby incorporated by reference. Currently at least four different chemistries, TaqMan® (Applied Biosystems, Foster City, Calif., USA), Molecular Beacons, Scorpions® and SYBR® Green (Molecular Probes), are available for real-time PCR. All of these chemistries allow detection of PCR products via the generation of a fluorescent signal. TaqMan probes, Molecular Beacons and Scorpions depend on Förster Resonance Energy Transfer (FRET) to generate the fluorescence signal via the coupling of a fluorogenic dye molecule and a quencher moiety to the same or different oligonucleotide substrates. SYBR Green is a fluorogenic dye that exhibits little fluorescence when in solution, but emits a strong fluorescent signal upon binding to double-stranded DNA.

Two strategies are commonly employed to quantify the results obtained by real-time RT-PCR; the standard curve method and the comparative threshold method. In this method, a standard curve is first constructed from an RNA of known concentration. This curve is then used as a reference standard for extrapolating quantitative information for mRNA targets of unknown concentrations. Another quantitation approach is termed the comparative Cₜ method. This involves comparing the Cₜ values of the samples of interest with a control or calibrator such as a non-treated sample or RNA from normal tissue. The Cₜ values of both the calibrator and the samples of interest are normalized to an appropriate endogenous housekeeping gene.

In one embodiment, elevated gene expression is detected using an RT-PCR assay to detect transcription levels or detected using a PCR assay to detect amplification of at least one gene from the mitotic network.

In some embodiments, elevated expression of mitotic network gene is detected using an immunochemical assay to detect protein levels. Such immunochemical assays are known throughout the art and include Western blots and ELISAs.

In one embodiment, using known methods of antibody production, antibodies to mitotic network gene are made. In some embodiments, elevated mitotic network gene expression is detected using an immunochemical (IHC) assay to detect mitotic network gene protein levels. Anti-mitotic network gene specific antibodies can be made by general methods known in the art. A preferred method of generating these antibodies is by first synthesizing peptide fragments. These peptide fragments should likely cover unique coding regions in the candidate gene. Since synthesized peptides are not always immunogenic by their own, the peptides should be conjugated to a carrier protein before use. Appropriate carrier proteins include but are not limited to Keyhole limpet hemacyanin (KLH). The conjugated phospho peptides should then be mixed with adjuvant and injected into a mammal, preferably a rabbit through intradermal injection, to elicit an immunogenic response. Samples of serum can be collected and tested by ELISA assay to determine the titer of the antibodies and then harvested.

Polyclonal (e.g., anti-mitotic network gene) antibodies can be purified by passing the harvested antibodies through an affinity column. Monoclonal antibodies are preferred over polyclonal antibodies and can be generated according to standard methods known in the art of creating an immortal cell line which expresses the antibody.

Nonhuman antibodies are highly immunogenic in human and that limits their therapeutic potential. In order to reduce their immunogenicity, nonhuman antibodies need to be humanized for therapeutic application. Through the years, many researchers have developed different strategies to humanize the nonhuman antibodies. One such example is using “HuMAb-Mouse” technology available from MEDAREX, Inc. and disclosed by van de Winkel, in U.S. Pat. No. 6,111,166 and hereby incorporated by reference in its entirety. “HuMAb-Mouse” is a strain of transgenic mice which harbor the entire human immunoglobin (Ig) loci and thus can be used to produce fully human monoclonal antibodies such as monoclonal anti-mitotic network gene antibodies.

In another embodiment, a prognostic method for predicting the outcome of a patient by detection of mitotic network gene over expression in a patient tissue or biopsy using an immunohistochemical assay as compared to normal levels in a control sample. Presence of or over expression of mitotic network gene detected can be used as an indicator of metastatic or invasive cells present in the patient tissue, which may likely lead to metastatic cancer in the near future. In another embodiment, over expression of mitotic network gene can be determined by comparison to a reference expression level (such as the average expression level of the gene in a cell line panel or a cancer cell or tumor panel, or the like).

In another embodiment, a prognostic method to provide more accurate prognosis for patients having non-invasive cancer (e.g., lymph-node negative cancer) previously determined based on morphology by a pathologist. A new biopsy can be taken or biopsies previously taken and preserved (e.g., in paraffin) can be used. In addition to observing morphology of a tumor (e.g., histological grade, stage and size), detection of mitotic network gene over expression can be carried out by IHC assay and a new prognosis determined, factoring in the finding of level of mitotic network gene expression levels. For example, a finding by IHC that mitotic network gene is present at an increased level as compared to a normal tissue, despite the morphology of a non-invasive cancer, will indicate that the tumor should be staged or graded higher as a tumor that will be invasive and aggressive, leading to metastasis.

In another embodiment, array comparative genomic hybridization (CGH) and expression profiling to localize aberrant genes in a patient is contemplated. Analysis of genome copy number abnormalities of mitotic network gene using array CGH (Hodgson, G. et al. Genome scanning with array CGH delineates regional alterations in mouse islet carcinomas. Nat Genet 29, 459-64 (2001); Snijders, A. M. et al. Assembly of microarrays for genome-wide measurement of DNA copy number. Nat Genet 29, 263-4 (2001)) can be performed. In another embodiment, gene expression of mitotic network gene is analyzed using an array such as the Affymetrix U133A array platform(Lancaster, J. M. et al. Gene expression patterns that characterize advanced stage serous ovarian cancers. J Soc Gynecol Investig 11, 51-9 (2004). In one embodiment, a finding of an increased expression profile of mitotic network gene by about 1.5-fold is indicative of over expression and indicates early detection of breast cancer. In another embodiment, a finding of an increased expression profile of mitotic network gene by about 1.5-fold is indicative of over expression and a prognosis of poor outcome in cancer.

The present invention further provides kits for use within any of the above diagnostic methods. Such kits typically comprise two or more components necessary for performing a diagnostic assay. Components may be compounds, reagents, containers and/or equipment.

In one embodiment, one container within a kit may contain a set of FISH probes for detection of amplification of mitotic network genes at different loci. One or more additional containers may enclose elements, such as reagents or buffers, to be used in the assay. Such kits may also, or alternatively, contain a detection reagent as described above that contains a reporter group suitable for direct or indirect detection of antibody binding.

In another embodiment, the kit may be comprised of a set of PCR primers to detect sequence amplification of genes in the mitotic network. The kit would also contain such reagents as buffers, polymerase, Magnesium, or other elements necessary to carry out quantitative PCR.

Mitotic Network 18-Gene or 22-Gene Set as Therapeutic Targets

Prognostic markers that identify subsets of patients with very poor survival prospects are of modest clinical importance unless therapies can be developed for these patients. Our approach to therapy for these patients is to develop inhibitors of genes that are over expressed in the regions of amplification associated with reduced survival. It is contemplated that these candidate genes may be over expressed in diseases including but not limited, cancers, lymphomas, cardiovascular diseases, cardiac hypertrophy, and infectious diseases.

In one embodiment, genome wide analyses of genome copy number and gene expression in serous breast cancers showed that mitotic network genes are amplified and over-expressed. The 18-gene and the 22-gene subset of the 54 mitotic network genes are considered to be therapeutic targets in diseases wherein they are over expressed and associated with short survival rates.

In some embodiments, mitotic network genes are targets for development of therapeutics and diagnostic assays. In one embodiment, an assay to detect elevated mitotic network gene expression as a predictor of poor response to current drugs based therapies, such as taxol plus platinum based therapies, in serous breast cancers. In such an assay, elevated mitotic network gene expression can be detected using methods known in the art or described above. It is contemplated that elevated mitotic network gene expression can be detected in a subject by testing various tissues and bodily fluids, including but not limited to blood and serum. Thus, detection of elevated mitotic network gene expression will indicate that the patient will likely respond poorly to current drug based therapies and is a candidate for use of other types of cancer therapies, combination therapies, and possibly require a therapeutic regimen usually reserved for later stage cancers.

In another embodiment, the detection of a mitotic network gene indicates that the patient should receive mitotic network gene-targeted therapeutics. Describe herein are several types of therapeutics which can be used and further developed to target mitotic network genes.

Inhibitor Oligonucleotides and RNA interference (RNAi). The approaches to be taken will depend on the detailed characteristics of the genes, but in some embodiments, will begin with strategies to inhibit RNA transcription since they can, in principal, be used to attack over expressed genes independent of their biochemical composition. Work in the past two decades on transcriptional inhibitors focused on oligodeoxynucleotides and ribozymes. These approaches have had some clinical success but delivery issues limited their clinical utility. Recently, however, advances in short interfering RNA (siRNA) technology and biological understanding have accelerated development of anti-gene therapies (Wall, N. R. & Shi, Y. Small RNA: can RNA interference be exploited for therapy? Lancet 362, 1401-3 (2003); Scanlon, K. J. Anti-genes: siRNA, ribozymes and antisense. Curr Pharm Biotechnol 5, 415-20 (2004); Buckingham, S. D., Esmaeili, B., Wood, M. & Sattelle, D. B. RNA interference: from model organisms towards therapy for neural and neuromuscular disorders. Hum Mol Genet 13 Spec No 2, R275-88 (2004)). Promising therapeutic approaches include siRNAs complexed with cationic liposomes (Liao, Y., et al., Enhanced paclitaxel cytotoxicity and prolonged animal survival rate by a nonviral-mediated systemic delivery of EIA gene in orthotopic xenograft human breast cancer. Cancer Gene Ther 11, 594-602 (2004); Yano, J. et al. Antitumor activity of small interfering RNA/cationic liposome complex in mouse models of cancer. Clin Cancer Res 10, 7721-6 (2004)), virus vector-mediated RNAi (Zhao, N. et al. Knockdown of Mouse Adult beta-Globin Gene Expression in MEL Cells by Retrovirus Vector-Mediated RNA Interference. Mol Biotechnol 28, 195-200 (2004); Sumimoto, H. et al. Gene therapy for human small-cell lung carcinoma by inactivation of Skp-2 with virally mediated RNA interference. Gene Ther (2004)) and nanoparticles adapted for siRNA (Schiffelers, R. M. et al. Cancer siRNA therapy by tumor selective delivery with ligand-targeted sterically stabilized nanoparticle. Nucleic Acids Res 32, e149 (2004)). In one embodiment, siRNAs against the high priority targets complexed with cationic liposomes and small molecule approaches to inhibit the over expressed candidate genes will allow rapid development of this line of attack.

In some embodiments, the expression of the mitotic network genes is manipulated. In one embodiment, such manipulation can be made using optimized siRNAs. See Hannon, G. J. RNA interference (2002); Plasterk, R. H. in Science 1263-5 (2002); and Elbashir, S. M. et al. in Nature 494-8 (2001). Strong Pearson correlations between target gene amplification/expression levels and pro-apoptotic effects of siRNAs will indicate that copy number/expression levels determine the extent of apoptotic responses to target gene inhibitors.

In another embodiment, treatment of amplified cells simultaneously with siRNAs against the mitotic network genes plus PLK1, CENPE or AURKB inhibitors, such as GSK461364 or GSK1070916 or GSK923295 respectively, should result in the inhibition of mitotic network gene activity and enhance patient response to inhibitors such as GSK461364 or GSK1070916 or GSK923295. Greater than additive induction of apoptosis in these dual treatment experiments will indicate a synergistic effect.

The invention further provides for compounds to treat patients with elevated mitotic network gene expression, more specifically elevated expression of the 18-gene set. In one embodiment, the compound is a mitotic network gene inhibitor such as, an antisense oligonucleotide; a siRNA oligonucleotide; a small molecule that interferes with mitotic network gene function; a viral vector producing a nucleic acid sequence that inhibits mitotic network gene; or an aptamer.

High throughput methods can be used to identify mitotic network gene inhibitors such as siRNA and/or small molecular inhibitor formulations to deliver mitotic network gene (and other) inhibitors efficiently to cultured cells and xenografts. Mitotic network gene inhibitory formulations will be preferentially effective against xenografts that are amplified at the target loci. In another embodiment, that 18-gene or 22-gene set inhibitors will enhance response to PLK1, CENPE or AURKB inhibitor compounds GSK461364 or GSK1070916 or GSK923295. Effective formulations using such methods as described above can be developed for clinical application.

In some embodiments, known methods are used to identify sequences that inhibit mitotic network gene candidate genes which are related to drug resistance and reduced survival rates. Such inhibitors may include but are not limited to, siRNA oligonucleotides, antisense oligonucleotides, peptide inhibitors and aptamer sequences that bind and act to inhibit mitotic network gene expression and/or function.

In one embodiment, RNA interference is used to generate small double-stranded RNA (small interference RNA or siRNA) inhibitors to affect the expression of a candidate gene generally through cleaving and destroying its cognate RNA. Small interference RNA (siRNA) is typically 19-22 nt double-stranded RNA. siRNA can be obtained by chemical synthesis or by DNA-vector based RNAi technology. Using DNA vector based siRNA technology, a small DNA insert (about 70 bp) encoding a short hairpin RNA targeting the gene of interest is cloned into a commercially available vector. The insert-containing vector can be transfected into the cell, and expressing the short hairpin RNA. The hairpin RNA is rapidly processed by the cellular machinery into 19-22 nt double stranded RNA (siRNA). In a preferred embodiment, the siRNA is inserted into a suitable RNAi vector because siRNA made synthetically tends to be less stable and not as effective in transfection.

siRNA can be made using methods and algorithms such as those described by Wang L, Mu F Y. (2004) A Web-based Design Center for Vector-based siRNA and siRNA cassette. Bioinformatics. (In press); Khvorova A, Reynolds A, Jayasena S D. (2003) Functional siRNAs and miRNAs exhibit strand bias. Cell. 115(2):209-16; Harborth J, Elbashir S M, Vandenburgh K, Manninga H, Scaringe S A, Weber K, Tuschl T. (2003) Sequence, chemical, and structural variation of small interfering RNAs and short hairpin RNAs and the effect on mammalian gene silencing. Antisense Nucleic Acid Drug Dev. 13(2):83-105; Reynolds A, Leake D, Boese Q, Scaringe S, Marshall W S, Khvorova A. (2004) Rational siRNA design for RNA interference. Nat Biotechnol. 22(3):326-30 and Ui-Tei K, Naito Y, Takahashi F, Haraguchi T, Ohki-Hamazaki H, Juni A, Ueda R, Saigo K. (2004) Guidelines for the selection of highly effective siRNA sequences for mammalian and chick RNA interference. Nucleic Acids Res. 32(3):936-48, which are hereby incorporated by reference.

Other tools for constructing siRNA sequences are web tools such as the siRNA Target Finder and Construct Builder available from GenScript (http://www.genscript.com), Oligo Design and Analysis Tools from Integrated DNA Technologies (URL:<http://www.idtdna.com/SciTools/SciTools.aspx>), or siDESIGN™ Center from Dharmacon, Inc. (URL:<http://design.dharmacon.com/defaulfaspx?source=0>). siRNA are suggested to be built using the ORF (open reading frame) as the target selecting region, preferably 50-100 nt downstream of the start codon. Because siRNAs function at the mRNA level, not at the protein level, to design an siRNA, the precise target mRNA nucleotide sequence may be required. Due to the degenerate nature of the genetic code and codon bias, it is difficult to accurately predict the correct nucleotide sequence from the peptide sequence. Additionally, since the function of siRNAs is to cleave mRNA sequences, it is important to use the mRNA nucleotide sequence and not the genomic sequence for siRNA design, the genomic sequence can be successfully used for siRNA design. However, designs using genomic information might inadvertently target introns and as a result the siRNA would not be functional for silencing the corresponding mRNA.

Rational siRNA design should also minimize off-target effects which often arise from partial complementarity of the sense or antisense strands to an unintended target. These effects are known to have a concentration dependence and one way to minimize off-target effects is often by reducing siRNA concentrations. Another way to minimize such off-target effects is to screen the siRNA for target specificity.

In one embodiment, the siRNA can be modified on the 5′-end of the sense strand to present compounds such as fluorescent dyes, chemical groups, or polar groups. Modification at the 5′-end of the antisense strand has been shown to interfere with siRNA silencing activity and therefore this position is not recommended for modification. Modifications at the other three termini have been shown to have minimal to no effect on silencing activity.

It is recommended that primers be designed to bracket one of the siRNA cleavage sites as this will help eliminate possible bias in the data (i.e., one of the primers should be upstream of the cleavage site, the other should be downstream of the cleavage site). Bias may be introduced into the experiment if the PCR amplifies either 5′ or 3′ of a cleavage site, in part because it is difficult to anticipate how long the cleaved mRNA product may persist prior to being degraded. If the amplified region contains the cleavage site, then no amplification can occur if the siRNA has performed its function.

In some embodiments, siRNAs are designed based upon the mRNA sequence identified in Table 4 from GenBank, or similar thereto.

In another embodiment, antisense oligonucleotides (“oligos”) can be designed to inhibit mitotic network gene and other candidate gene function. Antisense oligonucleotides are short single-stranded nucleic acids, which function by selectively hybridizing to their target mRNA, thereby blocking translation. Translation is inhibited by either RNase H nuclease activity at the DNA:RNA duplex, or by inhibiting ribosome progression, thereby inhibiting protein synthesis. This results in discontinued synthesis and subsequent loss of function of the protein for which the target mRNA encodes.

In some embodiments, antisense oligos are phosphorothioated upon synthesis and purification, and are usually 18-22 bases in length. It is contemplated that the mitotic network gene and other candidate gene antisense oligos may have other modifications such as 2′-O-Methyl RNA, methylphosphonates, chimeric oligos, modified bases and many others modifications, including fluorescent oligos.

In some embodiments, active antisense oligos should be compared against control oligos that have the same general chemistry, base composition, and length as the antisense oligo. These can include inverse sequences, scrambled sequences, and sense sequences. The inverse and scrambled are recommended because they have the same base composition, thus same molecular weight and Tm as the active antisense oligonucleotides. Rational antisense oligo design should consider, for example, that the antisense oligos do not anneal to an unintended mRNA or do not contain motifs known to invoke immunostimulatory responses such as four contiguous G residues, palindromes of 6 or more bases and CG motifs.

Antisense oligonucleotides can be used in vitro in most cell types with good results. However, some cell types require the use of transfection reagents to effect efficient transport into cellular interiors. It is recommended that optimization experiments be performed by using differing final oligonucleotide concentrations in the 1-5 μm range with in most cases the addition of transfection reagents. The window of opportunity, i.e., that concentration where you will obtain a reproducible antisense effect, may be quite narrow, where above that range you may experience confusing non-specific, non-antisense effects, and below that range you may not see any results at all. In a preferred embodiment, down regulation of the targeted mRNA (e.g. mitotic network gene mRNA SEQ ID NO: 1) will be demonstrated by use of techniques such as northern blot, real-time PCR, cDNA/oligo array or western blot. The same endpoints can be made for in vivo experiments, while also assessing behavioral endpoints.

For cell culture, antisense oligonucleotides should be re-suspended in sterile nuclease-free water (the use of DEPC-treated water is not recommended). Antisense oligonucleotides can be purified, lyophilized, and ready for use upon re-suspension. Upon suspension, antisense oligonucleotide stock solutions may be frozen at −20° C. and stable for several weeks.

In another embodiment, aptamer sequences which bind to specific RNA or DNA sequences can be made. Aptamer sequences can be isolated through methods such as those disclosed in co-pending U.S. Patent Appl. Publ. No. 20090075834, entitled, “Aptamers and Methods for their Invitro Selection and Uses Thereof,” which is hereby incorporated by reference.

It is contemplated that the sequences described herein may be varied to result in substantially homologous sequences which retain the same function as the original. As used herein, a polynucleotide or fragment thereof is “substantially homologous” (or “substantially similar”) to another if, when optimally aligned (with appropriate nucleotide insertions or deletions) with the other polynucleotide (or its complementary strand), using an alignment program such as BLASTN (Altschul, S. F., Gish, W., Miller, W., Myers, E. W. & Lipman, D. J. (1990) “Basic local alignment search tool.” J. Mol. Biol. 215:403-410), and there is nucleotide sequence identity in at least about 80%, preferably at least about 90%, and more preferably at least about 95-98% of the nucleotide bases.

It is further contemplated and would be well accepted by one with skill in the art that antibodies can be made to any mitotic network gene as described above in Tables 4 and 5. In one embodiment, a method of treatment using a humanized monoclonal antibody to down-regulate a mitotic network gene.

In one embodiment, high throughput screening (HTS) methods are used to identify compounds that inhibit mitotic network genes in Tables 4 and 5. HTS methods involve providing a combinatorial chemical or peptide library containing a large number of potential therapeutic compounds (i.e., compounds that inhibit mitotic network gene and other candidate genes which are related to drug resistance). Such “libraries” are then screened in one or more assays, as described herein, to identify those library members (particular peptides, chemical species or subclasses) that display the desired characteristic activity. The compounds thus identified can serve as conventional “lead compounds” or can themselves be used as potential or actual therapeutics.

A combinatorial chemical library is a collection of diverse chemical compounds generated by either chemical synthesis or biological synthesis, by combining a number of chemical “building blocks” such as reagents. For example, a linear combinatorial chemical library such as a polypeptide library is formed by combining a set of chemical building blocks (amino acids) in every possible way for a given compound length (i.e., the number of amino acids in a polypeptide compound). Millions of chemical compounds can be synthesized through such combinatorial mixing of chemical building blocks.

Preparation and screening of combinatorial chemical libraries is well known to those of skill in the art. Such combinatorial chemical libraries include, but are not limited to, peptide libraries (see, e.g., U.S. Pat. No. 5,010,175, Furka, Int. J. Pept. Prot. Res. 37:487-493 (1991) and Houghton et al., Nature 354:84-88 (1991)). Other chemistries for generating chemical diversity libraries can also be used. Such chemistries include, but are not limited to: peptoids (e.g., PCT Publication No. WO 91/19735), encoded peptides (e.g., PCT Publication WO 93/20242), random bio-oligomers (e.g., PCT Publication No. WO 92/00091), benzodiazepines (e.g., U.S. Pat. No. 5,288,514), diversomers such as hydantoins, benzodiazepines and dipeptides (Hobbs et al., Proc. Nat. Acad. Sci. USA 90:6909-6913 (1993)), vinylogous polypeptides (Hagihara et al., J. Amer. Chem. Soc. 114:6568 (1992)), nonpeptidal peptidomimetics with glucose scaffolding (Hirschmann et al., J. Amer. Chem. Soc. 114:9217-9218 (1992)), analogous organic syntheses of small compound libraries (Chen et al., J. Amer. Chem. Soc. 116:2661 (1994)), oligocarbamates (Cho et al., Science 261:1303 (1993)), and/or peptidyl phosphonates (Campbell et al., J. Org. Chem. 59:658 (1994)), nucleic acid libraries (see Ausubel, Berger and Sambrook, all supra), peptide nucleic acid libraries (see, e.g., U.S. Pat. No. 5,539,083), antibody libraries (see, e.g., Vaughn et al., Nature Biotechnology, 14(3):309-314 (1996) and PCT/US96/10287), carbohydrate libraries (see, e.g., Liang et al., Science, 274:1520-1522 (1996) and U.S. Pat. No. 5,593,853), small organic molecule libraries (see, e.g., benzodiazepines, Baum C&EN, January 18, page 33 (1993); isoprenoids, U.S. Pat. No. 5,569,588; thiazolidinones and metathiazanones, U.S. Pat. No. 5,549,974; pyrrolidines, U.S. Pat. Nos. 5,525,735 and 5,519,134; morpholino compounds, U.S. Pat. No. 5,506,337; benzodiazepines, U.S. Pat. No. 5,288,514, and the like).

Devices for the preparation of combinatorial libraries are commercially available (see, e.g., ECIS™, Applied BioPhysics Inc.,Troy, N.Y., MPS, 390 MPS, Advanced Chem Tech, Louisville Ky., Symphony, Rainin, Woburn, Mass., 433A Applied Biosystems, Foster City, Calif., 9050 Plus, Millipore, Bedford, Mass.). In addition, numerous combinatorial libraries are themselves commercially available (see, e.g., ComGenex, Princeton, N.J., Tripos, Inc., St. Louis, Mo., 3D Pharmaceuticals, Exton, Pa., Martek Biosciences, Columbia, Md., etc.).

Mitotic network gene inhibitors such as the siRNA mitotic network gene inhibitors described herein can also be expressed recombinantly. In general, the nucleic acid sequences encoding mitotic network gene inhibitors such as the siRNA mitotic network gene inhibitor and related nucleic acid sequence homologues can be cloned. This aspect of the invention relies on routine techniques in the field of recombinant genetics. Generally, the nomenclature and the laboratory procedures in recombinant DNA technology described herein are those well known and commonly employed in the art. Standard techniques are used for cloning, DNA and RNA isolation, amplification and purification. Generally enzymatic reactions involving DNA ligase, DNA polymerase, restriction endonucleases and the like are performed according to the manufacturer's specifications. Basic texts disclosing the general methods of use in this invention include Sambrook et al., Molecular Cloning, A Laboratory Manual (3d ed. 2001); Kriegler, Gene Transfer and Expression: A Laboratory Manual (1990); and Current Protocols in Molecular Biology (Ausubel et al., eds., 1994)).

To obtain high level expression of a cloned gene or nucleic acid sequence, such as those nucleic acid sequences encoding mitotic network gene inhibitors such as the siRNAs, one typically subclones an inhibitor peptide sequence (e.g., nucleic acid sequences encoding mitotic network gene inhibitors such as the siRNA mitotic network gene inhibitor and related nucleic acid sequence homologue) into an expression vector that is subsequently transfected into a suitable host cell. The expression vector typically contains a strong promoter or a promoter/enhancer to direct transcription, a transcription/translation terminator, and for a nucleic acid encoding a protein, a ribosome binding site for translational initiation. The promoter is operably linked to the nucleic acid sequence encoding mitotic network gene inhibitors such as the siRNA mitotic network gene inhibitor or a subsequence thereof. Suitable bacterial promoters are well known in the art and described, e.g., in Sambrook et al. and Ausubel et al. The elements that are typically included in expression vectors also include a replicon that functions in a suitable host cell such as E. coli, a gene encoding antibiotic resistance to permit selection of bacteria that harbor recombinant plasmids, and unique restriction sites in nonessential regions of the plasmid to allow insertion of eukaryotic sequences. The particular antibiotic resistance gene chosen is not critical, any of the many resistance genes known in the art are suitable.

The particular expression vector used to transport the genetic information into the cell is not particularly critical. Any of the conventional vectors used for expression in eukaryotic or prokaryotic cells may be used. Standard bacterial expression vectors include plasmids such as pBR322 based plasmids, pSKF, pET23D, and fusion expression systems such as GST and LacZ. Epitope tags can also be added to the recombinant mitotic network gene inhibitors peptides to provide convenient methods of isolation, e.g., His tags. In some case, enzymatic cleavage sequences (e.g., Met-(His)g-Ile-Glu-GLy-Arg which form the Factor Xa cleavage site) are added to the recombinant mitotic network gene inhibitor peptides. Bacterial expression systems for expressing the mitotic network gene inhibitor peptides and nucleic acids are available in, e.g., E. coli, Bacillus sp., and Salmonella (Palva et al., Gene 22:229-235 (1983); Mosbach et al., Nature 302:543-545 (1983). Kits for such expression systems are commercially available. Eukaryotic expression systems for mammalian cells, yeast, and insect cells are well known in the art and are also commercially available.

Standard transfection methods are used to produce cell lines that express large quantities of mitotic network gene inhibitor, which can then purified using standard techniques (see, e.g., Colley et al., J. Biol. Chem. 264:17619-17622 (1989); Guide to Protein Purification, in Methods in Enzymology, vol. 182 (Deutscher, ed., 1990)). Transformation of cells is performed according to standard techniques (see, e.g., Morrison, J. Bact. 132:349-351 (1977); Clark-Curtiss & Curtiss, Methods in Enzymology 101:347-362 (Wu et al., eds, 1983). For example, any of the well known procedures for introducing foreign nucleotide sequences into host cells may be used. These include the use of calcium phosphate transfection, lipofectamine, polybrene, protoplast fusion, electroporation, liposomes, microinjection, plasma vectors, viral vectors and any of the other well known methods for introducing cloned genomic DNA, cDNA, synthetic DNA or other foreign genetic material into a host cell (see, e.g., Sambrook et al., supra). It is only necessary that the particular genetic engineering procedure used be capable of successfully introducing at least one gene into the host cell capable of expressing mitotic network gene inhibitor peptides and nucleic acids.

After the expression vector is introduced into the cells, the transfected cells are cultured under conditions favoring expression of mitotic network gene inhibitors (e.g. siRNA or shRNA mitotic network gene inhibitors) and related nucleic acid sequence homologues.

RNAi is a naturally occurring gene regulatory mechanism, which has a number of advantages over other gene/antisense therapies including specificity of inhibition, potency, the small size of the molecules and the diminished risk of toxic effects, e.g., immune responses. Targeted, local delivery of RNAi to the lungs via inhalation offers in vivo delivery of siRNA or shRNA for the treatment of a range of diseases including cancer of the lungs, bronchea, esophagus, and other cancers within or tangential to or accessible from the airway path.

siRNA can be specifically synthesized and introduced into a cell to induce gene silencing. As this methodology exploits a naturally occurring pathway, it differs from other silencing technologies such as antisense oligonucleotides. In nature, RNAi is initiated when the cell encounters ectopic double stranded RNA (dsRNA), e.g., viral RNA, transposon or microRNA (miRNA). In the cytoplasm the RNase III-like protein dicer cleaves dsRNA from miRNAs or replicating viruses into siRNAs of 19-25 bases in length. The siRNA is then incorporated into the multiprotein RNA-induced silencing complex (RISC), which unwinds the duplex producing two strands; one strand (passenger) is discarded while the other (guide) can independently guide targeted mRNA recognition. The binding of siRNA results in a site-specific cleavage of the mRNA thereby silencing the message. The released cleavage products are degraded, and the siRNA:RISC complex is free to find another mRNA target. Degrading mRNA results in a profound reduction in the levels of the corresponding protein without altering the DNA. RNAi is therefore a highly promising therapeutic approach for diseases where aberrant protein production is a problem, such as cancers that over express mitotic network gene.

Effective site selection algorithms and several siRNA design guides are currently available. The majority of in vivo siRNA experiments to date reported the use of 21-mer duplexes with a 19-base central double-stranded region and terminal 2-base 3′ overhangs. This design mimics naturally occurring molecules produced by dicer processing in vivo. siRNA can be chemically synthesized or transcribed from a plasmid. In the case of the latter, a DNA insert of approximately 70 bp, encoding for a short hairpin RNA (shRNA) targeting the gene of interest, is cloned into a plasmid vector. The insert containing plasmid can then be transfected into a cell where the shRNA is expressed. The shRNA is rapidly processed by the cellular machinery into 19-22 nt siRNAs, which can then interfer with the expression of the target gene.

Several strategies are being explored to improve siRNA stability in vivo based on modifications previously used to improve the stability of antisense molecules. Commonly used modifications to improve stability include phosphorothioate (PS) or boranophosphate modification of the internucleoside linkage. Boranophosphate modifications confer significant nuclease resistance, but synthesis is complex, with modified bases being incorporated using in vitro transcription, making site-selective placement difficult. PS modifications are easier to position and will prolong the life of the siRNA when exposed to nucleases. It is important to note, however, that while limited PS modification preserves siRNA potency, over modification may decrease potency and/or increase toxicity.

A number of strategies can be used to prevent immune recognition and response, such as the use of delivery agents to avoid retention of siRNA within endosomes. Another common strategy is the modification of the nucleotides of siRNA, such as the replacement of the 2′-hydroxyl uridines with 2′-O-methyl uridines.

Careful design of siRNA is essential to prevent off-target effects. Nucleic acid—base pairing is highly specific, and mismatches at one or a small number of positions is often sufficient to completely prevent hybridization under physiological conditions. It is desirable therefore to synthesize more than one siRNA for each target to control for off-target effects.

In another embodiment, naked siRNA and shRNA delivery to tumor cells in vivo is also contemplated. RNA interference (RNAi) is a post-transcriptional gene silencing event in which short double-stranded RNA (siRNA) degrades target mRNA. Silencing oncogenes or other genes contributing to tumor progression by RNAi can be a therapeutic strategy for cancer. Delivery of RNAi effector to tumor cells is one of the key factors determining the efficacy, because the gene silencing is limited in cells reached by RNAi effector. In this study, we developed a tumor cell line stably expressing reporter genes to sensitively and quantitatively evaluate RNAi effect in tumor cells in vivo. Genetically labeled tumor cells were inoculated into the footpad or via the portal vein of mice to establish primary and metastatic tumor models, respectively. Intratumoral injection of either naked siRNA or naked short-hairpin RNA (shRNA)-expressing plasmid DNA followed by electroporation was effective in suppressing the expression of the target gene in tumor cells. Intravenous injection of naked RNAi effectors by the hydrodynamics-based procedure inhibited the gene expression in tumor cells colonizing in the liver. Then, shRNA-expressing plasmid DNA targeting β-catenin or hypoxia inducible factor-1α (HIF-1α) was delivered to tumor cells in order to inhibit tumor growth in vivo. In the primary tumor model, delivery of shRNA-expressing plasmid DNA targeting β-catenin or HIF-1α was effective in inhibiting tumor growth, whereas only shRNA-expressing pDNA targeting HIF-1α was effective in the hepatic metastasis model. We also found that HIF1 expression in liver cells is elevated by inoculation of tumor cells into the portal vein, and the silencing of the expression in normal liver cells is also effective in inhibiting tumor metastasis to the liver. Takahashi et al, (Grad. Sch. Pharm. Sci., Kyoto Univ.).

RNAi offers more specificity and flexibility than traditional drugs in treating diseases. When short pieces of double-stranded RNA (designed to target a particular gene) are introduced into cells, they are separated into single strands, with one binding to the target RNA and causing its demise. Thus the target RNA is no longer expressed.

In another embodiment, delivery of inhibitory nucleotides in particles or complexes.

A delivery reagent designed to lengthen the time of the RNA therapeutic agent in the body, facilitating its uptake into distal target sites is advantageous. Lipid nanoparticles that encapsulate siRNA for delivery to specific disease sites are commercially available and may find use in the application. An example is SNALP (stable nucleic acid-lipid particles) available from Tekmira (Burnaby, BC, Canada). For example, Tekmira's anti-cancer PLK1 SNALP is under development to deliver PLK1 RNAi drugs to silence PLK1, one of the mitotic network genes. Peptide based polymer nanoparticles for RNAi delivery can also be used to deliver RNA molecules to almost any body tissue as described in U.S. Pat. No. 7,534,878. A commercial example is Intradigm PolyTran™ peptide-based polymers to create nanoparticles for RNAi delivery (Intradigm Corporation, Palo Alto, Calif.). Agents for targeted in vivo delivery of RNAi is also commercially available using. Invitrogen's RNAi delivery reagent, Invivofectamine™ (Invitrogen, Life Technologies, Carlsbad, Calif.), facilitates systemic in vivo delivery and is non-toxic. It is especially effective when used together with their Stealth™ RNAi duplexes, which have been chemically modified so that only one strand participates in RNAi (reducing off-target effects), and the RNA evades the host immune response.

MDRNA (Bothell, Wash.) takes two approaches to design and delivery with their UsiRNA and meroduplex platforms. Their platform employs strategically placed non-nucleotide entities termed Unlocked Nucleobase Analogs (UNA) in addition to RNA to form a short double-stranded RNA-based oligonucleotide. UsiRNAs are protected from degradation and immune detection, and reduce off-target effects. Meroduplex is based on the concept that placing a nick or gap in the passenger strand will minimize off-target activity related to the passenger strand. A nicked or gapped passenger strand biases the siRNA to load the guide strand into the RNAi machinery, thus maximizing the likelihood that the guide strand will appropriately silence the gene target.

In some cases a decision needs to made whether to use an siRNA or an shRNA. For shRNAs, the procedure is to clone, verify insert, determine how much of the shRNA the target cells are expressing, and then preferably use viral vectors for delivery of shRNA. Nonviral vectors such as nanostructures, and microparticles also can be used.

The mechanism to which RNAi works in the cell is the same with shRNA and siRNA. Only the enzyme dicer will cleave the shRNA into an siRNA like oligo (removing the hairpin).The enzyme recognizes an oddly shaped hairpin structure and cleaves it.

Once dsRNA enters the cell, it is cleaved by an RNase III-like enzyme, Dicer, into double stranded small interfering RNAs (siRNA) 21-23 nucleotides in length that contain 2 nucleotide overhangs on the 3′ ends (9-11). In an ATP dependent step, the siRNAs become integrated into a multi-subunit protein complex, commonly known as the RNAi induced silencing complex (RISC), which guides the siRNAs to the target RNA sequence. At some point the siRNA duplex unwinds, and it appears that the antisense strand remains bound to RISC and directs degradation of the complementary mRNA sequence by a combination of endo and exonucleases.

Using synthetic, short double-stranded RNAs that mimic the siRNAs produced by the enzyme dicer, sequence specific gene silencing is achieved in mammalian cells without inducing the interferon response.

In another embodiment, delivery of DNA vector-based short hairpin RNA (shRNA) as a means of effecting RNA interference (RNAi) for the precise disruption of mitotic network gene expression to achieve a therapeutic effect is performed. (See Vorhies and Nemunaitis, 2009, Volume 480, Macromolecular Drug Delivery Humana Press10.1007/978-1-59745-429, 2978-1-59745-429-2). The clinical usage of shRNA therapeutics in cancer is limited by obstacles related to effective delivery into the nuclei of target cancer cells. Significant pre-clinical data have been amassed about biodegradable and non-biodegradable polymeric delivery vehicles that are relevant for shRNA delivery into humans. Some of the leading candidates for clinical usage have potential for usage in cancer shRNA therapeutics. Biodegradable and non-biodegradable delivery vehicles can be used.

An alternate to individual chemical synthesis of siRNA is to construct a sequence for insertion in an expression vector. Several companies offer RNAi vectors for the transcription of inserts. Some use an RNA polymerase III (Pol III) promoter to drive expression of both the sense and antisense strands separately, which then hybridize in vivo to make the siRNA. Other vectors are based on the use of Pol III to drive expression of short “hairpin” RNAs (shRNA), individual transcripts that adopt stem-loop structures, which are processed into siRNAs by the RNAi machinery. Typical shRNA design consists of two inverted repeats containing the sense and antisense target sequences separated by a loop sequence. Commonly used loop sequences contain 8-9 bases. A terminator sequence consisting of 5-6 poly dTs is present at the 3′ end and cloning sequences can be added to the 5′ ends of the complementary oligonucleotides.

In another embodiment, targeted nanoparticles incorporating siRNA offer promise for cancer treatment. Use of targeted nanoparticles offers promising techniques for cancer treatment. By using targeted nanoparticles, researchers have demonstrated that systemically delivered siRNA can slow the growth of tumors in mice without eliciting the toxicities often associated with cancer therapies. NSTI Nanotech 2007. siRNA are incorporated into nanoparticles that are formed completely by self-assembly, using cyclodextrin-containing polycations. Dosing schedules and surface modifications on the efficacy of these siRNA nanoparticles is determined before a clinical trial.

The mitotic network gene inhibitor peptides and nucleic acids of the present invention, such as the siRNA or shRNA mitotic network gene inhibitor, also can be used to treat or prevent a variety of disorders associated with reduced survival rate, especially as related to cancers. The peptides and nucleic acids are administered to a patient in an amount sufficient to elicit a therapeutic response in the patient (e.g., reduction of tumor size and growth rate, prolonged survival rate, reduction in concurrent cancer therapeutics administered to patient). An amount adequate to accomplish this is defined as “therapeutically effective dose or amount.”

The peptides and nucleic acids of the invention can be administered directly to a mammalian subject using any route known in the art, including e.g., by injection (e.g., intravenous, intraperitoneal, subcutaneous, intramuscular, or intradermal), inhalation, transdermal application, rectal administration, or oral administration.

The pharmaceutical compositions of the invention may comprise a pharmaceutically acceptable carrier. Pharmaceutically acceptable carriers are determined in part by the particular composition being administered, as well as by the particular method used to administer the composition. Accordingly, there are a wide variety of suitable formulations of pharmaceutical compositions of the present invention (see, e.g., Remington's Pharmaceutical Sciences, 17th ed., 1989).

As used herein, “carrier” includes any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutical active substances is well known in the art. Except insofar as any conventional media or agent is incompatible with the active ingredient, its use in the therapeutic compositions is contemplated. Supplementary active ingredients can also be incorporated into the compositions.

The phrase “pharmaceutically-acceptable” refers to molecular entities and compositions that do not produce an allergic or similar untoward reaction when administered to a human. The preparation of an aqueous composition that contains a protein as an active ingredient is well understood in the art. Typically, such compositions are prepared as injectables, either as liquid solutions or suspensions; solid forms suitable for solution in, or suspension in, liquid prior to injection can also be prepared. The preparation can also be emulsified.

Administration of the peptides and nucleic acids of the invention can be in any convenient manner, e.g., by injection, intratumoral injection, intravenous and arterial stents (including eluting stents), catheter, oral administration, inhalation, transdermal application, or rectal administration. In some cases, the peptides and nucleic acids are formulated with a pharmaceutically acceptable carrier prior to administration. Pharmaceutically acceptable carriers are determined in part by the particular composition being administered (e.g., nucleic acid or polypeptide), as well as by the particular method used to administer the composition. Accordingly, there are a wide variety of suitable formulations of pharmaceutical compositions of the present invention (see, e.g., Remington's Pharmaceutical Sciences, 17th ed., 1989).

The dose administered to a patient, in the context of the present invention should be sufficient to effect a beneficial therapeutic response in the patient over time. The dose will be determined by the efficacy of the particular vector (e.g. peptide or nucleic acid) employed and the condition of the patient, as well as the body weight or surface area of the patient to be treated. The size of the dose also will be determined by the existence, nature, and extent of any adverse side-effects that accompany the administration of a particular peptide or nucleic acid in a particular patient.

In determining the effective amount of the vector to be administered in the treatment or prophylaxis of diseases or disorder associated with the disease, the physician evaluates circulating plasma levels of the polypeptide or nucleic acid, polypeptide or nucleic acid toxicities, progression of the disease (e.g., breast cancer), and the production of antibodies that specifically bind to the peptide. Typically, the dose equivalent of a polypeptide is from about 0.1 to about 50 mg per kg, preferably from about 1 to about 25 mg per kg, most preferably from about 1 to about 20 mg per kg body weight. In general, the dose equivalent of a naked c acid is from about 1 μg to about 100 μg for a typical 70 kilogram patient, and doses of vectors which include a viral particle are calculated to yield an equivalent amount of therapeutic nucleic acid.

For administration, polypeptides and nucleic acids of the present invention can be administered at a rate determined by the LD-50 of the polypeptide or nucleic acid, and the side-effects of the polypeptide or nucleic acid at various concentrations, as applied to the mass and overall health of the patient. Administration can be accomplished via single or divided doses, e.g., doses administered on a regular basis (e.g., daily) for a period of time (e.g., 2, 3, 4, 5, 6, days or 1-3 weeks or more).

In certain circumstances it will be desirable to deliver the pharmaceutical compositions comprising the mitotic network gene inhibitor peptides and nucleic acids disclosed herein parenterally, intravenously, intramuscularly, or even intraperitoneally as described in U.S. Pat. No. 5,543,158; U.S. Pat. No. 5,641,515 and U.S. Pat. No. 5,399,363. Solutions of the active compounds as free base or pharmacologically acceptable salts may be prepared in water suitably mixed with a surfactant, such as hydroxypropylcellulose. Dispersions may also be prepared in glycerol, liquid polyethylene glycols, and mixtures thereof and in oils. Under ordinary conditions of storage and use, these preparations contain a preservative to prevent the growth of microorganisms.

The pharmaceutical forms suitable for injectable use include sterile aqueous solutions or dispersions and sterile powders for the extemporaneous preparation of sterile injectable solutions or dispersions (U.S. Pat. No. 5,466,468). In all cases the form must be sterile and must be fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants. The prevention of the action of microorganisms can be facilitated by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, a sterile aqueous medium that can be employed will be known to those of skill in the art in light of the present disclosure. For example, one dosage may be dissolved in 1 ml of isotonic NaCl solution and either added to 1000 ml of hypodermoclysis fluid or injected at the proposed site of infusion (see, e.g., Remington's Pharmaceutical Sciences, 15th Edition, pp. 1035-1038 and 1570-1580). Some variation in dosage will necessarily occur depending on the condition of the subject being treated. The person responsible for administration will, in any event, determine the appropriate dose for the individual subject. Moreover, for human administration, preparations should meet sterility, pyrogenicity, and the general safety and purity standards as required by FDA Office of Biologics standards.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

The compositions disclosed herein may be formulated in a neutral or salt form. Pharmaceutically-acceptable salts, include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like. Upon formulation, solutions will be administered in a manner compatible with the dosage formulation and in such amount as is therapeutically effective. The formulations are easily administered in a variety of dosage forms such as injectable solutions, drug-release capsules, and the like.

To date, most studies have been performed with siRNA formulated in sterile saline or phosphate buffered saline (PBS) that has ionic character similar to serum. There are minor differences in PBS compositions (with or without calcium, magnesium, etc.) and investigators should select a formulation best suited to the injection route and animal employed for the study. Lyophilized oligonucleotides and standard or siSTABLE siRNAs are readily soluble in aqueous solution and can be resuspended at concentrations as high as 2.0 mM. However, viscosity of the resultant solutions can sometimes affect the handling of such concentrated solutions.

While lipid formulations have been used extensively for cell culture experiments, the attributes for optimal uptake in cell culture do not match those useful in animals. The principle issue is that the cationic nature of the lipids used in cell culture leads to aggregation when used in animals and results in serum clearance and lung accumulation. Polyethylene glycol complexed-liposome formulations are currently under investigation for delivery of siRNA by several academic and industrial investigators, including Dharmacon, but typically require complex formulation knowledge. There are a few reports that cite limited success using lipid-mediated delivery of plasmids or oligonucleotides in animals.

Oligonucleotides can also be administered via bolus or continuous administration using an ALZET mini-pump (DURECT Corporation). Caution should be observed with bolus administration as studies of antisense oligonucleotides demonstrated certain dosing-related toxicities including hind limb paralysis and death when the molecules were given at high doses and rates of bolus administration. Studies with antisense and ribozymes have shown that the molecules distribute in a related manner whether the dosing is through intravenous (IV), subcutaneous (sub-Q), or intraperitoneal (IP) administration. For most published studies, dosing has been conducted by IV bolus administration through the tail vein. Less is known about the other methods of delivery, although they may be suitable for various studies. Any method of administration will require optimization to ensure optimal delivery and animal health.

For bolus injection, dosing can occur once or twice per day. The clearance of oligonucleotides appears to be biphasic and a fairly large amount of the initial dose is cleared from the urine in the first pass. Dosing should be conducted for a fairly long term, with a one to two week course of administration being preferred. This is somewhat dependent on the model being examined, but several metabolic disorder studies in rodents that have been conducted using antisense oligonucleotides have required this course of dosing to demonstrate clear target knockdown and anticipated outcomes.

In certain embodiments, the inventors contemplate the use of liposomes, nanocapsules, microparticles, microspheres, lipid particles, vesicles, and the like, for the administration of the mitotic network gene inhibitory peptides and nucleic acids of the present invention. In particular, the compositions of the present invention may be formulated for delivery either encapsulated in a lipid particle, a liposome, a vesicle, a nanosphere, or a nanoparticle or the like. In one embodiment, the mitotic network gene siRNA inhibitors are entrapped in a liposome for delivery.

The formation and use of liposomes is generally known to those of skill in the art (see for example, Couvreur et al., 1977; Couvreur, 1988; Lasic, 1998; which describes the use of liposomes and nanocapsules in the targeted antibiotic therapy for intracellular bacterial infections and diseases). Recently, liposomes were developed with improved serum stability and circulation half-times (Gabizon & Papahadjopoulos, 1988; Allen and Choun, 1987; U.S. Pat. No. 5,741,516). Further, various methods of liposome and liposome like preparations as potential drug carriers have been reviewed (Takakura, 1998; Chandran et al., 1997; Margalit, 1995; U.S. Pat. No. 5,567,434; U.S. Pat. No. 5,552,157; U.S. Pat. No. 5,565,213; U.S. Pat. No. 5,738,868 and U.S. Pat. No. 5,795,587).

Liposomes are formed from phospholipids that are dispersed in an aqueous medium and spontaneously form multilamellar concentric bilayer vesicles (also termed multilamellar vesicles (MLVs). MLVs generally have diameters of from 25 nm to 4 m. Sonication of MLVs results in the formation of small unilamellar vesicles (SUVs) with diameters in the range of 200 to 500 Å, containing an aqueous solution in the core.

Liposomes bear resemblance to cellular membranes and are contemplated for use in connection with the present invention as carriers for the peptide compositions. They are widely suitable as both water- and lipid-soluble substances can be entrapped, i.e. in the aqueous spaces and within the bilayer itself, respectively. It is possible that the drug-bearing liposomes may even be employed for site-specific delivery of active agents by selectively modifying the liposomal formulation.

Targeting is generally not a limitation in terms of the present invention. However, should specific targeting be desired, methods are available for this to be accomplished. For example, antibodies may be used to bind to the liposome surface and to direct the liposomes and its contents to particular cell types. Carbohydrate determinants (glycoprotein or glycolipid cell-surface components that play a role in cell-cell recognition, interaction and adhesion) may also be used as recognition sites as they have potential in directing liposomes to particular cell types.

Alternatively, the invention provides for pharmaceutically-acceptable nanocapsule formulations of the compositions of the present invention. Nanocapsules can generally entrap compounds in a stable and reproducible way (Henry-Michelland et al., 1987; Quintanar-Guerrero et al., 1998; Douglas et al., 1987). To avoid side effects due to intracellular polymeric overloading, such ultrafine particles (sized around 0.1 m) should be designed using polymers able to be degraded in vivo. Biodegradable polyalkyl-cyanoacrylate nanoparticles that meet these requirements are contemplated for use in the present invention. Such particles may be are easily made, as described (Couvreur et al., 1980; 1988; zur Muhlen et al., 1998; Zambaux et al. 1998; Pinto-Alphandry et al., 1995 and U.S. Pat. No. 5,145,684).

In certain embodiments, the nucleic acids encoding inhibitory mitotic network gene peptides and nucleic acids of the present invention can be used for transfection of cells in vitro and in vivo. These nucleic acids can be inserted into any of a number of well-known vectors for the transfection of target cells and organisms as described below. The nucleic acids are transfected into cells, ex vivo or in vivo, through the interaction of the vector and the target cell. The nucleic acid, under the control of a promoter, then expresses an inhibitory mitotic network gene peptides and nucleic acids of the present invention, thereby mitigating the effects of over amplification of a candidate gene associated with reduced survival rate.

Such gene therapy procedures have been used to correct acquired and inherited genetic defects, cancer, and other diseases in a number of contexts. The ability to express artificial genes in humans facilitates the prevention and/or cure of many important human diseases, including many diseases which are not amenable to treatment by other therapies (for a review of gene therapy procedures, see Anderson, Science 256:808-813 (1992); Nabel & Feigner, TIBTECH 11:211-217 (1993); Mitani & Caskey, TIBTECH 11:162-166 (1993); Mulligan, Science 926-932 (1993); Dillon, TIBTECH 11:167-175 (1993); Miller, Nature 357:455-460 (1992); Van Brunt, Biotechnology 6(10):1149-1154 (1998); Vigne, Restorative Neurology and Neuroscience 8:35-36 (1995); Kremer & Perricaudet, British Medical Bulletin 51(1):31-44 (1995); Haddada et al., in Current Topics in Microbiology and Immunology (Doerfler & Bohm eds., 1995); and Yu et al., Gene Therapy 1:13-26 (1994)).

For delivery of nucleic acids, viral vectors may be used. Suitable vectors include, for example, herpes simplex virus vectors as described in Lilley et al., Curr. Gene Ther. 1(4):339-58 (2001), alphavirus DNA and particle replicons as described in e.g., Polo et al., Dev. Biol. (Basel) 104:181-5 (2000), Epstein-Barr virus (EBV)-based plasmid vectors as described in, e.g., Mazda, Curr. Gene Ther. 2(3):379-92 (2002), EBV replicon vector systems as described in e.g., Otomo et al., J. Gene Med. 3(4):345-52 (2001), adeno-virus associated viruses from rhesus monkeys as described in e.g., Gao et al., PNAS USA. 99(18):11854 (2002), adenoviral and adeno-associated viral vectors as described in, e.g., Nicklin and Baker, Curr. Gene Ther. 2(3):273-93 (2002). Other suitable adeno-associated virus (AAV) vector systems can be readily constructed using techniques well known in the art (see, e.g., U.S. Pat. Nos. 5,173,414 and 5,139,941; PCT Publication Nos. WO 92/01070 and WO 93/03769; Lebkowski et al. (1988) Mol. Cell. Biol. 8:3988-3996; Vincent et al. (1990) Vaccines 90 (Cold Spring Harbor Laboratory Press); Carter (1992) Current Opinion in Biotechnology 3:533-539; Muzyczka (1992) Current Topics in Microbiol. and Immunol. 158:97-129; Kotin (1994) Human Gene Therapy 5:793-801; Shelling and Smith (1994) Gene Therapy 1:165-169; and Zhou et al. (1994) J. Exp. Med. 179:1867-1875). Additional suitable vectors include E1B gene-attenuated replicating adenoviruses described in, e.g., Kim et al., Cancer Gene Ther.9(9):725-36 (2002) and nonreplicating adenovirus vectors described in e.g., Pascual et al., J. Immunol. 160(9):4465-72 (1998) Exemplary vectors can be constructed as disclosed by Okayama et al. (1983) Mol. Cell. Biol. 3:280.

Molecular conjugate vectors, such as the adenovirus chimeric vectors described in Michael et al. (1993) J. Biol. Chem. 268:6866-6869 and Wagner et al. (1992) Proc. Natl. Acad. Sci. USA 89:6099-6103, can also be used for gene delivery according to the methods of the invention.

In one illustrative embodiment, retroviruses provide a convenient and effective platform for gene delivery systems. A selected nucleotide sequence encoding an inhibitory mitotic network gene nucleic acid or polypeptide can be inserted into a vector and packaged in retroviral particles using techniques known in the art. The recombinant virus can then be isolated and delivered to a subject. Suitable vectors include lentiviral vectors as described in e.g., Scherr and Eder, Curr. Gene Ther. 2(1):45-55 (2002). Additional illustrative retroviral systems have been described (e.g., U.S. Pat. No. 5,219,740; Miller and Rosman (1989) BioTechniques 7:980-990; Miller (1990) Human Gene Therapy 1:5-14; Scarpa et al. (1991) Virology 180:849-852; Burns et al. (1993) Proc. Natl. Acad. Sci. USA 90:8033-8037; and Boris-Lawrie and Temin (1993) Curr. Opin. Genet. Develop. 3:102-109.

Other known viral-based delivery systems are described in, e.g., Fisher-Hoch et al. (1989) Proc. Natl. Acad. Sci. USA 86:317-321; Flexner et al. (1989) Ann. N.Y. Acad. Sci. 569:86-103; Flexner et al. (1990) Vaccine 8:17-21; U.S. Pat. Nos. 4,603,112, 4,769,330, and 5,017,487; WO 89/01973; U.S. Pat. No. 4,777,127; GB 2,200,651; EP 0,345,242; WO 91/02805; Berkner (1988) Biotechniques 6:616-627; Rosenfeld et al. (1991) Science 252:431-434; Kolls et al. (1994) Proc. Natl. Acad. Sci. USA 91:215-219; Kass-Eisler et al. (1993) Proc. Natl. Acad. Sci. USA 90:11498-11502; Guzman et al. (1993) Circulation 88:2838-2848; Guzman et al. (1993) Cir. Res. 73:1202-1207; and Lotze and Kost, Cancer Gene Ther. 9(8):692-9 (2002).

In some embodiments, the inhibitory mitotic network gene polypeptides and nucleic acids are administered in combination with a second therapeutic agent for treating or preventing cancer, including breast and breast cancer. For example, an inhibitory mitotic network gene siRNA may be administered in conjunction with any of the standard treatments for breast cancer including, but not limited to, paclitaxel, cisplatin, carboplatin, chemotherapy, and radiation treatment. Or in another embodiment, a mitotic network gene siRNA is delivered with small-molecule inhibitors such as for PLK1 (GSK461364), CENPE(GSK923295) and AURKB (GSK1070916) (GlaxoSmithKline. Inc).

The inhibitory mitotic network gene polypeptides and nucleic acids and the second therapeutic agent may be administered simultaneously or sequentially. For example, the inhibitory mitotic network gene polypeptides and nucleic acids may be administered first, followed by the second therapeutic agent. Alternatively, the second therapeutic agent may be administered first, followed by the inhibitory mitotic network gene polypeptides and nucleic acids. In some cases, the inhibitory mitotic network gene polypeptides and nucleic acids and the second therapeutic agent are administered in the same formulation. In other cases the inhibitory mitotic network gene polypeptides and nucleic acids and the second therapeutic agent are administered in different formulations. When the inhibitory mitotic network gene polypeptides and nucleic acids and the second therapeutic agent are administered in different formulations, their administration may be simultaneous or sequential.

In some cases, the inhibitory mitotic network gene polypeptides and nucleic acids can be used to target therapeutic agents to cells and tissues expressing mitotic network gene and other candidate genes that are related to reduced survival rate.

The present invention further provides kits for use within any of the above diagnostic methods. Such kits typically comprise two or more components necessary for performing a diagnostic assay. Components may be compounds, reagents, containers and/or equipment. For example, one container within a kit may contain an inhibitory mitotic network gene polypeptides and nucleic acids. One or more additional containers may enclose elements, such as reagents or buffers, to be used in the assay. Such kits may also, or alternatively, contain a detection reagent as described above that contains a reporter group suitable for direct or indirect detection of antibody binding.

Kits can also be supplied for therapeutic uses. Thus, the subject composition of the present invention may be provided, usually in a lyophilized form, in a container. The inhibitory mitotic network gene polypeptides and nucleic acids described herein are included in the kits with instructions for use, and optionally with buffers, stabilizers, biocides, and inert proteins. Generally, these optional materials will be present at less than about 5% by weight, based on the amount of polypeptide or nucleic acid, and will usually be present in a total amount of at least about 0.001% by weight, based on the polypeptide or nucleic acid concentration. It may be desirable to include an inert extender or excipient to dilute the active ingredients, where the excipient may be present in from about 1 to 99% weight of the total composition. The kits may further comprise a second therapeutic agent, including for example, paclitaxel, carboplatin, a chemotherapeutic agent, or small-molecule inhibitors for PLK1 (GS K461364), CENPE(GSK923295) and AURKB (GSK1070916) were provided by GlaxoSmithKline. Inc.

### EXAMPLE 1

### Materials and Methods for Finding the Mitotic Network Genes

Cell culture: Human non-malignant and breast cancer cell lines have been established from normal and human breast cancer samples. The cell lines described in this study derived from 49 malignant and 4 non-malignant breast tissues and growth conditions for the cell lines have been reported previously This resource consists of nearly 54 well-characterized breast cell lines with information on genomic and gene expression signatures. The cell incubational condition of the cell lines was shown previously by some of the inventors in Neve, R. M. et al. Cancer Cell 10, 515-527 (2006).

Preparation of Compound: The small-molecule inhibitors for PLK1 (GSK461364), CENPE(GSK923295) and AURKB (GSK1070916) were provided by GlaxoSmithKline, Inc. Stock solutions were made at a concentration of 10 mM in DMSO and stored at −20° C. Compounds were diluted (1:5 serial dilution) to produce test drug concentrations ranging from 0.0768 nM to30 μM.

Cell viability/growth assay and Dose response (G150): Dose-response curves were determined according to the National Cancer Institute NIH guidelines. In brief, cell suspensions were aliquoted into 96-well plates in 100 μl growth media. Inoculates were allowed a preincubation period of 24 hours at 37° C. for stabilization. Cells were treated with 9 doses in triplicate for 72 hours with GSK461364 or GSK1070916. Cell proliferation was measured with CellTiter-Glo® Luminescent Cell Viability Assay (Promega, Madison, Wis.). After subtraction of the baseline (an estimate of the number of the cells just before treatment, time 0), the absorbance was plotted. Total growth inhibition doses and 50% growth inhibition doses GI50 were calculated by GraphPad Prism4 software (GraphPad Software, Inc., La Jolla, Calif.).

Datasets: The mitotic gene transcriptional network was assessed in several published microarray data sets profiled with Affymetrix GeneChip arrays (HG-U133A or HG-U133 Plus 2.0). These data included numerous tumor types including breast cancer (GEO accession numbers, GSE2034, GSE1456 and GSE4922), lung cancer (GEO accession number, GSE3141), ovarian cancer (GEO accession number, GSE3149 and GSE9891), Wilms' tumor (GEO accession number, GSE10320), prostate cancer (GEO accession number, GSE8128), glioma (GEO accession number, GSE13041), acute lymphoblastic leukemia (GEO accession number, GSE12995), acute myelogenous leukemia (GEO accession number, GSE12417), and lymphoblast cell lines (GEO accession number, GSE11582). The mitotic network activity was also examined in varous normal tissues (GEO accession number, GSE7307) and IDC (GEO accession number, GSE10780). The relationship between MNAI and survival among patients with breast cancer was examined in four data sets (Dataset 1: Chin et al¹⁴, Dataset 2: GSE2034¹⁵, Dataset 3: GSE1456¹⁶, and Dataset 4: GSE4922¹⁷). Data were pre-processed as described in the original publications.

An additional breast cancer dataset consisting of 824 fresh frozen tumors was employed for validation of the mitotic network gene signature and associations between copy number and expression(Curtis et al, In preparation). In this study, high-density Affymetrix SNP 6.0 arrays were employed to assay copy number and matched RNA was hybridized to Illumina HT-12 bead arrays for gene-expression analysis. The dimensionality of the copy number data was reduced by merging regions with similar profiles across samples based on the CGH regions algorithm [vanDeWiel, 2007], resulting in 3465 regions. The MNAI was computed by utilizing probes with a perfect transcriptomic match based on reannotation of the Illumina platform [Barbosa-Morais, 2010]. Averages were taken when multiple perfect probes were present on the array. Samples were classified into the five intrinsic subtypes based on PAM50 [Parker, 2009].

Statistical analysis: The correlation among the cellular GI50 values of GSK461364, GSK1070916 and GSK92325 was examined by Pearson correlation test. Tumor expression profiles were clustered using the mitotic network genes. Kaplan-Meier survival curves were generated for patients stratified into groups of high (upper tertile) and low (lower tertile) MNAI to evaluate differences in disease-free survival (DFS). All statistical analyses were performed using the Statistical Package for the Social Sciences version 11.5 (SPSS, Inc., Chicago, Ill.). Association analyses were performed by performing one at a time ANOVAs with copy number as the predictor variable for each mitotic net expression profile for both the Chin et al and Curtis et al datasets.

Network construction and functional annotation: Genes found to be significantly correlated (Pearson Correlation) with the mRNA expression levels of PLK1, CENPE, or AURKB were selected for inclusion in the mitotic network based on Affymetrix expression profiling of a panel of 53 human breast cancer cell lines. The correlation cut-off was determined based on 1000 permutations tests. The gene ontology statistics tool BiNGO²⁸ was employed to test for enrichment of specific functional groups. A relevance network was constructed based on the ExpressionCorrelation software tool <URL:http://baderlab.org/Software/ExpressionCorrelation>. Correlations exceeding a threshold were displayed as “edges” between two “nodes” (where nodes represent genes). Network figures were generated using Cytosc ape version 2.6.1 <URL:http://www.cytoscape.org>

### EXAMPLE 2

### Knockdown Studies of Mitotic Network Genes

We transiently transfect siRNA for MELK, SMC4, TEX10, AURKA, HJURP, BUB1, RFC3, and CCNB2 into MDAMB231 and BT549 breast cancer cell lines. Non-specific siRNA served as a negative control. Cell viability/proliferation was evaluated by CellTiter-Glo® luminescent cell viability assay (CTG, Promega), cell apoptosis was assayed using YoPro-1 and Hoechst staining and cell cycle inhibition was assessed by measuring BrdU incorporation. All cellular measurements were made in adhered cells using the Cellomics high content scanning instrument. All assays were run at 3, 4, 5 and 6 days post transfection.

siRNA transfection and efficiency of knockdown: siRNAs targeting mitotic genes (two siRNAs targeting different sequences of each gene) and AllStars Negative Control siRNA were purchased from Qiagen Inc. The AllStars Negative Control siRNA, which has no homology to any known mammalian gene is the most thoroughly tested and validated negative control siRNA currently available. MDAMB231 cells were seeded at 3000 cells per well in 96-well plates one day prior to transfection. Cells were transfected with 10 nM siRNAs using Dharmafect1 transfection regent (Dharmacon) according to the manufacturer's instructions. After transfection with siRNAs for 72 hours, cell viability was measured using the CellTiter-Glo® assay (Promega). The RNA level of each gene and the actin control were measured with QuantiGene® 2.0 Reagent System (Panomics). The RNA levels relative to actin were compared to mRNA levels normalized to AllStars Negative control siRNA.

Briefly, an siRNA transfection protocol is as follows. Cells are plated and grown to 50-70% confluency and transfected using DharmaFECT1. In tubes, mix: Tube A: total volume 10 μl 9.5 μL SFM media+0.5 siRNA(varied according to the experiment design); Tube B: total volume 10 ul 9.8 uL SFM media+0.2 DharmaFECT1. Incubate tubes for 5 min. During this incubation, remove media from target cells and replace with SFM in each well. Add contents of Tube B to Tube A and mix gently. Incubate for 20 min at room temperature. Add 20 uL mixture solution dropwise to each well (final volume=100 μL). Leave for 4 h, aspirate off media and replace with full growth media and allow cells to grow for several days.

Cell growth analysis is carried out using the CellTiterGlo® Luminescent Cell Viability Assay (Promega Cat#G7571/2/3). The luminescence signal of viable cells measures the amount of ATP detected in the plates were read using a custom plate reader and program.

BrdU Staining and Fixation for Cellomics were used to measure cell proliferation and cell cycle analysis. To incorporate BrdU and fix the cells 10 μM final concentration of BrdU (Sigma #B5002) was added directly to cell media and pulsed for 30 minutes in tissue culture incubator. The media was removed and the cells washed 2× with 1× PBS and then 70% EtOH added to cover cells and fix for overnight at 4° C. Next day the 70% EtOH was removed and cells allowed to dry. Then 2N HCl was added and cells incubated at room temperature for 5-10 minutes, then removed and 1× PBS added to neutralize. Diluted anti-BrdU antibody (Mouse anti-BrdU Clone 3D4 (BD Pharmingen #555627)) 1:100 in 1× PBS/0.5% Tween-20. Anti-BrdU was added to cells (50 ul-96 well plate; 200 ul-24 well plate) and incubated for 45-60 minutes at room temperature on a rocker. Antibody was aspirated and cells washed 2× with 1× PBS/0.5% Tween-20. Rabbit Anti-mouse Alexa Fluor 488 (Invitrogen #A-11059) was diluted 1:250 in 1× PBS/0.5% Tween-20. Secondary antibody was added to cells and incubated 30-60 minutes at room temperature on a rocker then washed 3× with 1× PBS/0.5% Tween-20. After the last wash was removed and cells were incubated with 1 μg/ml Hoechst 33342 (Sigma #B2261) diluted in 1× PBS for 45 minutes at room temperature on a rocker. Cells were washed and covered with 1× PBS. Plates were scanned or stored at 4° C. for later scanning on Cellomics.

YoPro-1 Staining for Cellomics was used for cell apoptosis analysis. Add YoPro-1(Final use at 1 ug/ml) and Hoechst (Final use at 10 ug/ml) directly to cell media. Place in 37° C. incubator for 30 min Read directly on Cellomics.

Significant knockdown of MELK, SMC4, TEX10, AURKA, HJURP, BUB1, RFC3, and CCNB2 was achieved in BT549 and MDAMB231 cells transfected with siRNA (Data and staining images not shown). Silencing of these mitotic network genes significantly reduced the proliferation of breast cancer cells and inhibited the BrdU incorporation after treatment with siRNA compared to controls. The current results suggested that silencing expression of MELK, SMC4, TEX10, AURKA, HJURP, BUB1, RFC3, and CCNB2 is a novel approach for inhibition of breast cancer cell growth and these genes may serve as a new candidate therapeutic target for treatment of breast cancer with poor outcome.

### EXAMPLE 3

### Detection of a Mitotic Network Gene in a Patient for Prognosis

A patient biopsy is taken from a tissue such as breast and immunohistochemical analysis is performed using a monoclonal antibody to a mitotic network gene from Table 4 or 5. A positive level or increased level of expressed protein of the mitotic network gene indicates that the patient tissue likely contains malignant cells of basal subtype. The patient prognosis can be determined as possibly poor and the clinician advised so that aggressive treatment can be administered.

### EXAMPLE 4

### siRNA Treatment of a Mitotic Network Gene in a Patient

In Vivo Studies in human subjects. shRNA Preparation and Treatment: Suspensions of the siRNAs of Example 2 can be prepared by combining the oligonucleotides and a buffer or detergent to prepare suspensions in a therapeutic concentration range. The siRNA is synthesized, weighed and can be dissolved in low salt buffer through mixing and sonication. Solubilizing and delivery agents can be added to the solution. Dilutions can be made from a stock solution and the final excipient, such as 0.9% NaCl at 37° C., is added to each dose formulation just prior to dosing. The final ratio of liquid components (e.g., buffer, siRNA, and saline) can be, for example, 5:5:90, respectively. Subjects having been diagnosed with aggressive cancers where a mitotic gene from Table 4 or 5 is detected as expressed ectopically in malignant cells, can be given a therapeutically effective amount of the solution interstitially or intratumorally. A sample dosage may be 0.1 to 0.5 ml, one to five times/week, using a syringe and a needle.

After sufficient period of siRNA administration, a noticeable decrease in the tumor cell growth and cell division should be observed. Administration of the shRNA should cause depletion of SATB1 in the tumor cells, thereby prohibiting the metastasis and growth characteristic of aggressive tumor cells.

### EXAMPLE 5

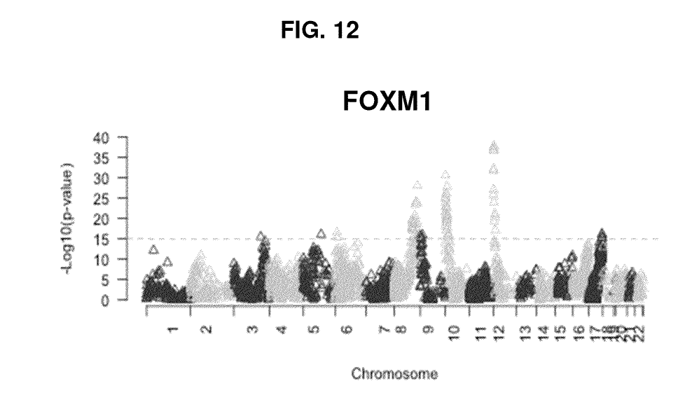

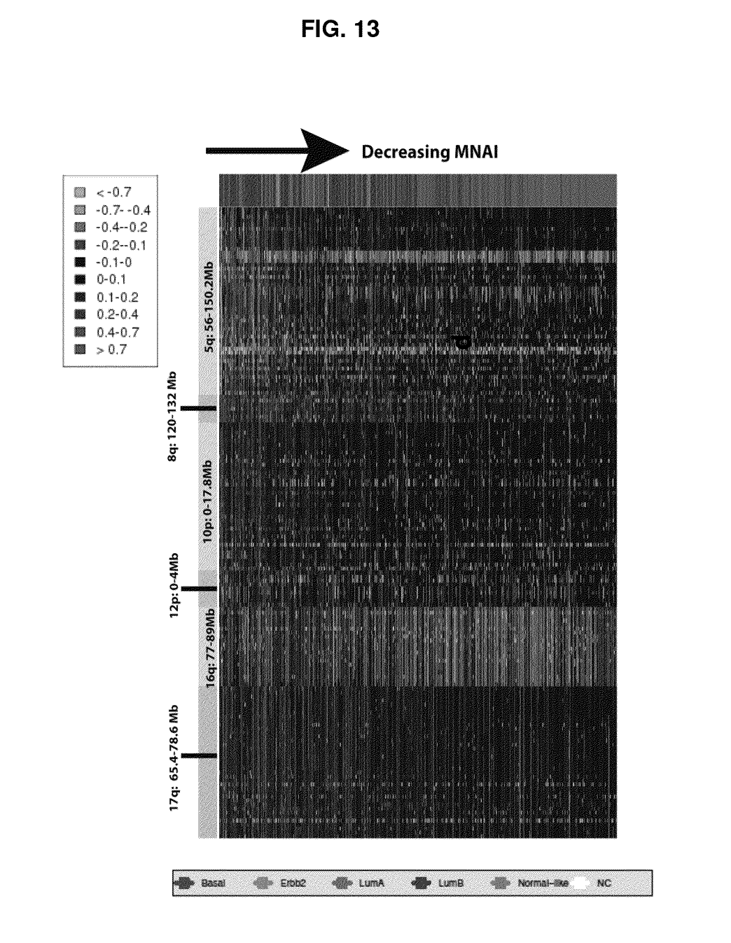

In another experiment, we transiently transfected siRNA for mitotic network genes.into MDAMB231, HCC1569 and BT549 breast cancer cell lines. Non-specific siRNA served as a negative control. Cell viability/proliferation was evaluated by CellTiter-Glo® Referring now to FIG. 12, 22 mitotic network genes prove to be candidate siRNA therapeutic targets. The 22 genes include: PLK1, SMC4, PBK, KIF14, NCAPD2, RRM2, CENPA, CENPE, CENPN, KNTC2, KIF23, RFC3, EXO1, LMNB2, TEX10, DEPDC1, DDX39, MAD2L1, MAD2L1BP, C10orf13, FAM64A, TPX2, AURKA, and TTK.

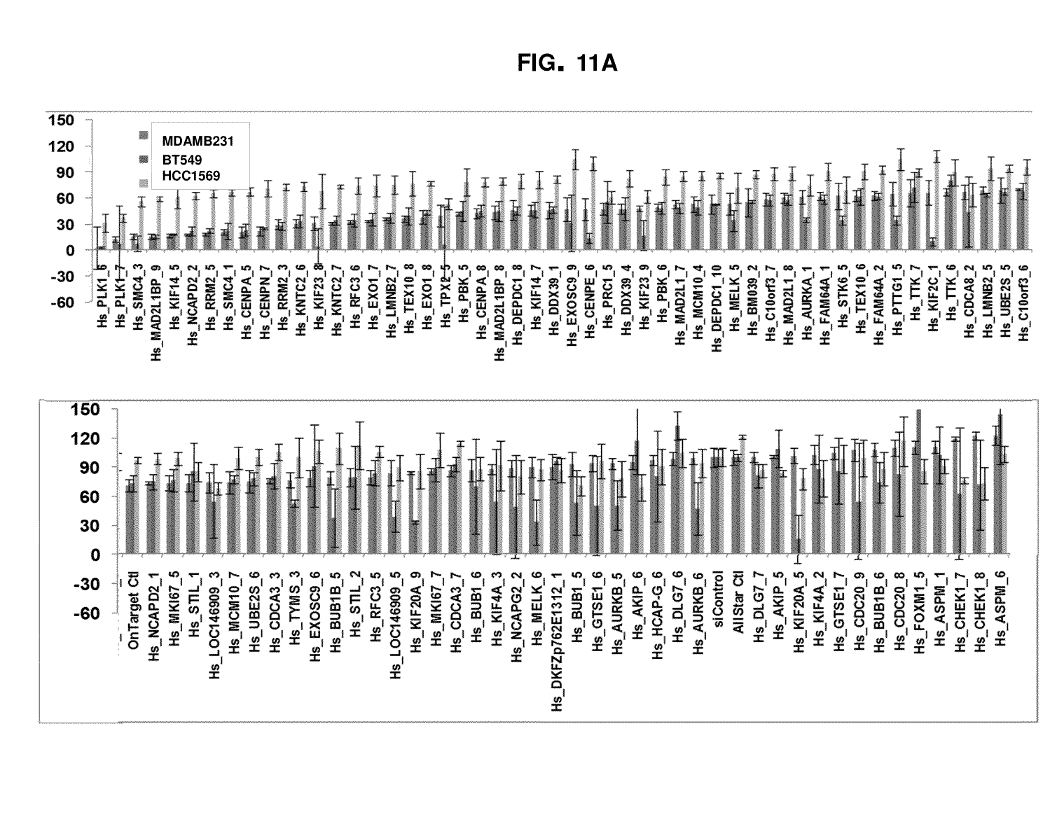

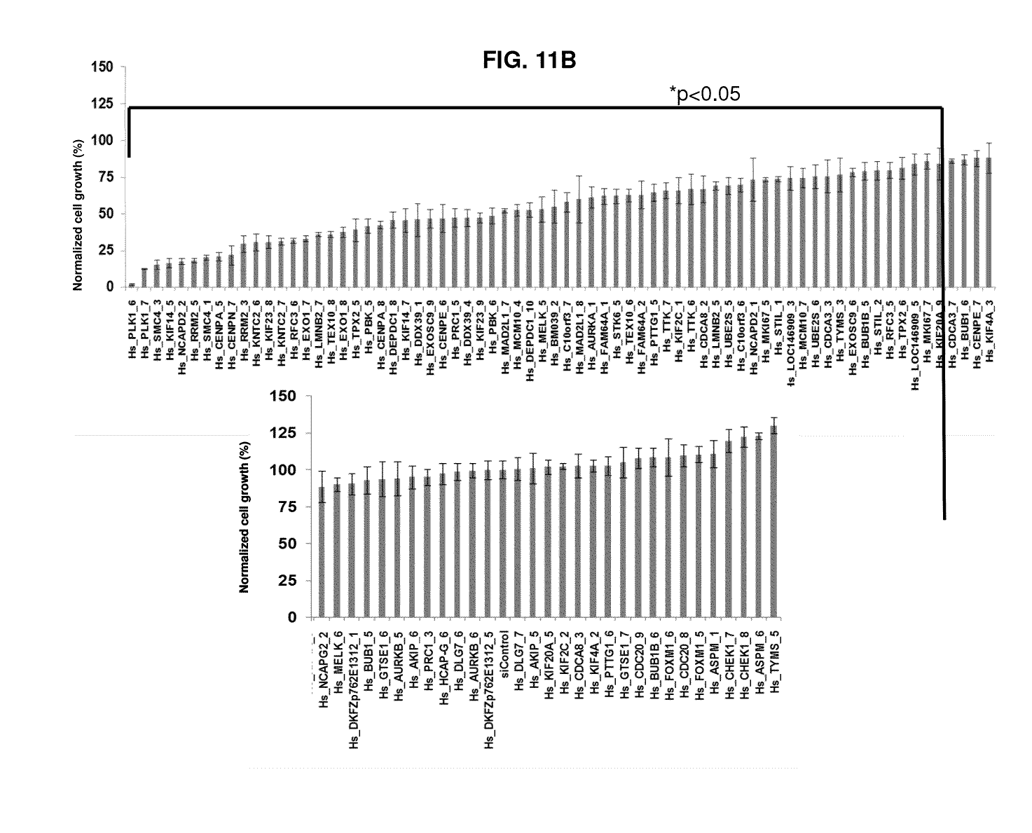

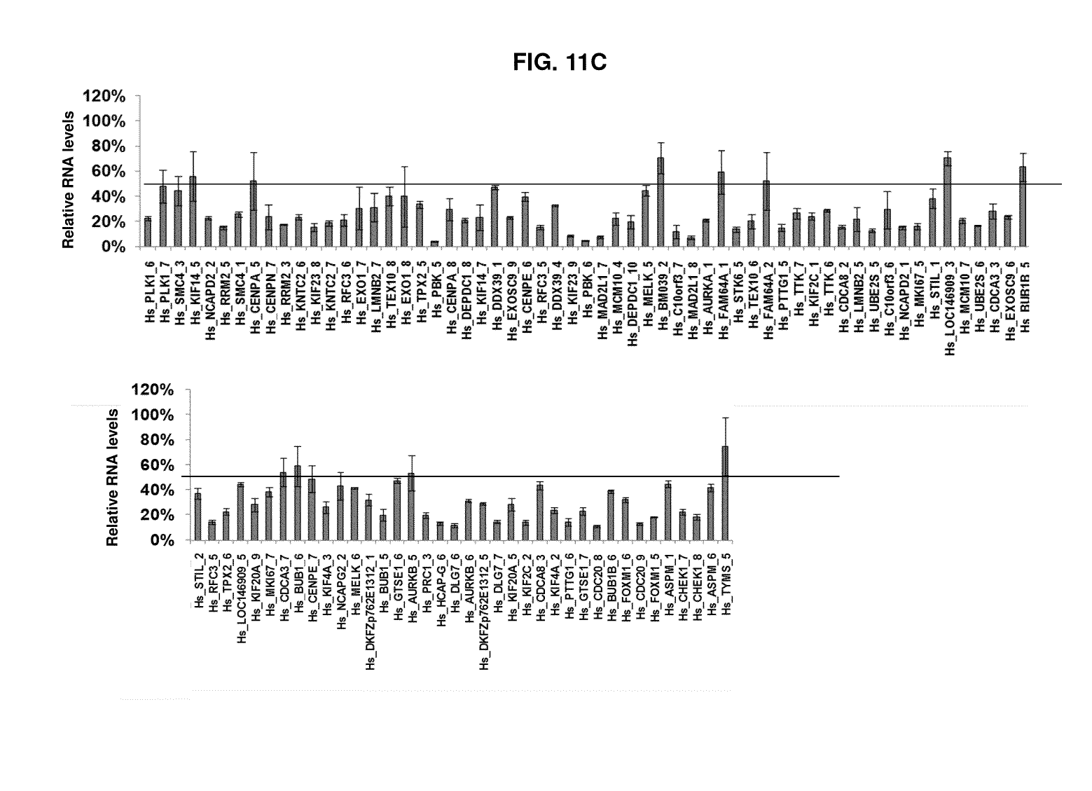

In order to identify additional therapeutic targets, siRNAs were employed to knock down the expression of the 54 genes that comprise the mitotic apparatus network in MDAMB231 cells, which was chosen because of its high MNAI. Greater than 50% knockdown of mRNA levels for 40 mitotic network genes was achieved in the MDAMB231 cell-line (FIG. 11c). FIG. 11b shows that siRNAs targeting 22 genes produced statistically significant decreases in growth at 72 hours relative to that for a scrambled siRNA. The five most inhibitory siRNAs targeted PLK1; the condensin complex component, SMC4; the kinesin family member, KIF14; the condensin complex regulatory subunit, NCAPD2; and the ribonucleotide reductase M2 subunit, RRM2. Interesting, siRNAs against AURKB produced relatively modest growth inhibition in spite of the fact that good mRNA knockdown was achieved. This may explain the somewhat weaker association between mitotic activity and response observed for the AUKB inhibitor, GSK1070916. Protein motif analysis suggests that several of the 22 candidate therapeutic targets defined here are druggable including the mitotic checkpoint protein kinase, TTK; the MAPKK-like protein kinase, PBK (Table 4) and a small molecular inhibitor is already available for AURKA (MLN8054).

Table 6 below shows the siRNA sequences used for each gene. The sequence listing, also shown in Table 7 attached, shows the gene sequence and Accession number of each gene as well.

TABLE 6
siRNA Sequences used for 22 Mitotic Network Genes

| SEQ | | | |
|---|---|---|---|
| ID | Gene | | |
| NO: | Symbol | siRNAs | siRNAs Sequence |
| 1 | CENPA | Hs_CENPA_5 | CACCGTTCCAAAGGCCTGAAA |
| 2 | | Hs_CENPA_8 | CAGAGCCATGACTAGATCCAA |
| 4 | CENPE | Hs_CENPE_6 | CAGGTTAATCCTACCACACAA |
| 6 | CENPN | Hs_CENPN_7 | ATCAGTGATGCTGCCCTGTTA |
| 8 | DDX39 | Hs_DDX39_1 | CCAGGTGATAATCTTCGTCAA |
| 9 | | Hs_DDX39_4 | CAGGACCGGTTTGAAGTTAAT |
| 11 | DEPDC1 | Hs_DEPDC1_8 | TTCCGTAGTCTAAGATAACTA |
| 13 | EXO1 | Hs_EXO1_7 | ATGGATGTACTTTACCTTCTA |
| 14 | | Hs_EXO1_8 | CAGATGTAGCACGTAATTCAA |
| 16 | EXOSC9 | Hs_EXOSC9_9 | TGGCAAATACGTGTAGACCTA |
| 18 | KIF14 | Hs_KIF14_5 | ATGGTTAATCGTGCTCCAGAA |
| 19 | | Hs_KIF14_7 | TAGGGTCTTAGTAACATTCTT |
| 21 | KIF23 | Hs_KIF23_8 | AAGGCTGAAGATTATGAAGAA |
| 22 | | Hs_KIF23_9 | CAGAAGTTGAAGTGAAATCTA |
| 24 | LMNB2 | Hs_LMNB2_7 | CGCCTACAAGTTCACGCCCAA |
| 26 | MAD2L1 | Hs_MAD2L1_7 | ATGGATATTTGTACTGTTTAA |
| 28 | MAD2L1BP | Hs_MAD2L1BP_8 | GAGGAGATGCTGAAGAAGAAA |
| 29 | | Hs_MAD2L1BP_9 | CTCCCAGATAGAACTACTTGA |
| 31 | NCAPD2 | Hs_NCAPD2_2 | CACCCGAATTGTCCAGCAGAA |
| 33 | NDC80 | Hs_KNTC2_6 | CCGAGACCACTTAATGACAAA |
| 34 | | Hs_KNTC2_7 | TCCCTGGGTCGTGTCAGGAAA |
| 36 | PBK | Hs_PBK_5 | AAGTGTGGCTTGCGTAAATAA |
| 37 | | Hs_PBK_6 | TCAGTAGTTATTAGACTCTAA |
| 39 | PLK1 | Hs_PLK1_6 | CCGGATCAAGAAGAATGAATA |
| 40 | | Hs_PLK1_7 | CGCGGGCAAGATTGTGCCTAA |
| 42 | PRC1 | Hs_PRC1_5 | AAGCTTCAGATCCAAATCGAT |
| 44 | RFC3 | Hs_RFC3_6 | TAGCACCATTGCAAGTAACTA |
| 46 | RRM2 | Hs_RRM2_3 | CACACCATGAATTGTCCGTAA |
| 47 | | Hs_RRM2_5 | GCGGGATTAAACAGTCCTTTA |
| 49 | SMC4 | Hs_SMC4_1 | TACCATCGTAGAAATCAATAA |
| 50 | | Hs_SMC4_3 | CAGCGTTTAATAGAGCAAGAA |
| 52 | TEX10 | Hs_TEX10_8 | CTCCGAATTTATGATCCACAA |
| 54 | TPX2 | Hs_TPX2_5 | AAGGCTAATAATGAGATGTAA |

Table 8 below shows the association of genomic aberration and mitotic gene expression in breast cancer. Genetic loci was associated with mitotic network gene expression levels (data not shown). Genetic losses or gains associated with the expression of mitotic network genes in breast cancer. A chromosome locus defined by a BAC on the CGH array used to interrogate copy number. Gain is defined as Log 2(copy number ratio)>0.3]. Loss is defined as Log 2(copy number ratio)<−0.3.

TABLE 8

| | | | | | CORRELATIVE MITOSIS |
|---|---|---|---|---|---|
| BAC probes | chro | start | end | gains/loss | NETWORK GENES |
| CTD-2128D14 | 1 | 21668125 | 21668333 | loss | GTSE1 |
| RP11-6B16 | 1 | 82255369 | 82255623 | loss | CENPN |
| RP11-32F23 | 3 | 4016396 | 4198468 | gain | FOXM1 BUB1B NDC80 GTSE1 TTK |
| | | | | | CENPA BUB1 TPX2 NCAPH GTSE1 |
| | | | | | LMNB2 CEP55 NCAPG HJURP |
| | | | | | MCM10 CDCA3 KIF18B |
| RP11-128A5 | 3 | 8686064 | 8857271 | gain | FOXM1 CENPA CHEK1 NCAPH |
| | | | | | LMNB2 CEP55 MCM10 CDCA3 |
| JG_003A05 | 3 | | | loss | FOXM1 KIF2C KIF2C CDCA8 |
| | | | | | KIF18B |
| JG_005C12 | 3 | | | loss | DDX39 NCAPD2 FOXM1 CDC20 |
| | | | | | GTSE1 CENPA KIF2C AURKB TPX2 |
| | | | | | KIF2C GTSE1 LMNB2 PRC1 CEP55 |
| | | | | | NCAPG CDCA3 CDCA8 FAM64A |
| | | | | | KIF18B |
| RP11-146E16 | 3 | 67739513 | 67813082 | loss | GTSE1 |
| JG_002C02 | 3 | | | gain | CHEK1 |
| RP11-237C24 | 4 | 4538586 | 4538856 | loss | GTSE1 KIF14 FAM64A |
| RP11-53C1 | 4 | 139539118 | 139570264 | loss | GTSE1 |
| RP11-128J2 | 4 | 139773624 | 139773796 | loss | GTSE1 |
| RP11-22O8 | 4 | 141028895 | 141088687 | loss | GTSE1 |
| CTD-2076G21 | 4 | 144747800 | 144748025 | loss | GTSE1 |
| RP11-210F12 | 4 | 147136851 | 147137255 | loss | GTSE1 |
| RP11-130I2 | 4 | 169652616 | 169653013 | loss | SMC4 CENPA KIF14 MNB2 CEP55 |
| | | | | | KIF18B |
| CTD-2100H15 | 4 | 185710628 | 185711125 | loss | KIF18B |
| RP11-5N8 | 5 | 14926849 | 15108182 | gain | TTK |
| RP11-5N11 | 5 | 31657457 | 31800465 | gain | FOXM1 CCNA2 TTK KIF2C |
| | | | | | NCAPH CEP55 |
| RP11-204D12 | 5 | 95832945 | 95833289 | loss | CENPA LMNB2 TEX10 KIF18B |
| RP11-203J7 | 5 | 102680445 | 102841325 | loss | FOXM1 CHEK1 LMNB2 CEP55 |
| | | | | | MCM10 |
| RP11-58G19 | 5 | 113663712 | 113804301 | loss | NCAPD2 FOXM1 LMNB2 |
| JG_005B10 | 5 | | | loss | PLK1 FOXM1 CCNB2 KIF23 CENPA |
| | | | | | KIF2C LMNB2 CDCA8 |
| CTD-2141C20 | 5 | 133509407 | 133509775 | loss | FOXM1 MAD2L1 CCNA2 KIF23 |
| | | | | | CENPA BUB1 TPX2 KIF2C LMNB2 |
| | | | | | CEP55 PBK MCM10 |
| RP11-21J3 | 5 | 134423056 | 134591494 | loss | FOXM1 CHEK1 LMNB2 |
| RP11-170L13 | 5 | 155123958 | 155288478 | loss | PLK1 |
| RP11-94C16 | 6 | | | loss | CHEK1 |
| RP11-115G23 | 7 | 34986452 | 35140107 | gain | CCNA2 CHEK1 LMNB2 MCM10 |
| GS1-77L23 | 8 | | | loss | AURKA |
| RP11-117P11 | 8 | 2057935 | 2058285 | loss | AURKA |
| JG_003G09 | 8 | | | loss | KIF23 CENPE AURKA AURKB |
| | | | | | CCNB1 PRC1 KIF4A CENPN CDCA3 |
| | | | | | KIF18B |
| RP11-246G24 | 8 | 2355757 | 2392700 | loss | AURKA |
| RP11-277K10 | 8 | 9673615 | 9673724 | loss | AURKA NCAPH CDCA3 CDCA8 |
| | | | | | FAM64A KIF18B |
| RP11-262B15 | 8 | 9852835 | 9989112 | loss | AURKA |
| RP11-241I4 | 8 | 10208528 | 10365071 | loss | AURKA |
| RP11-2S2K12 | 8 | 10893274 | 11073332 | loss | AURKA |
| JG_001C05 | 8 | | | loss | AURKA KIF18B |
| JG_002C06 | 8 | | | loss | AURKA |
| CTD-2105I3 | 8 | 17544587 | 17544903 | loss | KIF18B |
| RP11-51C1 | 8 | 19297575 | 19417220 | loss | AURKA |
| RP11-191P9 | 8 | 19651026 | 19798273 | loss | AURKA |
| JG_002G03 | 8 | | | loss | AURKA |
| RP11-238H10 | 8 | 98989769 | 98990075 | gain | TPX2 KIF18B |
| RP11-238H10 | 8 | | | gain | TPX2 KIF18B |
| RP11-131O16 | 8 | 99117291 | 99117617 | gain | MKI67 |
| RP11-102K7 | 8 | 101238454 | 101417646 | gain | TPX2 |
| RP11-10G10 | 8 | 101279027 | 101431772 | gain | CDC20 TPX2 KIF18B |
| RP11-10G10 | 8 | | | gain | CDC20 TPX2 KIF18B |
| RP11-7F12 | 8 | 131174424 | 131333910 | loss | DDX39 NCAPH |
| RP11-44N11 | 8 | 123818629 | 123977782 | gain | PLK1 CENPE KIF18B |
| DMPC-HFF#1-71E5 | 8 | 128822386 | 128822827 | gain | KIF18B |
| DMPC-HFF#1-71E5 | 8 | | | gain | KIF18B |
| RP11-642A1 | 8 | 141602644 | 141809117 | gain | FOXM1 CHEK1 KIF18B |
| GS1-261I1 | 8 | 147000 | | gain | CENPE KIF18B |
| CTB-41L13 | 9 | 335734 | 336205 | gain | DDX39 FOXM1 UBE2S CCNA2 |
| | | | | | BUB1B DLGAP5 NDC80 GTSE1 |
| | | | | | KIF23 TTK CENPA KIF14 KIF2C |
| | | | | | BUB1 TPX2 KIF2C NCAPH EXOSC9 |
| | | | | | LMNB2 CEP55 HJURP ASPM |
| | | | | | DEPDC1 MCM10 CDCA3 FAM64A |
| | | | | | KIF18B |
| RP11-62H18 | 9 | 4575558 | 4575778 | gain | GTSE1 CDCA3 |
| RP11-125K10 | 9 | 4819655 | 4991841 | loss | GTSE1 |
| RP11-165O14 | 9 | 5873408 | 6029621 | loss | NCAPD2 FOXM1 MELK KIF2C |
| | | | | | GTSE1 ASPM DEPDC1 CDCA3 |
| RP11-132G20 | 9 | 15858419 | 15858778 | loss | GTSE1 FAM64A |
| RP11-111O7 | 10 | 1613880 | 1614195 | gain | MCM10 |
| RP11-23O12 | 10 | 3658177 | 3829073 | gain | MCM10 |
| RP11-59D4 | 10 | 4336670 | 4491362 | gain | MCM10 |
| RP11-5B23 | 10 | 6668422 | 6700235 | gain | MCM10 |
| RP11-85M7 | 10 | 6685013 | 6762260 | gain | PLK1 KIF2C DEPDC1 MCM10 |
| RP11-33J8 | 10 | 7235611 | 7353296 | gain | MCM10 |
| RP11-72C6 | 10 | 7937407 | 8047073 | gain | MCM10 |
| RP11-72C6 | 10 | | | Gain | MCM10 |
| RP11-40D12 | 10 | 13729752 | 13730163 | gain | MCM10 |
| JG_002E06 | 10 | | | gain | SMC4 GTSE1 |
| RP11-8O10 | 10 | 127832427 | 127985179 | loss | SMC4 PLK1 MAD2L1 GTSE1 |
| | | | | | CENPA KIF14 LMNB2 CDCA8 |
| | | | | | KIF18B |
| RP11-27F2 | 10 | 133470856 | 133471230 | loss | KIF2C CDCA8 KIF18B |
| RP1-44H16 | 11 | 800000 | 800000 | loss | PLK1 KIF23 DEPDC1 |
| JG_002F11 | 11 | | | loss | DDX39 FOXM1 KIF2C NCAPH |
| | | | | | GTSE1 HJURP CDCA3 |
| GS-137C7 | 11 | 524257 | 524320 | loss | CHEK1 |
| JG_002A04 | 11 | | | loss | GTSE1 KIF23 KIF14 |
| RP11-133H19 | 11 | 8555485 | 8732332 | loss | GTSE1 MCM10 |
| RP11-170H2 | 11 | 12608554 | 12781974 | loss | GTSE1 |
| RP11-245K9 | 11 | 12678356 | 12761390 | loss | CCNA2 GTSE1 KIF14 BUB1 KIF2C |
| | | | | | MKI67 LMNB2 ASPM KIF18B |
| RP11-21L19 | 11 | 14198466 | 14382320 | loss | GTSE1 KIF20A |
| CTC-352E23 | 11 | 76048916 | 76049278 | loss | AURKA |
| JG_001F12 | 11 | | | loss | DDX39 AURKA |
| JG_003H01 | 11 | | | loss | DDX39 AURKB NCAPH |
| JG_001H12 | 11 | | | loss | AURKA |
| RP11-51M23 | 11 | 104324007 | 104480911 | loss | CHEK1 |
| CTD-2059P15C | 11 | 112785546 | 112786169 | loss | GTSE1 AURKA |
| JG_003C03 | 11 | | | loss | AURKA |
| JG_005C03 | 11 | | | loss | DDX39 UBE2S |
| RP11-35P15 | 11 | 117022814 | 117194571 | loss | DDX39 AURKA NCAPH GTSE1 |
| | | | | | FAM64A |
| JG_001H11 | 11 | | | loss | DDX39 CHEK1 |
| JG_002H04 | 11 | | | Loss | CHEK1 |
| RP11-45N4 | 11 | 117978549 | 118170478 | loss | DDX39 |
| RP11-62A14 | 11 | 118829643 | 118992466 | loss | DDX39 NCAPH |
| RP11-117K21 | 11 | 119773356 | 119939639 | loss | CHEK1 |
| RP11-145I11 | 11 | 121670407 | 121828933 | loss | CHEK1 |
| RP11-15J15 | 11 | 125422188 | 125586057 | loss | DDX39 |
| RP11-112M22 | 11 | 127647353 | 127788923 | loss | DDX39 NCAPH MCM10 |
| RP11-24N12 | 12 | 2977825 | 3145569 | gain | NCAPD2 FOXM1 CCNA2 BUB1 |
| | | | | | TPX2 EXOSC9 TEX10 |
| | | | | | CDCA3 KIF18B |
| RP11-74M9 | 12 | 4177492 | 4279837 | gain | NCAPD2 FOXM1 RFC3 BUB1 |
| | | | | | LMNB2 MCM10 CDCA3 |
| CTC-298G6 | 12 | 4177492 | 4279837 | loss | NCAPD2 FOXM1 MCM10 |
| RP11-15L3 | 12 | 68217524 | 68403913 | gain | CENPN |
| RP11-34K15 | 13 | 42360122 | 42360372 | loss | SMC4 CENPE |
| RP11-52B21 | 13 | 46118238 | 46284283 | loss | CENPE |
| RP11-288G5 | 14 | 38438528 | 38438793 | gain | KIF23 KIF18B |
| RP11-94K16 | 14 | 48148555 | 48298851 | loss | STIL |
| RP11-63G22 | 14 | 64450951 | 64468781 | loss | GTSE1 |
| CTD-2055A23 | 14 | 64611597 | 64638980 | loss | KIF23 KIF14 |
| RP11-59M15 | 14 | 69101594 | 69152329 | loss | KIF23 KIF14 |
| RP11-92H20 | 14 | 74381659 | 74551240 | loss | KIF14 |
| RP11-84G6 | 14 | 87697387 | 87697728 | loss | GTSE1 KIF23 CENPA KIF14 |
| | | | | | MCM10 KIF18B |
| RP11-83I2 | 14 | 88149356 | 88149603 | loss | FOXM1 BUB1B GTSE1 KIF23 TTK |
| | | | | | MELK CENPA CENPE STIL KIF14 |
| | | | | | KIF2C BUB1 LMNB2 CEP55 NCAPG |
| | | | | | HJURP ASPM MCM10 CDCA3 |
| | | | | | CDCA8 KIF18B |
| RP11-40P23 | 14 | 88958250 | 88958551 | loss | GTSE1 KIF14 GTSE1 LMNB2 |
| | | | | | KIF18B |
| RP11-16O4 | 14 | 90296988 | 90466301 | loss | GTSE1 |
| RP11-26J5 | 14 | 93784993 | 93937440 | loss | GTSE1 |
| CTD-2119E15 | 14 | 95384632 | 95385419 | loss | KIF23 KIF14 KIF2C MCM10 KIF18B |
| RP11-86O9 | 14 | 99137698 | 99137859 | loss | GTSE1 |
| RP11-123M6 | 14 | 100295974 | 100461023 | gain | GTSE1 |
| RP11-30I7 | 15 | 54201403 | 54362671 | loss | CENPN |
| JG_002C05 | 15 | | | gain | KIF2C |
| RP11-380F1 | 16 | 3718150 | 3718301 | gain | CCNA2 DLGAP5 KIF23 KIF2C |
| | | | | | NCAPH MCM10 CDCA8 |
| RP11-160D13 | 16 | 6468000 | 6603117 | gain | NCAPD2 DEPDC1 |
| RP11-165B11 | 16 | 12029008 | 12211311 | gain | CENPN |
| RP11-141E3 | 16 | 23458654 | 23647789 | gain | NCAPG |
| RP11-253O10 | 16 | 74101075 | 74101477 | loss | CENPN |
| RP11-131C4 | 17 | 47644854 | 47645096 | gain | STIL CHEK1 |
| RP11-102E12 | 18 | 4457862 | 4614130 | loss | LMNB2 |
| RP11-4B17 | 18 | 72860583 | 72909206 | loss | GTSE1 |
| JG_002E09 | 19 | | | gain | PLK1 CENPA STIL CHEK1 KIF14 |
| JG_004G08 | 20 | | | loss | CDCA8 |
| JG_001E01 | 20 | | | gain | CHEK1 KIF2C LMNB2 DEPDC1 |
| | | | | | MCM10 CDCA8 KIF18B |
| JG_004H04 | 20 | | | gain | CHEK1 |
| CTA-799F10 | 22 | 49369027 | 49436279 | loss | NCAPD2 FOXM1 GTSE1 LMNB2 |
| | | | | | DEPDC1 |

Table 9 below shows the association of expression of 54 mitotic genes and DNA variants in CEPH with eQTL analysis. This table uses the Dataset GSE12626 used in Smirnov, D. A., Morley, M., Shin, E., Spielman, R. S., Cheung, V. G. Genetic analysis of radiation-induced changes in human gene expression. Nature 459:587-91(2009). eQTL analysis of genes in mitotic network using the expression from 15 CEPH famlies. Table 9 shows the loci significantly associated with the expression of gene in mitotic network (p<0.0001).

TABLE 9
Association of expression of 54 mitotic genes
and DNA variants in CEPH with eQTL analysis.

| | Region | |
|---|---|---|
| Chr | (Mb) | Genes |
| 1 | 190-195 | CENPE, AURKA, CCNB1, CDCA8, CENPA, PLK1, TPX2 |
| 2 | 18-23 | CCNA2, GTSE1, UBE2S, CDCA3, CHEK1, KIF2C, SMC4, CCNB2 |
| | 137-143 | AURKA, AURKB, CDC20, CENPE, KIF20A, KIF2C, MKI67, PLK1, |
| | | ASPM, KIF14, KIF18B, CEP55, GTSE1, HJURP, NCAPG, NCAPH, |
| | | NDC80, PRC1, STIL, TPX2, TTK, CCNB1, CDCA8, CENPA, PTTG1, |
| | | RRM2 |
| 4 | 12-17 | GTSE1, AURKA, CCNB1, CDC20, CDCA8, CENPA, CENPE, KIF2C, |
| | | NCAPG, PLK1, AURKB, HJURP, KIF23, KIF4A, NCAPH, PRC1, |
| | | TPX2, NDC80, RRM2, STIL |
| 6 | 2-7 | CENPA, CENPE, KIF23, CEP55, KIF14, KIF4A, PRC1, RRM2, |
| | | STIL, TYMS |
| | 160-165 | AURKA, AURKB, CDC20, CDCA3, HJURP, KIF2C, NCAPG, NCAPH, |
| | | PTTG1, UBE2S, GTSE1 |
| 7 | 2-7 | PTTG1, AURKA, BUB1, CDCA8, KIF2C, PLK1 |
| 12 | 114-119 | EXOSC9, CCNB1, CDC20, CENPE, EXO1, GTSE1, KIF20A, PTTG1, |
| | | TEX10, TYMS |
| 14 | 97-102 | BUB1B, CEP55, GTSE1, NCAPG, NCAPH, FOXM1, KIF18B, KIF2C, |
| | | PRC1, TPX2 |
| 16 | 0-5 | CDC20, FOXM1, GTSE1, KIF14, KIF18B, NCAPD2, NCAPG, NCAPH, |
| | | PRC1, KIF2C, TPX2 |
| 19 | 70-75 | AURKB, CENPE, GTSE1, KIF14, KIF20A, KIF23, KIF2C, KIF4A, |
| | | PRC1 |
| 21 | 23-28 | DDX39, FOXM1, GTSE1, LMNB2, PLK1, CDC20, KIF20A, NCAPD2 |
| 22 | 33-38 | AURKA, AURKB, BUB1, CDC20, GTSE1, HJURP, KIF20A, NCAPG, |
| | | NCAPH, TPX2, UBE2S |

### EXAMPLE 6

### Genomic drivers for the Mitotic Apparatus Activity

The existence of a genetically determined mitotic apparatus network in mice⁴ and immortalized lymphocytes raised the possibility that genomic aberrations in tumors might contribute to increased mitotic activity in human cancers. This possibility was explored by identifying genomic losses and gains associated with elevated mitotic network genes expression in breast cancer in two separate studes (Chin et al, Genomic and transcriptional aberrations linked to breast cancer pathophysiologies. Cancer Cell 10, 529-541 (2006); and Curtis et al, in preparation). Loci that were significantly associated with the expression of multiple mitotic network genes are illustrated in FIG. 12. These include associations with genomic copy number aberrations involving regions on chromosome 5q (56-150 Mbp), 8q (120-132 Mbp), 10p (0-18 Mbp), 12p (0-4 Mbp), and 17q (65.4-78.6 Mbp) (Table 5, FIG. 12). The strength of association for genome-wide copy number and expression of each of the 54 mitotic apparatus genes was determined for a cohort of 824 breast cancers (Curtis et al dataset; association strength not shown). FIG. 12a shows significance of associations along the genome for regions of copy number abnormality associated with expression of the transcription factor, FOXM1; one member of the mitotic apparatus network. All but 6 genes in the mitotic apparatus were significantly associated with genomic aberrations in two separate primary breast cancer datasets (Table 8,). The associations with the overall mitotic network activity as defined by the MNAI are particularly strong for narrowly defined regions of copy number increase involving chromosomes 8q24, 10p, 12p13 and 17q24. This led us to examine these alteration hotspots in greater detail. These regions encode the transcription factors, MYC, ZEB1, FOXM1, and SOX9 each of which has predicted binding sites in multiple genes comprising the 54 mitotic apparatus network. The amplification of these transcription factors may combine to “explain” the transcriptional regulation of each of the mitotic network genes suggesting that amplification or deletion of these loci may directly modulate mitotic network activity.

### EXAMPLE 6

### Therapeutic Approaches to High MNAI Tumors

High MNAI is strongly associated with reduced overall survival so that effective therapies are needed for tumors of this class. The existence of a genomic and genetically determined mitotic network suggests that cancers with high mitotic activity may have evolved to be dependent on elevated mitotic activity and so would be more sensitive to lower concentrations of mitotic apparatus inhibitors than cells with lower mitotic activity. In support of this hypothesis, the GI₅₀ values for GSK1070916, GSK461364 and GSK923295 were found to be significantly lower in cell lines with a high MNAI as compared to cells with a low MNAI (FIG. 6). This result combined with the finding that high MNAI is associated with reduced survival suggests that early clinical trials of drugs targeting the mitotic apparatus network would be best directed toward tumors with high mitotic network activity. These results also suggest that normal tissues with high mitotic activity (bone marrow, testes, and endometrium) are likely to experience significant toxicity when targeted by mitotic apparatus inhibitors (data not shown).

Analysis suggests that drugs that target the mitotic apparatus may be clinically equivalent since they modulate the same biological process despite the fact that they inhibit different proteins involved in mitotic function. This suggests that combinations of mitotic apparatus inhibitors might not be expected to show additive or synergistic effects. This was tested by treating a sensitive (HCC38) or resistant (MDAMB175) breast cancer cell line with GSK461364, GSK1070916 and GSK923295 either alone or in combination. As shown in FIG. 8a, the combination of compounds against two different mitotic apparatus proteins did not increase the response in either cell type. Since toxicity does not appear to be additive, combinations of drugs targeting the mitotic apparatus might be deployed either together or sequentially to counter therapeutic resistance. This suggests that drugs targeting other genes in the mitotic apparatus network might further contribute to development of a multi-drug mitotic apparatus therapeutic strategy that could effectively counter the development of drug resistance.

### EXAMPLE 7

### Validation of HJURP Expression and Protein Levels as a Marker

We measured HJURP expression level in human breast cancer cell lines and primary breast cancers by Western blot and/or by Affymetrix Microarray; and determined its associations with clinical variables using standard statistical methods. Validation was performed with the use of published microarray data. We assessed cell growth and apoptosis of breast cancer cells after radiation using high-content image analysis. This example also described in Hu et al., “The expression level of HJURP has an independent prognostic impact and predicts the sensitivity to radiotherapy in breast cancer,” Breast Cancer Res. 2010; 12(2): R18, published online 2010 Mar. 8, and hereby incorporated by reference for all purposes.

HJURP was expressed at higher level in breast cancer than in normal breast tissue. HJURP mRNA levels were significantly associated with estrogen receptor (ER), progesterone receptor (PR), Scarff-Bloom-Richardson (SBR) grade, age and Ki67 proliferation indices, but not with pathologic stage, ERBB2, tumor size, or lymph node status. Higher HJURP mRNA levels significantly decreased disease-free and overall survival. HJURP mRNA levels predicted the prognosis better than Ki67 proliferation indices. In a multivariate Cox proportional-hazard regression, including clinical variables as covariates, HJURP mRNA levels remained an independent prognostic factor for disease-free and overall survival. In addition HJURP mRNA levels were an independent prognostic factor over molecular subtypes (normal like, luminal, Erbb2 and basal). Poor clinical outcomes among patients with high HJURP expression were validated in five additional breast cancer cohorts. Furthermore, the patients with high HJURP levels were much more sensitive to radiotherapy. In vitro studies in breast cancer cell lines showed that cells with high HJURP levels were more sensitive to radiation treatment and had a higher rate of apoptosis than those with low levels. Knock down of HJURP in human breast cancer cells using shRNA reduced the sensitivity to radiation treatment. HJURP mRNA levels were significantly correlated with CENPA mRNA levels.

HJURP mRNA level is a prognostic factor for disease-free and overall survival in patients with breast cancer and is a predictive biomarker for sensitivity to radiotherapy.

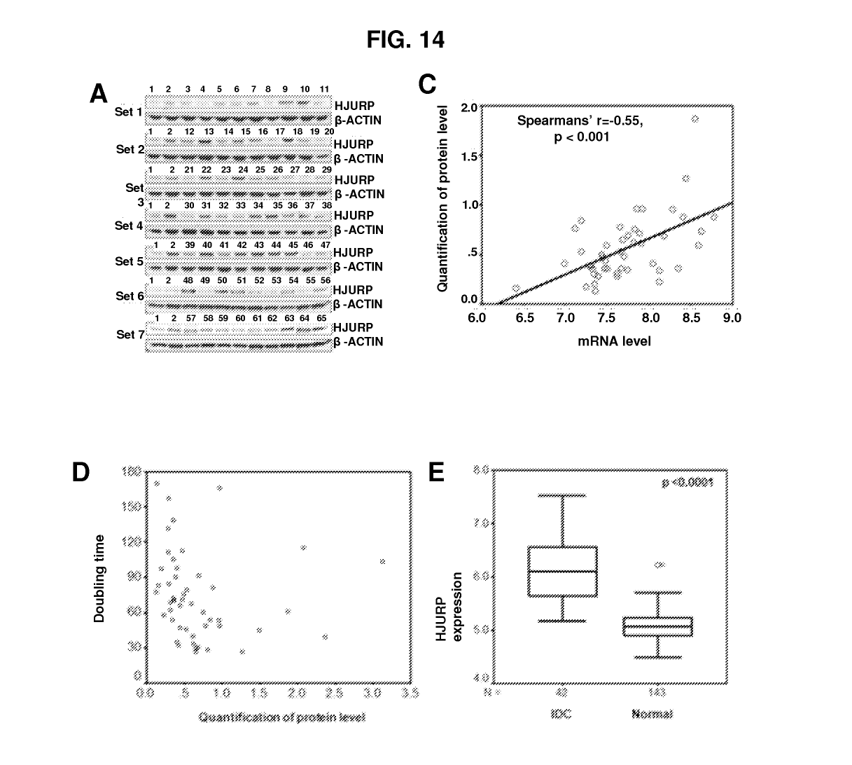

We examined the protein levels of HJURP in a large panel of human breast cancer cell lines and immortalized non-malignant mammary epithelial cells, which have been analyzed for genomic aberrations by comparative genomic hybridization (CGH) and for gene-expression profiles using Affymetrix microarrays [Neve R M, Chin K, Fridlyand J, Yeh J, Baehner F L, Fevr T, Clark L, Bayani N, Coppe J P, Tong F, Speed T, Spellman P T, DeVries S, Lapuk A, Wang N J, Kuo W L, Stilwell J L, Pinkel D, Albertson D G, Waldman F M, McCormick F, Dickson R B, Johnson M D, Lippman M, Ethier S, Gazdar A, Gray J W. A collection of breast cancer cell lines for the study of functionally distinct cancer subtypes. Cancer Cell. 2006;10:515-527. doi: 10.1016/j.ccr.2006.10.008]. Although we found few genetic alterations in the HJURP locus by inspection of these CGH microarray data, the protein levels of HJURP were elevated in about 50% of these breast cancer cell lines when compared to immortalized but non-malignant mammary epithelial cells 184A1N4, 184B5, and S1 (FIGS. 14A, B). In order to determine whether mRNA expression reflected protein levels, we quantified and normalized HJURP protein expression in each cell line and demonstrated a significant correlation between mRNA expression and protein levels (the Affymetrix probe for HJURP is 218726_at: Spearman's correlation coefficient R=0.55, P<0.001; FIG. 14C). Next we examined whether HJURP protein level is associated with cell proliferation. In order to do so, we measured the doubling time for each cell line and found that the doubling time of cell lines was negatively correlated with HJURP protein levels (Spearman's correlation coefficient R=−0.395, P=0.005; FIG. 14D). Furthermore, HJURP mRNA levels in invasive ductal carcinomas (IDC) were statistically significantly higher than its levels in the normal breast ducts (P<0.0001). FIG. 14E.

### Materials and Methods.

Cell lines and cell lysates. The names of cell lines used in our investigations are listed previously. The derivation, sources, and maintenance of most of the breast cancer cell lines used in this study have been reported previously [13] or were provided in Table 2 of Hu et al. Breast Cancer Res. 2010; 12(2): R18. These cell lines have been previously analyzed for genomic aberrations by comparative genomic hybridization (CGH) and for gene-expression profiles using Affymetrix microarrays (Santa Clara, Calif., USA) [13]. The information on growth conditions of additional cell lines was listed in Table 2 of Hu et al., Breast Cancer Res. 2010; 12(2): R18. Cells at 50% to 75% confluence were washed in ice-cold phosphate buffered saline (PBS). Then cells were extracted with a lysis buffer (containing 50 mM HEPES (pH 7.5), 150 mM NaCl, 25 mM β-glycerophosphate, 25 mM NaF, 5 mM EGTA, 1 mM EDTA, 15 mM pyrophosphate, 2 mM sodium orthovanadate, 10 mM sodium molybdate, 1% Nonidet-P40, 10 mg/ml leupeptin, 10 mg/ml aprotinin, and 1 mM PMSF). Cell lysates were then clarified by centrifugation and frozen at −80° C. Protein concentrations were determined using the Bio-Rad BCA protein assay kit (Cat#23227, Pierce Biotechnology, Rockford, Ill., USA).

Western blot. For Western blots, 10 μg of protein extracts per lane were electrophoresed with denaturing sodium doedecyl sulfate (SDS)-polyacrylamide gels (4% to 12%), transferred to PVDF membranes (Millipore, Temecula, Calif., USA), and incubated with HJURP antibody 1:500 (Rabbit, HPA008436, Sigma-Aldrich, St. Louis, Mo., USA) and actin (goat, sc-1616, Santa Cruz Biotechnology, Santa Cruz, Calif., USA) diluted with blocking buffer (927-40000, LI-COR Biosciences, Lincoln, Nebr., USA) The membranes were washed four times with TBST and treated with 1:10,000 dilution of Alex Fluor 680 donkey anti-rabbit (A10043, Invitrogen, Carlsbad, Calif., USA) and IRDye 800CW conjugated donkey anti-goat (611-731-127, Rockland, Gilbertsville, Pa., USA) to detect HJURP and actin respectively. The signals were detected by infrared imaging (LI-COR Biosciences, Lincoln, Nebr., USA). Images were recorded as TIFF files for quantification.

Protein Quantification. Protein levels were measured by quantifying infrared imaging recorded from labeled antibodies using Scion Image [14]. For each protein, the blots were made for 7 sets of 11 cell lines, each set including the same pair (SKBR3 and MCF12A) to permit intensity normalization across sets. A basic multiplicative normalization was carried out by fitting a linear mixed effects model to log intensity values, and adjusting within each set to equalize the log intensities of the pair of reference cell lines across the sets.

Tumor Samples. Detailed patient information has been described in our previous studies [15]. This analysis is based on previously reported comparative genomic hybridization (CGH) and a gene expression profile of 130 tumors from UC San Francisco and the California Pacific Medical Center collected between 1989 and 1997.

Validation. The association of HJURP expression levels and survival among patients with breast tumors was examined in existing microarray data sets of primary tumor samples that had been profiled with an Affymetrix microarray assay (either HG-U133A or HG U133 Plus 2.0) ((GEO:GSE1456), (GEO:GSE7390), (GEO:GSE2034), (GEO:GSE4922)) or Agilent oligo microarray (Santa Clara, Calif., USA)(Table USA)(Table 11). Probe 218726_at and 20366 (GenBank: NM_{—}018410) were used to measure HJURP expression in Affymetrix and Agilent GeneChip, respectively. The process data from GEO website were downloaded for analysis.

HJURP shRNA construct. The shRNA sequences were (forward) 5′-GATCCCC GAGCGATTCATCTTCATCA TTCAAGAGA TGATGAAGATGAATCGCTC TTTTTGGAAA-3′ (SEQ ID NO: 56) and (reverse) 5′-AGCT TTTCCAAAAA GAGCGATTCATCTTCATCA TCTCTTGAA TGATGAAGATGAATCGCTC GGG-3′ (SEQ ID NO: 57) synthesized from IDT (Integrated DNA Technologies, Inc., San Diego, Calif., USA). HJURP shRNA was cloned into BglII and HindIII cleavage sites of pSUPER.retro.puro vector based on manufactory's instruction (OligoEngine, Seattle, Wash., USA). HJURP shRNA expression vector were confirmed by direct DNA sequencing.

Retroviral packaging and infection. HJURP shRNA (or empty) retroviral vectors along with packaging system pHit60 and pVSVG vectors were then co-transfected into the HEK 293 Phoenix ampho packaging cells (ATCC, Manassas, Va., USA) by using FuGENE6 transfection reagent (Roche, Lewes, UK) according to the instruction to produce retroviral supernatants. Forty-eight hours after transfection, the virus-containing supernatant was filtered through a 0.45 μm syringe filter. Retroviral infection was performed by adding filtered supernatant to a MDAMB231 cell line cultured on 10 cm dishes with 50% confluent in the presence 4 ug/ml of polybrene (Sigma, St. Louis, Mo., USA). Six hours after infection, the medium was changed with fresh medium. After 48 hours, infected cells were selected by adding 5 μg/ml puromycin (Sigma) to the culture medium for 72 hours and then maintained in complete medium with 2 μg/ml puromycin. Down-regulation of HJURP expression was confirmed by Western blot analysis.

High content imaging to assess cell number and apoptotic cells. The effects on cell growth and apoptosis were assessed by a Cellomics high-content image screening system (Cellomics, Thermo Fisher Scientfic Inc., Pittsburgh, Pa., USA) after breast cancer cells exposed to a single dose of 0 (sham), 1, 2, 4, 6, 8 or 10 Gy X-ray radiation emitted from an irradiator (model 43855F, Faxitron X-ray Corporation, Lincolnshire, Ill., USA). Live cells in 96 well plates with six replicates from each treatment were stained with 1 nmol/L YO-PRO-1 positive cells.

Statistical analysis. Spearman's correlation coefficient and test were used to examine the relationship between HJURP mRNA level and its protein level in the cell line studies, and the relationship with age, tumor size in the tumor studies, and CENPA mRNA level. The association between HJURP mRNA level and clinical factors, such as estrogen receptor (ER), progesterone receptor (PR), ERBB2 and lymph node status, pathological stage, Scarff-Bloom-Richardson (SBR) grade, was analyzed by Mann-Whitney U (for two groups) or Kruskal-Wallis H (for more than two groups) test. Kaplan-Meier plots were constructed and a long-rank test was used to determine differences among disease free and overall survival curves according to HJURP expression level or radiotherapy. Multivariate analyses were carried out to examine whether HJURP expression is an independent prognostic factor for survival when adjusting for other covariates (age, ER, PR, lymph node, pathologic stage, SBR grade, tumor size) or the molecular subtypes (normal like, luminal, Erbb2 and Basal) using Cox proportional-hazard regression. In addition, the relation between HJURP expression and survival was explored in microarray data sets by dividing the cases from each cohort into a group with high (top one-third), moderate (middle one-third), and low (bottom one-third) level of expression. All analyses were performed by SPSS 11.5.0 for Windows. A two-tailed P-value of less than 0.05 was considered to indicate statistical significance.

### Results

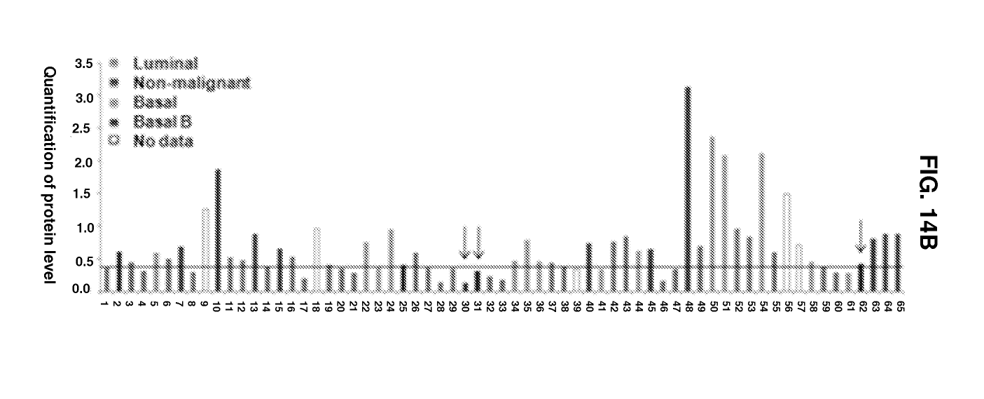

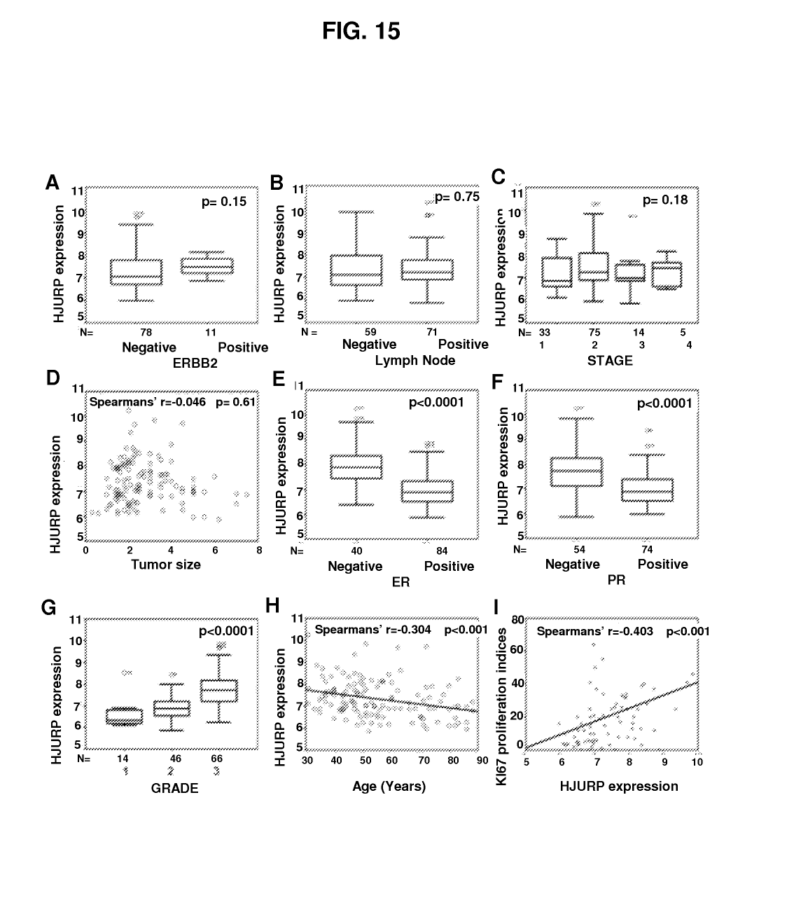

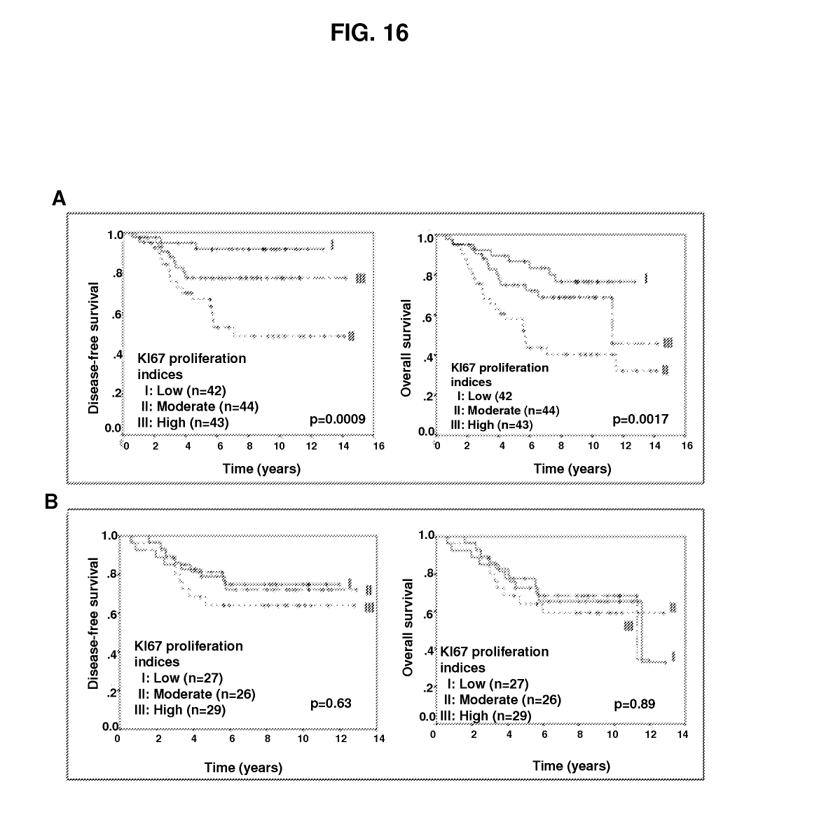

HJURP mRNA level is an independent prognostic biomarker for poor clinical outcome. We assessed the association between HJURP mRNA levels and clinical factors and outcomes using a cohort of breast cancer patients in our previous studies [Chin K, DeVries S, Fridlyand J, Spellman P T, Roydasgupta R, Kuo W L, Lapuk A, Neve R M, Qian Z, Ryder T, Chen F, Feiler H, Tokuyasu T, Kingsley C, Dairkee S, Meng Z, Chew K, Pinkel D, Jain A, Ljung B M, Esserman L, Albertson D G, Waldman F M, Gray J W. Genomic and transcriptional aberrations linked to breast cancer pathophysiologies. Cancer Cell. 2006;10:529-541. doi: 10.1016/j.ccr.2006.10.009]. HJURP expression level is measured as log₂ (probe intensities) by Affymetrix microarray. In univariate analysis, HJURP mRNA levels were not associated with pathological stage, tumor size, ERBB2 positive, or lymph node positive status (FIGS. 15a, b, c, d). However, high HJURP mRNA levels were significantly associated with estrogen-receptor negative (ER−) (P<0.0001), progesterone-receptor negative (PR−) P<0.0001), advanced SBR grade (P<0.0001), young age (P<0.001) and Ki67 proliferation indices (P<0.001) (FIGS. 15e, f, g, h, i). When we divided HJURP expression levels into three groups (low=bottom third, moderate=middle third, and high=top third), patients whose tumor with high HJURP expression levels had significantly shorter disease free survival (P=0.0009) and overall survival (P=0.0017) period using a Kaplan-Meier log rank analysis (FIG. 15A). Interestingly, although HJURP expression significantly correlated with Ki67 proliferation indices, Ki67 proliferation indices are not significantly associated with both disease-free and overall survival (FIG. 15B).

In multivariate analyses (including age, pathological stage, SBR grade, ER status, PR status, lymph node status, tumor size, HJURP mRNA levels), lymph node positive and high pathological stage were associated with poor disease free survival, whereas lymph node positive, big tumor size, and age were associated with poor overall survival (Table 10). HJURP expression level is an indicator of a poor prognosis for disease-free survival (hazard ratio, 2.05; 95% CI, 1.18 to 3.58; P=0.011), and for overall survival (hazard ratio, 1.83; 95% CI, 1.11 to 3.01; P=0.018) (Table 10).

TABLE 10
Results of multivariate analysis of independent prognostic
factors in patients with breast cancer using Cox regression

| | Disease-Free survival | | Overall survival | |
|---|---|---|---|---|
| Factor | Hazard ratio (95% CI) | P value | Hazard ratio (95% CI) | P value |
| HJURP expression⁺ | 2.05 (1.18 to 3.58) | 0.011 | 1.83 (1.11 to 3.01) | 0.018 |
| Lymph node (positive) | 3.76 (1.16 to 12.25) | 0.028 | 2.72 (1.08 to 6.88) | 0.035 |
| High Stage | 2.23 (1.08 to 4.59) | 0.030 | 1.85 (0.94 to 3.63) | 0.075 |
| Tumor size | 1.32 (0.97 to 1.79) | 0.079 | 1.34 (1.02 to 1.77) | 0.038 |
| Age (year) | 1.01 (0.99 to 1.05) | 0.33 | 1.03 (1.004 to 1.053) | 0.022 |
| High SBR Grade | 0.76 (0.33 to 1.75) | 0.52 | 1.00 (0.50 to 2.00) | 0.99 |
| ER (positive) | 0.63 (0.21 to 1.94) | 0.42 | 0.86 (0.33 to 2.25) | 0.75 |
| PR (positive) | 0.90 (0.33 to 2.50) | 0.84 | 0.95 (0.40 to 2.26) | 0.91 |

⁺HJURP expression is measured as log2 (probe intensities) by Affymetrix microarray

To validate our findings, we used several independent breast cancer cohorts with previously reported microarray data deposited in the Gene Expression Omnibus (GEO) database [Gene Expression Omnibus (GEO) website], to compare mRNA level of HJURP in tumor tissue with patient survival (Table 11).

TABLE 11
Information of gene expression datasets used in this study

| | GEO access | | |
|---|---|---|---|
| | number or web | | |
| Dataset | location | Radiotherapy | Reference |
| 1 | GSE1456 | Not available | Pawitan Y, Gene expression profiling spares early breast |
| | | | cancer patients from adjuvant therapy: derived and |
| | | | validated in two population-based cohorts. Breast |
| | | | Cancer Res. 2005; 7: R953-964. doi: 10.1186/bcr1325 |
| 2 | GSE7390 | Not available | Desmedt C, et al., TRANSBIG Consortium. Strong time |
| | | | dependence of the 76-gene prognostic signature for |
| | | | node-negative breast cancer patients in the TRANSBIG |
| | | | multicenter independent validation series. Clin Cancer |
| | | | Res. 2007; 13: 3207-3214. doi: 10.1158/1078-0432.CCR- |
| | | | 06-2765 |
| 3 | NKI * | 82.4% | Vijver M J van de, et al., A gene-expression signature as |
| | | patients | a predictor of survival in breast cancer. N Engl J Med. |
| | | | 2002; 347: 1999-2009. doi: 10.1056/NEJMoa021967 |
| 4 | GSE2034 | 86.7% | Wang Y, et al., Gene-expression profiles to predict |
| | | patients | distant metastasis of lymph-node-negative primary |
| | | | breast cancer. Lancet. 2005; 365: 671-679 |
| 5 | GSE4922 | Not available | Ivshina A V, et al., Genetic reclassification of histologic |
| | | | grade delineates new clinical subtypes of breast cancer. |
| | | | Cancer Res. 2006; 66: 10292-10301. doi: 10.1158/0008- |
| | | | 5472.CAN-05-4414 |

* Howard Y. Chang, et al., website companion for “Robustness, Scalability, and Integration of a Wound-Reponse Gene Expression Signature in Predicting Breast Cancer Survival”, Proc Natl Acad Sci USA, Feb. 8, 2005 published online, and found at Microarray-pubs page at Stanford University website.

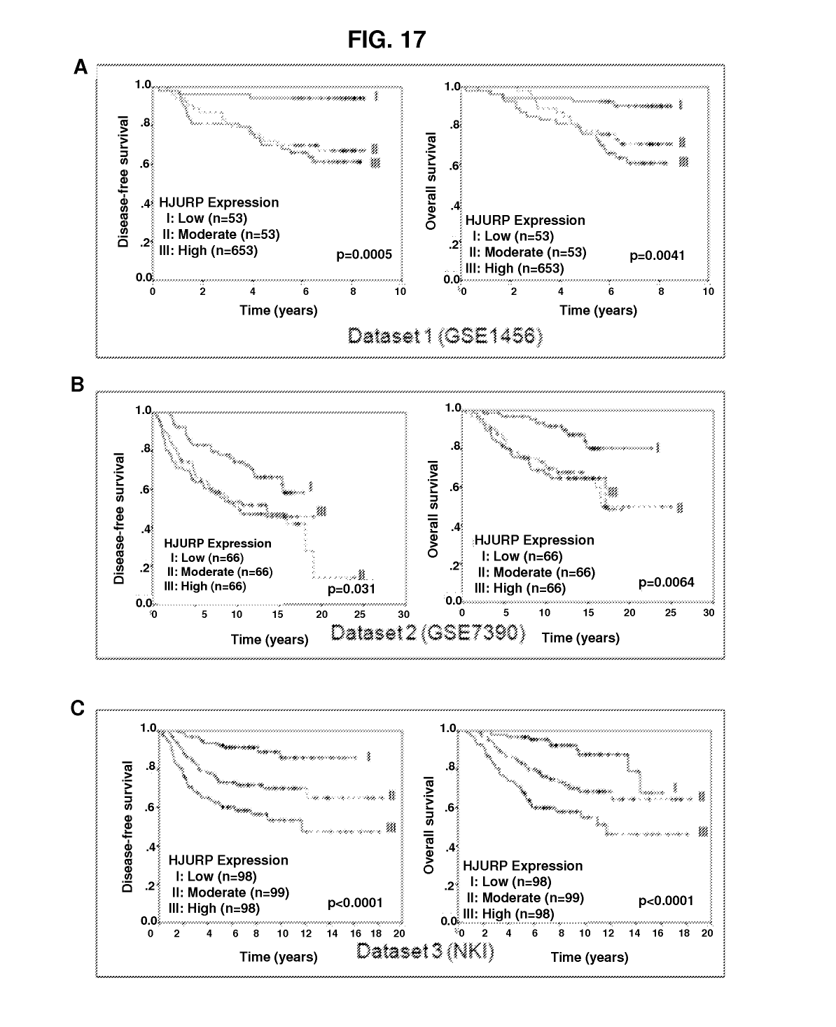

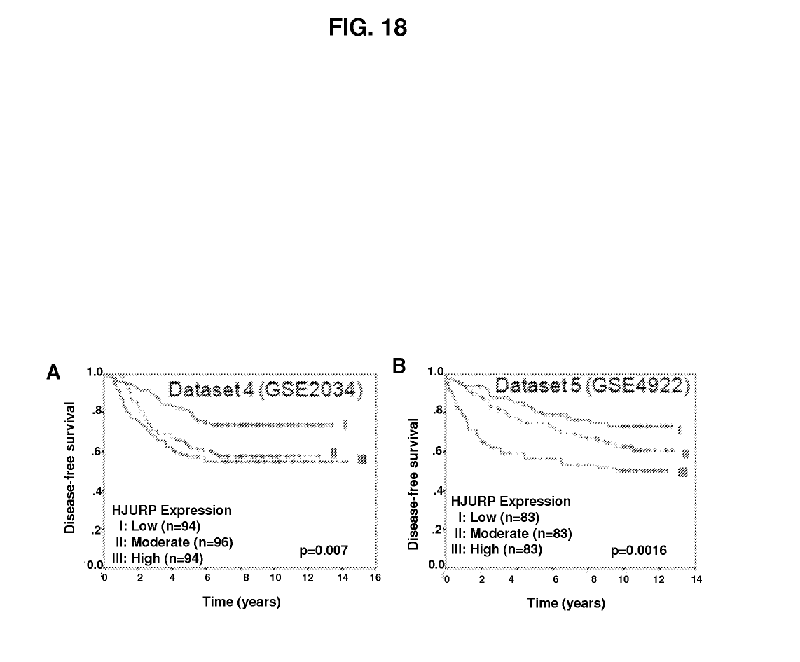

In agreement with our initial findings, decreased disease-free and overall survival rate was associated with high mRNA level of HJURP in all of the datasets (FIGS. 17 and 18).

Finally, we investigated whether HJURP mRNA levels were an independent prognostic factor over molecular subtypes (normal like, luminal, Erbb2 and basal) using Cox regression. In order to do so, three data sets (Scion Corporation Home Page, Dataset 1 and 3), in which the information of the molecular subtypes was available, were combined because there were few patients in each subtype using each data set. As shown in Table 12, both HJURP mRNA levels and molecular subtypes were independently significantly associated with survival.

TABLE 12
Both HJURP mRNA levels and molecular subtypes are independent prognostic
factors in patients with breast cancer using Cox regression^{#}

| | Disease-Free survival | | Overall survival | |
|---|---|---|---|---|
| Factor | Hazard ratio (95% CI) | P value | Hazard ratio (95% CI) | P value |
| HJURP | | 6.19E-07 | | 0.00011 |
| High vs Low | 3.26 (2.01 to 5.28) | 1.72E-06 | 3.23 (1.85 to 5.62) | 3.65E-05 |
| Moderate vs Low | 3.34 (2.11 to 5.27) | 2.3E-07 | 2.89 (1.68 to 4.95) | 0.00012 |
| Molecular Subtypes | | 0.0069 | | 0.00012 |

^{#}The results are obtained from the combination of three data sets (reference 14, Dataset 1 and 3) where the information of the molecular subtypes (Normal like, Luminal, Erbb2 and Basal) was available.

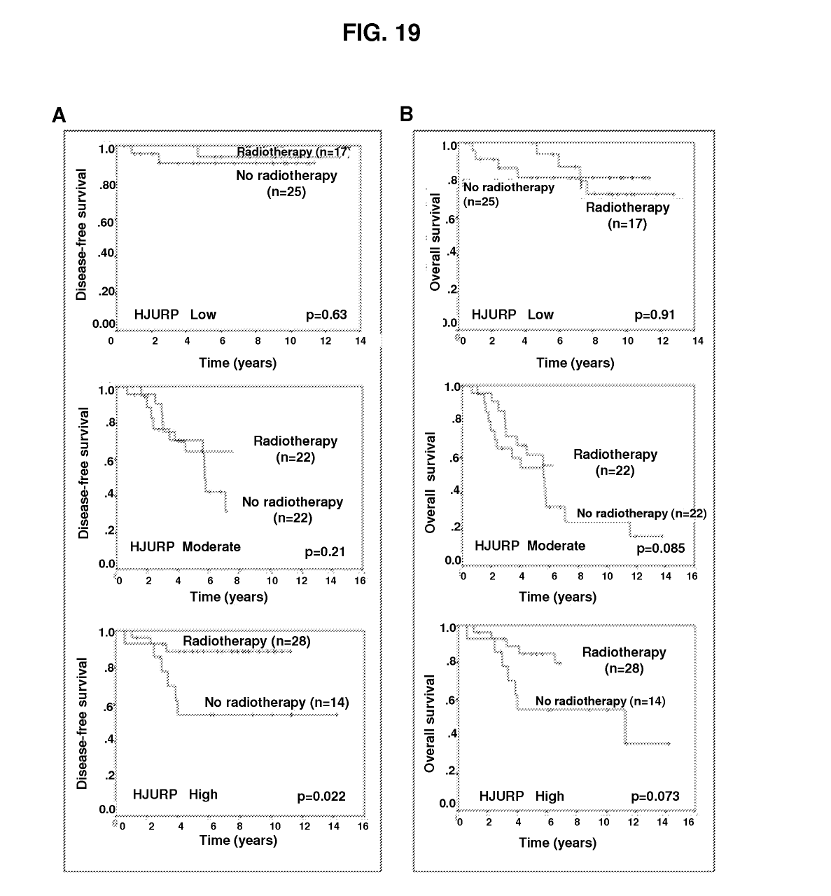

HJURP mRNA level predicts the sensitivity to radiation treatment in breast cancer patients and cell lines. It has been reported that HJURP is involved in the DNA repair pathway, thus next we investigated whether the HJURP mRNA level is a predictive marker for radiotherapy in our cohort of breast cancer patients. As shown in FIG. 19A, the radiotherapy significantly increased disease-free survival of patients within the high HJURP mRNA level group (P=0.022) whereas radiotherapy did not within the low HJURP mRNA level group. The data showed a trend toward increased overall survival within the high and moderate HJURP mRNA level group (FIG. 19B).

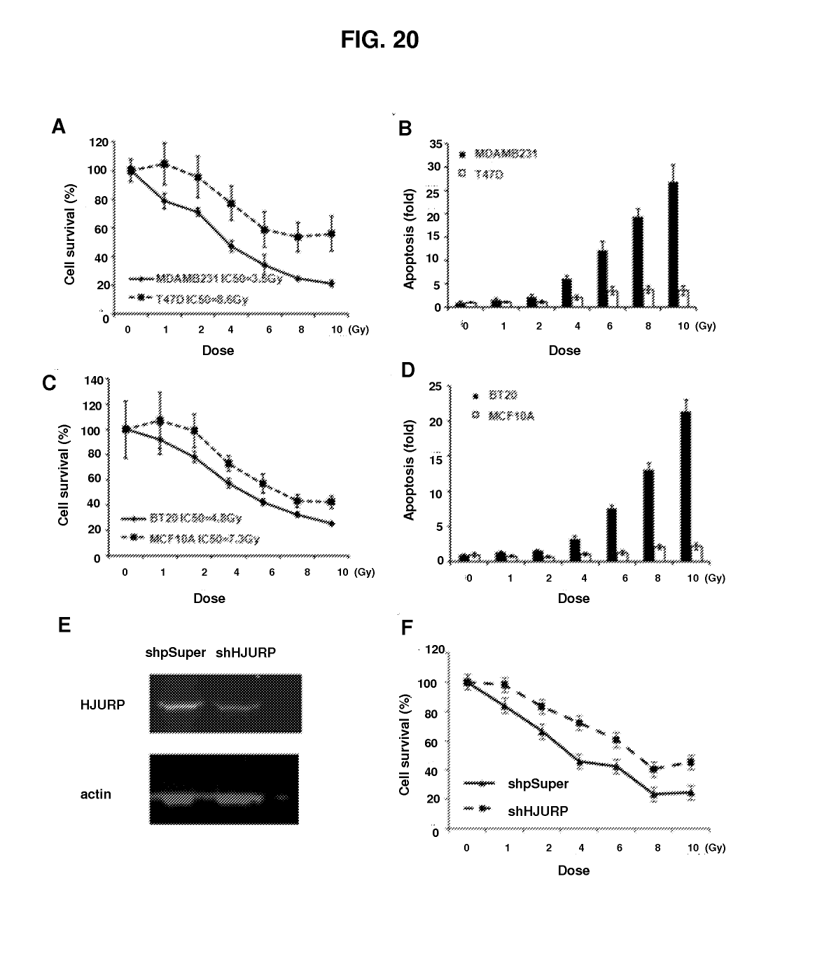

In order to confirm the relationship between HJURP mRNA levels and radiation sensitivity, we selected two cell lines, one had high levels of HJURP (MDAMB231), the other had a low level of HJURP (T47D), and treated them with different doses of x-ray irradiation. Seventy-two hours after radiation, we measured cell growth and apoptosis using high-content image analysis. Our data showed that the response to radiation in breast cancer cell line MDAMB231 (IC50=3.5 Gy) was more sensitive than T47D (IC50=8.6 Gy) (FIG. 20A). Consistent with radiation sensitivity, MDAMB231 cells had a higher rate of apoptosis than T47D cells (FIG. 20B). Similar results were found in additional cell lines BT20 with high levels of HJURP and MCF10A with low levels of HJURP (FIG. 20c, d). Finally we designed small interfering RNA (shRNA) against HJURP and generated stable transfectants in a human breast cancer cell line (MDAMB231). The shRNA down-regulated HJURP protein levels by 75%, as assessed by Western blotting assays (FIG. 20e). Knockdown of the HJURP gene reduced the sensitivity to radiation (FIG. 200.

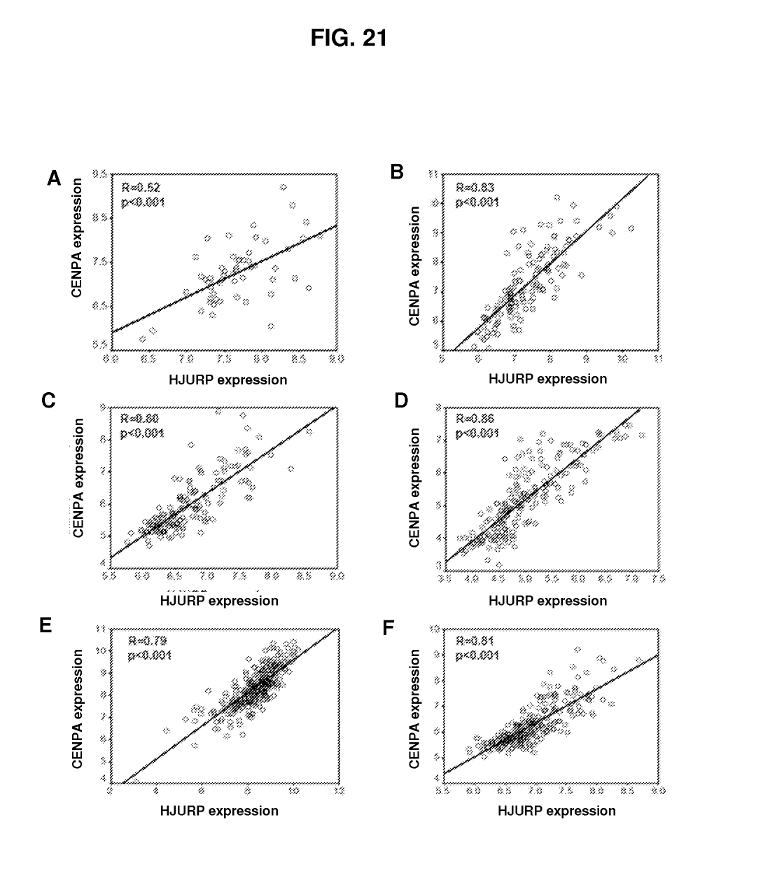

Co-overexpression of HJURP and CENPA in breast cancer. Recently it has been shown that HJURP interacts with CENPA for localization to centromeres and for accurate chromosome segregation. Thus we examined the expression pattern between HJURP and CENPA at the mRNA level. Surprisingly, HJURP levels were significantly and positively correlated with CENPA levels in human breast cancer cell lines (FIG. 21A) and primary breast tumors (FIG. 21B). Such highly significant correlation was confirmed in four independent cohorts with breast tumors (FIGS. 21c, d, e, f).

The current study is the first to report that HJURP is overexpressed in breast cancer cell lines and primary human breast cancer compared to non-malignant human mammary epithelial cells and normal breast tissues. High HJURP mRNA expression is significantly associated with both shorter disease-free and overall survival which were validated in five independent clinical datasets for breast cancer. Furthermore, HJURP is a predictive marker for sensitivity of radiotherapy, indicating levels of HJURP mRNA and protein in breast cancer patients are clinically relevant.

Although we found HJURP mRNA levels were not associated with ERBB2 status, the mRNA levels of HJURP was still found significantly higher in triple-negative (ER negative, PR negative, ERBB2/HER2/neu not overexpressed) breast cancer, possibly due to the fact that a higher HJURP mRNA level is significantly associated with ER or PR negative status. Triple negative breast cancer has distinct clinical and pathological features, and also has relatively poor prognosis and aggressive behavior [Sørlie T, Perou C M, Tibshirani R, Aas T, Geisler S, Johnsen H, Hastie T, Eisen M B, Rijn M van de, Jeffrey S S, Thorsen T, Quist H, Matese J C, Brown P O, Botstein D, Eystein Lønning P, Børresen-Dale A L. Gene expression patterns of breast carcinomas distinguish tumor subclasses with clinical implications. Proc Natl Acad Sci USA. 2001,98 :10869-10874; Cheang M C, Voduc D, Bajdik C, Leung S, McKinney S, Chia S K, Perou C M, Nielsen T O. Basal-like breast cancer defined by five biomarkers has superior prognostic value than triple-negative phenotype. Clin Cancer Res. 2008; 14:1368-1376; Dent R, Trudeau M, Pritchard K I, Hanna W M, Kahn H K, Sawka C A, Lickley L A, Rawlinson E, Sun P, Narod S A. Triple-Negative Breast Cancer: Clinical Features and Patterns of Recurrence. Clin Cancer Res. 2007; 13:4429-4434], consistent with our finding that high HJURP expression is associated with a bad prognosis. Furthermore, our studies showed that the prognostic effect of HJURP mRNA level on survival is independent of the clinical factors, such as age, lymph node, pathological stage, SBR grade, ER, PR, tumor size, and the molecular subtypes. In addition, we found there is a significant correlation between HJURP expression and Ki67 proliferation indices; however, HJURP expression is a better biomarker than Ki67 proliferation indices for the predication of prognosis.

Our results showed that patients with low mRNA levels of HJURP already had a good prognosis and could not get further benefit from radiotherapy, suggesting these patients may not necessarily benefit from receiving radiotherapy. However, patients with high HJURP mRNA levels could increase their survival with radiotherapy, but they still had a worse prognosis than those with low levels as found in Dataset 3 (FIG. 17c) and Dataset 4 (FIG. 18a) where almost all patients received radiotherapy with or without additional benefit. Thus a high level of HJURP is overall associated with poor prognosis. Although we note our findings will require replication in additional independent and larger cohorts, our in vitro studies further confirmed that breast cancer cells with high levels of HJURP are more sensitive to radiation treatment, and even more convincingly, knock down of HJURP by shRNA reduces the sensitivity to radiation. The radiation induced more apoptosis in these cells, consistent with clinical findings. A previous report showed that HJURP interacts with proteins hMSH5 and NBS1, suggesting HJURP is involved in the DNA double-strand break repair process [Kato T, Sato N, Hayama S, Yamabuki T, Ito T, Miyamoto M, Kondo S, Nakamura Y, Daigo Y. Activation of Holliday junction recognizing protein involved in the chromosomal stability and immortality of cancer cells. Cancer Res. 2007; 67:8544-8553. doi: 10.1158/0008-5472.CAN-07-1307.]. The understanding of the roles that HJURP plays in DNA repair and cell death in response to DNA damage may provide new insights into the molecular mechanisms of breast tumor development and may help to improve breast cancer therapies. In addition, we found that cells with HJURP shRNA grew slowly (data not shown), which is consistent with the finding that the double time of cell lines was negatively correlated with HJURP protein level, indicating HJURP plays an important role in cell proliferation. Thus one of the reasons why the ability of HJURP to act as a marker for prognosis and response to radiotherapy may be linked to its control of cell proliferation.

HJURP has recently been reported to interact with CENP-A for the purpose of localizing CENP-A and loading new CENP-A nucleosomes on the centromere [Dunleavy E M, Roche D, Tagami H, Lacoste N, Ray-Gallet D, Nakamura Y, Daigo Y, Nakatani Y, Almouzni-Pettinotti G. HJURP is a cell-cycle-dependent maintenance and deposition factor of CENP-A at centromeres. Cell. 2009; 137:485-497. doi: 10.1016/j.cell.2009.02.040.; Foltz D R, Jansen L E, Bailey A O, Yates J R III, Bassett E A, Wood S, Black B E, Cleveland D W. Centromere-specific assembly of CENP-a nucleosomes is mediated by HJURP. Cell. 2009; 137:472-484. doi: 10.1016/j.cell.2009.02.039.1. CENP-A is the key determinant of centromere formation and kinetochore assembly, which regulate the complex job of attaching chromosomes to the mitotic spindle; ensuring that those attachments are correct; signalling a delay in mitotic progression if they are not, and regulating the movements of the chromosomes towards the spindle poles in anaphase. Thus overexpression of HJURP in human breast cancer may be similar to overexpression of mitotic kinases, such as Aurora kinases, which induce genomic instability that is one of the hallmarks for tumor development. In this study we showed that HJURP mRNA levels are highly significantly correlated with CENPA mRNA levels in human breast cancer cell lines and primary breast tumors. Such correlation is also found in other types of human cancer, such as cancers from lung, ovary, prostate (data not shown), suggesting that compatible mRNA levels of HJURP and CENPA might be required for tumor progression. Further investigation of the interaction between HJURP and CENPA for breast cancer development will be carried out in our future studies.

Although the foregoing invention has been described in some detail by way of illustration and example, for purposes of clarity of understanding, it will be obvious that various alternatives, modifications and equivalents may be used and the above description should not be taken as limiting in scope of the invention which is defined by the appended. In addition, where possible, combinations of the various embodiments, or combinations of the aspects of certain embodiments is considered to be within the scope of the disclosure.

The subject methods may include each of the activities associated with the assay and use of the information derived from the assay. As such, methodology implicit to the use of the assay and live cell device forms part of the invention. Such methodology may include using the assay or the live cell device in contexts not specifically detailed herein, and other applications.

More particularly, a number of methods according to the present invention involve the manner in which the assay is applied to a particular disease or condition, or cell type, or receptor-mediated activation pathway. Other methods concern the manner in which the system develops the physical context of the assay, and how the receptor redistribution is detected. Any method herein may be carried out in any order of the recited events which is logically possible, as well as in the recited order of events, or slight modifications of those events or the event order.

Also, it is contemplated that any optional feature of the inventive variations described may be set forth and claimed independently, or in combination with any one or more of the features described herein.

Reference to a singular item, includes the possibility that there is a plurality of the same items present. More specifically, as used herein and in the appended claims, the singular forms “a” and “the” include plural referents unless specifically stated otherwise. In other words, use of the articles allow for at least one molecule of the subject item in the description above as well as the claims below. Number identifying quantity, such as 54, or 25 can be “about 54” or “about 25”, meaning that a couple less or a couple more items can be included, so that “about 25” can include a range from 22 to 28, for example. Accordingly, a number can represent an approximate value having a range of numbers close to the stated number and still be considered within the approximation intended with the term “about X”.

Without the use of such exclusive terminology, the term comprising in the claims shall allow for the inclusion of any additional element irrespective of whether a given number of elements are enumerated in the claim, or the addition of a feature could be regarded as transforming the nature of an element set forth in the claims. Except as specifically defined herein, all technical and scientific terms used herein are to be given as broad a commonly understood meaning as possible while maintaining claim validity.

The breadth of the present invention is not to be limited to the examples provided and/or the subject specification, but rather only by the scope of the claim language.

All references cited are incorporated by reference in their entirety. Although the foregoing invention has been described in detail for purposes of clarity of understanding, it is contemplated that certain modifications may be practiced within the scope of the appended claims.

### REFERENCES

1. Toyoshima-Morimoto, F., Taniguchi, E., Shinya, N., Iwamatsu, A. & Nishida, E. Nature 410, 215-20 (2001).
2. Barr, F. A., Sillje, H. H. & Nigg, E. A. Nat. Rev. Mol. Cell. Biol. 5, 429-440 (2004).
3. McInnes, C. et al. Nat. Chem. Biol. 2, 608-617 (2006).
4. Winkles, J. A. & Alberts, G. F. Oncogene 24, 260-266 (2005).
5. Yamada, S. et al. Oncogene 23, 5901-5911(2004).
6. Sortie, T. et al. Proc. Natl. Acad. Sci. 98, 10869-10874 (2001).
7. Cheang, M. C. et al. Clin. Cancer Res. 14, 1368-1376 (2008).
8. Neve, R. M. et al. Cancer Cell 10, 515-527 (2006).
9. Seki, A., Coppinger, Jang, C., Yates, J. R. & Fang, G. Science 20 320, 1655-1658 (2008).
10. Mac [symbol] rek, L. et al. Nature 455, 119-123 (2008).
11. Weaver, B. A., Bonday, Z. Q., Putkey, F. R., Kops, G. J., Silk, A. D. & Cleveland D. W. J. Cell Biol 162, 551-563 (2003).
12. Bernard, P., Hardwick, K. & Javerzat, J. P. J. Cell Biol. 143, 1775-1787 (1998).
13. Chin, K. et al. Cancer Cell 10, 529-541 (2006).
14. Wang, Y. et al. Lancet 365, 671-679 (2005).
15. Quigley, D. A. et al. Nature (2009).
16. Strebhardt, K. & Ullrich, A. Nat. Rev. Cancer 6, 321-330 (2006).
17. Jackson, J. R., Patrick, D. R., Dar, M. M. & Huang, P. S. Nature Reviews Cancer 7, 107-117 (2007)
18. Maere, S., Heymans, K. & Kuiper, M. Bioinformatics 21, 3448-3449 (2005).
19. Pawitan Y, Bjöhle J, Amler L, et al. Gene expression profiling spares early breast cancer patients from adjuvant therapy: derived and validated in two population-based cohorts. Breast Cancer Res 2005; 7:R953-R964.
20. Ivshina A V, George J, Senko O, et al. Genetic reclassification of histologic grade delineates new clinical subtypes of breast cancer. Cancer Res 2006; 66:10292-10301.
21. Takaki T, Trenz K, Costanzo V, Petronczki M. Polo-like kinase 1 reaches beyond mitosis-cytokinesis, DNA damage response, and development. Curr Opin Cell Biol. 2008 December; 20(6):650-60.
22. Keen N and Taylor S, Mitotic drivers-inhibitors of the Aurora B Kinase. Cancer Metastasis Rev 2009 June; 28(1-2):185-95.
23. Anderson K, Lai Z, McDonald O B, Stuart J D, Nartey E N, Hardwicke M A, Newlander K, Dhanak D, Adams J, Patrick D, Copeland R A, Tummino P J, Yang J. characterization of GSK1070916, a potent and selective inhibitor of Aurora B and Aurora C Kinases with an extremely long residence time. Biochem J. 2009 Mar. 11.
24. Wood K W, Chua P, Sutton D, Jackson J R. Centromere-associated protein E: a motor that puts the brakes on the mitotic checkpoint. Clin Cancer Res. 2008 Dec. 1; 14(23):7588-92.
25. Bild A H, Yao G, Chang J T, et al. Oncogenic pathway signatures in human cancers as a guide to targeted therapies. Nature 2006; 439:353-357.
26. Lee Y, Scheck A C, Cloughesy T F, Lai A et al. Gene expression analysis of glioblastomas identifies the major molecular basis for the prognostic benefit of younger age. BMC Med Genomics 2008 Oct. 21; 1:52.
27. Metzeler K H, Hummel M, Bloomfield C D, Spiekermann K et al. An 86-probe-set gene-expression signature predicts survival in cytogenetically normal acute myeloid leukemia. Blood 2008 Nov. 15; 112(10):4193-201.
28. Choy E, Yelensky R, Bonakdar S, Plenge R M et al. Genetic analysis of human traits in vitro: drug response and gene expression in lymphoblastoid cell lines. PLoS Genet 2008 November; 4(11):e1000287.

1-49. (canceled)

50. A method comprising,
providing a cell from tissue of a human patient;
measuring the expression levels of a signature set of genes comprising all or a subset of at least 6 to 22 of the mitotic network genes provided in Table 4, wherein at least 6 genes in the signature are selected from the group consisting of MELK, SMC4, TEX10, AURKA, HJURP, BUB1, RFC3, and CCNB2, and at least one gene selected is HJURP,
wherein elevated expression levels of the mitotic network genes as compared to a reference level prognoses poor outcome for said patient;
and administering to the patient an inhibitor of mitosis.

51. The method of claim 50, wherein the cell from patient tissue is suspected of being a cancer cell.

52. The method of claim 51, wherein the patient tissue is breast.

53. The method of claim 50, wherein the inhibitor of mitosis is an agent adapted to inhibit a gene selected in claim 50 from Table 4.

54. The method of claim 50, wherein the inhibitor of mitosis is an agent selected from GSK461364, GSK1070916, and GSK929325.

55. The method of claim 50, wherein the remaining mitotic network genes of the signature are selected from the group consisting of: PLK1, SMC4, PBK, KIF14, NCAPD2, RRM2, CENPA, CENPE, CENPN, KNTC2, KIF23, RFC3, EXO1, LMNB2, TEX10, DEPDC1, DDX39, MAD2L1, MAD2L1BP, C10orf13, FAM64A, TPX2, AURKA, and TTK.

56. The method of claim 50, wherein the signature comprising 18 mitotic network genes, said signature comprising HJURP, AURKA, AURKB, BUB1, CENPE, CHEK1, FOXM1, PBK, PLK1, MELK, TTK, TPX2, TYMS, KIF23, KIF20, KIF2C, EXOSC9, PTTG1 and PRC1.

57. The method of claim 50, the signature comprising mitotic network genes comprising genes normally associated with prophase.

58. The method of claim 50, measuring all the signature comprising 54 mitotic network genes of Table 4.

59. The method of claim 50, wherein the signature informs cancer cell subtype determination.

60. The method of claim 55, wherein the gene CENPA is selected for the signature.
